SEQUENCE LISTING (73 sequences)

SEQ ID NO: 1 - skipped

SEQ ID NO: 2 - DNA, 10 bp, synthetic construct
  source 1..10: mol_type = other DNA; note = CpG oligo
aacgttcgag

SEQ ID NO: 3 - skipped

SEQ ID NO: 4 - DNA, 22 bp, synthetic construct
  source 1..22: mol_type = other DNA; note = CpG oligo
tgactgtgaacgttcgagatga

SEQ ID NO: 5 - protein, 85 aa, synthetic construct
  source 1..85: note = Tetramerization domain from SEPPALLATA-like MADS domain transcription factor from Arabidopsis thaliana (SMDTF)
VELSSQQEYLKLKERYDALQRTQRNLLGEDLGPLSTKELESLERQLDSSLKQIRALRTQFMLDQSKERMLTETNKTLRLRLADGY

SEQ ID NO: 6 - protein, 113 aa, synthetic construct
  source 1..113: note = Sendai virus phosphoprotein tetramerization domain (SPP)
ENTSSMKEMATLLTSLGVIQSAQEFESSRDASYVFARRALKSANYAEMTFNVCGLILSAEKSSARKVDENKQLLKIQESVESFRDIYKRFSEYQKEQNSLLMSNLSTLHIITD

SEQ ID NO: 7 - protein, 24 aa, synthetic construct
  source 1..24: note = PiLZ structure from Xanthomonas campestris
LLVQRMDAKLDLILALIGRLVRQS

SEQ ID NO: 8 - protein, 74 aa, synthetic construct
  source 1..74: note = Measles virus phosphoprotein tetramerization domain (MPP)
GDHYDDELFSDVQDIKTALAKIHEDNQKIISKLESLLLLKGEVESIKKQINRQNISISTLEGHLSSIMIAIPGL

SEQ ID NO: 9 - protein, 51 aa, synthetic construct
  source 1..51: note = Dictyocaulus viviparus ACE tetramerization domain
AVADVGDPFLLWKQQMDKWQNEYITDWQYHFEQYKKYQTYRHLDSDSCSGS

SEQ ID NO: 10 - protein, 454 aa, Influenza A virus
  source 1..454: note = A/PR/8/34 (N1_PR8)
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 11 - protein, 469 aa, Influenza A virus
  source 1..469: note = A/Michigan/45/15 (N1-Mich15)
MNPNQKIITIGSICMTIGMANLILQIGNIISIWVSHSIQIGNQSQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 12 - protein, 453 aa, Influenza A virus
  source 1..453: note = A/WSN/33 (N1_WSN)
MNPNQKIITIGSICMVVGIISLILQIGNIISIWISHSIQTGNQNHTGICNQGSITYKVVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGMGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 13 - protein, 469 aa, Influenza A virus
  source 1..469: note = A/Hong Kong/4801/14 (N2_HK14)
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNXVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 14 - protein, 469 aa, Influenza A virus
  source 1..469: note = A/chicken/Bangladesh/19870/13 (N2_ck)
MNPNQKIIALGSASLTIAIVCLLIQIAILATTMTLHFTQSEYTNSSTNKVVSCESTIIERNITEIVHLNGTIIERESCPKSAEYKNWSKPQCQITGFAPFSKDNSIRLSAGGDIWVTREPYVSCSLNKCYQFALGQGTTLNNRHSNGTTHDRSPYRTLLMSELGVPFHLGTKQVCIAWSSSSCHDGRAWLHVCVTGDDKNATASVIYDGMLTDSIVSWSKNILRTQESECVCINGTCTVVMTDGSASGMADTRILFIREGKIVHISPLSGSAQHVEECSCYPRYPEVRCVCRDNWKGSNRPVLYINMADYNIDSNYVCSGLVGDTPRSDDSSSSSNCRDPNNERGGPGVKGWAFDXGDDVWMGRTIKKDSRAGYETFRVVDGWTVANSKSQTNRQVIVESDNRSGYSGIFSVEGKTCINRCFYVELIRGRPQETRVWWTSNSIIVFCGTSGTYGTGSWPDGANINFMSI

SEQ ID NO: 15 - protein, 469 aa, Influenza A virus
  source 1..469: note = A/swine/Missouri/4296424/06A (N3_sw)
MNPNQKIITIGVVNTTLSTIALLIGVGNLIFNTVIHEKIGDHQIVTYPTITTPAVPNCSDTIITYNNTVINNITTTIITEEERPFKSPLPLCPFRGFFPFHKDNAIRLGENKDVIVTREPYVSCDNDNCWSFALTQGALLGTKHSNGTIKDRTPYRSLIRFPIGTAPVLGNYKEICIAWSSSSCFDGKEWMHVCMTGNDNDASAQIIYGGRMTDSIKSWRKDILRTQESECQCIDGTCVVAVTDGPAANSADYRVYWIREGKIIKYENVPKTKIQHLEECSCYVDIDVYCICRDNWKGSNRPWMRINNETILETGYVCSKFHSDTPRPADPSTMSCDSPSNVNGGPGVKGFGFKAGDDVWLGRTVSTSGRSGFEIIKVTEGWINSPNHVKSITQTLVSNNDWSGYSGSFIVKAKDCFQPCFYVELIRGRPNKNDDVSWTSNSIVTFCGLDNEPGSGNWPDGSNIGFMPK

SEQ ID NO: 16 - protein, 469 aa, Influenza A virus
  source 1..469: note = A/blue-winged teal/Guatemala/CIP049H113-74/13 (N3_bwt)
MNPNQKIITIGVVNTTLSTIALLIGVGNLVFNTVIHEKIGDHQTVTHPTITTPAIPNCSDTIITYNNTVINNITTTIITEAERPFKSPLPLCPFRGFFPFHKDNAIRLGENKGVIVTREPYISCDNDNCWSFALAQGALLGTKHSNGTIKDRTPYRSLIRFPIGTAPVLGNYKEICIAWSSSSCFDGKEWMHVCMTGNDNDASAQIIYAGRMTDSIKSWRKDILRTQESECQCIDGTCVVAVTDGPAANSADHRVYWIREGKIIKYEDVPKTKIQHLEECSCYVDIDVYCICRDNWKGSNRPWMRINNETILETGYVCSKFHSDTPRPADPSTMSCDSPSNVNGGPGVKGFGFKAGNDVWLGRTVSTSGRSGFEIIKVTEGWINSPNHAKSITQTLVSNNDWSGYSGSFIVKTKDCFQPCFYVELIRGRPNKNDDVSWTSNSIVTFCGLDNEPGSGNWPDGSNIGFMPK

SEQ ID NO: 17 - protein, 469 aa, Influenza A virus
  source 1..469: note = A/duck/Jiangxi/22676/13 (N3_duck)
MNPNQKIITIGVVNTTLSTIALLIGIGNLIFNTVIHEKIGVHQTVVYPTITPPVIPNCSDTTITYNNTVVNNITATIINKAEKQFKPSLPLCPFRGFFPFHKDNAIRLGENKDVIVTREPYVSCDNDDCWSFALAQGALLGTKHSNGTIKDRTPYRSLIRFPIGTAPVLGNYKEICVAWSSSSCFDGKEWMHVCMTGNDNDASGQIIYAGKMTDSIKSWRRDILRTQESECQCIDGTCIVAVTDGPAASSADHRIYWIRKGKIIKYEDIPKTKIQHLEECSCYVDTDVYCICRDNWKGSNRPWMRINNETILETGYVCSKFHSDTPRPADPSTVSCDSPSNINGGPGVKGFGFRAGNDVWLGRTVSTTGRSGFEVIKVTEGWINSLNHAKSITQTLVSNNDWSGYSGSFIVENNGCFQPCFYIELIRGRPNRNDDVSWTSNSIVTFCGLDNEPGSGNWPDGSNIGFMPK

SEQ ID NO: 18 - protein, 470 aa, Influenza A virus
  source 1..470: note = A/duck/Hokkaido/222/14 (N4_duck)
MNPNQKIITIGSVSIVLTTIGLLLQITSLCSIWFSHYNQVTQTSEQSCSNNTTNYYNETFVNVTNVQNNYTTITEPSSPQVIHYSSGRDLCPVKGWAPLSKDNGIRIGSRGEVFVIREPFISCSINECRTFFLTQGALLNDKHSNGTVKDRSPFRTLMSCPIGVAPSPSNSRFESVAWSATACSDGPGWLTLGITGPDSTAVAVIKYNGIITDTLKSWKGNIMRTQESECVCQDEFCYTLITDGPSDAQAFYKILKIRKGKIMSVKDVDATGFHFEECSCYPSGENVECVCRDNWRGSNRPWIRFNSDLDYQIGYVCSGVFGDNPRPVDGTGSCSGPINNGKGRYGVKGFSFRYGDGVWIGRTKSLESRSGFEMVWDANGWVSTDKDSNGVQDIIDNDNWSGYSGSFSIRGETTGKNCTVPCFWVEMIRGQPKEKTIWTSGSSIAFCGVDSDTTGWSWPDGALLPFDIDK

SEQ ID NO: 19 - protein, 470 aa, Influenza A virus
  source 1..470: note = A/ruddy turnstone/Delaware/AI03-378/03 (N4_rt)
MNPNQKIITIGSVSIILTTIGLLLQITSLCSIWFSHYNQVTQTHEQPCSNNTTNYYNETFVNVTNVQNNYTTVTEPSTPDVVHYSSGRDLCPIRGWAPLSKDNGIRIGSRGEVFVIREPFISCSISECRTFFLTQGALLNDKHSNGTVKDRSPFRTLMSCPIGVAPSPSNSRFESVAWSATACSDGPGWLTLGITGPDTTAVAVLKYNGIITDTFKSWKGNIMRTQESECVCQDEFCYTLITDGPSDAQAFYKILKIRKGKIVSMKDVDATGFHFEECSCYPSRTDIECVCRDNWRGSNRPWIRFNSDLDYQIGYVCSGIFGDNPRPVDGTGSCNSPVNNGKGRYGVKGFSFRYGDGVWIGRTKSLEFRSGFEMVWDANGWVSTDKDSNGVQDIIDNDNWSGYSGSFSIRGEKTGRNCTVPCFWVEMIRGQPKEKTIWTSGSSIAFCGVNSDTTGWSWPDGALLPFDIDK

SEQ ID NO: 20 - protein, 473 aa, Influenza A virus
  source 1..473: note = A/American wigeon/California/HS007B/15 (N5_aw)
MNPNQKIITIGSVSLALVVFNILLHIASIVIGIISVTKEISVSSTCNTTEVYNETVRLETITIPINNTVYIERESHQEPEFLNNTEPLCNVSGFAIVSKDNGIRIGSRGHVFVIREPFVACGPTECRTFFLTQGALLNDKHSNNTVKDRSPYRALMSVPLGSSPNAYQAKFESVAWSATACHDGKGWLAVGISGADDDAYAVIHYGGMPTDVVRSWRKQILRTQESSCVCMTGNCYWVMTDGPANSQASYKIFKSHRGMVTNEREVSFQGGHIEECSCYPNLGKVECVCRDNWNGMNRPVLTFDEDLNYEVGYLCAGIPTDTPRVQDNSFTGSCTNAVGGSGTNNYGVKGFGFRQGNSVWAGRTVSISSRSGFEILLIEDGWIKTSKNVVKKVEVLNNKNWSGYSGAFTIPITMTSKQCLVPCFWLEMIRGKPEERTSIWTSSSSTVFCGVSSEVPGWSWDDGAILPFDIDKM

SEQ ID NO: 21 - protein, 472 aa, Influenza A virus
  source 1..472: note = A/migratory duck/Jiangxi/6847/03 (N5_md)
MNPNQKIITIGSISLGLVVFNILLHVASIVLGIISVTKDHGAYTCNTTEVYNETVRVETVTIPVNNTIYIERELTHEPEFLNNTEPLCEVSGFAIVSKDNGIRIGSRGHVFVIREPFVACGPSECRTFFLTQGALLNDKHSNNTVKDRSPYRALMSVPLGSSPNAYQAKFESVGWSATACHDGKEWMAIGVSGADDDAYAVIHYGGIPTDVVRSWRKQILRTQESSCVCMKGECYWVMTDGPANNQASYKIFKSQKGLVVDEKEISFQGGHIEECSCYPNMGKVECVCRDNWNGMNRPILTFDENLEYEVGYLCAGIPTDTPRVQDSSFTGSCTNAVGGSGTNNYGVKGFGFRQGTSVWAGRTISISSRSGFEVLLIKDGWIRPSKTISKKVEVLDNKNWSGYSGSFTIPTAMTSKSCLVPCFWLEMIRGKPEERTSIWTSSSSTVFCGVSSEVPGWSWDDGAILPFDIDKM

SEQ ID NO: 22 - protein, 459 aa, Influenza A virus
  source 1..459: note = A/Shenzhen/1/16 (N6_Sz)
MNPNQKITCISATGVTLSVVSLLIGIANLGLNIGLHYKVSDSTTINIPNMNETNSTTTNITNIIVNKNEERTFLNLTKPLCEVNSWHILSKDNAIRIGEDAHILVTREPYLSCDPQGCRMFALSQGTTLRGRHANGTIHDRGPFRALISWEMGQAPSPYNTRVECIGWSSTSCHDGISRMSICISGPNDNASAVVWYRGRPVTEIPSWVGNILRTQESECVCHKGICPVVMTDGPANNKAATKIIYFKEGKIQKIEELQGNAQHIEECSCYGAAGMIKCVCRDNWKGANRPIITIDPEMMTHTSKYLCSKILTDTSRPNDPTNGNCDAPITGGSPDPGVKGFAFLDGENSWLGRTISKDSRSGYEMLKVPNAETDTQSGPTSYQLIVNNQNWSGYSGAFIDYWANKGCFNPCFYVELIRGRPKEIDVLWTSSSMVALCGSRERLGSWSWHDGAEIIYFK

SEQ ID NO: 23 - protein, 470 aa, Influenza A virus
  source 1..470: note = A/Caspian seal/Russia/T1/12 (N6_cs)
MNPNQKIICISATGMTLSVVSLLIGIANLGLNIGLHYKVGDTPDVNIPSMNETNSTTTIINNNTQNNFTNITNIIVNKEEGRTFLNLTKPLCEVNSWHILSKDNAIRIGEDAHILVTREPYLSCDPQGCRMFALSQGTTLRGRHANGTIHDRSPFRALVSWEMGQAPSPYNVRVECIGWSSTSCHDGISRMSICMSGPNNNASAVVWYGGRPVTEIPSWAGNILRTQESECVCHKGICPVVMTDGPANNRAATKIIYFKEGKIQKIEELEGNAQHIEECSCYGAAGVIKCICRDNWKGANRPVIIIDPEMMTHTSKYLCSRVLTDTSRPNDPTSGNCDAPITGGSPDPGVKGFAFLDGENSWLGRTISKDSRSGYEMLKVPNAETDTQSGPTSHQVIVNNQNWSGYSGAFIDYWANKECFNPCFYVELIRGRPKESSVLWTSNSIVALCGSKERLGSWSWHDGAEIIYFK

SEQ ID NO: 24 - protein, 471 aa, Influenza A virus
  source 1..471: note = A/chicken/Netherlands/1/03 (N7_cN)
MNPNQKLFALSGVAIALSVLNLLIGISNVGLNVSLHLKEKGPKQEENLTCTTINQNNTTVVENTYVNNTTIITKGTDLKTPSYLLLNKSLCNVEGWVVIAKDNAVRFGESEQIIVTREPYVSCDPTGCKMYALHQGTTIRNKHSNGTIHDRTAFRGLISTPLGTPPTVSNSDFMCVGWSSTTCHDGIARMTICIQGNNDNATATVYYNRRLTTTIKTWARNILRTQESECVCHNGTCAVVMTDGSASSQAYTKVMYFHKGLVVKEEELRGSARHIEECSCYGHNQKVTCVCRDNWQGANRPIIEIDMNTLEHTSRYVCTGILTDTSRPGDKSSGDCSNPITGSPGAPGVKGFGFLNGDNTWLGRTISPRSRSGFEMLKIPNAGTDPNSRIAERQEIVDNNNWSGYSGSFIDYWNDNSECYNPCFYVELIRGRPEEAKYVWWTSNSLIALCGSPFPVGPGSFPDGAQIQYFS

SEQ ID NO: 25 - protein, 471 aa, Influenza A virus
  source 1..471: note = A/chicken/Germany/R28/03 (N7_cG)
MNPNQKLFALSGVAIALSVLNLLIGISNVGLNVSLHLKEKGPKQEENLTCTTINQNNTTVVENTYVNNTTIITKGTDLKTPSYLLLNKSLCNVEGWVVIAKDNAVRFGESEQIIVTREPYVSCDPTGCKMYALHQGTTIRNKHSNGTIHDRTAFRGLISTPLGTPPTVSNSDFMCVGWSSTTCHDGIARMTICIQGNNDNATATVYYNRRLTTTIKTWARNILRTQESECVCHNGTCAVVMTDGSASSQAYTKVMYFHKGLVVKEEELRGSARHIEECSCYGHNQKVTCVCRDNWQGANRPIIEIDMNTLEHTSRYVCTGILTDTSRPGDKSSGDCSNPITGSPGAPGVKGFGFLNGDNTWLGRTISPRSRSGFEMLKIPNAGTDPNSRIAERQEIVDNNNWSGYSGSFIDYWNDNSECYNPCFYVELIRGRPEEAKYVWWASNSLIALCGSPFPVGSGSFPDGAQIQYFS

SEQ ID NO: 26 - protein, 470 aa, Influenza A virus
  source 1..470: note = A/chicken/Netherlands/14015531/14 (N8_cN)
MNPNQKIVTIGSISLGLVVFNVLLHAVSIILTVLALGKSENNGICNGTVVREYNETVRIEKVTQWYNTSVVEYVPHWNEGTYINNTEPICDVKGFAPFSKDNGIRVGSRGHIFVIREPFVSCSPVECRTFFLTQGSLLNDKHSNGTVKDRSPFRTLMSVEVGQSPNVYQARFEAVAWSATACHDGKKWMTIGVTGPDSKAVAVVHYGGVPTDVVNSWAGDILRTQESSCTCIQGNCYWVMTDGPANRQAQYRIYKANQGKIIGRTDVSFSGGHIEECSCYPNDGKVECVCRDNWTGTNRPVLIISPDLSYRVGYLCAGLPSDTPRGEDTQFAGSCTSPMGNQGYGVKGFGFRQGTDVWVGRTISRTSRSGFEIIRIKNGWTQTSKEQIRRQVVVDNSNWSGYSGSFTLPVELSGRECLVPCFWVEMIRGRPEERTIWTSSSSIVMCGVDYEIADWSWHDGAILPFDIDKT

SEQ ID NO: 27 - protein, 470 aa, Influenza A virus
  source 1..470: note = A/chicken/Laos/A0573/07 (N8_cL)
MNPNQKIITIGSVSLGLVVLNILLHIVSITVTVLVLPRNGNNGSCNETVIREYNETVRVEKVTQWHNTNVIEYIERPENDHFMNNTEALCDAKGFAPFSKDNGIRIGSRGHVFVIREPFVSCSPTECRTFFLTQGSLLNDKHSNGTVKDRSPYRTLMSVEIGQSPNVYQARFEAVAWSATACHDGKKWMTIGVTGPDAKAVAVVHYGGIPTDVINSWAGDILRTQESSCTCIQGECFWVMTDGPANRQAQYRAFKAKQGKIVGQAEISFNGGHIEECSCYPNEGKVECVCRDNWTGTNRPVLVISPDLSYRVGYLCAGIPSDTPRGEDSQFTGSCTSPMGNQGYGVKGFGFRQGSDVWMGRTISRTSRSGFEILKVRNGWVQNSKEQIKRQVVVDNLNWSGYSGSFTLPVELTKRNCLVPCFWVEMIRGKPEEKTIWTSSSSIVMCGVDHEIADWSWHDGAILPFDIDKM

SEQ ID NO: 28 - protein, 465 aa, Influenza A virus
  source 1..465: note = A/Anhui/2/13 (N9_AH13)
MNPNQKILCTSATAIIIGAIAVLIGIANLGLNIGLHLKPGCNCSHSQPETTNTSQTIINNYYNETNITNIQMEERTSRNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

SEQ ID NO: 29 - protein, 465 aa, Influenza A virus
  source 1..465: note = A/chicken/Dongguan/1143/14 (N9_ck)
MNPNQKILCTSATAIIIGAIAVLIGIANLGLNIGLHLKPGCNCSHSQPETTNTSQTIINNYYNETNITNIQMEERTSKNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAKGDCYRACFYVELIRGRPKEDKVWWTSNSIVSMCSSTEFLGQWNWPDGAKIEYFL

SEQ ID NO: 30 - protein, 466 aa, Influenza B virus
  source 1..466: note = B/Malaysia/2506/04 (B_Mal)
MLPSTIQTLTLFLTSGGVLLSLYVSASLSYLPYSDILLKFPSTEITAPTMPLDCANASNVQAVNRSATKGVTLLLPEPEWTYPRLSCPGSTFQKALLISPHRFGETKGNSAPLIIREPFIACGPKECKHFALTHYAAQPGGYYNGTRGDRNKLRHLISVKLGKIPTVENSIFHMAAWSGSACHDGKEWTYIGVDGPDNNALLKIKYGEAYTDTYHSYANNILRTQESACNCIGGNCYLMITDGSASGVSECRFLKIREGRIIKEIFPTGRIKHTEECTCGFASNKTIECACRDNSYTAKRPFVKLNVETDTAEIRLMCTETYLDTPRPDDGSITGPCESNGDKGSGGIKGGFVHQRMASKIGRWYSRTMSKTKRMGMGLYVKYDGDPWADSDALAFSGVMVSMEEPGWYSFGFEIKDKKCDVPCIGIEMVHDGGKETWHSAATAIYCLMGSGQLLWDTVTGVNMAL

SEQ ID NO: 31 - protein, 388 aa, synthetic construct
  source 1..388: note = Globular head domain of NA of influenza virus A/Michigan/45/15
SVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 32 - protein, 393 aa, synthetic construct
  source 1..393: note = Globular head domain of NA of influenza virus A/Kansas/14/2017
ICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 33 - protein, 394 aa, synthetic construct
  source 1..394: note = Globular head domain of NA of influenza virus B/Colorado/06/2017
LLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 34 - protein, 41 aa, synthetic construct
  source 1..41: note = Signal peptide
MLLVNQSHQGFNKEHTSKMVSAIVLYVLLAAAAHSAFAADP

SEQ ID NO: 35 - protein, 6 aa, synthetic construct
  source 1..6: note = His tag
HHHHHH

SEQ ID NO: 36 - protein, 7 aa, synthetic construct
  source 1..7: note = Cleavage site
LVPRGSP

SEQ ID NO: 37 - protein, 10 aa, synthetic construct
  source 1..10: note = Cleavage site
SLVPRGSPSR

SEQ ID NO: 38 - protein, 7 aa, synthetic construct
  source 1..7: note = Cleavage site
  VARIANT 7: note = X is G or S
ENLYFQX

SEQ ID NO: 39 - skipped

SEQ ID NO: 40 - protein, 503 aa, synthetic construct
  source 1..503: note = N1-MPP (with signal peptide; no 6xHis; no cleavage, no spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 41 - skipped

SEQ ID NO: 42 - protein, 506 aa, synthetic construct
  source 1..506: note = N1-MPP (with signal peptide; no 6xHis; no cleavage, with spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLGGGSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 43 - DNA, 1521 bp, synthetic construct
  source 1..1521: mol_type = other DNA; note = N1-MPP (with signal peptide; no 6xHis; no cleavage, with spacer)
atgctgctcgtcaaccaatcccaccagggcttcaacaaggaacacacttctaagatggtctccgctatcgtgctctacgtgctgctcgctgccgctgcccactcagctttcgctgccgacccaggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgggcggcggctccgtgaaattagcgggcaattcctctctctgccctgttagtggatgggctatatacagtaaagacaacagtgtaagaatcggttccaagggggatgtgtttgtcataagggaaccattcatatcatgctctcccttggaatgcagaaccttcttcttgactcaaggggccttgctaaatgacaaacattccaatggaaccattaaagacaggagcccataccgaaccctaatgagctgtcctattggtgaagttccctctccatacaactcaagatttgagtcagtcgcttggtcagcaagtgcttgtcatgatggcatcaattggctaacaattggaatttctggcccagacagtggggcagtggctgtgttaaagtacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtgtgcagggataactggcatggctcgaatcgaccgtgggtgtctttcaaccagaatctggaatatcagatgggatacatatgcagtggggttttcggagacaatccacgccctaatgataagacaggcagttgtggtccagtatcgtctaatggagcaaatggagtaaaaggattttcattcaaatacggcaatggtgtttggatagggagaactaaaagcattagttcaagaaaaggttttgagatgatttgggatccgaatggatggactgggactgacaataaattctcaataaagcaagatatcgtaggaataaatgagtggtcagggtatagcgggagttttgttcagcatccagaactaacagggctggattgtataagaccttgcttctgggttgaactaataagagggcgacccgaagagaacacaatctggactagcgggagcagcatatccttttgtggtgtaaacagtgacactgtgggttggtcttggccagacggtgctgagttgccatttaccattgacaagtaa

SEQ ID NO: 44 - protein, 513 aa, synthetic construct
  source 1..513: note = N1-MPP (with signal peptide; no 6xHis; with cleavage, no spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 45 - skipped

SEQ ID NO: 46 - protein, 512 aa, synthetic construct
  source 1..512: note = N1-MPP (with signal peptide; with 6xHis; no cleavage, with spacer)
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLGGGSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 47 - DNA, 1539 bp, synthetic construct
  source 1..1539: mol_type = other DNA; note = N1-MPP (with signal peptide; with 6xHis; no cleavage, with spacer)
atgctgctcgtcaaccaatcccaccagggcttcaacaaggaacacacttctaagatggtctccgctatcgtgctctacgtgctgctcgctgccgctgcccactcagctttcgctgccgacccacaccaccaccaccaccacggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgggcggcggctccgtgaaattagcgggcaattcctctctctgccctgttagtggatgggctatatacagtaaagacaacagtgtaagaatcggttccaagggggatgtgtttgtcataagggaaccattcatatcatgctctcccttggaatgcagaaccttcttcttgactcaaggggccttgctaaatgacaaacattccaatggaaccattaaagacaggagcccataccgaaccctaatgagctgtcctattggtgaagttccctctccatacaactcaagatttgagtcagtcgcttggtcagcaagtgcttgtcatgatggcatcaattggctaacaattggaatttctggcccagacagtggggcagtggctgtgttaaagtacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtgtgcagggataactggcatggctcgaatcgaccgtgggtgtctttcaaccagaatctggaatatcagatgggatacatatgcagtggggttttcggagacaatccacgccctaatgataagacaggcagttgtggtccagtatcgtctaatggagcaaatggagtaaaaggattttcattcaaatacggcaatggtgtttggatagggagaactaaaagcattagttcaagaaaaggttttgagatgatttgggatccgaatggatggactgggactgacaataaattctcaataaagcaagatatcgtaggaataaatgagtggtcagggtatagcgggagttttgttcagcatccagaactaacagggctggattgtataagaccttgcttctgggttgaactaataagagggcgacccgaagagaacacaatctggactagcgggagcagcatatccttttgtggtgtaaacagtgacactgtgggttggtcttggccagacggtgctgagttgccatttaccattgacaagtaa

SEQ ID NO: 48 - protein, 519 aa, synthetic construct
  source 1..519: note = N1-MPP (with signal peptide; with 6xHis; with cleavage, no spacer
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 49 - skipped

SEQ ID NO: 50 - protein, 508 aa, synthetic construct
  source 1..508: note = N2-MPP (with signal peptide; no 6xHis; no cleavage, no spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 51 - skipped

SEQ ID NO: 52 - protein, 517 aa, synthetic construct
  source 1..517: note = N2-MPP (with signal peptide; no 6xHis; no cleavage, with spacer)
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLGGGICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 53 - skipped

SEQ ID NO: 54 - protein, 518 aa, synthetic construct
  source 1..518: note = N2-MPP (with signal peptide; no 6xHis; with cleavage, no spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLSLVPRGSPSRICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 55 - skipped

SEQ ID NO: 56 - protein, 517 aa, synthetic construct; identical to SEQ ID NO: 52
  source 1..517: note = N2-MPP (with signal peptide; with 6xHis; no cleavage, with spacer)

SEQ ID NO: 57 - skipped

SEQ ID NO: 58 - protein, 524 aa, synthetic construct
  source 1..524: note = N2-MPP (with signal peptide; with 6xHis; with cleavage, no spacer)
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLSLVPRGSPSRICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 59 - DNA, 1575 bp, synthetic construct
  source 1..1575: mol_type = other DNA; note = N2-MPP (with signal peptide; with 6xHis; with cleavage, no spacer)
atgctgctcgtcaaccaatcccaccagggcttcaacaaggaacacacttctaagatggtctccgctatcgtgctctacgtgctgctcgctgccgctgcccactcagctttcgctgccgacccacaccaccaccaccaccacggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgtctctcgtgcccaggggatcaccttctagaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcctgacaagtgttatcaatttgcccttggacagggaacaacaataaacaacgtgcattcaaataacacagcacgtgataggacccctcatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagtttcatttacaatgggaggcttgtagatagtgttgtttcatggtccaaagatattctcaggacccaggagtcagaatgcgtttgtatcaatggaacttgtacagtagtaatgactgatggaaatgctacaggaaaagctgatactaaaatattattcattgaggaggggaaaatcgttcatactagcaaattgtcaggaagtgctcagcatgtcgaagagtgctcttgctatcctcgataccctggtgtcagatgtgtctgcagagacaactggaaaggatccaaccggcccatcgtagatataaacataaaggatcatagcattgtttccagttatgtgtgttcaggacttgttggagacacacccagaaaaaccgacagctccagcagcagccattgcttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatatataa

SEQ ID NO: 60 - protein, 509 aa, synthetic construct
  source 1..509: note = B-NA-MPP (with signal peptide; no 6xHis; no cleavage, no spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 61 - skipped

SEQ ID NO: 62 - protein, 512 aa, synthetic construct
  source 1..512: note = B-NA -MPP (with signal peptide; no 6xHis; no cleavage, with spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLGGGLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 63 - skipped

SEQ ID NO: 64 - protein, 519 aa, synthetic construct
  source 1..519: note = B-NA -MPP (with signal peptide; no 6xHis; with cleavage, no spacer)
MLLVNQSHQGFNKEHTSKMVSAIVLYVLLAAAAHSAFAADPGDHYDDELFSDVQDIKTALAKIHEDNQKIISKLESLLLLKGEVESIKKQINRQNISISTLEGHLSSIMIAIPGLSLVPRGSPSRLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 65 - skipped

SEQ ID NO: 66 - protein, 518 aa, synthetic construct
  source 1..518: note = B-NA-MPP (with signal peptide; with 6xHis; no cleavage, with spacer)
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLGGGLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 67 - skipped

SEQ ID NO: 68 - protein, 525 aa, synthetic construct
  source 1..525: note = B-NA-MPP (with signal peptide; with 6xHis; with cleavage, no spacer)
MLLVNQSHQGFNKEHTSKMVSAIVLYVLLAAAAHSAFAADPHHHHHHGDHYDDELFSDVQDIKTALAKIHEDNQKIISKLESLLLLKGEVESIKKQINRQNISISTLEGHLSSIMIAIPGLSLVPRGSPSRLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 69 - DNA, 1578 bp, synthetic construct
  source 1..1578: mol_type = other DNA; note = B-NA-MPP (with signal peptide; with 6xHis; with cleavage, no spacer)
atgctgctcgtcaaccaatcccaccagggcttcaacaaggaacacacttctaagatggtctccgctatcgtgctctacgtgctgctcgctgccgctgcccactcagctttcgctgccgacccacaccaccaccaccaccacggcgatcactacgacgacgaactgttctccgacgtgcaggacatcaagaccgctctggctaagatccacgaggacaaccagaagatcatctccaagctggaatccctgctgctgctgaagggcgaagtcgagtccatcaagaagcagatcaaccgccagaacatctccatctccaccttggagggtcacctgtcctccatcatgatcgctatccctggcctgtctctcgtgcccaggggatcaccttctagacttcttctcccagaaccggagtggacatacccgcgtttatcttgcccgggctcaacctttcagaaagcactcctaattagccctcatagattcggagaaaccaaaggaaactcagctcccttgataataagggaaccttttgttgcttgtggaccaaatgaatgcaaacactttgctttaacccattatgcagcccaaccagggggatactacaatggaacaagaggagacagaaacaagctgaggcatctaatttcagtcaaattgggcaaaatcccaacagtagagaactccattttccacatggcagcatggagcgggtccgcgtgccatgatggtaaggaatggacatatatcggagttgatggccctgacaataatgcattgctcaaagtaaaatatggagaagcatatactgacacataccattcctatgcaaacaacatcctaagaacacaagaaagtgcctgcaattgcatcgggggaaattgttatctaatgataactgatggctcagcttcaggtgttagtgaatgcagatttcttaagattcgagagggccgaataataaaagaaatatttccaacaggaagagtaaaacacactgaggaatgcacatgcggatttgccagcaataaaaccatagaatgtgcctgtagagacaacaggtacacagcaaaaagaccttttgtcaaattaaacgtggagactgatacagcagaaataaggttgatgtgcacagatacctatttggacacccccagaccaaatgatggaagcataacaggcccttgtgaatctgatggggacaaagggagtggaggcatcaagggaggatttgttcatcaaagaatgaaatccaagattggaaggtggtactctcgaacgatgtctcaaactgaaaggatggggatgggactgtatgtcaagtatggtggagacccatgggctgacagtgatgccctagcttttagtggagtaatggtttcaatgaaagaacctggttggtattcctttggcttcgaaataaaagataagaaatgcgatgtcccctgtattgggatagagatggtacatgatggtggaaaagagacttggcactcagcagcaacagccatttactgtttaatgggctcaggacagctgctgtgggacactgtcacaggtgttgacatggctctgtaa

SEQ ID NO: 70 - protein, 530 aa, synthetic construct
  source 1..530: note = SMDTF-N1 (with signal peptide; with 6xHis; with cleavage, no spacer)
MLLVNQSHQGFNKEHTSKMVSAIVLYVLLAAAAHSAFAADPHHHHHHVELSSQQEYLKLKERYDALQRTQRNLLGEDLGPLSTKELESLERQLDSSLKQIRALRTQFMLDQSKERMLTETNKTLRLRLADGYSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 71 - protein, 558 aa, synthetic construct
  source 1..558: note = SPP-N1 (with signal peptide; with 6xHis; with cleavage, no spacer)
MLLVNQSHQGFNKEHTSKMVSAIVLYVLLAAAAHSAFAADPHHHHHHENTSSMKEMATLLTSLGVIQSAQEFESSRDASYVFARRALKSANYAEMTFNVCGLILSAEKSSARKVDENKQLLKIQESVESFRDIYKRFSEYQKEQNSLLMSNLSTLHIITDSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 72 - protein, 469 aa, synthetic construct
  source 1..469: note = PiLZ-N1 (with signal peptide; with 6xHis; with cleavage, no spacer)
MLLVNQSHQGFNKEHTSKMVSAIVLYVLLAAAAHSAFAADPHHHHHHLLVQRMDAKLDLILALIGRLVRQSSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 73 - protein, 496 aa, synthetic construct
  source 1..496: note = ACE-N1 (with signal peptide; with 6xHis; with cleavage, no spacer)
MLLVNQSHQGFNKEHTSKMVSAIVLYVLLAAAAHSAFAADPHHHHHHAVADVGDPFLLWKQQMDKWQNEYITDWQYHFEQYKKYQTYRHLDSDSCSGSSLVPRGSPSRSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

(19) United States
(12) Patent Application Publication
(10) Pub. No.: US 2025/0161430 A1
(43) Pub. Date: May 22, 2025

(54) IMMUNOGENIC COMPOSITIONS COMPRISING A RECOMBINANT NEURAMINIDASE AND CpG OLIGONUCLEOTIDE ADJUVANT, AND USES THEREOF

(71) Applicants: ICAHN SCHOOL OF MEDICINE AT MOUNT SINAI, New York, NY (US); DYNAVAX TECHNOLOGIES CORPORATION, Emeryville, CA (US)

(72) Inventors: Florian KRAMMER, New York, NY (US); Peter PALESE, New York, NY (US); Adolfo GARCIA-SASTRE, New York, NY (US); John D. CAMPBELL, San Francisco, CA (US); Paula TRAQUINA, San Ramon, CA (US); Robert L. COFFMAN, Portola Valley, CA (US)

(73) Assignees: ICAHN SCHOOL OF MEDICINE AT MOUNT SINAI, New York, NY (US); DYNAVAX TECHNOLOGIES CORPORATION, Emeryville, CA (US)

(21) Appl. No.: 18/838,947

(22) PCT Filed: Feb. 28, 2023
(86) PCT No.: PCT/US2023/014150

§ 371 Date: Aug. 15, 2024

Related U.S. Application Data
(60) Provisional application No. 63/315,437, filed on Mar. 1, 2022.

(51) Int. Cl. A61K 39/145; A61K 39/00; A61P 37/04

(52) U.S. Cl. CPC A61K 39/145; A61P 37/04; A61K 2039/55505; A61K 2039/55561

(57) ABSTRACT

In one aspect, provided herein are immunogenic compositions comprising CpG oligonucleotide adjuvant, and a recombinant neuraminidase, wherein the recombinant neuraminidase comprises a globular head domain of influenza virus neuraminidase and a tetramerization domain, and wherein the recombinant neuraminidase lacks influenza virus neuraminidase stalk, transmembrane and cytoplasmic domains. In another aspect, provided herein are methods of immunizing a subject against influenza virus using such immunogenic compositions. In another aspect, provided herein are methods of immunizing a subject against influenza virus using such immunogenic compositions.

### CROSS-REFERENCE TO RELATED APPLICATIONS

This application claims the benefit of U.S. Provisional Patent Application No. 63/315,437, filed Mar. 1, 2022, the disclosure of which is incorporated by reference herein in its entirety.

### STATEMENT REGARDING FEDERALLY SPONSORED RESEARCH

This invention was made with government support under award 75N93019C00051 awarded by the National Institutes of Health. The government has certain rights in this invention.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “06923-380-228_SEQ_LISTING.xml”, was created on Feb. 24, 2023 and is 94,520 bytes in size.

### 1. INTRODUCTION

In one aspect, provided herein are immunogenic compositions comprising a recombinant neuraminidase and a CpG oligonucleotide adjuvant (e.g., as described herein Section 5.2 or Example 1), wherein the recombinant neuraminidase comprises a globular head domain of influenza virus neuraminidase and a tetramerization domain, and wherein the recombinant neuraminidase lacks influenza virus neuraminidase stalk, transmembrane and cytoplasmic domains. In another aspect, provided herein are methods of immunizing a subject against influenza virus using such immunogenic compositions.

### 2. BACKGROUND

Influenza viruses are enveloped RNA viruses that belong to the family of Orthomyxoviridae [1]. Influenza A and B viruses are major human pathogens, causing a respiratory disease that ranges in severity from sub-clinical infection to primary viral pneumonia which can result in death. The clinical effects of infection vary with the virulence of the influenza strain and the exposure, history, age, and immune status of the host. The cumulative morbidity and mortality caused by seasonal influenza is substantial due to the relatively high attack rate.

According to the World Health Organization (WHO), seasonal influenza viruses can cause up to 5 million cases of severe influenza virus infection, including up to 650,000 deaths each year globally. Seasonal vaccination is currently the most effective intervention against influenza [1-7], yet overall vaccine effectiveness was only 36% in the recent 2017-2018 season [8]. However, current vaccination approaches rely on achieving a good match between circulating strains and the isolates included in the vaccine. Such a match is often difficult to attain due to a combination of factors. First, influenza viruses are constantly undergoing change: every 3-5 years the predominant strain of influenza A virus is replaced by a variant that has undergone sufficient antigenic drift to evade existing antibody responses. Isolates to be included in vaccine preparations must therefore be selected each year based on the intensive surveillance efforts of WHO collaborating centers. Second, to allow sufficient time for vaccine manufacture and distribution; strains must be selected approximately six months prior to the initiation of the influenza season. Often, the predictions of the vaccine strain selection committee are inaccurate, resulting in a substantial drop in the efficacy of vaccination.

The influenza virus contains two glycoproteins on its viral surface, which include the immunodominant hemagglutinin (HA) and the immunosubdominant neuraminidase (NA). Currently available seasonal influenza virus vaccines mostly target the HA, as the NA content in the vaccines is not standardized and can vary significantly. While the HA of influenza virus is known to induce a strong neutralizing antibody response in humans, it is more susceptible to antigenic drift. This can lead to a mismatch between vaccine strains and circulating strains, resulting in a significant loss of effectiveness of the annual vaccines. Over the past few years, the NA has emerged as an attractive target for vaccine development since it is less prone to undergo antigenic drift and therefore is antigenically more stable. In addition, it has been demonstrated in animal models as well as in humans that anti-NA immunity correlates with protection and reduces viral shedding [9-13].

The obstacles of using NA as a vaccine antigen include its lack of standardization in seasonal vaccine preparations as well as its fragile stability. Indeed, the amount of NA in seasonal vaccines usually varies in quality and quantity and it is likely that the structural integrity in current vaccine formulations is suboptimal. Additionally, it has been hypothesized that antigenic competition occurs between HA and NA in vaccine formulations, making the NA immunosubdominant [15]. The anti-NA immunity acquired after vaccination with live attenuated or inactivated vaccines is mediocre at best [16].

In general, the efficiency of a vaccine depends on the magnitude, duration and quality of the immune response that is induced. However, recombinant protein vaccines usually induce a lower immune response compared to whole virus vaccines, due to the lack of molecules that engage innate immune receptors. Thus, there is a need for improved recombinant NA immunogenic compositions for vaccination against influenza virus infections.

### 3. SUMMARY

In an aspect, provided herein is an immunogenic composition, comprising: (a) a recombinant neuraminidase described herein (see, e.g., Section 5.3); and (b) a CpG oligonucleotide adjuvant described herein (see, e.g., Section 5.2 or 6). In specific embodiments, the recombinant neuraminidase comprises an influenza virus neuraminidase globular head domain, and a paramyxovirus phosphoprotein tetramerization domain. In a specific embodiment, provided herein is an immunogenic composition, comprising: (a) a recombinant neuraminidase, wherein the recombinant neuraminidase (i) comprises an influenza virus neuraminidase globular head domain, and a paramyxovirus phosphoprotein tetramerization domain; and (b) a CpG oligonucleotide adjuvant described herein (see, e.g., Section 5.2 or 6), in an admixture with a pharmaceutically acceptable carrier. In some embodiments, the paramyxovirus phosphoprotein tetramerization domain comprises a measles virus phosphoprotein tetramerization domain. In a specific embodiment, the measles virus phosphoprotein tetramerization domain comprises the amino acid sequence of SEQ ID NO:8. In specific embodiments, the measles virus phosphoprotein tetramerization domain comprises an amino acid sequence having at least 80% identity to the amino acid sequence of SEQ ID NO:8. In some embodiments, the influenza virus neuraminidase globular head domain comprises an influenza A virus neuraminidase globular head domain. In specific embodiments, the influenza A virus neuraminidase is subtype N1 or subtype N2. In specific embodiments, the subtype N1 is influenza virus A/Michigan/45/2015. In specific embodiments, the subtype N2 is influenza virus A/Kansas/14/2017. In some embodiments, the influenza virus neuraminidase globular head domain comprises an influenza B virus neuraminidase globular head domain. In specific embodiments, the influenza B virus is influenza virus B/Colorado/6/2017. In specific embodiments, the influenza virus neuraminidase globular head domain comprises the amino acid sequence of SEQ ID NO:31, 32, or 33. In specific embodiments, the influenza virus neuraminidase globular head domain comprises an amino acid sequence having at least 80% identity to SEQ ID NO:31, 32, or 33. In some embodiments, the recombinant neuraminidase further comprises a cleavage site. In some embodiments, the cleavage site in the recombinant neuraminidase is between the tetramerization domain and influenza virus neuraminidase globular head domain. In a specific embodiment, the cleavage site comprises a thrombin cleavage site. In a specific embodiment, the cleavage site comprises the amino acid sequence of SEQ ID NO:36 or 37. In some embodiments, the recombinant neuraminidase further comprises a signal peptide. In a specific embodiment, the signal peptide comprises the amino acid sequence of SEQ ID NO:34. In some embodiments, the recombinant neuraminidase further comprises a purification tag. In a specific embodiment, the purification tag is a histidine tag or flag tag. In specific embodiments, the recombinant neuraminidase is enzymatically active as assessed by a technique described herein or known to one of skill in the art (e.g., an NA-Star assay).

In some embodiments, provided herein is an immunogenic composition, comprising: (a) a recombinant neuraminidase, wherein the recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain, and (b) a CpG oligonucleotide adjuvant (see, e.g., Section 5.2 or 6), in an admixture with a pharmaceutically acceptable carrier. In some embodiments, the paramyxovirus phosphoprotein tetramerization domain comprises a measles virus phosphoprotein tetramerization domain. In a specific embodiment, the measles virus phosphoprotein tetramerization domain comprises the amino acid sequence of SEQ ID NO:8. In specific embodiments, the measles virus phosphoprotein tetramerization domain comprises an amino acid sequence having at least 80% identity to the amino acid sequence of SEQ ID NO:8. In some embodiments, the influenza virus neuraminidase globular head domain comprises an influenza A virus neuraminidase globular head domain. In specific embodiments, the influenza A virus neuraminidase is subtype N1 or subtype N2. In specific embodiments, the subtype N1 is influenza virus A/Michigan/45/2015. In specific embodiments, the subtype N2 is influenza virus A/Kansas/14/2017. In some embodiments, the influenza virus neuraminidase globular head domain comprises an influenza B virus neuraminidase globular head domain. In specific embodiments, the influenza B virus is influenza virus B/Colorado/6/2017. In specific embodiments, the influenza virus neuraminidase globular head domain comprises the amino acid sequence of SEQ ID NO:31, 32, or 33. In specific embodiments, the influenza virus neuraminidase globular head domain comprises an amino acid sequence having at least 80% identity to SEQ ID NO:31, 32, or 33. In some embodiments, the recombinant neuraminidase further comprises a cleavage site. In some embodiments, the cleavage site in the recombinant neuraminidase is between the tetramerization domain and influenza virus neuraminidase globular head domain. In a specific embodiment, the cleavage site comprises a thrombin cleavage site. In a specific embodiment, the cleavage site comprises the amino acid sequence of SEQ ID NO:36 or 37. In some embodiments, the recombinant neuraminidase further comprises a signal peptide. In a specific embodiment, the signal peptide comprises the amino acid sequence of SEQ ID NO:34. In some embodiments, the recombinant neuraminidase further comprises a purification tag. In a specific embodiment, the purification tag is a histidine tag or flag tag. In specific embodiments, the recombinant neuraminidase is enzymatically active as assessed by a technique described herein or known to one of skill in the art (e.g., an NA-Star assay).

In a specific embodiment, provided herein is an immunogenic composition comprising: a) a recombinant neuraminidase comprising an amino acid sequence having at least 80% identity to the amino acid sequence of SEQ ID NO:40, 42, 44, 46 or 48, or SEQ ID NO:40, 42, 44, 46 or 48 without the signal sequence; and b) a CpG oligonucleotide adjuvant (see, e.g., Section 5.2 or 6), in an admixture with a pharmaceutically acceptable carrier. In a specific embodiment, provided herein is an immunogenic composition comprising: a) a recombinant neuraminidase comprising the amino acid sequence of SEQ ID NO:40, 42, 44, 46 or 48, or SEQ ID NO:40, 42, 44, 46 or 48 without the signal sequence; and b) a CpG oligonucleotide adjuvant (see, e.g., Section 5.2 or 6), in an admixture with a pharmaceutically acceptable carrier. In a specific embodiment, provided herein is an immunogenic composition comprising: (a) a recombinant neuraminidase comprising an amino acid sequence having at least 80% identity to SEQ ID NO:50, 52, 54, 56 or 58, or SEQ ID NO:50, 52, 54, 56 or 58 without the signal sequence; and (b) a CpG oligonucleotide adjuvant (see, e.g., Section 5.2 or 6), in an admixture with a pharmaceutically acceptable carrier. In a specific embodiment, provided herein is an immunogenic composition comprising: (a) a recombinant neuraminidase comprising the amino acid sequence of SEQ ID NO:50, 52, 54, 56 or 58, or SEQ ID NO: 50, 52, 54, 56 or 58 without the signal sequence; and (b) a CpG oligonucleotide adjuvant (see, e.g., Section 5.2 or 6), in an admixture with a pharmaceutically acceptable carrier. In a specific embodiment, an immunogenic composition comprising: (a) a recombinant neuraminidase comprising an amino acid sequence having at least 80% identity to SEQ ID NO:60, 62, 64, 66 or 68, or SEQ ID NO:60, 62, 64, 66 or 68 without the signal sequence; and (b) a CpG oligonucleotide adjuvant (see, e.g., Section 5.2 or 6), in an admixture with a pharmaceutically acceptable carrier. In a specific embodiment, an immunogenic composition comprising: (a) a recombinant neuraminidase comprising the amino acid sequence of SEQ ID NO:60, 62, 64, 66 or 68, or SEQ ID NO:60, 62, 64, 66 or 68 without the signal sequence; and (b) a CpG oligonucleotide adjuvant (see, e.g., Section 5.2 or 6), in an admixture with a pharmaceutically acceptable carrier.

In a specific embodiment, provided herein is an immunogenic composition, comprising: (a) two or three recombinant neuraminidases, wherein each recombinant neuraminidase: (ii) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain, and (b) a CpG oligonucleotide adjuvant (see, e.g., Section 5.2 or 6), in an admixture with a pharmaceutically acceptable carrier. In a specific embodiment, the paramyxovirus phosphoprotein tetramerization domain comprises a measles virus phosphoprotein tetramerization domain. In a specific embodiment, the measles virus phosphoprotein tetramerization domain comprises the amino acid sequence of SEQ ID NO:8. In specific embodiments, the measles virus phosphoprotein tetramerization domain comprises an amino acid sequence having at least 80% identity to the amino acid sequence of SEQ ID NO:8. In some embodiments, the immunogenic composition comprises two recombinant neuraminidases. In some embodiments, the immunogenic composition comprises three recombinant neuraminidases. In a specific embodiment, each of the recombinant neuraminidases comprises an influenza A virus neuraminidase globular head domain. In specific embodiments, one of the recombinant neuraminidases comprises an influenza A virus neuraminidase globular head domain of subtype N1, and the second recombinant neuraminidase comprises an influenza A virus neuraminidase domain of subtype N2. In a specific embodiment, the subtype N1 is influenza virus A/Michigan/45/2015. In a specific embodiment, the subtype N2 is influenza virus is influenza virus A/Kansas/14/2017. In a specific embodiment, the influenza virus neuraminidase globular head domain of one recombinant neuraminidase comprises the amino acid sequence of SEQ ID NO:31, and the influenza virus neuraminidase globular head domain of the second recombinant neuraminidase comprises the amino acid sequence of SEQ ID NO:32. In specific embodiments, the influenza virus neuraminidase globular head domain of one recombinant neuraminidase comprises an amino acid sequence having at least 80% identity to SEQ ID NO:31, and the influenza virus neuraminidase globular head domain of the second recombinant neuraminidase comprises an amino acid sequence having at least 80% identity to SEQ ID NO:32. In a specific embodiment, one of the recombinant neuraminidases comprises an influenza A virus neuraminidase globular head domain of subtype N1, the second recombinant neuraminidase comprises an influenza A virus neuraminidase domain of subtype N2, and the third recombinant neuraminidase comprises an influenza B virus neuraminidase globular head domain. In a specific embodiment, the subtype N1 is influenza virus A/Michigan/45/2015, the subtype N2 is influenza virus A/Kansas/14/2017, and the influenza B virus is influenza virus B/Colorado/6/2017. In a specific embodiment, the influenza virus neuraminidase globular head domain of one recombinant neuraminidase comprises the amino acid sequence of SEQ ID NO:31, the influenza virus neuraminidase globular head domain of the second recombinant neuraminidase comprises the amino acid sequence of SEQ ID NO:32, and the influenza virus neuraminidase globular head domain of the third recombinant neuraminidase comprises the amino acid sequence of SEQ ID NO:33. In specific embodiments, the influenza virus neuraminidase globular head domain of one recombinant neuraminidase comprises an amino acid sequence having at least 80% identity to SEQ ID NO:31, the influenza virus neuraminidase globular head domain of the second recombinant neuraminidase comprises an amino acid sequence having at least 80% identity to SEQ ID NO:32, and the influenza virus neuraminidase globular head domain of the third recombinant neuraminidase comprises an amino acid sequence having at least 80% identity to SEQ ID NO:33. In some embodiments, each recombinant neuraminidase further comprises a cleavage site. In specific embodiments, the cleavage site is between the tetramerization domain and influenza virus neuraminidase globular head domain. In specific embodiments, the cleavage site comprises a thrombin cleavage site. In specific embodiments, the cleavage site comprises the amino acid sequence of SEQ ID NO:36 or 37. In some embodiments, each recombinant neuraminidase further comprises a signal peptide. In specific embodiments, the signal peptide comprises the amino acid sequence of SEQ ID NO:34. In some embodiments, each recombinant neuraminidase further comprises a purification tag. In a specific embodiment, the purification tag is a histidine tag or flag tag. In specific embodiments, each recombinant neuraminidase is enzymatically active as assessed by a technique described herein or known to one of skill in the art (e.g., an NA-Star assay).

In a specific embodiment, provided herein is an immunogenic composition, comprising: (a) three recombinant neuraminidases, wherein: (i) the first recombinant neuraminidase comprises the amino acid sequence of SEQ ID NO:40, 42, 44, 46, or 48, or SEQ ID NO:40, 42, 44, 46, or 48 without the signal sequence; (ii) the second recombinant neuraminidase comprises the amino acid sequence of SEQ ID NO:50, 52, 54, 56, or 58, or SEQ ID NO:50, 52, 54, 56, or 58 without the signal sequence; and (iii) the third recombinant neuraminidase comprises the amino acid sequence of SEQ ID NO:60, 62, 64, 66, or 68, or SEQ ID NO:60, 62, 64, 66, or 68 without the signal sequence, and (b) a CpG oligonucleotide adjuvant described herein (e.g., in Section 5.2 or 6), in an admixture with a pharmaceutically acceptable carrier.

In some embodiments, provided herein is a method of immunizing a subject against influenza virus, comprising administering to the subject an immunogenic composition and a CpG oligonucleotide adjuvant described herein, wherein the immunogenic composition comprises a recombinant neuraminidase described herein in an admixture with a pharmaceutically acceptable carrier. In some embodiments, provided herein is a method of inducing an immune response in a subject against influenza virus, comprising administering to the subject an immunogenic composition and a CpG oligonucleotide adjuvant described herein, wherein the immunogenic composition comprises a recombinant neuraminidase described herein in an admixture with a pharmaceutically acceptable carrier. In some embodiments, comprising administering to the subject an immunogenic composition and a CpG oligonucleotide adjuvant described herein, wherein the immunogenic composition comprises a recombinant neuraminidase described herein in an admixture with a pharmaceutically acceptable carrier. In some embodiments, the immunogenic composition comprises two or more (e.g., two or three) recombinant neuraminidases described herein. In some embodiments, the immunogenic composition and the CpG oligonucleotide adjuvant are administered by the same route of administration. In some embodiments, the immunogenic composition and the CpG oligonucleotide adjuvant are administered by different routes of administration. In specific embodiments, the immunogenic composition and the CpG oligonucleotide adjuvant are administered to the same area or location of the subject. In some embodiments, the immunogenic composition, the CpG oligonucleotide adjuvant or both are administered intramuscularly to the subject. In some embodiments, the immunogenic composition, the CpG oligonucleotide adjuvant, or both are administered intranasally to the subject. In a specific embodiment, the subject is human.

In one aspect, provided herein is an immunogenic composition, comprising: (a) a recombinant neuraminidase, wherein the recombinant neuraminidase (i) comprises an influenza virus neuraminidase globular head domain, and a paramyxovirus phosphoprotein tetramerization domain; and (b) an adjuvant, wherein the adjuvant comprises an oligonucleotide of from 10 to 35 nucleotides in length comprising the nucleotide sequence of 5′-GAACGTTCG-3′ (SEQ ID NO:3), in an admixture with a pharmaceutically acceptable carrier. In some embodiments, provided herein is an immunogenic composition, comprising: (a) a recombinant neuraminidase, wherein the recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain, and (b) an adjuvant, wherein the adjuvant comprises an oligonucleotide of from 10 to 35 nucleotides in length comprising the nucleotide sequence of 5′-GAACGTTCG-3′ (SEQ ID NO:3), in an admixture with a pharmaceutically acceptable carrier. In some embodiments, the paramyxovirus phosphoprotein tetramerization domain comprises a measles virus phosphoprotein tetramerization domain. In a specific embodiment, the measles virus phosphoprotein tetramerization domain comprises the amino acid sequence of SEQ ID NO:8. In specific embodiments, the measles virus phosphoprotein tetramerization domain comprises an amino acid sequence having at least 80% identity to the amino acid sequence of SEQ ID NO:8. In some embodiments, the influenza virus neuraminidase globular head domain comprises an influenza A virus neuraminidase globular head domain. In specific embodiments, the influenza A virus neuraminidase is subtype N1 or subtype N2. In specific embodiments, the subtype N1 is influenza virus A/Michigan/45/2015. In specific embodiments, the subtype N2 is influenza virus A/Kansas/14/2017. In some embodiments, the influenza virus neuraminidase globular head domain comprises an influenza B virus neuraminidase globular head domain. In specific embodiments, the influenza B virus is influenza virus B/Colorado/6/2017. In specific embodiments, the influenza virus neuraminidase globular head domain comprises the amino acid sequence of SEQ ID NO:31, 32, or 33. In specific embodiments, the influenza virus neuraminidase globular head domain comprises an amino acid sequence having at least 80% identity to SEQ ID NO:31, 32, or 33. In some embodiments, the recombinant neuraminidase further comprises a cleavage site. In some embodiments, the cleavage site in the recombinant neuraminidase is between the tetramerization domain and influenza virus neuraminidase globular head domain. In a specific embodiment, the cleavage site comprises a thrombin cleavage site. In a specific embodiment, the cleavage site comprises the amino acid sequence of SEQ ID NO:36 or 37. In some embodiments, the recombinant neuraminidase further comprises a signal peptide. In a specific embodiment, the signal peptide comprises the amino acid sequence of SEQ ID NO:34. In some embodiments, the recombinant neuraminidase further comprises a purification tag. In a specific embodiment, the purification tag is a histidine tag or flag tag. In specific embodiments, the recombinant neuraminidase is enzymatically active as assessed by a technique described herein or known to one of skill in the art (e.g., an NA-Star assay).

In a specific embodiment, provided herein is an immunogenic composition comprising: a) a recombinant neuraminidase comprising an amino acid sequence having at least 80% identity to the amino acid sequence of SEQ ID NO:40, 42, 44, 46 or 48, or SEQ ID NO:40, 42, 44, 46 or 48 without the signal sequence; and b) an adjuvant, wherein the adjuvant comprises an oligonucleotide of from 10 to 35 nucleotides in length comprising the nucleotide sequence of SEQ ID NO:3, in an admixture with a pharmaceutically acceptable carrier. In a specific embodiment, provided herein is an immunogenic composition comprising: a) a recombinant neuraminidase comprising the amino acid sequence of SEQ ID NO:40, 42, 44, 46 or 48, or SEQ ID NO:40, 42, 44, 46 or 48 without the signal sequence; and b) an adjuvant, wherein the adjuvant comprises an oligonucleotide of from 10 to 35 nucleotides in length comprising the nucleotide sequence of SEQ ID NO:3, in an admixture with a pharmaceutically acceptable carrier. In a specific embodiment, provided herein is an immunogenic composition comprising: (a) a recombinant neuraminidase comprising an amino acid sequence having at least 80% identity to SEQ ID NO:50, 52, 54, 56 or 58, or SEQ ID NO:50, 52, 54, 56 or 58 without the signal sequence; and (b) an adjuvant, wherein the adjuvant comprises an oligonucleotide of from 10 to 35 nucleotides in length comprising the nucleotide sequence of SEQ ID NO:3, in an admixture with a pharmaceutically acceptable carrier. In a specific embodiment, provided herein is an immunogenic composition comprising: (a) a recombinant neuraminidase comprising the amino acid sequence of SEQ ID NO:50, 52, 54, 56 or 58, or SEQ ID NO: 50, 52, 54, 56 or 58 without the signal sequence; and (b) an adjuvant, wherein the adjuvant comprises an oligonucleotide of from 10 to 35 nucleotides in length comprising the nucleotide sequence of SEQ ID NO:3, in an admixture with a pharmaceutically acceptable carrier. In a specific embodiment, an immunogenic composition comprising: (a) a recombinant neuraminidase comprising an amino acid sequence having at least 80% identity to SEQ ID NO:60, 62, 64, 66 or 68, or SEQ ID NO:60, 62, 64, 66 or 68 without the signal sequence; and (b) an adjuvant, wherein the adjuvant comprises an oligonucleotide of from 10 to 35 nucleotides in length comprising the nucleotide sequence of SEQ ID NO:3, in an admixture with a pharmaceutically acceptable carrier. In a specific embodiment, an immunogenic composition comprising: (a) a recombinant neuraminidase comprising the amino acid sequence of SEQ ID NO:60, 62, 64, 66 or 68, or SEQ ID NO:60, 62, 64, 66 or 68 without the signal sequence; and (b) an adjuvant, wherein the adjuvant comprises an oligonucleotide of from 10 to 35 nucleotides in length comprising the nucleotide sequence of SEQ ID NO:3, in an admixture with a pharmaceutically acceptable carrier.

In a specific embodiment, provided herein is an immunogenic composition, comprising: (a) two or three recombinant neuraminidases, wherein each recombinant neuraminidase: (ii) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain, and (b) an adjuvant, wherein the adjuvant comprises an oligonucleotide of from 10 to 35 nucleotides in length comprising the nucleotide sequence of SEQ ID NO:3, in an admixture with a pharmaceutically acceptable carrier. In a specific embodiment, the paramyxovirus phosphoprotein tetramerization domain comprises a measles virus phosphoprotein tetramerization domain. In a specific embodiment, the measles virus phosphoprotein tetramerization domain comprises the amino acid sequence of SEQ ID NO:8. In specific embodiments, the measles virus phosphoprotein tetramerization domain comprises an amino acid sequence having at least 80% identity to the amino acid sequence of SEQ ID NO:8. In some embodiments, the immunogenic composition comprises two recombinant neuraminidases. In some embodiments, the immunogenic composition comprises three recombinant neuraminidases. In a specific embodiment, each of the recombinant neuraminidases comprises an influenza A virus neuraminidase globular head domain. In specific embodiments, one of the recombinant neuraminidases comprises an influenza A virus neuraminidase globular head domain of subtype N1, and the second recombinant neuraminidase comprises an influenza A virus neuraminidase domain of subtype N2. In a specific embodiment, the subtype N1 is influenza virus A/Michigan/45/2015. In a specific embodiment, the subtype N2 is influenza virus is influenza virus A/Kansas/14/2017. In a specific embodiment, the influenza virus neuraminidase globular head domain of one recombinant neuraminidase comprises the amino acid sequence of SEQ ID NO:31, and the influenza virus neuraminidase globular head domain of the second recombinant neuraminidase comprises the amino acid sequence of SEQ ID NO:32. In specific embodiments, the influenza virus neuraminidase globular head domain of one recombinant neuraminidase comprises an amino acid sequence having at least 80% identity to SEQ ID NO:31, and the influenza virus neuraminidase globular head domain of the second recombinant neuraminidase comprises an amino acid sequence having at least 80% identity to SEQ ID NO:32. In a specific embodiment, one of the recombinant neuraminidases comprises an influenza A virus neuraminidase globular head domain of subtype N1, the second recombinant neuraminidase comprises an influenza A virus neuraminidase domain of subtype N2, and the third recombinant neuraminidase comprises an influenza B virus neuraminidase globular head domain. In a specific embodiment, the subtype N1 is influenza virus A/Michigan/45/2015, the subtype N2 is influenza virus A/Kansas/14/2017, and the influenza B virus is influenza virus B/Colorado/6/2017. In a specific embodiment, the influenza virus neuraminidase globular head domain of one recombinant neuraminidase comprises the amino acid sequence of SEQ ID NO:31, the influenza virus neuraminidase globular head domain of the second recombinant neuraminidase comprises the amino acid sequence of SEQ ID NO:32, and the influenza virus neuraminidase globular head domain of the third recombinant neuraminidase comprises the amino acid sequence of SEQ ID NO:33. In specific embodiments, the influenza virus neuraminidase globular head domain of one recombinant neuraminidase comprises an amino acid sequence having at least 80% identity to SEQ ID NO:31, the influenza virus neuraminidase globular head domain of the second recombinant neuraminidase comprises an amino acid sequence having at least 80% identity to SEQ ID NO:32, and the influenza virus neuraminidase globular head domain of the third recombinant neuraminidase comprises an amino acid sequence having at least 80% identity to SEQ ID NO:33. In some embodiments, each recombinant neuraminidase further comprises a cleavage site. In specific embodiments, the cleavage site is between the tetramerization domain and influenza virus neuraminidase globular head domain. In specific embodiments, the cleavage site comprises a thrombin cleavage site. In specific embodiments, the cleavage site comprises the amino acid sequence of SEQ ID NO:36 or 37. In some embodiments, each recombinant neuraminidase further comprises a signal peptide. In specific embodiments, the signal peptide comprises the amino acid sequence of SEQ ID NO:34. In some embodiments, each recombinant neuraminidase further comprises a purification tag. In a specific embodiment, the purification tag is a histidine tag or flag tag. In specific embodiments, each recombinant neuraminidase is enzymatically active as assessed by a technique described herein or known to one of skill in the art (e.g., an NA-Star assay).

In a specific embodiment, provided herein is an immunogenic composition, comprising: (a) three recombinant neuraminidases, wherein: (i) the first recombinant neuraminidase comprises the amino acid sequence of SEQ ID NO:40, 42, 44, 46, or 48, or SEQ ID NO:40, 42, 44, 46, or 48 without the signal sequence; (ii) the second recombinant neuraminidase comprises the amino acid sequence of SEQ ID NO:50, 52, 54, 56, or 58, or SEQ ID NO:50, 52, 54, 56, or 58 without the signal sequence; and (iii) the third recombinant neuraminidase comprises the amino acid sequence of SEQ ID NO:60, 62, 64, 66, or 68, or SEQ ID NO:60, 62, 64, 66, or 68 without the signal sequence, and (b) an adjuvant, wherein the adjuvant comprises an oligonucleotide comprising the nucleotide sequence of SEQ ID NO:3, in an admixture with a pharmaceutically acceptable carrier.

In specific embodiments, the oligonucleotide of an adjuvant described herein comprises the nucleotide sequence of 5′-TGACTGTGAACGTTCGAGATGA-3′ (SEQ ID NO: 4). In some embodiments, the oligonucleotide of the adjuvant is 22, 23, 24, 25, 26, 27, 28, 29, 30, 31, 32, 33, 34, or 35 nucleotides in length. In some embodiments, the oligonucleotide of an adjuvant described herein is a single stranded oligodeoxynucleotide. In some embodiments, the oligonucleotide of an adjuvant described herein comprises only phosphorothioate linkages, or a combination of one or more phosphodiester linkages and one or more phosphorothioate linkages. In some embodiments, the oligonucleotide of an adjuvant described herein is fully RNA. In some embodiments, the oligonucleotide of an adjuvant described herein is an RNA/DNA chimera.

In some embodiments, an adjuvant of the immunogenic composition described herein comprises an aluminum salt adjuvant. In a specific embodiment, the aluminum salt adjuvant comprises aluminum hydroxide, amorphous aluminum hydroxyphosphate sulfate, aluminum hydroxide, aluminum phosphate, or potassium aluminum sulfate, or a combination thereof. In some embodiments, the aluminum salt adjuvant comprises one or both of aluminum hydroxide and aluminum phosphate. In some embodiments, the aluminum salt adjuvant consists of aluminum hydroxide. In some embodiments, an immunogenic composition described herein comprises from about 0.25 to about 1.25 mg Al³⁺, preferably from about 0.50 to about 1.00 mg Al³⁺. In some embodiments, the immunogenic composition comprises about 0.375 mg Al³⁺, about 0.75 mg Al³⁺, or about 1.00 mg Al³⁺. In some embodiments, the immunogenic composition comprises about 0.375 mg Al³⁺.

In a specific embodiment, the adjuvant of an immunogenic composition described herein comprises a CpG oligonucleotide adjuvant described herein (e.g., in Section 5.2 or 6).

In another aspect, provided herein are methods of immunizing a subject against influenza virus, comprising administering to the subject a dose of an immunogenic composition described herein. In some embodiments, the subject is administered at least one subsequent dose of the immunogenic composition. In some embodiments, the subject is administered two subsequent doses of the immunogenic composition. In one embodiment, the immunogenic composition is administered intramuscularly to the subject. In another embodiment, the immunogenic composition is administered intranasally to the subject. In a specific embodiment, the subject is human.

In another aspect, provided herein are methods of inducing an immune response in a subject against influenza virus, comprising administering to the subject a dose of an immunogenic composition described herein. In some embodiments, the subject is administered at least one subsequent dose of the immunogenic composition. In some embodiments, the subject is administered two subsequent doses of the immunogenic composition. In one embodiment, the immunogenic composition is administered intramuscularly to the subject. In another embodiment, the immunogenic composition is administered intranasally to the subject. In a specific embodiment, the subject is human.

In another aspect, provided herein are methods of preventing an influenza virus disease in a subject, comprising administering to the subject a dose of an immunogenic composition described herein. In some embodiments, the subject is administered at least one subsequent dose of the immunogenic composition. In some embodiments, the subject is administered two subsequent doses of the immunogenic composition. In one embodiment, the immunogenic composition is administered intramuscularly to the subject. In another embodiment, the immunogenic composition is administered intranasally to the subject. In a specific embodiment, the subject is human.

In some embodiments, provided herein is a method of immunizing a subject against influenza virus, comprising administering to the subject an immunogenic composition and an adjuvant, wherein the immunogenic composition comprises a recombinant neuraminidase described herein in an admixture with a pharmaceutically acceptable carrier, wherein the adjuvant comprises an oligonucleotide of from 10 to 35 nucleotides in length comprising the nucleotide sequence of 5′-GAACGTTCG-3′ (SEQ ID NO:3). In some embodiments, provided herein is a method of inducing an immune response in a subject against influenza virus, comprising administering to the subject an immunogenic composition and an adjuvant, wherein the immunogenic composition comprises a recombinant neuraminidase described herein in an admixture with a pharmaceutically acceptable carrier, wherein the adjuvant comprises an oligonucleotide of from 10 to 35 nucleotides in length comprising the nucleotide sequence of 5′-GAACGTTCG-3′ (SEQ ID NO:3). In some embodiments, provided herein is a method of preventing an influenza virus disease in a subject, comprising administering to the subject an immunogenic composition and an adjuvant, wherein the immunogenic composition comprises a recombinant neuraminidase described herein in an admixture with a pharmaceutically acceptable carrier, wherein the adjuvant comprises an oligonucleotide of from 10 to 35 nucleotides in length comprising the nucleotide sequence of 5′-GAACGTTCG-3′ (SEQ ID NO:3). In some embodiments, the immunogenic composition comprises two or more recombinant neuraminidases described herein (e.g., two or three recombinant neuraminidases described herein). In some embodiments, the oligonucleotide is 22, 23, 24, 25, 26, 27, 28, 29, 30, 31, 32, 33, 34, or 35 nucleotides in length. In some embodiments, the oligonucleotide is a single stranded oligodeoxynucleotide. In some embodiments, the oligonucleotide comprises only phosphorothioate linkages, or a combination of one or more phosphodiester linkages and one or more phosphorothioate linkages. In some embodiments, the oligonucleotide is fully RNA or is an RNA/DNA chimera. In some embodiments, the adjuvant further comprises an aluminum salt (e.g., an aluminum salt described herein). In some embodiments, the aluminum salt comprises aluminum hydroxide, aluminum phosphate, or both. In some embodiments, the immunogenic composition does not comprise an aluminum salt. In some embodiments, the immunogenic composition and the adjuvant are administered by the same route of administration. In some embodiments, the immunogenic composition and the adjuvant are administered by different routes of administration. In specific embodiments, the immunogenic composition and the adjuvant are administered to the same area or location of the subject. In some embodiments, the immunogenic composition, the adjuvant, or both are administered intramuscularly to the subject. In some embodiments, the immunogenic composition, the adjuvant, or both are administered intranasally to the subject. In a specific embodiment, the subject is human.

In some embodiments, provided herein is a method of immunizing a subject against influenza virus, comprising administering to the subject an immunogenic composition and an adjuvant, wherein the immunogenic composition comprises a recombinant neuraminidase in an admixture with a pharmaceutically acceptable carrier, wherein the adjuvant comprises an oligonucleotide of from 10 to 35 nucleotides in length comprising the nucleotide sequence of 5′-GAACGTTCG-3′ (SEQ ID NO:3), and wherein recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain. In some embodiments, provided herein is a method of inducing an immune response in a subject against influenza virus, comprising administering to the subject an immunogenic composition and an adjuvant, wherein the immunogenic composition comprises a recombinant neuraminidase in an admixture with a pharmaceutically acceptable carrier, wherein the adjuvant comprises an oligonucleotide of from 10 to 35 nucleotides in length comprising the nucleotide sequence of 5′-GAACGTTCG-3′ (SEQ ID NO:3), and wherein recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain. In some embodiments, provided herein is a method of preventing an influenza virus disease in a subject, comprising administering to the subject an immunogenic composition and an adjuvant, wherein the immunogenic composition comprises a recombinant neuraminidase in an admixture with a pharmaceutically acceptable carrier, wherein the adjuvant comprises an oligonucleotide of from 10 to 35 nucleotides in length comprising the nucleotide sequence of 5′-GAACGTTCG-3′ (SEQ ID NO:3), and wherein recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain. In some embodiments, the paramyxovirus phosphoprotein tetramerization domain comprises a measles virus phosphoprotein tetramerization domain. In a specific embodiment, the measles virus phosphoprotein tetramerization domain comprises the amino acid sequence of GDHYDDELFSDVQDIKTALAKIHEDNQKIISKLESLLLLKGEVESIKKQINRQNISISTL EGHLSSIMIAIPGL (SEQ ID NO:8). In some embodiments, the measles virus phosphoprotein tetramerization domain comprises an amino acid sequence having at least 80% identity to the amino acid sequence of SEQ ID NO:8. In some embodiments, the influenza virus neuraminidase globular head domain comprises an influenza A virus neuraminidase globular head domain. In a specific embodiment, wherein the influenza A virus neuraminidase is subtype N1 or subtype N2. In a specific embodiments, the subtype N1 is influenza virus A/Michigan/45/2015. In a specific embodiment, the subtype N2 is influenza virus A/Kansas/14/2017. In some embodiments, the influenza virus neuraminidase globular head domain comprises an influenza B virus neuraminidase globular head domain. In a specific embodiment, the influenza B virus is influenza virus B/Colorado/6/2017. In a specific embodiment, the influenza virus neuraminidase globular head domain comprises the amino acid sequence of SEQ ID NO: 31, 32, or 33. In some embodiments the influenza virus neuraminidase globular head domain comprises an amino acid sequence having at least 80% identity to SEQ ID NO:31, 32, or 33. In some embodiments, the recombinant neuraminidase further comprises a signal peptide (e.g., SEQ ID NO: 34). In a specific embodiment, the recombinant neuraminidase is enzymatically active as assessed by an assay described herein (e.g., NA-Star assay) or known in the art. In some embodiments, the oligonucleotide comprises the nucleotide sequence of 5′-TGACTGTGAACGTTCGAGATGA-3′ (SEQ ID NO: 4). In some embodiments, the oligonucleotide is 22, 23, 24, 25, 26, 27, 28, 29, 30, 31, 32, 33, 34, or 35 nucleotides in length. In some embodiments, the oligonucleotide is a single stranded oligodeoxynucleotide. In some embodiments, the oligonucleotide comprises only phosphorothioate linkages, or a combination of one or more phosphodiester linkages and one or more phosphorothioate linkages. In some embodiments, the oligonucleotide is fully RNA or is an RNA/DNA chimera. In some embodiments, the adjuvant further comprises an aluminum salt (e.g., an aluminum salt described herein). In some embodiments, the aluminum salt comprises aluminum hydroxide, aluminum phosphate, or both. In some embodiments, the immunogenic composition does not comprise an aluminum salt. In some embodiments, the immunogenic composition and the adjuvant are administered by the same route of administration. In some embodiments, the immunogenic composition and the adjuvant are administered by different routes of administration. In specific embodiments, the immunogenic composition and the adjuvant are administered to the same area or location of the subject. In some embodiments, the immunogenic composition, the adjuvant, or both are administered intramuscularly to the subject. In some embodiments, the immunogenic composition, the adjuvant, or both are administered intranasally to the subject. In a specific embodiment, the subject is human.

### 3.1 Terminology

As used herein, the term “nucleic acid” is intended to include DNA molecules (e.g., cDNA or genomic DNA) and RNA molecules (e.g., mRNA) and analogs of the DNA or RNA generated using nucleotide analogs. In a specific embodiment, the nucleic acid may be self-replicating RNA.

As used interchangeably herein, the terms “polynucleotide” and “oligonucleotide” are oligomers of nucleic acids and include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine.

The terms “CpG,” “CpG motif,” and “cytosine-phosphate-guanosine,” as used herein, refer to an unmethylated cytidine-phospho-guanosine dinucleotide, which when present in an oligonucleotide contributes to a measurable immune response in vitro, in vivo and/or ex vivo. Examples of measurable immune responses include, but are not limited to, antigen-specific antibody production, secretion of cytokines, activation, or expansion of lymphocyte populations, such as NK cells, CD4+T lymphocytes, CD8+T lymphocytes, B lymphocytes, and the like. Preferably, the CpG oligonucleotide preferentially activates a Th1-type response.

As used herein, the terms “purified” and “isolated” when used in the context of a polypeptide (including an antibody) that is obtained from a natural source, e.g., cells, refers to a polypeptide which is substantially free of contaminating materials from the natural source, e.g., minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide (including an antibody) that is chemically synthesized refers to a polypeptide which is substantially free of chemical precursors or other chemicals which are involved in the syntheses of the polypeptide. In a specific embodiment, a recombinant influenza virus NA is chemically synthesized. In another specific embodiment, a recombinant influenza virus NA is isolated.

As used herein, terms “subject” or “patient” are used interchangeably to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant.

As used herein, the term “premature human infant” refers to a human infant born at less than 37 weeks of gestational age.

As used herein, the term “seasonal influenza virus strain” refers to a strain of influenza virus to which a subject population is exposed to on a seasonal basis. In specific embodiments, the term seasonal influenza virus strain refers to a strain of influenza A virus. In specific embodiments, the term seasonal influenza virus strain refers to a strain of influenza virus that belongs to the H1 or the H3 subtype, i.e., the two subtypes that presently persist in the human subject population. In other embodiments, the term seasonal influenza virus strain refers to a strain of influenza B virus. In a specific embodiment, a seasonal influenza vaccine comprising one or more antigens (e.g., influenza virus hemagglutinin) to induce an immune response to seasonal influenza virus strain.

The terms “tertiary structure” and “quaternary structure” have the meanings understood by those of skill in the art. Tertiary structure refers to the three-dimensional structure of a single polypeptide chain. Quaternary structure refers to the three dimensional structure of a polypeptide having multiple polypeptide chains.

As used herein, in some embodiments, the phrase “wild-type” in the context of a viral polypeptide refers to a viral polypeptide that is found in nature and is associated with a naturally occurring virus.

As used herein, in some embodiments, the phrase “wild-type” in the context of a virus refers to the types of a virus that are prevalent, circulating naturally and producing typical outbreaks of disease. In other embodiments, the term “wild-type” in the context of a virus refers to a parental virus.

As used herein, the term “about” or “approximately” when used in conjunction with a number refers to any number within 1%, 5%, or 10% of the referenced number and includes the referenced number.

### 4. DESCRIPTION OF THE FIGURES

FIGS. 1A-1E depict alignments of the neuraminidases of the following influenza viruses: A/PR/8/34 (N1 PR8; SEQ ID NO:10), A/Michigan/45/15 (N1_Mich15; SEQ ID NO:11), A/WSN/33 (N1_WSN; SEQ ID NO:12), A/Hong Kong/4801/14 (N2_HK14; SEQ ID NO:13), A/chicken/Bangladesh/19870/13 (N2_ck; SEQ ID NO:14), A/swine/Missouri/4296424/06A (N3_sw; SEQ ID NO:15), A/blue-winged teal/Guatemala/CIP049H113-74/13 (N3_bwt; SEQ ID NO:16), A/duck/Jiangxi/22676/13 (N3_duck; SEQ ID NO:17), A/duck/Hokkaido/222/14 (N4_duck; SEQ ID NO:18), A/ruddy turnstone/Delaware/AI03-378/03 (N4_rt; SEQ ID NO:19), A/American wigeon/California/HS007B/15 (N5_aw; SEQ ID NO:20), A/migratory duck/Jiangxi/6847/03 (N5_md; SEQ ID NO:21), A/Shenzhen/1/16 (N6_Sz; SEQ ID NO:22), A/Caspian seal/Russia/T1/12 (N6_cs; SEQ ID NO:23), A/chicken/Netherlands/1/03 (N7_cN; SEQ ID NO:24), A/chicken/Germany/R28/03 (N7_cG; SEQ ID NO:25), A/chicken/Netherlands/14015531/14 (N8_cN; SEQ ID NO:26), A/chicken/Laos/A0573/07 (N8_cL; SEQ ID NO:27), A/Anhui/2/13 (N9_AH13; SEQ ID NO:28), A/chicken/Dongguan/1143/14 (N9_ck; SEQ ID NO:29), and B/Malaysia/2506/04 (B_Mal; SEQ ID NO:30). C49 is shown in bold and underlined for N1_PR8.

FIGS. 2A-2E depict proof of principle experiments to assess if one vaccination (prime-only) is sufficient to induce a robust and protective immune response in vivo compared to a two vaccination (prime-boost) regimen. FIG. 2A depicts study design and vaccination scheme. Mice (n=5 per group, except for Neg. Contr. 2×Vacc where n=3) were vaccinated either once (prime-only) or twice (prime-boost) with 3 μg of N1-MPP, 3 μg N1-MPP+ODN1018 or irrelevant protein following a challenge with 10×mLD₅₀ of A/Singapore/GP1908/2015 H1N1 (IVR-180, which is antigenically equivalent to 109 A/Michigan/45/15). Weight loss and survival were monitored over a 14-day time period. Mice were bled on day 21 and 42 post prime for serological analysis. FIG. 2B depicts weight loss curves (mean plus standard deviation) and FIG. 2C depicts survival after challenge with 10×mLD₅₀ of A/Singapore/GP1908/2015 H1N1 (IVR-180). Differences in survival between the vaccine groups and respective control groups as well as between matched prime-only and prime-boost groups were analyzed using a Mantel-Cox log rank test: N1-MPP+ODN1018 1×Vacc vs. Neg. Contr. 1×Vacc p=0.0298; N1-MPP 1×Vacc vs. N1-MPP+ODN1018 1×Vacc p=0.0143; N1-MPP 2×Vacc vs Neg. Contr. 2×Vacc p=0.0067; N1-MPP+ODN1018 2×Vacc vs. Neg. Contr. 2×Vacc p=0.0067; N1-MPP 1×Vacc vs. N1-MPP 2×Vacc p=0.0027; N1-MPP+ODN1018 1×Vacc vs. N1-MPP+ODN1018 2×Vacc p=0.0494. The difference between the remaining curves were not statistically significant p>0.05 or were not compared. FIG. 2D depicts ELISA titers in serum pre-challenge (mean plus standard deviation). FIG. 2E depicts NI titers 42 days post prime using H7N1_{Mich15} virus (geometric mean plus standard deviation of the geometric mean). Statistical analysis in FIG. 2E was performed using an one-way ANOVA corrected for multiple comparisons.

FIGS. 3A-3B depict assays for protection from lethal influenza challenge protection by passive serum transfer of recombinant neuraminidase N1-MPP. BALB/c mice (n=5 per group except for Neg. Contr. where n=3) received a passive serum transfer intraperitoneally and were challenged with 5×mLD₅₀ of A/Singapore/GP1908/2015 H1N1 (IVR-180). FIG. 3A depicts weight loss curves monitored over a 14-day period (shown is mean plus standard deviation) and the FIG. 3B depicts survival of each group. Differences in survival were analyzed using a Mantel-Cox log rank test: N1-MPP vs Neg. Ctrl. p=0.0419; N1-MPP+ODN1018 vs Neg. Ctrl. p=0.0034; the differences between remaining curves were not statistically significant (p>0.05).

FIGS. 4A-4E depict results from preclinical assessment of N1-MPP and seasonal QIV in combination with ODN1018. FIG. 4A depicts study design and vaccination scheme. Mice (n=5 per group) were vaccinated in a prime-boost regimen and then challenged either with 25×mLD₅₀ of A/Singapore/GP1908/2015 H1N1 (IVR-180) to monitor weight loss and survival over a 14-day period or with 1×mLD₅₀ of A/Singapore/GP1908/2015 H1N1 (IVR-180) to determine viral lung titers. FIG. 4B depicts weight loss curves (mean plus standard deviation) and FIG. 4C depicts survival after viral challenge. Differences in survival were analyzed using a Mantel-Cox log rank test: N1-MPP vs Neg. Ctrl. p=0.0404; all other groups vs. Neg. Ctrl. p=0.0082. The differences between remaining curves were not statistically significant (p>0.05). FIG. 4D and FIG. 4E depict viral lung titers obtained on day 3 and day 6 post challenge, respectively. Statistical analysis in FIGS. 4D and 4E was performed using an one-way ANOVA corrected for multiple comparisons, shown is mean plus standard deviation.

FIGS. 5A-5H depict the results from studies assessing the cross-reactivity of serum antibodies against homologous N1-subtypes, as assessed using a neuraminidase inhibition (NI) assay and ELISA. FIGS. 5A-5B depict the results from NI assays performed. FIG. 6A depicts the results from NI assays performed using a reassortant H7N1_{Mich15} virus containing the N1 of A/Michigan/45/2015, and FIG. 5B depicts the results from NI assays performed using a reassortant H7N1_{Mich15} virus containing the N1 of A/California/04/09. Geometric mean plus standard deviation is shown. Statistical analysis in FIGS. 5A and 5B was performed using an one-way ANOVA corrected for multiple comparisons, shown is mean plus standard deviation. FIGS. 5C-5H show the results of ELISA cross-reactivity testing of antibodies against Mich15 N1-VASP protein (FIG. 5C), NC99 N1-VASP protein (FIG. 5D), PR8 N1-VASP protein (FIG. 5E), Cal09 N1-VASP protein (FIG. 5F), Bris09 N1-VASP protein (FIG. 5G), and Vn04 N1-VASP protein (FIG. 5H). For FIGS. 5C-5H, mean and standard deviation are shown.

FIGS. 6A-6J depict the results from dose testing of recombinant neuraminidase N1-MPP. Mice (n=4-5 per group) were vaccinated in a prime-boost regimen with different dosages of recombinant neuraminidase N1-MPP (3 μg, FIGS. 6A-6B; 1 μg, FIGS. 6C-6D; 0.3 μg, FIGS. 6E-6F; 0.1 μg, FIGS. 6G-6H) or an irrelevant protein, and then challenged with 25×mLD₅₀ of A/Singapore/GP1908/2015 H1N1 (IVR-180). Weight loss (mean plus standard deviation) and survival were monitored over a 14-day period. Blood was obtained 42 days after prime and serological testing via ELISA and NI assay was performed. FIGS. 6A, 6C, 6E, and 6G depict weight loss curves, and FIGS. 6B, 6D, 6F, and 6H depict survival curves for the respective dose groups. Differences in survival were analyzed using a Mantel-Cox log rank test: 3 μg N1-MPP vs 3 μg Neg. Ctrl. p=0.0116; 3 μg N1-MPP+ODN1018 vs 1 μg Neg. Ctrl. p=0.0031; 1 μg N1-MPP vs 1 μg Neg. Ctrl. p=0.0143; 1 μg N1-MPP+ODN1018 vs 1 μg Neg. Ctrl. p=0.0027; 0.1 μg N1-MPP vs 0.1 μg Neg. Ctrl. p=0.0495; 0.1 μg N1-MPP+ODN1018 vs 0.1 μg Neg. Ctrl. p=0.0143; the differences between the remaining curves were not statistically significant (p>0.05). FIG. 6I depicts the results of pre-challenge antibody titers (NI assay) against H7N1_{Mich15} virus from mice vaccinated with the indicated recombinant protein or combination. FIG. 6J depicts the results of ELISA against recombinant Mich15 N1-VASP protein with serum from mice vaccinated for the dose de-escalation experiment. Statistical analysis in FIGS. 6I and 6J was performed using an one-way ANOVA corrected for multiple comparisons, shown is mean plus standard deviation.

FIGS. 7A-7E depict the results from the structural analysis of recombinant neuraminidase constructs N1-MPP, N2-MPP, and B-NA-MPP. In SDS-PAGE under denaturing conditions (FIG. 7A), all proteins show monomeric structures at an expected size of about 60 kDa. In SDS-PAGE following BS3 cross-linking (FIG. 7B), each of N1-MPP, N2-MPP and B-NA-MPP show tetrameric structures at around 240 kDa. BSA was included as a monomer control. FIG. 7C depicts the results of ELISA against recombinant N2-MPP using a broad panel of human anti-N2 mAbs to verify the correct presentation of epitopes. FIG. 7D depicts the results of ELISA against recombinant B-NA-MPP using a broad panel of human anti-B-NA mAbs to verify the correct presentation of epitopes. FIG. 7E depicts the results of an NA-Star assay to assess the enzymatic activity of N1-MPP, N2-MPP, B-NA-MPP. Assays in FIGS. 7C-7E were run once in duplicates and the duplicates were used to calculate one area under the curve (AUC) value.

FIGS. 8A-8I show results of the assessment of the in vivo protective potential of N1-MPP, N2-MPP, and B-NA-MPP alone or in combination with adjuvant ODN1018. Female 6-8 week old BALB/c (FIGS. 8A-8C and 8G-8I) or DBA.2 (FIGS. 8D-8F) mice were vaccinated in a prime-boost regimen with the respective recombinant neuraminidase. Blood was obtained 42 days after the prime and used for serological analysis. FIG. 8A-8B depict weight loss curves (FIG. 8A) and percent survival (FIG. 8B) of BALB/c mice immunized with N1-MPP alone or a combination of N1-MPP and ODN1018 after challenge with 25×LD50 of A/Singapore/GP1908/2015 (H1N1). Differences in survival were analyzed using a Mantel-Cox log rank test: N1-MPP vs Neg. Contr. p=0.0035; N1-MPP+ODN1018 vs Neg. Contr. p=0.0035; other differences were not statistically significant (p>0.05). FIG. 8C depicts the results of the serological analysis of pre-challenge serum antibodies by ELISA against recombinant Mich15 N1-VASP, after immunizing BALB/c mice with N1-MPP alone or in combination with adjuvant ODN1018. FIG. 8D-8E depict weight loss curves (FIG. 8D) and percent survival (FIG. 8E) of DBA.2 mice immunized with N2-MPP alone or a combination of N2-MPP and ODN1018 after challenge with 25×mLD50 of A/Switzerland/9715293/2013 (H3N2). Differences in survival were analyzed using a Mantel-Cox log rank test: N2-MPP vs Neg. Contr. p=0.0143; N2-MPP+ODN1018 vs Neg. Contr. p=0.0027; other differences were not statistically significant (p>0.05). FIG. 8F depicts the results of the serological analysis of pre-challenge serum antibodies by ELISA against recombinant Kansas17 N2-VASP, after immunizing DBA.2 mice with N2-MPP alone or in combination with adjuvant ODN1018. FIG. 8G-8H depict weight loss curves (FIG. 8G) and percent survival (FIG. 8H) of BALB/c mice immunized with B-NA-MPP alone or a combination of B-NA-MPP and ODN1018 after challenge with 25×LD50 of B/New York/PV01181/2018. Differences in survival were analyzed using a Mantel-Cox log rank test: B-MPP+ODN1018 vs Neg. Contr. p=0.0035; other differences were not statistically significant (p>0.05). FIG. 8I depicts the results of the serological analysis of pre-challenge serum antibodies by ELISA against recombinant Colorado17-VASP, after immunizing BALB/c mice with B-NA-MPP alone or in combination with adjuvant ODN1018. For FIGS. 8A, 8C, 8D, 8F, 8G and 8I the mean plus standard deviation is shown.

FIGS. 9A-9F depict the results from the in vivo testing of a trivalent NA-MPP vaccination mix (N1-MPP, N2-MPP, and B-NA-MPP). Female, 6-8 week old BALB/c mice were vaccinated in a prime-boost regimen with respective protein(s) with or without ODN1018. Blood was obtained 42 days after the prime and used for serological analysis. FIG. 9A depicts weight loss curves and FIG. 9B depicts survival curves after challenge with 25×mLD₅₀ of A/Singapore/GP1908/2015 H1N1 (IVR-180). Differences in survival were analyzed using a Mantel-Cox log rank test: N1-MPP vs Neg. Ctrl. p=0.0143; all other vaccinated groups vs Neg. Ctrl. p=0.0027; other differences were not statistically significant (p>0.05). FIG. 9C depicts weight loss curves and FIG. 9D depicts survival curves after challenge with 5×mLD₅₀ of A/Vietnam/1203/04 H5N1 (6:2 PR8 reassortant, polybasic cleavage site removed). Differences in survival were analyzed using a Mantel-Cox log rank test: N1+N2+B-NA-MPP+ODN1018 vs. Neg. Ctrl. (p=0.0116) was statistically significant, the differences between remaining curves were not statistically significant (p>0.05). FIG. 9E depict the results of ELISA against recombinant Mich15 N1-VASP protein. FIG. 9F depicts the NI using the reassortant virus H7N1_{Mich15}. For FIGS. 9A, 9C, 9E and 9F the mean plus standard deviation is shown.

### 5. DETAILED DESCRIPTION

In one aspect, provided herein are immunogenic compositions comprising a CpG oligonucleotide adjuvant (e.g., as described herein Section 5.2 or Example 1) and a recombinant neuraminidase, which forms a stable tetramer. The immunogenic compositions may be used to immunize a subject against influenza virus.

### 5.1 Immunogenic Compositions

In a specific aspect, provided herein are compositions comprising a recombinant neuraminidase described herein (e.g., in Section 5.3 or 6) and a CpG oligonucleotide adjuvant described herein (e.g., in Section 5.2 or Example 1). In some embodiments, the composition comprises two or more (e.g., two or three) recombinant neuraminidases described herein. In specific embodiments, the composition further comprises a influenza vaccine (e.g., seasonal influenza vaccine), such as, e.g., described in Example 1. In some embodiments, the composition further comprises an aluminum salt (e.g., amorphous aluminum hydroxyphosphate sulfate, aluminum hydroxide, aluminum phosphate, or potassium aluminum sulfate, or a combination thereof). The composition may comprise about 0.25 to about 1.25 mg Al³⁺ or about 0.50 to about 1.00 mg Al³⁺. In some embodiments, the composition comprises about 0.375 mg Al³⁺, about 0.75 mg Al³⁺, or about 1.00 mg Al³⁺. In some embodiments, the immunogenic composition comprises about 0.375 mg Al³⁺. In some embodiments, the composition comprises about 0.05% to 3% (e.g., 1%, 2%, or 3%) of an aluminum salt. In some embodiments, the composition does not comprise an aluminum salt. In a specific embodiment, the composition is an immunogenic composition (e.g., a vaccine formulation). In certain embodiments, an immunogenic composition may comprise one or more other active ingredients (e.g., immunogenic agents). In specific embodiments, an immunogenic composition only comprises a recombinant neuraminidase described herein as the active ingredient. In specific embodiments, the compositions include a pharmaceutically acceptable carrier. The immunogenic compositions provided herein can be in any form that allows for the composition to be administered to a subject. In a specific embodiment, the immunogenic compositions are suitable for veterinary and/or human administration. The compositions may be used to induce an immune response (e.g., antibodies) against influenza virus neuraminidase (e.g., influenza A virus NA, influenza B virus NA, or both). The compositions may be used in methods of preventing an influenza virus disease in a subject (e.g., human). The compositions may be used in methods to induce an immune response against influenza virus. The compositions may be used in methods to immunize a subject (e.g., human) against influenza virus.

In a specific embodiment, an immunogenic composition comprises a recombinant neuraminidase described herein (e.g., in Section 5.3 or 6), and a CpG oligonucleotide adjuvant described herein (e.g., in Section 5.2 or Example 1). In a specific embodiment, an immunogenic composition comprises a CpG oligonucleotide adjuvant described herein (e.g., in Section 5.2 or Example 1) and a recombinant neuraminidase described herein (e.g., in Section 5.3 or 6), in an admixture with a pharmaceutically acceptable carrier. In some embodiments, the immunogenic composition further comprises an aluminum salt adjuvant. In some embodiments, the immunogenic composition does not comprise an aluminum salt adjuvant.

In another aspect, provided herein are compositions comprising a recombinant neuraminidase described herein. In another aspect, provided herein are compositions comprising a recombinant neuraminidase described herein (e.g., in Section 5.3 or 6), and a CpG oligonucleotide adjuvant described herein (e.g., in Section 5.2 or Example 1). In some embodiments, the compositions further comprises an aluminum salt adjuvant (e.g., aluminum hydroxide). In some embodiments, the compositions do not comprise an aluminum salt adjuvant. In some embodiments, the CpG oligonucleotide is the only adjuvant in the composition. In specific embodiments, the compositions further comprise a influenza vaccine (e.g., seasonal influenza vaccine), such as, e.g., described in Example 1. The influenza vaccine may be an inactivated or split virus vaccine. In a specific embodiment, the compositions are immunogenic compositions (e.g., vaccine formulations). The immunogenic compositions can be in any form that allows for the composition to be administered to a subject. In a specific embodiment, the immunogenic compositions are suitable for veterinary and/or human administration. The compositions may be used to induce an immune response (e.g., antibodies) against influenza virus neuraminidase (e.g., influenza A virus NA, influenza B virus NA, or both). The compositions may be used in methods of preventing an influenza virus disease. The compositions may be used in methods to induce an immune response against influenza virus. The compositions may be used in methods to immunize against influenza virus. In specific embodiments, the compositions may be used in combination with a CpG oligonucleotide adjuvant described herein (e.g., in Section 5.2 or Example 1) to prevent an influenza virus disease in a subject (e.g., human). In specific embodiments, the compositions may be used in combination with a CpG oligonucleotide adjuvant described herein (e.g., in Section 5.2 or Example 1) to induce an immune response against influenza virus. In specific embodiments, the compositions may be used in combination with a CpG oligonucleotide adjuvant described herein (e.g., in Section 5.2 or Example 1) to immunize a subject (e.g., human) against influenza virus.

In specific embodiments, immunogenic compositions described herein are monovalent formulations. In some embodiments, immunogenic compositions described herein are bivalent formulations. For example, in some embodiments, an immunogenic composition described herein comprises: (1) a recombinant neuraminidase comprising the globular head domain of an influenza A virus NI subtype and a tetramerization domain, and (2) a recombinant neuraminidase comprising the globular head domain of an influenza A virus N2 subtype and a tetramerization domain. In some embodiments, an immunogenic composition described herein comprises: (1) a recombinant neuraminidase comprising the globular head domain of an influenza A virus NI subtype and a tetramerization domain, and (2) a recombinant neuraminidase comprising the globular head domain of an influenza B virus NA and a tetramerization domain. In some embodiments, an immunogenic composition described herein comprises: (1) a recombinant neuraminidase comprising the globular head domain of an influenza A virus N2 subtype and a tetramerization domain, and (2) a recombinant neuraminidase comprising the globular head domain of an influenza B virus NA and a tetramerization domain. In some embodiments, immunogenic compositions described herein are multivalent formulations. For example, an immunogenic composition described herein may comprise 3, 4, 5, or more recombinant neuraminidases described herein. In some embodiments, an immunogenic composition described herein comprises: (1) a recombinant neuraminidase comprising the globular head domain of an influenza A virus N1 subtype and a tetramerization domain, (2) a recombinant neuraminidase comprising the globular head domain of an influenza A virus N2 subtype and a tetramerization domain; and (3) a recombinant neuraminidase comprising the globular head domain of an influenza B virus NA and a tetramerization domain In one example, a multivalent formulation comprises more than three influenza virus comprising a recombinant neuraminidase described herein. For example, a multivalent formulation may be quadrivalent.

In specific embodiments, an immunogenic composition described herein comprises a recombinant neuraminidase described in Example 1. In specific embodiments, an immunogenic composition described herein comprises two recombinant neuraminidases described in Example 1. In specific embodiments, an immunogenic composition described herein comprises three recombinant neuraminidases described in Example 1.

In a specific embodiment, provided herein is an immunogenic composition comprising a recombinant neuraminidase described herein (e.g., in Section 5.3 or 6), a CpG oligonucleotide adjuvant described herein (e.g., in Section 5.2 or Example 1), and a seasonal influenza vaccine, such as described in Example 1. In a specific embodiment, provided herein is an immunogenic composition comprising two recombinant neuraminidases described herein (e.g., in Section 5.3 or 6), a CpG oligonucleotide adjuvant described herein (e.g., in Section 5.2 or Example 1), and a seasonal influenza vaccine, such as described in Example 1. In a specific embodiment, provided herein is an immunogenic composition comprising three recombinant neuraminidases described herein (e.g., in Section 5.3 or 6), a CpG oligonucleotide adjuvant described herein (e.g., in Section 5.2 or Example 1), and a seasonal influenza vaccine, such as described in Example 1. In some embodiments, such an immunogenic composition further comprises an aluminum salt adjuvant. In some embodiments, such an immunogenic composition does not comprise an aluminum salt adjuvant. In some embodiments, the CpG oligonucleotide adjuvant is the only adjuvant in the immunogenic composition.

In a specific embodiment, provided herein is an immunogenic composition comprising a recombinant neuraminidase described herein (e.g., in Section 5.3 or 6), a CpG oligonucleotide adjuvant described herein (e.g., in Section 5.2 or Example 1), and a seasonal influenza vaccine, such as described in Example 1, in an admixture with a pharmaceutically acceptable carrier. In a specific embodiment, provided herein is an immunogenic composition comprising two recombinant neuraminidases described herein (e.g., in Section 5.3 or 6), a CpG oligonucleotide adjuvant described herein (e.g., in Section 5.2 or Example 1), and a seasonal influenza vaccine, such as described in Example 1, in an admixture with a pharmaceutically acceptable carrier. In a specific embodiment, provided herein is an immunogenic composition comprising three recombinant neuraminidases described herein (e.g., in Section 5.3 or 6), a CpG oligonucleotide adjuvant described herein (e.g., in Section 5.2 or Example 1), and a seasonal influenza vaccine, such as described in Example 1, in an admixture with a pharmaceutically acceptable carrier. In some embodiments, such an immunogenic composition further comprises an aluminum salt adjuvant. In some embodiments, such an immunogenic composition does not comprise an aluminum salt adjuvant. In some embodiments, the CpG oligonucleotide adjuvant is the only adjuvant in the immunogenic composition.

In specific embodiments, the influenza vaccine (e.g., seasonal influenza vaccine) comprises influenza virus hemagglutinin (HA) or a fragment thereof (e.g., at least 10, at least 20, at least 30, at least 40, at least 50, at least 75, at least 100 contiguous amino acid residues of HA). In some embodiments, the influenza vaccine (e.g., seasonal influenza vaccine) is an inactivated influenza vaccine. In some embodiments, the influenza vaccine (e.g., seasonal influenza vaccine) is a split virus vaccine. In some embodiments, the influenza vaccine (e.g., seasonal influenza vaccine) is a trivalent flu vaccine. In some embodiments, the influenza vaccine (e.g., seasonal influenza vaccine) is a quadrivalent flu vaccine. For example, the influenza vaccine may be AFLURIA Quadrivalent, Fluarix Quadrivalent, FluLaval Quadrivalent, Flucelvax Quadrivalent and Fluzone Quadrivalent, or FluMist Quadrivalent. The influenza vaccine (e.g., a seasonal influenza vaccine) may be an FDA or EMA approved influenza vaccine.

In some embodiments, an immunogenic composition described herein comprises from about 5 μg to about 150 μg of a recombinant neuraminidase described herein (e.g., in Section 5.3 or Example 1). In some embodiments, an immunogenic composition described herein comprises from about 5 μg to about 50 μg of a recombinant neuraminidase described herein (e.g., in Section 5.3 or Example 1). In some embodiments, an immunogenic composition described herein comprises from about 15 μg to about 45 μg of a recombinant neuraminidase described herein (e.g., in Section 5.3 or Example 1). In some embodiments, an immunogenic composition described herein comprises about 5 μg, about 10 μg, about 15 μg, about 20 μg, about 25 μg, about 30 μg, about 35 μg, about 40 μg, about 45 μg, or about 50 μg of a recombinant neuraminidase described herein (e.g., in Section 5.3 or Example 1). In some embodiments, an immunogenic composition comprises about 5 μg of a recombinant neuraminidase described herein. In some embodiments, an immunogenic composition comprises about 15 μg of a recombinant neuraminidase described herein. In some embodiments, an immunogenic composition comprises about 30 μg of a recombinant neuraminidase described herein. In some embodiments, an immunogenic composition comprises about 45 μg a recombinant neuraminidase described herein. In other embodiments, the immunogenic composition comprises about 75 μg, about 100 μg, about 125 μg or about 150 μg of a recombinant neuraminidase described herein.

In some embodiments, an immunogenic composition described herein comprises from about 5 μg to about 150 μg of recombinant neuraminidases described herein (e.g., in Section 5.3 or Example 1). In other embodiments, the immunogenic composition comprises about 75 μg, about 100 μg, about 125 μg or about 150 μg of recombinant neuraminidases described herein. In some embodiments, an immunogenic composition described herein comprises from about 5 μg to about 50 μg of each recombinant neuraminidase described herein (e.g., in Section 5.3 or Example 1) included in the composition. In some embodiments, an immunogenic composition described herein comprises from about 15 μg to about 45 μg of each recombinant neuraminidase described herein (e.g., in Section 5.3 or Example 1) included in the composition. In some embodiments, an immunogenic composition described herein comprises about 5 μg, about 10 μg, about 15 μg, about 20 μg, about 25 μg, about 30 μg, about 35 μg, about 40 μg, about 45 μg, or about 50 μg of each recombinant neuraminidase described herein (e.g., in Section 5.3 or Example 1) included in the composition. In some embodiments, an immunogenic composition comprises about 5 μg of each recombinant neuraminidase described herein included in the composition. In some embodiments, an immunogenic composition comprises about 15 μg of each recombinant neuraminidase described herein included in the composition. In some embodiments, an immunogenic composition comprises about 30 μg of each recombinant neuraminidase described herein included in the composition. In some embodiments, an immunogenic composition comprises about 45 μg of each recombinant neuraminidase described herein included in the composition.

In some embodiments, an immunogenic composition described herein comprises from about 375 μg to about 6000 μg of a CpG oligonucleotide of a CpG oligonucleotide adjuvant described herein (e.g., in Section 5.2 or Example 1), preferably from about 500 μg to about 5000 μg of the CpG oligonucleotide, preferably from about 750 μg to about 3000 μg of the CpG oligonucleotide. In some embodiments, an immunogenic composition described herein comprises greater than about 250 μg, about 500 μg, about 750 μg, about 1000 μg, or about 1250 μg of the CpG oligonucleotide of a CpG oligonucleotide adjuvant described herein (e.g., in Section 5.2 or Example 1), and less than about 6000 μg, about 5000 μg, about 4000 μg, about 3000 μg, or about 2000 μg of the CpG oligonucleotide. In some embodiments, an immunogenic composition described herein comprises about 375 μg, about 750 μg, about 1500 μg, about 3000 μg or about 6000 μg of the CpG oligonucleotide of a CpG oligonucleotide adjuvant. In some embodiments, an immunogenic composition comprises about 750 μg of the CpG oligonucleotide adjuvant described herein (e.g., in Section 5.2 or Example 1). In some embodiments, an immunogenic composition described herein comprises about 1500 μg of the CpG oligonucleotide of a CpG oligonucleotide adjuvant described herein (e.g., in Section 5.2 or Example 1). In some embodiments, an immunogenic composition described herein comprises about 3000 μg of the CpG oligonucleotide of a CpG oligonucleotide adjuvant described herein (e.g., in Section 5.2 or Example 1). In some embodiments, an immunogenic composition described herein comprises about 6000 μg of the CpG oligonucleotide of a CpG oligonucleotide adjuvant described herein (e.g., in Section 5.2 or Example 1).

In some embodiments, the aluminum salt adjuvant comprises one or more selected from the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant consists of aluminum hydroxide. In some embodiments, an immunogenic composition described herein comprises from about 0.25 to about 1.25 mg Al³⁺. In some embodiments, an immunogenic composition comprises from about 0.50 to about 1.00 mg Al³⁺. In some embodiments, the immunogenic composition comprises about 0.375 mg Al³⁺, about 0.75 mg Al³⁺, or about 1.00 mg Al³⁺. In some embodiments, the immunogenic composition comprises about 0.375 mg Al³⁺.

In some embodiments, an immunogenic composition described herein comprises about 0.05% to about 3%, or about 1% to about 3% of aluminum salt. In some embodiments, an immunogenic composition described herein comprises about 1%, about 1.5%, about 2%, about 2.5%, or about 3% of aluminum salt. In some embodiments, an immunogenic composition described herein comprises about 1% or about 2% of an aluminum salt. In some embodiments, the aluminum salt comprises one or more selected from the group consisting of amorphous aluminum hydroxyphosphate sulfate, aluminum hydroxide, aluminum phosphate, and potassium aluminum sulfate. In some embodiments, the aluminum salt comprises one or both of aluminum hydroxide and aluminum phosphate. In some embodiments, the aluminum salt is aluminum hydroxide. In some embodiments, the immunogenic composition described herein does not comprise an aluminum salt.

In some embodiments, an immunogenic composition described herein has a volume of about 0.5 mL to about 2 mL. In some embodiments, an immunogenic composition described herein has a volume of about 0.5 mL, about 1 mL, about 1.5 mL, or about 2 mL.

As used herein, the term “pharmaceutically acceptable” means approved by a regulatory agency of the Federal or a state government or listed in the U.S. Pharmacopeia or other generally recognized pharmacopoeia for use in animals, and more particularly in humans. The term “carrier” refers to a diluent, adjuvant, excipient, or vehicle with which the pharmaceutical composition is administered. In specific embodiments, the pharmaceutically acceptable carrier is a diluent, excipient, or vehicle. For example, the pharmaceutically acceptable carrier is a diluent (e.g., saline). Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, an immunogenic compositions described herein are formulated to be suitable for the intended route of administration to a subject. For example, an immunogenic composition may be formulated to be suitable for parenteral, oral, intradermal, transdermal, or intraperitoneal administration. In a specific embodiment, an immunogenic composition may be formulated for intravenous, oral, intraperitoneal, intranasal, intratracheal, subcutaneous, intramuscular, topical, intradermal, transdermal, or pulmonary administration. In a specific embodiment, an immunogenic composition may be formulated for intramuscular administration. In another specific embodiment, an immunogenic composition may be formulated for subcutaneous administration. In another specific embodiment, an immunogenic composition may be formulated for intranasal administration.

An immunogenic composition described herein may be used to immunize a subject (e.g., human subject) against influenza virus. An immunogenic composition described herein may also be used to prevent an influenza virus disease in a subject (e.g., human subject). In a specific embodiment, an immunogenic composition described herein may be used in a method described herein.

The immunogenic compositions described herein can be included in a container, pack, or dispenser together with instructions for administration.

In a specific embodiment, provided herein is a composition comprising an antibody(ies) that binds to influenza virus neuraminidase, which was generated using an immunogenic composition described herein. The composition may further comprise a pharmaceutically acceptable carrier. In some embodiments, the antibody is human. In some embodiments, the antibody is humanized. The antibody(ies) may be used to passively immunize a subject (e.g., a human subject).

### 5.2 CpG Oligonucleotide Adjuvants

Toll-like receptors are expressed in and on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010). Based on structure and biological function, CpG-ODNs have been divided into three general classes: CpG-A, CpG-B, and CpG-C(Campbell, Methods Mol Biol, 1494:15-27, 2017). The degree of B cell activation varies between the classes with CpG-A ODNs being weak, CpG-C ODNs being good, and CpG-B ODNs being strong B cell activators.

In specific embodiments, the compositions described herein comprise, or are administered in combination with, a CpG oligonucleotide adjuvant. The CpG oligonucleotide adjuvants of the present disclosure are TLR9 agonists. In specific embodiments, CpG oligonucleotide adjuvants of the present disclosure are good B cell activators (CpG-C ODN) or more preferably strong (CpG-B ODN) B cell activators.

Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, a CpG oligonucleotide adjuvant comprises an oligonucleotide of from 8 to 35 nucleotides in length comprising the sequence 5′-AACGTTCG-3′ (SEQ ID NO:1). In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, a CpG oligonucleotide adjuvant comprises an oligonucleotide of from 10 to 35 nucleotides in length comprising the sequence 5′-AACGTTCGAG-3′ (SEQ ID NO:2). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, a CpG oligonucleotide adjuvant comprises an oligonucleotide of from 10 to 35 nucleotides in length comprising the sequence 5′-GAACGTTCG-3′ (SEQ ID NO:3). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

Researchers at Dynavax Technologies Corporation (Emeryville, CA) have identified a 22-mer phosphorothioate linked oligodeoxynucleotide, CpG 1018® adjuvant, which contains specific sequences that can substantially enhance the immune response to co-administered antigens across species [17]. CpG 1018® adjuvant has a nucleotide sequence of 5′-TGACTGTGAACGTTCGAGATGA-3′ (SEQ ID NO:4) and was chosen after screening a broad panel of oligonucleotides for immunostimulatory activity in vitro and in vivo. CpG 1018® adjuvant is a CpG-B ODN that is active in mice, rabbits, dogs, baboons, cynomolgus monkeys, and humans. CpG 1018® adjuvant is also referred to by the term ODN 1018. In specific embodiments, a CpG oligonucleotide adjuvant (e.g., as described herein Section 5.2 or Example 1) comprises an oligonucleotide comprising the sequence of SEQ ID NO:4. In some embodiments, a CpG oligonucleotide adjuvant comprises an oligonucleotide of from 22 to 35 nucleotides in length comprising the sequence of SEQ ID NO:4. In some embodiments, the oligonucleotide is greater than 22, 23, 24, 25, 26, 27, 28, 29, 30 or 31 nucleotides in length, and the oligonucleotide is less than 35, 34, 33, 32, 31, 30, 29, 28, 27, 26, 25, or 24 nucleotides in length.

Although the exemplary CpG oligonucleotide adjuvant, CpG 1018®, is a CpG-ODN, the present disclosure is not restricted to fully DNA molecules. That is, in some embodiments, the oligonucleotide of the CpG oligonucleotide adjuvant is a DNA/RNA chimeric molecule in which the CpG(s) and the palindromic sequence are deoxyribonucleic acids and one or more nucleic acids outside of these regions are ribonucleic acids. In some embodiments, the oligonucleotide of the CpG oligonucleotide adjuvant is linear. In other embodiments, the oligonucleotide of the CpG oligonucleotide adjuvant is circular or includes hairpin loop(s). The oligonucleotide of the CpG oligonucleotide adjuvant may be single stranded. Alternatively, the oligonucleotide of the CpG oligonucleotide adjuvant may be double stranded.

In some embodiments, the CpG oligonucleotide of the CpG oligonucleotide adjuvant may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′ substituted-arabinoguanosine, and 2′-O-substituted-arabinoguanosine.

The CpG oligonucleotide of the CpG oligonucleotide adjuvant may contain a modification of the phosphate group. For example, in addition to phosphodiester linkages, phosphate modifications include, but are not limited to, phosphorothioate (e.g., phosphoromonothioate), phosphorodithioate, methyl phosphonate, phosphoramidate (bridging or non-bridging), and phosphotriester, and may be used in any combination. Other non-phosphate oligomer linkages may also be used. In some embodiments, the oligonucleotides comprise only phosphorothioate backbones. In some embodiments, the oligonucleotides comprise only phosphorodithioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. In some embodiments, the oligonucleotide comprises a combination of phosphate linkages in the phosphate backbone such as a combination of phosphodiester, phosphorothioate, and phosphorodithioate linkages. Oligonucleotides with thioated phosphate (e.g., phosphorothioate) backbones can be more immunogenic than those with phosphodiester backbones and appear to be more resistant to degradation after injection into the host [18, 19]. The CpG oligonucleotide of an CpG oligonucleotide adjuvant of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

In some embodiments, the CpG oligonucleotide of the CpG oligonucleotide adjuvant is produced synthetically, or is an isolated natural product or fragment thereof. In some embodiments, a CpG oligonucleotide is synthesized using H-phosphonate, phosphotriester, phosphodiester, phosphite-triester, and/or phosphoramidite chemistry. In some embodiments, a the CpG oligonucleotide of the CpG oligonucleotide adjuvant is synthesized using a solid support, optionally controlled pore glass. In some embodiments, a CpG oligonucleotide is thioated using environmental sulfur (S8), Beaucage reagent, a thioazoline derivative (i.e., DtsNH, EDITH, MEDITH, ADTT, DDTT), or sulfurizing reagent II (Glen Research). In some embodiments, the CpG oligonucleotide of the CpG oligonucleotide adjuvant is purified using column chromatography (e.g., HPLC).

The CpG oligonucleotides are in their pharmaceutically acceptable salt form unless otherwise indicated. In specific embodiments, a CpG oligonucleotide adjuvant described herein comprises a pharmaceutically acceptable salt form. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy) propyl]-N,N,N-trimethylammonium chloride, choline, tromethamine, dicyclohexylamines, t-butyl amines, and salts with amino acids such as arginine, lysine and the like. In some embodiment, the CpG oligonucleotides are in the ammonium, sodium, lithium, or potassium salt form. In one preferred embodiment, the CpG oligonucleotides are in the sodium salt form. In a specific embodiment, a CpG oligonucleotide adjuvant described herein comprises an aluminum salt. In some embodiments, the aluminum salt comprises one or more selected from the group consisting of amorphous aluminum hydroxyphosphate sulfate, aluminum hydroxide, aluminum phosphate, and potassium aluminum sulfate. The aluminum salt may be aluminum hydroxide or aluminum phosphate. In a specific embodiment, the aluminum salt comprises aluminum hydroxide and aluminum phosphate. In a specific embodiment, the aluminum salt comprises aluminum phosphate. In a specific embodiment, the aluminum salt is aluminum hydroxide. In some embodiments, the CpG oligonucleotide adjuvant described herein does not comprise an aluminum salt.

In a specific embodiment, an adjuvant comprises a CpG oligonucleotide adjuvant described herein. In a specific embodiment, an adjuvant comprises CpG 1018®. In a specific embodiment, a CpG oligonucleotide adjuvant is one described in Section 6, infra.

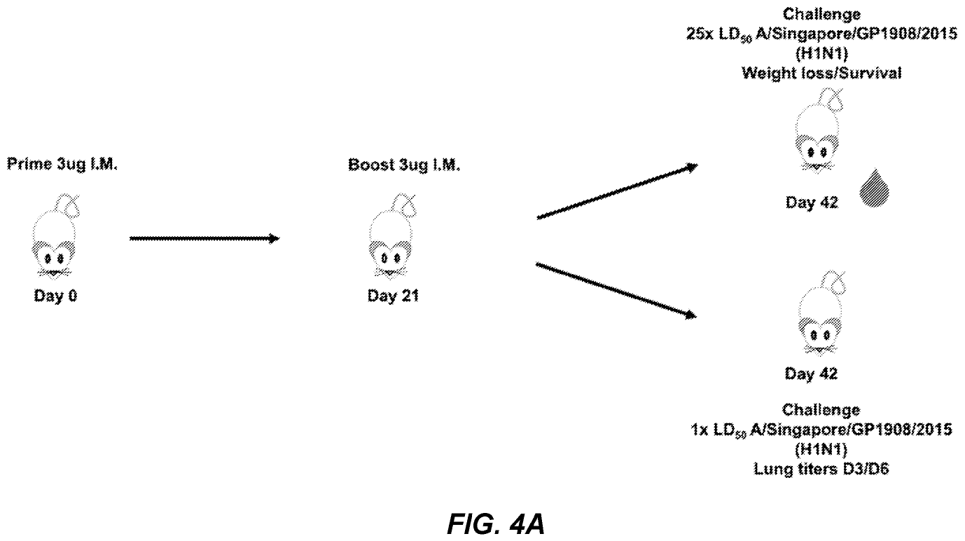

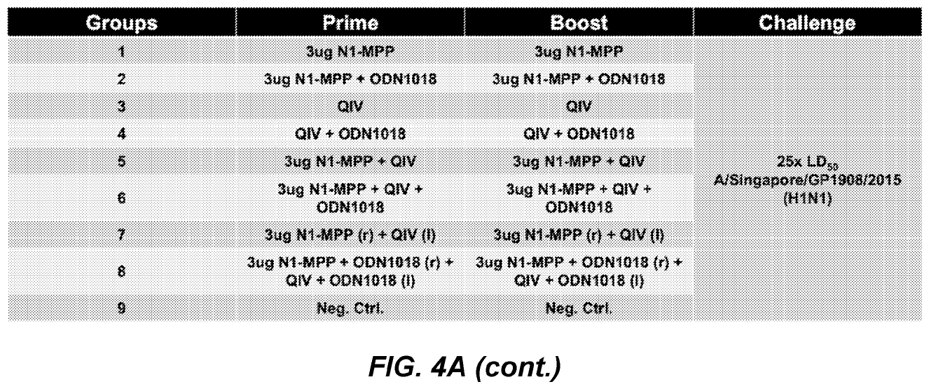

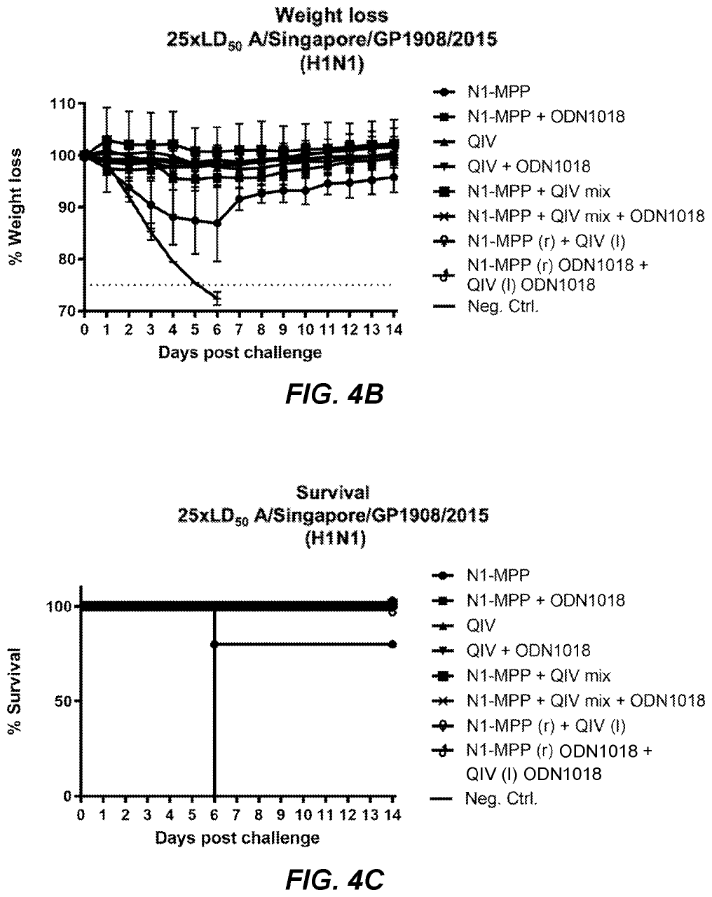

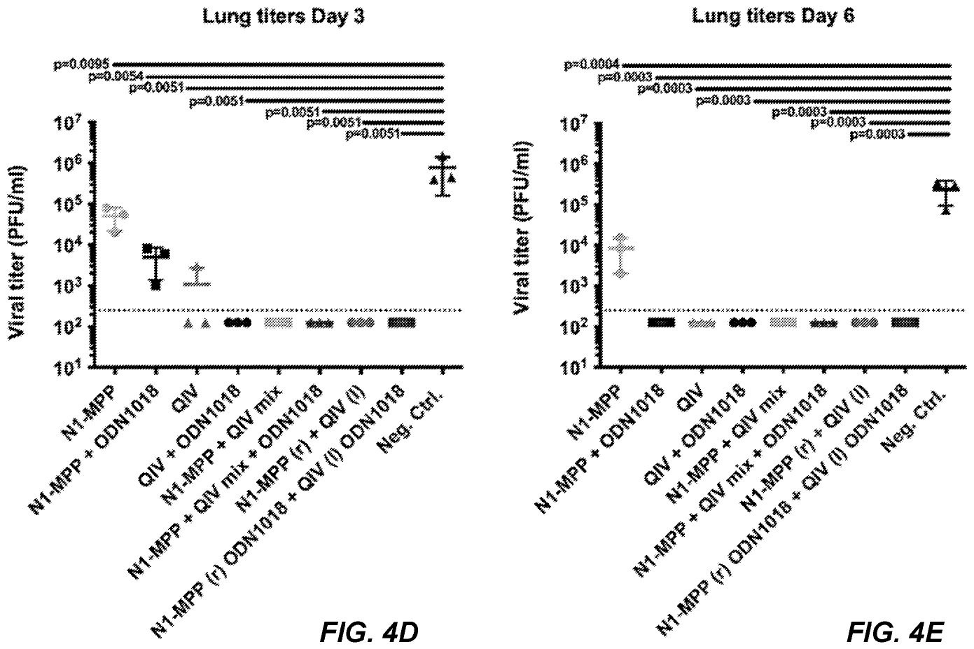

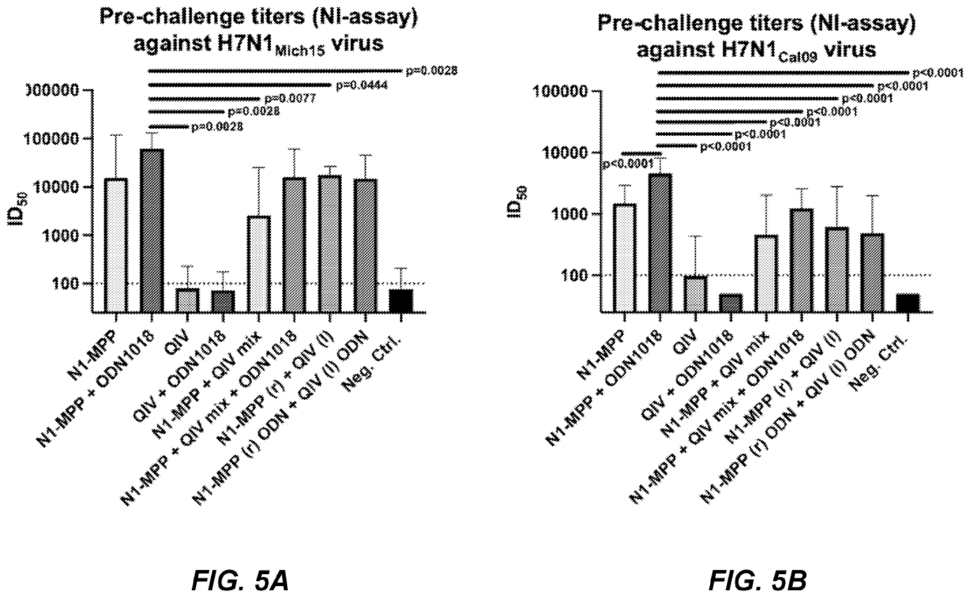

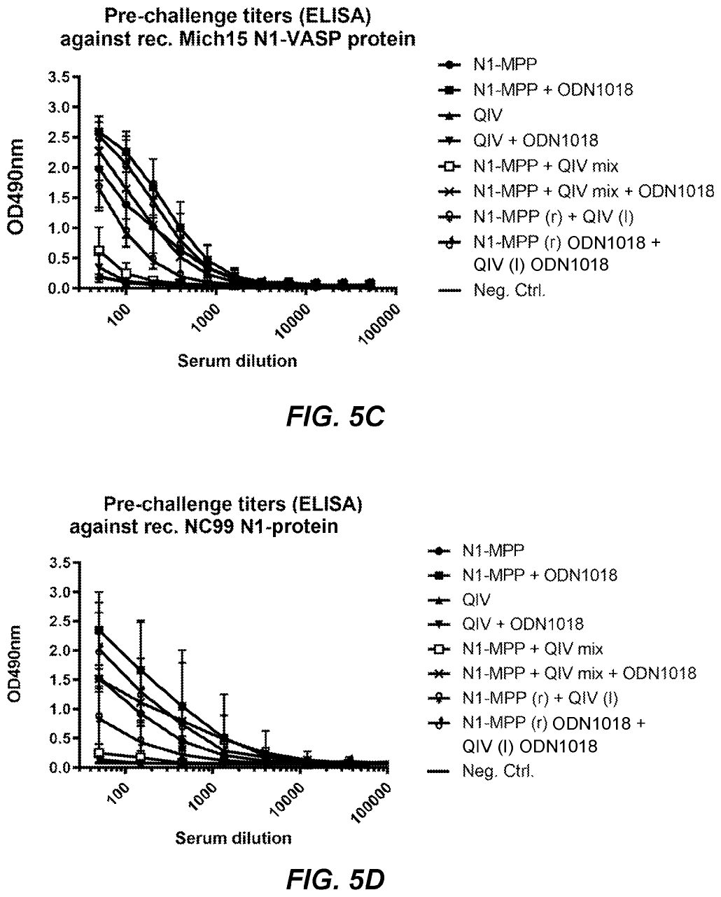

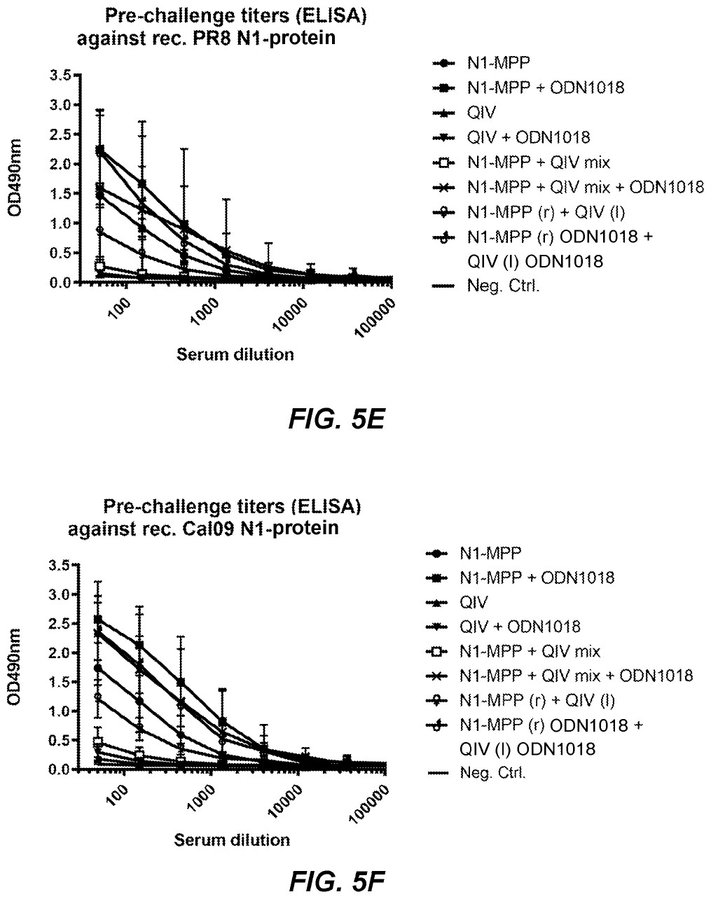

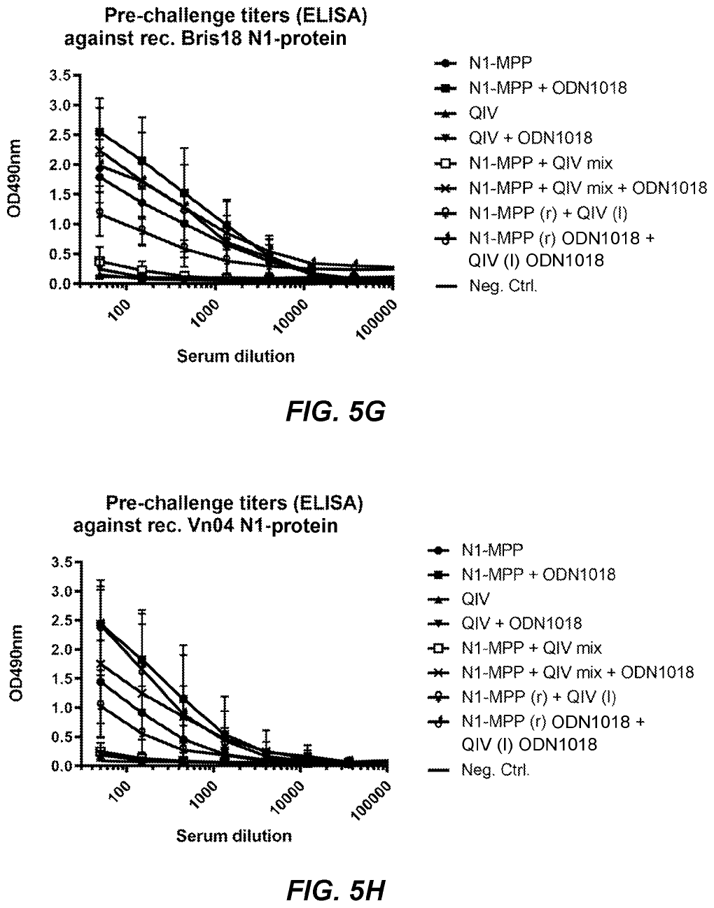

In some embodiments, a CpG oligonucleotide adjuvant described herein (e.g., in Section 5.2 or Example 1) enhances or boosts an immune response to influenza virus and does not produce an allergy or other adverse reaction. For example, the CpG oligonucleotide adjuvant can enhance an immune response by several mechanisms including, e.g., lymphocyte recruitment, stimulation of B and/or T cells, and stimulation of macrophages. In certain embodiments, a CpG oligonucleotide adjuvant described herein (e.g., in Section 5.2 or Example 1) augments the intrinsic response to a recombinant neuraminidase without causing conformational changes in the recombinant neuraminidase that affect the qualitative form of the response. In specific embodiments, the presence of a CpG oligonucleotide adjuvant described herein (e.g., in Section 5.2 or Example 1) in an immunogenic composition comprising a recombinant neuraminidase described herein and an influenza vaccine at least partially breaks the immunodominance of influenza virus hemagglutinin (HA) in the influenza vaccine (e.g., a seasonal influenza vaccine) over influenza neuraminidase. See Example 1 (e.g., FIGS. 4A-4E and FIGS. 5A-5H).

### 5.3 Recombinant Influenza Virus Neuraminidase

Provided herein are recombinant influenza virus neuraminidases (NA), which may be used as immunogens in immunogenic compositions. In a specific embodiment, a recombinant neuraminidase comprises the amino acid sequence of a neuraminidase described in Section 5.3.1 or Section 6, infra. In another specific embodiment, a recombinant neuraminidase comprises the amino acid sequence of a neuraminidase described in Section 5.3.1, infra, without one, two or more of the following: the signal sequence, purification tag, or cleavage site.

The neuraminidase (NA) of influenza viruses is a homo-tetrameric, type II transmembrane protein [20-21]. The tetrameric form of the NA has the enzymatic function of removing sialic acids from mucus, which is crucial to prevent immobilization of the virus in the respiratory tract, and to release virus progenies from infected cells [22]. Four identical monomeric polypeptides each approximately 470 amino acids assemble to form tetrameric NA [23]. The four monomers each have four distinct structural domains: the cytoplasmic tail, the transmembrane region, the stalk, and the catalytic head (id.). The ectodomain of NA is composed of the globular head domain and stalk domain of NA.

In certain embodiments, a recombinant neuraminidase described herein retains one, two, or more, or all of the functions of a wild-type influenza virus neuraminidase. In a specific embodiment, a recombinant neuraminidase described herein cleaves sialic acid. Assays known to one skilled in the art can be utilized to assess the ability of a recombinant neuraminidase to cleave sialic acid. See Section 5.7 as well as Section 6 for examples of assays to assess the functional activity of such a recombinant neuraminidase.

It will be understood by those of skill in the art that a recombinant neuraminidase provided herein can be prepared according to any technique known by and deemed suitable to those of skill in the art, including the techniques described herein. In certain embodiments a recombinant neuraminidase described herein is isolated.

5.3.1 Recombinant Neuraminidase with Tetramerization Domain

In one aspect, provided herein are recombinant neuraminidases comprising an influenza virus neuraminidase globular head domain and a tetramerization domain. In a specific embodiment, provided herein is a recombinant neuraminidase comprising an influenza virus neuraminidase globular head domain and a tetramerization domain, wherein the recombinant neuraminidase lacks influenza virus neuraminidase transmembrane and cytoplasmic domains. In specific embodiments, the recombinant neuraminidase does not have any transmembrane domain. In certain embodiments, the recombinant neuraminidase includes a stalk domain or a fragment thereof of the same influenza virus neuraminidase as the globular head domain. The fragment of the stalk domain of the influenza virus neuraminidase may consist of or comprise 2, 3, 4, 5, 6, 7, 8, 9, 10, 11, 12, 13, 14, 15, 16, 17, 18, 19, 20, 25, 30, 35, 40, 45, or 50 amino acid residues of the stalk domain of the influenza virus neuraminidase. Alternatively, the fragment of the stalk domain of the influenza virus neuraminidase may consist of or comprise 2 to 5, 5 to 10, 5 to 15, 10 to 15, 5 to 20, 10 to 20, 15 to 20, 20 to 30, 20 to 40, 25 to 30, 25 to 40, 25 to 45, 25 to 50, 30 to 40, or 40 to 50 amino acid residues of the stalk domain of the influenza virus neuraminidase. In specific embodiments, the recombinant neuraminidase does not include the stalk domain of an influenza virus neuraminidase.

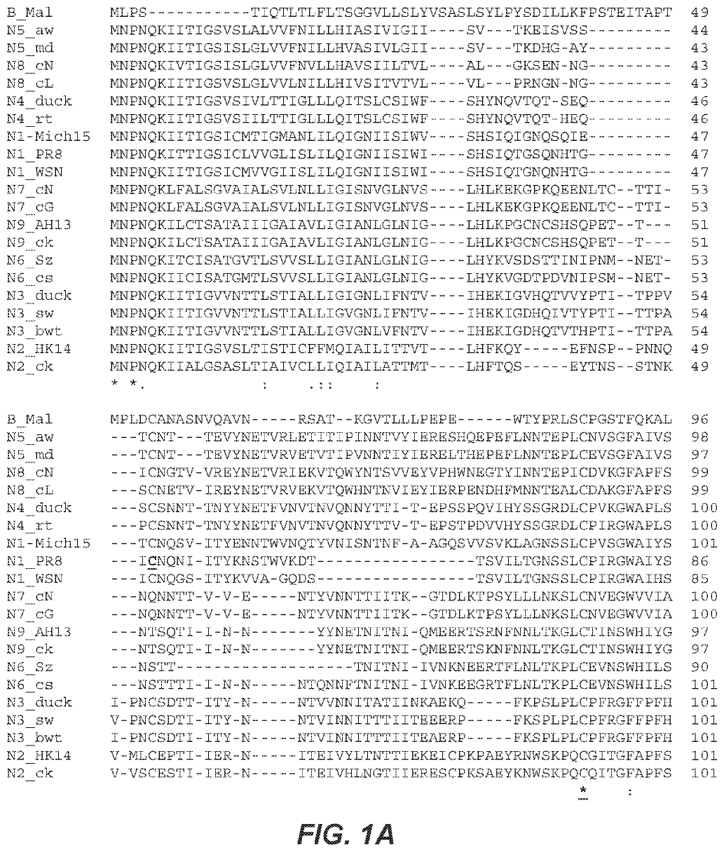

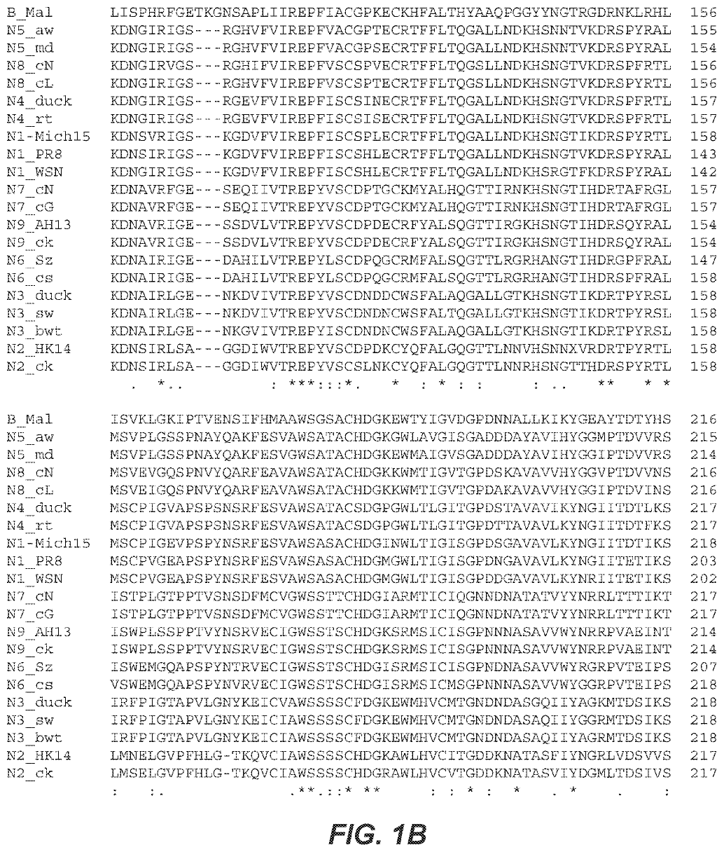

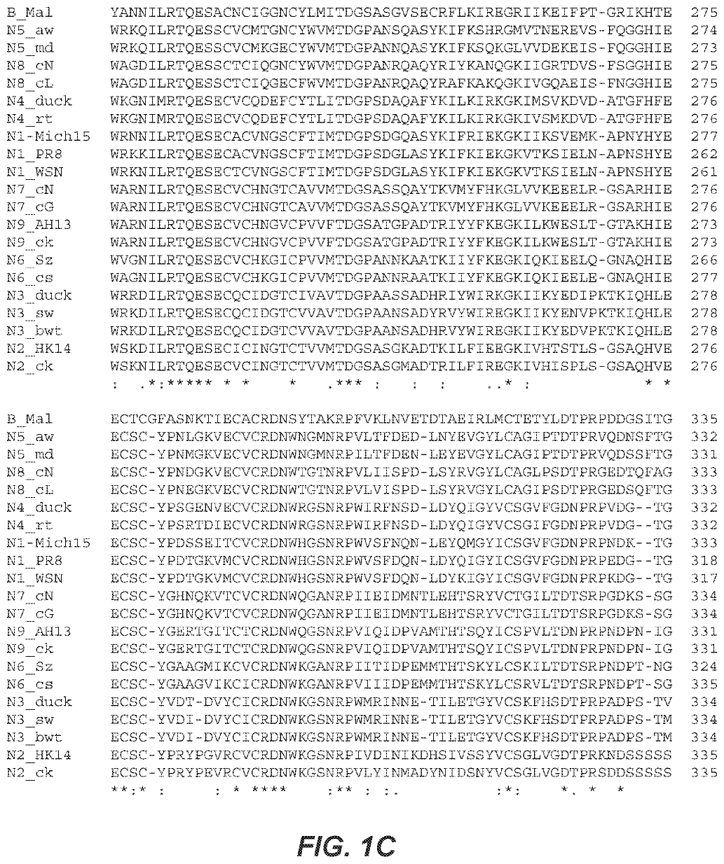

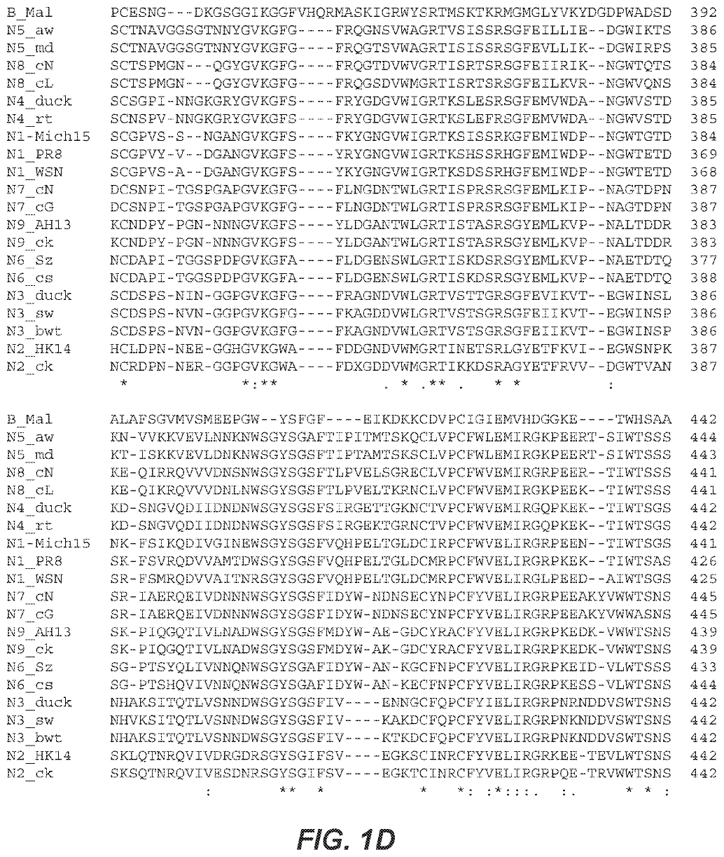

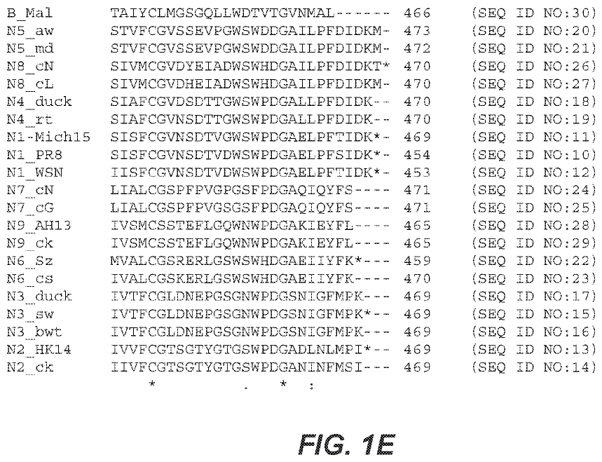

Those of skill in the art will recognize that the delineation of the domains of an influenza virus neuraminidase may be determined from, e.g., crystal structure and/or by using structure prediction software (for example, the website for the Center for Biological Sequence Analysis, Technical University of Denmark DTU, or Pymol) in conjunction with protein alignments. In a specific embodiment, the first cysteine of the globular head domain of influenza virus neuraminidase corresponds to the amino acid residue indicated by the bold and underlined asterisk in FIGS. 1A-1D.

In some embodiments, provided herein is a recombinant neuraminidase comprising an influenza virus neuraminidase globular head domain and a tetramerization domain. In a specific embodiment, provided herein is a recombinant neuraminidase comprising an influenza virus neuraminidase globular head domain and a tetramerization domain, wherein the recombinant neuraminidase lacks influenza virus neuraminidase transmembrane and cytoplasmic domains. In specific embodiments, the recombinant neuraminidase does not have any transmembrane domain. In some embodiments, the recombinant neuraminidase includes a stalk domain or a fragment thereof of the same influenza virus neuraminidase as the globular head domain. The fragment of the stalk domain of the influenza virus neuraminidase may consist of or comprise 2, 3, 4, 5, 6, 7, 8, 9, 10, 11, 12, 13, 14, 15, 16, 17, 18, 19, 20, 25, 30, 35, 40, 45, or 50 amino acid residues of the stalk domain of the influenza virus neuraminidase. Alternatively, the fragment of the stalk domain of the influenza virus neuraminidase may consist of or comprise 2 to 5, 5 to 10, 5 to 15, 10 to 15, 5 to 20, 10 to 20, 15 to 20, 20 to 30, 20 to 40, 25 to 30, 25 to 40, 25 to 45, 25 to 50, 30 to 40, or 40 to 50 amino acid residues of the stalk domain of the influenza virus neuraminidase. In specific embodiments, the recombinant neuraminidase does not include the stalk domain of an influenza virus neuraminidase. In specific embodiments, the recombinant neuraminidase does not include any cytoplasmic domain of an influenza virus neuraminidase.

In a specific embodiment, provided herein is a recombinant neuraminidase comprising an influenza virus neuraminidase globular head domain and a tetramerization domain, wherein the recombinant neuraminidase lacks influenza virus neuraminidase transmembrane and cytoplasmic domains. In some embodiments, the recombinant neuraminidase includes a stalk domain or a fragment thereof of the same influenza virus neuraminidase as the globular head domain. The fragment of the stalk domain of the influenza virus neuraminidase may consist of or comprise 2, 3, 4, 5, 6, 7, 8, 9, 10, 11, 12, 13, 14, 15, 16, 17, 18, 19, 20, 25, 30, 35, 40, 45, or 50 amino acid residues of the stalk domain of the influenza virus neuraminidase. Alternatively, the fragment of the stalk domain of the influenza virus neuraminidase may consist of or comprise 2 to 5, 5 to 10, 5 to 15, 10 to 15, 5 to 20, 10 to 20, 15 to 20, 20 to 30, 20 to 40, 25 to 30, 25 to 40, 25 to 45, 25 to 50, 30 to 40, or 40 to 50 amino acid residues of the stalk domain of the influenza virus neuraminidase. In specific embodiments, the recombinant neuraminidase does not include the stalk domain of an influenza virus neuraminidase. In a specific embodiment, provided herein is a recombinant neuraminidase comprising an influenza virus neuraminidase globular head domain and a paramyxovirus phosphoprotein tetramerization domain, wherein the recombinant neuraminidase lacks influenza virus neuraminidase stalk, transmembrane and cytoplasmic domains.

In some embodiments, the recombinant neuraminidase comprises a tetramerization domain from SEPPALLATA-like MADS domain transcription factor from Arabidopsis thaliana (SMDTF), PiLZ structure from Xanthomonas campestris, or Dictyocaulus viviparus ACE tetramerization domain. In specific embodiments, the recombinant neuraminidase comprises a tetramerization domain from a paramyxovirus phosphoprotein (e.g., a measles virus phosphoprotein, a Nipah virus phosphoprotein, a Hendra virus phosphoprotein, a respiratory syncytial virus phosphoprotein, human parainfluenza virus (hPIV) phosphoprotein, bovine parainfluenza virus phosphoprotein, a mumps virus phosphoprotein, a Cedar virus phosphoprotein, a Ghana virus phosphoprotein, a Newcastle disease virus phosphoprotein, a canine distemper virus phosphoprotein, or a Peste des petits ruminants virus (PPRV) phosphoprotein). In certain embodiments, the recombinant neuraminidase does not comprise a GCN4 leucine zipper, a bacterial tetrabrachion tetramerization domain or the human vasodilator stimulated phosphoprotein (VASP) tetramerization domain. In a specific embodiment, the recombinant neuraminidase comprises a measles virus phosphoprotein tetramerization domain. In a specific embodiment, the recombinant neuraminidase comprises a tetramerization domain described in Example 1.

TABLE 1
Exemplary Sequence Information.

| SEQ ID | | |
|---|---|---|
| NO. | NAME | SEQUENCE |
| 1 | CpG oligonucleotide | AACGTTCG |
| 2 | CpG oligonucleotide | AACGTTCGAG |
| 3 | CpG oligonucleotide | GAACGTTCG |
| 4 | CpG oligonucleotide | TGACTGTGAACGTTCGAGATGA |
| 5 | Tetramerization | VELSSQQEYLKLKERYDALQRTQRNLLGEDLGPLSTK |
| | domain from | ELESLERQLDSSLKQIRALRTQFMLDQSKERMLTETN |
| | SEPPALLATA-like | KTLRLRLADGY |
| | MADS domain | |
| | transcription factor | |
| | from Arabidopsis | |
| | thaliana (SMDTF) | |
| 6 | Sendai virus | ENTSSMKEMATLLTSLGVIQSAQEFESSRDASYVFAR |
| | phosphoprotein | RALKSANYAEMTFNVCGLILSAEKSSARKVDENKQL |
| | tetramerization | LKIQESVESFRDIYKRFSEYQKEQNSLLMSNLSTLHIIT |
| | domain (SPP) | D |
| 7 | PiLZ structure from | LLVQRMDAKLDLILALIGRLVRQS |
| | Xanthomonas | |
| | campestris | |
| 8 | Measles virus | GDHYDDELFSDVQDIKTALAKIHEDNQKIISKLESLLL |
| | phosphoprotein | LKGEVESIKKQINRQNISISTLEGHLSSIMIAIPGL |
| | tetramerization | |
| | domain (MPP) | |
| 9 | Dictyocaulus | AVADVGDPFLLWKQQMDKWQNEYITDWQYHFEQY |
| | viviparus ACE | KKYQTYRHLDSDSCSGS |
| | tetramerization | |
| | domain | |
| 10-30 | Influenza virus | See FIGS. 1A-1E |
| | neuraminidase of | |
| | different strains | |
| 31 | Globular head | SVKLAGNSSLCPVSGWAIYSKDNSVRIGSKGDVFVIR |
| | domain of NA of | EPFISCSPLECRTFFLTQGALLNDKHSNGTIKDRSPYRT |
| | influenza virus | LMSCPIGEVPSPYNSRFESVAWSASACHDGINWLTIGI |
| | A/Michigan/45/15 | SGPDSGAVAVLKYNGIITDTIKSWRNNILRTQESECAC |
| | | VNGSCFTIMTDGPSDGQASYKIFRIEKGKIIKSVEMKA |
| | | PNYHYEECSCYPDSSEITCVCRDNWHGSNRPWVSFN |
| | | QNLEYQMGYICSGVFGDNPRPNDKTGSCGPVSSNGA |
| | | NGVKGFSFKYGNGVWIGRTKSISSRKGFEMIWDPNG |
| | | WTGTDNKFSIKQDIVGINEWSGYSGSFVQHPELTGLD |
| | | CIRPCFWVELIRGRPEENTIWTSGSSISFCGVNSDTVG |
| | | WSWPDGAELPFTIDK |
| 32 | Globular head | ICPKPAEYRNWSKPQCGITGFAPFSKDNSIRLSAGGDI |
| | domain of NA of | WVTREPYVSCDPDKCYQFALGQGTTINNVHSNNTAR |
| | influenza virus | DRTPHRTLLMNELGVPFHLGTKQVCIAWSSSSCHDG |
| | A/Kansas/14/2017 | KAWLHVCITGDDKNATASFIYNGRLVDSVVSWSKDI |
| | | LRTQESECVCINGTCTVVMTDGNATGKADTKILFIEE |
| | | GKIVHTSKLSGSAQHVEECSCYPRYPGVRCVCRDNW |
| | | KGSNRPIVDINIKDHSIVSSYVCSGLVGDTPRKTDSSSS |
| | | SHCLNPNNEKGGHGVKGWAFDDGNDVWMGRTINET |
| | | SRLGYETFKVVEGWSNPKSKLQINRQVIVDRGDRSGY |
| | | SGIFSVEGKSCINRCFYVELIRGRKEETEVLWTSNSIVV |
| | | FCGTSGTYGTGSWPDGADLNLMHI |
| 33 | Globular head | LLLPEPEWTYPRLSCPGSTFQKALLISPHRFGETKGNS |
| | domain of NA of | APLIIREPFVACGPNECKHEALTHYAAQPGGYYNGTR |
| | influenza virus | GDRNKLRHLISVKLGKIPTVENSIFHMAAWSGSACHD |
| | B/Colorado/06/2017 | GKEWTYIGVDGPDNNALLKVKYGEAYTDTYHSYAN |
| | | NILRTQESACNCIGGNCYLMITDGSASGVSECRFLKIR |
| | | EGRIIKEIFPTGRVKHTEECTCGFASNKTIECACRDNR |
| | | YTAKRPFVKLNVETDTAEIRLMCTDTYLDTPRPNDGS |
| | | ITGPCESDGDKGSGGIKGGFVHQRMKSKIGRWYSRT |
| | | MSQTERMGMGLYVKYGGDPWADSDALAFSGVMVS |
| | | MKEPGWYSFGFEIKDKKCDVPCIGIEMVHDGGKETW |
| | | HSAATAIYCLMGSGQLLWDTVTGVDMAL |
| 34 | Signal peptide | MLLVNQSHQGFNKEHTSKMVSAIVLYVLLAAAAHSA |
| | | FAADP |
| 35 | His tag | HHHHHH |
| 36 | Cleavage site | LVPRGSP |
| 37 | Cleavage site | SLVPRGSPSR |
| 38 | Cleavage site | ENLYFQX (X = G/S) |
| 39 | Spacer peptide | (Gly)ₙ, where n = 3 |
| 40 | N1-MPP (with signal | MLLVNQSHQGFNKEHTSKMVSAIVLYVLLAAAAHSAFAA |
| | peptide; no 6xHis; no | DPGDHYDDELFSDVQDIKTALAKIHEDNQKIISKLESL |
| | cleavage, no spacer) | LLLKGEVESIKKQINRQNISISTLEGHLSSIMIAIPGLSV |
| | | KLAGNSSLCPVSGWAIYSKDNSVRIGSKGDVFVIRE |
| | | PFISCSPLECRTFFLTQGALLNDKHSNGTIKDRSPY |
| | | RTLMSCPIGEVPSPYNSRFESVAWSASACHDGINW |
| | | LTIGISGPDSGAVAVLKYNGIITDTIKSWRNNILRTQ |
| | | ESECACVNGSCFTIMTDGPSDGQASYKIFRIEKGKI |
| | | IKSVEMKAPNYHYEECSCYPDSSEITCVCRDNWHG |
| | | SNRPWVSFNQNLEYQMGYICSGVFGDNPRPNDKT |
| | | GSCGPVSSNGANGVKGFSFKYGNGVWIGRTKSISS |
| | | RKGFEMIWDPNGWTGTDNKFSIKQDIVGINEWSG |
| | | YSGSFVQHPELTGLDCIRPCFWVELIRGRPEENTI |
| | | WTSGSSISFCGVNSDTVGWSWPDGAELPFTIDK |
| | | (signal peptide is italicized; MPP tetramerization |
| | | domain is underlined; NA globular head domain of |
| | | influenza virus A/Michigan/45/15 is bold) |
| 42 | N1-MPP (with signal | MLLVNQSHQGFNKEHTSKMVSAIVLYVLLAAAAHSAFAA |
| | peptide; no 6xHis; no | DPGDHYDDELFSDVQDIKTALAKIHEDNQKIISKLESL |
| | cleavage, with | LLLKGEVESIKKQINRQNISISTLEGHLSSIMIAIPGLGG |
| | spacer) | GSVKLAGNSSLCPVSGWAIYSKDNSVRIGSKGDVF |
| | | VIREPFISCSPLECRTFFLTQGALLNDKHSNGTIKD |
| | | RSPYRTLMSCPIGEVPSPYNSRFESVAWSASACHDG |
| | | INWLTIGISGPDSGAVAVLKYNGIITDTIKSWRNNIL |
| | | RTQESECACVNGSCFTIMTDGPSDGQASYKIFRIEK |
| | | GKIIKSVEMKAPNYHYEECSCYPDSSEITCVCRDN |
| | | WHGSNRPWVSFNQNLEYQMGYICSGVFGDNPRPN |
| | | DKTGSCGPVSSNGANGVKGFSFKYGNGVWIGRTK |
| | | SISSRKGFEMIWDPNGWTGTDNKFSIKQDIVGINE |
| | | WSGYSGSFVQHPELTGLDCIRPCFWVELIRGRPEE |
| | | NTIWTSGSSISFCGVNSDTVGWSWPDGAELPFTIDK |
| | | (signal peptide is italicized; MPP tetramerization domain is |
| | | underlined; spacer residues are double underline; NA |
| | | globular head domain of influenza virus |
| | | A/Michigan/45/2015 is bold) |
| 43 | N1-MPP (with signal | atgctgctcgtcaaccaatcccaccagggcttcaacaaggaacacacttctaagatg |
| | peptide; no 6xHis; no | gtctccgctatcgtgctctacgtgctgctcgctgccgctgcccactcagctttcgctgc |
| | cleavage, with | cgacccaggcgatcactacgacgacgaactgttctccgacgtgcaggacatcaaga |
| | spacer) | ccgctctggctaagatccacgaggacaaccagaagatcatctccaagctggaatcc |
| | | ctgctgctgctgaagggcgaagtcgagtccatcaagaagcagatcaaccgccaga |
| | | acatctccatctccaccttggagggtcacctgtcctccatcatgatcgctatccctggc |
| | | ctgggcggcggctccgtgaaattagcgggcaattcctctctctgccctgttagtggat |
| | | gggctatatacagtaaagacaacagtgtaagaatcggttccaagggggatgtgtttgt |
| | | cataagggaaccattcatatcatgctctcccttggaatgcagaaccttcttcttgactca |
| | | aggggccttgctaaatgacaaacattccaatggaaccattaaagacaggagcccata |
| | | ccgaaccctaatgagctgtcctattggtgaagttccctctccatacaactcaagatttga |
| | | gtcagtcgcttggtcagcaagtgcttgtcatgatggcatcaattggctaacaattggaa |
| | | tttctggcccagacagtggggcagtggctgtgttaaagtacaatggcataataacaga |
| | | cactatcaagagttggaggaacaatatattgagaacacaagagtctgaatgtgcatgt |
| | | gtaaatggttcttgctttaccataatgaccgatggaccaagtgatggacaggcctcata |
| | | caaaatcttcagaatagaaaagggaaagataatcaaatcagtcgaaatgaaagcccc |
| | | taattatcactatgaggaatgctcctgttaccctgattctagtgaaatcacatgtgtgtgc |
| | | agggataactggcatggctcgaatcgaccgtgggtgtctttcaaccagaatctggaat |
| | | atcagatgggatacatatgcagtggggttttcggagacaatccacgccctaatgataa |
| | | gacaggcagttgtggtccagtatcgtctaatggagcaaatggagtaaaaggattttca |
| | | ttcaaatacggcaatggtgtttggatagggagaactaaaagcattagttcaagaaaag |
| | | gttttgagatgatttgggatccgaatggatggactgggactgacaataaattctcaata |
| | | aagcaagatatcgtaggaataaatgagtggtcagggtatagcgggagttttgttcagc |
| | | atccagaactaacagggctggattgtataagaccttgcttctgggttgaactaataaga |
| | | gggcgacccgaagagaacacaatctggactagcgggagcagcatatccttttgtgg |
| | | tgtaaacagtgacactgtgggttggtcttggccagacggtgctgagttgccatttacca |
| | | ttgacaagtaa |
| 44 | N1-MPP (with signal | MLLVNQSHQGFNKEHTSKMVSAIVLYVLLAAAAHSAFAA |
| | peptide; no 6xHis; | DPGDHYDDELFSDVQDIKTALAKIHEDNQKIISKLESL |
| | with cleavage, no | LLLKGEVESIKKQINRQNISISTLEGHLSSIMIAIPGLSL |
| | spacer) | VPRGSPSRSVKLAGNSSLCPVSGWAIYSKDNSVRIG |
| | | SKGDVFVIREPFISCSPLECRTFFLTQGALLNDKHS |
| | | NGTIKDRSPYRTLMSCPIGEVPSPYNSRFESVAWSA |
| | | SACHDGINWLTIGISGPDSGAVAVLKYNGIITDTIKS |
| | | WRNNILRTQESECACVNGSCFTIMTDGPSDGQASY |
| | | KIFRIEKGKIIKSVEMKAPNYHYEECSCYPDSSEITC |
| | | VCRDNWHGSNRPWVSFNQNLEYQMGYICSGVFG |
| | | DNPRPNDKTGSCGPVSSNGANGVKGFSFKYGNGV |
| | | WIGRTKSISSRKGFEMIWDPNGWTGTDNKFSIKQD |
| | | IVGINEWSGYSGSFVQHPELTGLDCIRPCFWVELIR |
| | | GRPEENTIWTSGSSISFCGVNSDTVGWSWPDGAEL |
| | | PFTIDK |
| | | (signal peptide is italicized; MPP tetramerization domain is |
| | | underlined; thrombin cleavage site plus flanking residues are |
| | | double underlined; NA globular head domain of influenza |
| | | virus A/Michigan/45/15 is bold) |
| 46 | N1-MPP (with signal | MLLVNQSHQGFNKEHTSKMVSAIVLYVLLAAAAHSAFAA |
| | peptide; with 6xHis; | DPHHHHHHGDHYDDELFSDVQDIKTALAKIHEDNQK |
| | no cleavage, with | IISKLESLLLLKGEVESIKKQINRQNISISTLEGHLSSIMI |
| | spacer) | AIPGLGGGSVKLAGNSSLCPVSGWAIYSKDNSVRIG |
| | | SKGDVFVIREPFISCSPLECRTFFLTQGALLNDKHS |
| | | NGTIKDRSPYRTLMSCPIGEVPSPYNSRFESVAWSA |
| | | SACHDGINWLTIGISGPDSGAVAVLKYNGIITDTIKS |
| | | WRNNILRTQESECACVNGSCFTIMTDGPSDGQASY |
| | | KIFRIEKGKIIKSVEMKAPNYHYEECSCYPDSSEITC |
| | | VCRDNWHGSNRPWVSFNQNLEYQMGYICSGVFG |
| | | DNPRPNDKTGSCGPVSSNGANGVKGFSFKYGNGV |
| | | WIGRTKSISSRKGFEMIWDPNGWTGTDNKFSIKQD |
| | | IVGINEWSGYSGSFVQHPELTGLDCIRPCFWVELIR |
| | | GRPEENTIWTSGSSISFCGVNSDTVGWSWPDGAEL |
| | | PFTIDK |
| | | (signal peptide and his tag are italicized; MPP |
| | | tetramerization domain is underlined; spacer residues are |
| | | double underlined; NA globular head domain of influenza |
| | | virus A/Michigan/45/2015 is bold) |
| 47 | N1-MPP (with signal | atgctgctcgtcaaccaatcccaccagggcttcaacaaggaacacacttctaagatg |
| | peptide; with 6xHis; | gtctccgctatcgtgctctacgtgctgctcgctgccgctgcccactcagctttcgctgc |
| | no cleavage, with | cgacccacaccaccaccaccaccacggcgatcactacgacgacgaactgttctccg |
| | spacer) | acgtgcaggacatcaagaccgctctggctaagatccacgaggacaaccagaagat |
| | | catctccaagctggaatccctgctgctgctgaagggcgaagtcgagtccatcaagaa |
| | | gcagatcaaccgccagaacatctccatctccaccttggagggtcacctgtcctccatc |
| | | atgatcgctatccctggcctgggcggcggctccgtgaaattagcgggcaattcctctc |
| | | tctgccctgttagtggatgggctatatacagtaaagacaacagtgtaagaatcggttcc |
| | | aagggggatgtgtttgtcataagggaaccattcatatcatgctctcccttggaatgcag |
| | | aaccttcttcttgactcaaggggccttgctaaatgacaaacattccaatggaaccatta |
| | | aagacaggagcccataccgaaccctaatgagctgtcctattggtgaagttccctctcc |
| | | atacaactcaagatttgagtcagtcgcttggtcagcaagtgcttgtcatgatggcatca |
| | | attggctaacaattggaatttctggcccagacagtggggcagtggctgtgttaaagta |
| | | caatggcataataacagacactatcaagagttggaggaacaatatattgagaacaca |
| | | agagtctgaatgtgcatgtgtaaatggttcttgctttaccataatgaccgatggaccaa |
| | | gtgatggacaggcctcatacaaaatcttcagaatagaaaagggaaagataatcaaat |
| | | cagtcgaaatgaaagcccctaattatcactatgaggaatgctcctgttaccctgattcta |
| | | gtgaaatcacatgtgtgtgcagggataactggcatggctcgaatcgaccgtgggtgt |
| | | ctttcaaccagaatctggaatatcagatgggatacatatgcagtggggttttcggaga |
| | | caatccacgccctaatgataagacaggcagttgtggtccagtatcgtctaatggagca |
| | | aatggagtaaaaggattttcattcaaatacggcaatggtgtttggatagggagaacta |
| | | aaagcattagttcaagaaaaggttttgagatgatttgggatccgaatggatggactgg |
| | | gactgacaataaattctcaataaagcaagatatcgtaggaataaatgagtggtcaggg |
| | | tatagcgggagttttgttcagcatccagaactaacagggctggattgtataagaccttg |
| | | cttctgggttgaactaataagagggcgacccgaagagaacacaatctggactagcg |
| | | ggagcagcatatccttttgtggtgtaaacagtgacactgtgggttggtcttggccaga |
| | | cggtgctgagttgccatttaccattgacaagtaa |
| 48 | N1-MPP (with signal | MLLVNQSHQGFNKEHTSKMVSAIVLYVLLAAAAHSAFAA |
| | peptide; with 6xHis; | DPHHHHHHGDHYDDELFSDVQDIKTALAKIHEDNQK |
| | with cleavage, no | IISKLESLLLLKGEVESIKKQINRQNISISTLEGHLSSIMI |
| | spacer | AIPGLSLVPRGSPSRSVKLAGNSSLCPVSGWAIYSKD |
| | | NSVRIGSKGDVFVIREPFISCSPLECRTFFLTQGALL |
| | | NDKHSNGTIKDRSPYRTLMSCPIGEVPSPYNSRFES |
| | | VAWSASACHDGINWLTIGISGPDSGAVAVLKYNGII |
| | | TDTIKSWRNNILRTQESECACVNGSCFTIMTDGPSD |
| | | GQASYKIFRIEKGKIIKSVEMKAPNYHYEECSCYPD |
| | | SSEITCVCRDNWHGSNRPWVSFNQNLEYQMGYICS |
| | | GVFGDNPRPNDKTGSCGPVSSNGANGVKGFSFKY |
| | | GNGVWIGRTKSISSRKGFEMIWDPNGWTGTDNKF |
| | | SIKQDIVGINEWSGYSGSFVQHPELTGLDCIRPCFW |
| | | VELIRGRPEENTIWTSGSSISFCGVNSDTVGWSWPD |
| | | GAELPFTIDK |
| | | (signal peptide and his tag are italicized; MPP |
| | | tetramerization domain is underlined; thrombin cleavage site |
| | | plus flanking residues are double underlined; NA globular |
| | | head domain of influenza virus A/Michigan/45/2015 is bold) |
| 50 | N2-MPP (with signal | MLLVNQSHQGFNKEHTSKMVSAIVLYVLLAAAAHSAFAA |
| | peptide; no 6xHis; no | DPGDHYDDELFSDVQDIKTALAKIHEDNQKIISKLESL |
| | cleavage, no spacer) | LLLKGEVESIKKQINRQNISISTLEGHLSSIMIAIPGLIC |
| | | PKPAEYRNWSKPQCGITGFAPFSKDNSIRLSAGGDI |
| | | WVTREPYVSCDPDKCYQFALGQGTTINNVHSNNT |
| | | ARDRTPHRTLLMNELGVPFHLGTKQVCIAWSSSSC |
| | | HDGKAWLHVCITGDDKNATASFIYNGRLVDSVVS |
| | | WSKDILRTQESECVCINGTCTVVMTDGNATGKAD |
| | | TKILFIEEGKIVHTSKLSGSAQHVEECSCYPRYPGV |
| | | RCVCRDNWKGSNRPIVDINIKDHSIVSSYVCSGLVG |
| | | DTPRKTDSSSSSHCLNPNNEKGGHGVKGWAFDDG |
| | | NDVWMGRTINETSRLGYETFKVVEGWSNPKSKLQ |
| | | INRQVIVDRGDRSGYSGIFSVEGKSCINRCFYVELIR |
| | | GRKEETEVLWTSNSIVVFCGTSGTYGTGSWPDGA |
| | | DLNLMHI |
| | | (signal peptide is italicized; MPP tetramerization domain is |
| | | underlined; NA globular head domain of influenza virus |
| | | A/Kansas/14/2017 is bold) |
| 52 | N2-MPP (with signal | MLLVNQSHQGFNKEHTSKMVSAIVLYVLLAAAAHSAFAA |
| | peptide; no 6xHis; no | DPHHHHHHGDHYDDELFSDVQDIKTALAKIHEDNQK |
| | cleavage, with | IISKLESLLLLKGEVESIKKQINRQNISISTLEGHLSSIMI |
| | spacer) | AIPGLGGGICPKPAEYRNWSKPQCGITGFAPFSKDN |
| | | SIRLSAGGDIWVTREPYVSCDPDKCYQFALGQGTT |
| | | INNVHSNNTARDRTPHRTLLMNELGVPFHLGTKQ |
| | | VCIAWSSSSCHDGKAWLHVCITGDDKNATASFIYN |
| | | GRLVDSVVSWSKDILRTQESECVCINGTCTVVMTD |
| | | GNATGKADTKILFIEEGKIVHTSKLSGSAQHVEECS |
| | | CYPRYPGVRCVCRDNWKGSNRPIVDINIKDHSIVSS |
| | | YVCSGLVGDTPRKTDSSSSSHCLNPNNEKGGHGVK |
| | | GWAFDDGNDVWMGRTINETSRLGYETFKVVEGW |
| | | SNPKSKLQINRQVIVDRGDRSGYSGIFSVEGKSCIN |
| | | RCFYVELIRGRKEETEVLWTSNSIVVFCGTSGTYG |
| | | TGSWPDGADLNLMHI |
| | | (signal peptide is italicized; MPP tetramerization domain is |
| | | underlined; spacer residues are double underline; NA |
| | | globular head domain of influenza virus A/Kansas/14/2017 |
| | | is bold) |
| 54 | N2-MPP (with signal | MLLVNQSHQGFNKEHTSKMVSAIVLYVLLAAAAHSAFAA |
| | peptide; no 6xHis; | DPGDHYDDELFSDVQDIKTALAKIHEDNQKIISKLESL |
| | with cleavage, no | LLLKGEVESIKKQINRQNISISTLEGHLSSIMIAIPGLSL |
| | spacer) | VPRGSPSRICPKPAEYRNWSKPQCGITGFAPFSKDN |
| | | SIRLSAGGDIWVTREPYVSCDPDKCYQFALGQGTT |
| | | INNVHSNNTARDRTPHRTLLMNELGVPFHLGTKQ |
| | | VCIAWSSSSCHDGKAWLHVCITGDDKNATASFIYN |
| | | GRLVDSVVSWSKDILRTQESECVCINGTCTVVMTD |
| | | GNATGKADTKILFIEEGKIVHTSKLSGSAQHVEECS |
| | | CYPRYPGVRCVCRDNWKGSNRPIVDINIKDHSIVSS |
| | | YVCSGLVGDTPRKTDSSSSSHCLNPNNEKGGHGVK |
| | | GWAFDDGNDVWMGRTINETSRLGYETFKVVEGW |
| | | SNPKSKLQINRQVIVDRGDRSGYSGIFSVEGKSCIN |
| | | RCFYVELIRGRKEETEVLWTSNSIVVFCGTSGTYG |
| | | TGSWPDGADLNLMHI |
| | | (signal peptide is italicized; MPP tetramerization domain is |
| | | underlined; thrombin cleavage site plus flanking residues are |
| | | double underlined; NA globular head domain of influenza |
| | | virus A/Kansas/14/2017 is bold) |
| 56 | N2-MPP (with signal | MLLVNQSHQGFNKEHTSKMVSAIVLYVLLAAAAHSAFAA |
| | peptide; with 6xHis; | DPHHHHHHGDHYDDELFSDVQDIKTALAKIHEDNQK |
| | no cleavage, with | IISKLESLLLLKGEVESIKKQINRQNISISTLEGHLSSIMI |
| | spacer) | AIPGLGGGICPKPAEYRNWSKPQCGITGFAPFSKDN |
| | | SIRLSAGGDIWVTREPYVSCDPDKCYQFALGQGTT |
| | | INNVHSNNTARDRTPHRTLLMNELGVPFHLGTKQ |
| | | VCIAWSSSSCHDGKAWLHVCITGDDKNATASFIYN |
| | | GRLVDSVVSWSKDILRTQESECVCINGTCTVVMTD |
| | | GNATGKADTKILFIEEGKIVHTSKLSGSAQHVEECS |
| | | CYPRYPGVRCVCRDNWKGSNRPIVDINIKDHSIVSS |
| | | YVCSGLVGDTPRKTDSSSSSHCLNPNNEKGGHGVK |
| | | GWAFDDGNDVWMGRTINETSRLGYETFKVVEGW |
| | | SNPKSKLQINRQVIVDRGDRSGYSGIFSVEGKSCIN |
| | | RCFYVELIRGRKEETEVLWTSNSIVVFCGTSGTYG |
| | | TGSWPDGADLNLMHI |
| | | (signal peptide and his tag are italicized; MPP |
| | | tetramerization domain is underlined; spacer residues are |
| | | double underlined; NA globular head domain of influenza |
| | | virus A/Kansas/14/2017 is bold) |
| 58 | N2-MPP (with signal | MLLVNQSHQGFNKEHTSKMVSAIVLYVLLAAAAHSAFAA |
| | peptide; with 6xHis; | DPHHHHHHGDHYDDELFSDVQDIKTALAKIHEDNQK |
| | with cleavage, no | IISKLESLLLLKGEVESIKKQINRQNISISTLEGHLSSIMI |
| | spacer) | AIPGLSLVPRGSPSRICPKPAEYRNWSKPQCGITGFA |
| | | PFSKDNSIRLSAGGDIWVTREPYVSCDPDKCYQFA |
| | | LGQGTTINNVHSNNTARDRTPHRTLLMNELGVPF |
| | | HLGTKQVCIAWSSSSCHDGKAWLHVCITGDDKNA |
| | | TASFIYNGRLVDSVVSWSKDILRTQESECVCINGTC |
| | | TVVMTDGNATGKADTKILFIEEGKIVHTSKLSGSA |
| | | QHVEECSCYPRYPGVRCVCRDNWKGSNRPIVDINI |
| | | KDHSIVSSYVCSGLVGDTPRKTDSSSSSHCLNPNNE |
| | | KGGHGVKGWAFDDGNDVWMGRTINETSRLGYET |
| | | FKVVEGWSNPKSKLQINRQVIVDRGDRSGYSGIFS |
| | | VEGKSCINRCFYVELIRGRKEETEVLWTSNSIVVFC |
| | | GTSGTYGTGSWPDGADLNLMHI |
| | | (signal peptide and his tag are italicized; MPP |
| | | tetramerization domain is underlined; thrombin cleavage site |
| | | plus flanking residues are double underlined; NA globular |
| | | head domain of influenza virus A/Kansas/14/2017 is bold) |
| 59 | N2-MPP (with signal | atgctgctcgtcaaccaatcccaccagggcttcaacaaggaacacacttctaagatg |
| | peptide; with 6xHis; | gtctccgctatcgtgctctacgtgctgctcgctgccgctgcccactcagctttcgctgc |
| | with cleavage, no | cgacccacaccaccaccaccaccacggcgatcactacgacgacgaactgttctccg |
| | spacer) | acgtgcaggacatcaagaccgctctggctaagatccacgaggacaaccagaagat |
| | | catctccaagctggaatccctgctgctgctgaagggcgaagtcgagtccatcaagaa |
| | | gcagatcaaccgccagaacatctccatctccaccttggagggtcacctgtcctccatc |
| | | atgatcgctatccctggcctgtctctcgtgcccaggggatcaccttctagaatatgccc |
| | | caaaccagcagaatacagaaattggtcaaaaccgcaatgtggcattacaggatttgc |
| | | acctttctctaaggacaattcgattaggctttccgctggtggggacatctgggtgacaa |
| | | gagaaccttatgtgtcatgcgatcctgacaagtgttatcaatttgcccttggacaggga |
| | | acaacaataaacaacgtgcattcaaataacacagcacgtgataggacccctcatcgg |
| | | actctattgatgaatgagttgggtgttcctttccatctggggaccaagcaagtgtgcata |
| | | gcatggtccagctcaagttgtcacgatggaaaagcatggctgcatgtttgtataacgg |
| | | gggatgataaaaatgcaactgctagtttcatttacaatgggaggcttgtagatagtgtt |
| | | gtttcatggtccaaagatattctcaggacccaggagtcagaatgcgtttgtatcaatgg |
| | | aacttgtacagtagtaatgactgatggaaatgctacaggaaaagctgatactaaaatat |
| | | tattcattgaggaggggaaaatcgttcatactagcaaattgtcaggaagtgctcagcat |
| | | gtcgaagagtgctcttgctatcctcgataccctggtgtcagatgtgtctgcagagaca |
| | | actggaaaggatccaaccggcccatcgtagatataaacataaaggatcatagcattg |
| | | tttccagttatgtgtgttcaggacttgttggagacacacccagaaaaaccgacagctc |
| | | cagcagcagccattgcttgaatcctaacaatgaaaaaggtggtcatggagtgaaagg |
| | | ctgggcctttgatgatggaaatgacgtgtggatggggagaacaatcaacgagacgt |
| | | cacgcttagggtatgaaaccttcaaagtcgttgaaggctggtccaaccctaagtccaa |
| | | attgcagataaataggcaagtcatagttgacagaggtgataggtccggttattctggta |
| | | ttttctctgttgaaggcaaaagctgcatcaatcggtgcttttatgtggagttgattaggg |
| | | gaagaaaagaggaaactgaagtcttgtggacctcaaacagtattgttgtgttttgtggc |
| | | acctcaggtacatatggaacaggctcatggcctgatggggcggacctcaatctcatg |
| | | catatataa |
| 60 | B-NA-MPP or B- | MLLVNQSHQGFNKEHTSKMVSAIVLYVLLAAAAHSAFAA |
| | MPP (with signal | DPGDHYDDELFSDVQDIKTALAKIHEDNQKIISKLESL |
| | peptide; no 6xHis; no | LLLKGEVESIKKQINRQNISISTLEGHLSSIMIAIPGLLL |
| | cleavage, no spacer) | LPEPEWTYPRLSCPGSTFQKALLISPHRFGETKGNS |
| | | APLIIREPFVACGPNECKHEALTHYAAQPGGYYNG |
| | | TRGDRNKLRHLISVKLGKIPTVENSIFHMAAWSGS |
| | | ACHDGKEWTYIGVDGPDNNALLKVKYGEAYTDT |
| | | YHSYANNILRTQESACNCIGGNCYLMITDGSASGV |
| | | SECRFLKIREGRIIKEIFPTGRVKHTEECTCGFASN |
| | | KTIECACRDNRYTAKRPFVKLNVETDTAEIRLMCT |
| | | DTYLDTPRPNDGSITGPCESDGDKGSGGIKGGFVH |
| | | QRMKSKIGRWYSRTMSQTERMGMGLYVKYGGDP |
| | | WADSDALAFSGVMVSMKEPGWYSFGFEIKDKKCD |
| | | VPCIGIEMVHDGGKETWHSAATAIYCLMGSGQLL |
| | | WDTVTGVDMAL |
| | | (signal peptide is italicized; MPP tetramerization domain is |
| | | underlined; NA globular head domain of influenza virus |
| | | B/Colorado/06/2017 is bold) |
| 62 | B-NA-MPP or B- | MLLVNQSHQGFNKEHTSKMVSAIVLYVLLAAAAHSAFAA |
| | MPP (with signal | DPGDHYDDELFSDVQDIKTALAKIHEDNQKIISKLESL |
| | peptide; no 6xHis; no | LLLKGEVESIKKQINRQNISISTLEGHLSSIMIAIPGLGG |
| | cleavage, with | GLLLPEPEWTYPRLSCPGSTFQKALLISPHRFGETK |
| | spacer) | GNSAPLIIREPFVACGPNECKHEALTHYAAQPGGY |
| | | YNGTRGDRNKLRHLISVKLGKIPTVENSIFHMAAW |
| | | SGSACHDGKEWTYIGVDGPDNNALLKVKYGEAYT |
| | | DTYHSYANNILRTQESACNCIGGNCYLMITDGSAS |
| | | GVSECRFLKIREGRIIKEIFPTGRVKHTEECTCGFA |
| | | SNKTIECACRDNRYTAKRPFVKLNVETDTAEIRLM |
| | | CTDTYLDTPRPNDGSITGPCESDGDKGSGGIKGGF |
| | | VHQRMKSKIGRWYSRTMSQTERMGMGLYVKYG |
| | | GDPWADSDALAFSGVMVSMKEPGWYSFGFEIKDK |
| | | KCDVPCIGIEMVHDGGKETWHSAATAIYCLMGSG |
| | | QLLWDTVTGVDMAL |
| | | (signal peptide is italicized; MPP tetramerization domain is |
| | | underlined; spacer residues are double underline; NA |
| | | globular head domain of influenza virus |
| | | B/Colorado/06/2017 is bold) |
| 64 | B-NA-MPP or B- | MLLVNQSHQGFNKEHTSKMVSAIVLYVLLAAAAHSAFAA |
| | MPP (with signal | DPGDHYDDELFSDVQDIKTALAKIHEDNQKIISKLESL |
| | peptide; no 6xHis; | LLLKGEVESIKKQINRQNISISTLEGHLSSIMIAIPGLSL |
| | with cleavage, no | VPRGSPSRLLLPEPEWTYPRLSCPGSTFQKALLISPH |
| | spacer) | RFGETKGNSAPLIIREPFVACGPNECKHEALTHYA |
| | | AQPGGYYNGTRGDRNKLRHLISVKLGKIPTVENSI |
| | | FHMAAWSGSACHDGKEWTYIGVDGPDNNALLKV |
| | | KYGEAYTDTYHSYANNILRTQESACNCIGGNCYL |
| | | MITDGSASGVSECRFLKIREGRIIKEIFPTGRVKHT |
| | | EECTCGFASNKTIECACRDNRYTAKRPFVKLNVET |
| | | DTAEIRLMCTDTYLDTPRPNDGSITGPCESDGDKG |
| | | SGGIKGGFVHQRMKSKIGRWYSRTMSQTERMGM |
| | | GLYVKYGGDPWADSDALAFSGVMVSMKEPGWYS |
| | | FGFEIKDKKCDVPCIGIEMVHDGGKETWHSAATAI |
| | | YCLMGSGQLLWDTVTGVDMAL |
| | | (signal peptide is italicized; MPP tetramerization domain is |
| | | underlined; thrombin cleavage site plus flanking residues are |
| | | double underlined; NA globular head domain of influenza |
| | | virus B/Colorado/06/2017 is bold) |
| 66 | B-NA-MPP or B- | MLLVNQSHQGFNKEHTSKMVSAIVLYVLLAAAAHSAFAA |
| | MPP (with signal | DPHHHHHHGDHYDDELFSDVQDIKTALAKIHEDNQK |
| | peptide; with 6xHis; | IISKLESLLLLKGEVESIKKQINRQNISISTLEGHLSSIMI |
| | no cleavage, with | AIPGLGGGLLLPEPEWTYPRLSCPGSTFQKALLISP |
| | spacer) | HRFGETKGNSAPLIIREPFVACGPNECKHEALTHY |
| | | AAQPGGYYNGTRGDRNKLRHLISVKLGKIPTVENS |
| | | IFHMAAWSGSACHDGKEWTYIGVDGPDNNALLKV |
| | | KYGEAYTDTYHSYANNILRTQESACNCIGGNCYL |
| | | MITDGSASGVSECRFLKIREGRIIKEIFPTGRVKHT |
| | | EECTCGFASNKTIECACRDNRYTAKRPFVKLNVET |
| | | DTAEIRLMCTDTYLDTPRPNDGSITGPCESDGDKG |
| | | SGGIKGGFVHQRMKSKIGRWYSRTMSQTERMGM |
| | | GLYVKYGGDPWADSDALAFSGVMVSMKEPGWYS |
| | | FGFEIKDKKCDVPCIGIEMVHDGGKETWHSAATAI |
| | | YCLMGSGQLLWDTVTGVDMAL |
| | | (signal peptide and his tag are italicized; MPP |
| | | tetramerization domain is underlined; spacer residues are |
| | | double underlined; NA globular head domain of influenza |
| | | virus B/Colorado/06/2017 is bold) |
| 68 | B-NA-MPP or B- | MLLVNQSHQGFNKEHTSKMVSAIVLYVLLAAAAHSAFAA |
| | MPP (with signal | DPHHHHHHGDHYDDELFSDVQDIKTALAKIHEDNQK |
| | peptide; with 6xHis; | IISKLESLLLLKGEVESIKKQINRQNISISTLEGHLSSIMI |
| | with cleavage, no | AIPGLSLVPRGSPSRLLLPEPEWTYPRLSCPGSTFQK |
| | spacer) | ALLISPHRFGETKGNSAPLIIREPFVACGPNECKHF |
| | | ALTHYAAQPGGYYNGTRGDRNKLRHLISVKLGKI |
| | | PTVENSIFHMAAWSGSACHDGKEWTYIGVDGPDN |
| | | NALLKVKYGEAYTDTYHSYANNILRTQESACNCIG |
| | | GNCYLMITDGSASGVSECRFLKIREGRIIKEIFPTG |
| | | RVKHTEECTCGFASNKTIECACRDNRYTAKRPFVK |
| | | LNVETDTAEIRLMCTDTYLDTPRPNDGSITGPCESD |
| | | GDKGSGGIKGGFVHQRMKSKIGRWYSRTMSQTE |
| | | RMGMGLYVKYGGDPWADSDALAFSGVMVSMKEP |
| | | GWYSFGFEIKDKKCDVPCIGIEMVHDGGKETWHS |
| | | AATAIYCLMGSGQLLWDTVTGVDMAL |
| | | (signal peptide and his tag are italicized; MPP |
| | | tetramerization domain is underlined; thrombin cleavage site |
| | | plus flanking residues are double underlined; NA globular |
| | | head domain of influenza virus B/Colorado/06/2017 is bold) |
| 69 | B-NA-MPP or B- | atgctgctcgtcaaccaatcccaccagggcttcaacaaggaacacacttctaagatg |
| | MPP (with signal | gtctccgctatcgtgctctacgtgctgctcgctgccgctgcccactcagctttcgctgc |
| | peptide; with 6xHis; | cgacccacaccaccaccaccaccacggcgatcactacgacgacgaactgttctccg |
| | with cleavage, no | acgtgcaggacatcaagaccgctctggctaagatccacgaggacaaccagaagat |
| | spacer) | catctccaagctggaatccctgctgctgctgaagggcgaagtcgagtccatcaagaa |
| | | gcagatcaaccgccagaacatctccatctccaccttggagggtcacctgtcctccatc |
| | | atgatcgctatccctggcctgtctctcgtgcccaggggatcaccttctagacttcttctc |
| | | ccagaaccggagtggacatacccgcgtttatcttgcccgggctcaacctttcagaaa |
| | | gcactcctaattagccctcatagattcggagaaaccaaaggaaactcagctcccttga |
| | | taataagggaaccttttgttgcttgtggaccaaatgaatgcaaacactttgctttaaccc |
| | | attatgcagcccaaccagggggatactacaatggaacaagaggagacagaaacaa |
| | | gctgaggcatctaatttcagtcaaattgggcaaaatcccaacagtagagaactccattt |
| | | tccacatggcagcatggagcgggtccgcgtgccatgatggtaaggaatggacatat |
| | | atcggagttgatggccctgacaataatgcattgctcaaagtaaaatatggagaagcat |
| | | atactgacacataccattcctatgcaaacaacatcctaagaacacaagaaagtgcctg |
| | | caattgcatcgggggaaattgttatctaatgataactgatggctcagcttcaggtgttag |
| | | tgaatgcagatttcttaagattcgagagggccgaataataaaagaaatatttccaacag |
| | | gaagagtaaaacacactgaggaatgcacatgcggatttgccagcaataaaaccata |
| | | gaatgtgcctgtagagacaacaggtacacagcaaaaagaccttttgtcaaattaaac |
| | | gtggagactgatacagcagaaataaggttgatgtgcacagatacctatttggacacc |
| | | cccagaccaaatgatggaagcataacaggcccttgtgaatctgatggggacaaagg |
| | | gagtggaggcatcaagggaggatttgttcatcaaagaatgaaatccaagattggaag |
| | | gtggtactctcgaacgatgtctcaaactgaaaggatggggatgggactgtatgtcaa |
| | | gtatggtggagacccatgggctgacagtgatgccctagcttttagtggagtaatggttt |
| | | caatgaaagaacctggttggtattcctttggcttcgaaataaaagataagaaatgcgat |
| | | gtcccctgtattgggatagagatggtacatgatggtggaaaagagacttggcactca |
| | | gcagcaacagccatttactgtttaatgggctcaggacagctgctgtgggacactgtca |
| | | caggtgttgacatggctctgtaa |
| 70 | SMDTF-N1 (with | MLLVNQSHQGFNKEHTSKMVSAIVLYVLLAAAAHSAFAA |
| | signal peptide; with | DPHHHHHHVELSSQQEYLKLKERYDALQRTQRNLLG |
| | 6xHis; with cleavage, | EDLGPLSTKELESLERQLDSSLKQIRALRTQFMLDQSK |
| | no spacer) | ERMLTETNKTLRLRLADGYSLVPRGSPSRSVKLAGNS |
| | | SLCPVSGWAIYSKDNSVRIGSKGDVFVIREPFISCSP |
| | | LECRTFFLTQGALLNDKHSNGTIKDRSPYRTLMSC |
| | | PIGEVPSPYNSRFESVAWSASACHDGINWLTIGISG |
| | | PDSGAVAVLKYNGIITDTIKSWRNNILRTQESECAC |
| | | VNGSCFTIMTDGPSDGQASYKIFRIEKGKIIKSVEM |
| | | KAPNYHYEECSCYPDSSEITCVCRDNWHGSNRPW |
| | | VSFNQNLEYQMGYICSGVFGDNPRPNDKTGSCGPV |
| | | SSNGANGVKGFSFKYGNGVWIGRTKSISSRKGFEM |
| | | IWDPNGWTGTDNKFSIKQDIVGINEWSGYSGSFVQ |
| | | HPELTGLDCIRPCFWVELIRGRPEENTIWTSGSSISF |
| | | CGVNSDTVGWSWPDGAELPFTIDK |
| | | (signal peptide and his tag are italicized; SMDTF |
| | | tetramerization domain is underlined; thrombin cleavage site |
| | | plus flanking residues are double underlined; NA globular |
| | | head domain of influenza virus A/Michigan/45/2015 is bold) |
| 71 | SPP-N1 (with signal | MLLVNQSHQGFNKEHTSKMVSAIVLYVLLAAAAHSAFAA |
| | peptide; with 6xHis; | DPHHHHHHENTSSMKEMATLLTSLGVIQSAQEFESSR |
| | with cleavage, no | DASYVFARRALKSANYAEMTFNVCGLILSAEKSSAR |
| | spacer) | KVDENKQLLKIQESVESFRDIYKRFSEYQKEQNSLLM |
| | | SNLSTLHIITDSLVPRGSPSRSVKLAGNSSLCPVSGWA |
| | | IYSKDNSVRIGSKGDVFVIREPFISCSPLECRTFFLT |
| | | QGALLNDKHSNGTIKDRSPYRTLMSCPIGEVPSPY |
| | | NSRFESVAWSASACHDGINWLTIGISGPDSGAVAVL |
| | | KYNGIITDTIKSWRNNILRTQESECACVNGSCFTIM |
| | | TDGPSDGQASYKIFRIEKGKIIKSVEMKAPNYHYEE |
| | | CSCYPDSSEITCVCRDNWHGSNRPWVSFNQNLEYQ |
| | | MGYICSGVFGDNPRPNDKTGSCGPVSSNGANGVK |
| | | GFSFKYGNGVWIGRTKSISSRKGFEMIWDPNGWT |
| | | GTDNKFSIKQDIVGINEWSGYSGSFVQHPELTGLD |
| | | CIRPCFWVELIRGRPEENTIWTSGSSISFCGVNSDT |
| | | VGWSWPDGAELPFTIDK |
| | | (signal peptide and his tag are italicized; SPP tetra- |
| | | merization domain is underlined; thrombin cleavage site plus |
| | | flanking residues are double underlined; NA globular head |
| | | domain of influenza virus A/Michigan/45/2015 is bold) |
| 72 | PiLZ-N1 (with signal | MLLVNQSHQGFNKEHTSKMVSAIVLYVLLAAAAHSAFAA |
| | peptide; with 6xHis; | DPHHHHHHLLVQRMDAKLDLILALIGRLVRQSSLVPR |
| | with cleavage, no | GSPSRSVKLAGNSSLCPVSGWAIYSKDNSVRIGSKG |
| | spacer) | DVFVIREPFISCSPLECRTFFLTQGALLNDKHSNGTI |
| | | KDRSPYRTLMSCPIGEVPSPYNSRFESVAWSASACH |
| | | DGINWLTIGISGPDSGAVAVLKYNGIITDTIKSWRN |
| | | NILRTQESECACVNGSCFTIMTDGPSDGQASYKIFR |
| | | IEKGKIIKSVEMKAPNYHYEECSCYPDSSEITCVCR |
| | | DNWHGSNRPWVSFNQNLEYQMGYICSGVFGDNPR |
| | | PNDKTGSCGPVSSNGANGVKGFSFKYGNGVWIGR |
| | | TKSISSRKGFEMIWDPNGWTGTDNKFSIKQDIVGIN |
| | | EWSGYSGSFVQHPELTGLDCIRPCFWVELIRGRPE |
| | | ENTIWTSGSSISFCGVNSDTVGWSWPDGAELPFTID |
| | | K |
| | | (signal peptide and his tag are italicized; PiLZ |
| | | tetramerization domain is underlined; thrombin cleavage site |
| | | plus flanking residues are double underlined; NA globular |
| | | head domain of influenza virus A/Michigan/45/2015 is bold) |
| 73 | ACE-N1 (with signal | MLLVNQSHQGFNKEHTSKMVSAIVLYVLLAAAAHSAFAA |
| | peptide; with 6xHis; | DPHHHHHHAVADVGDPFLLWKQQMDKWQNEYITD |
| | with cleavage, no | WQYHFEQYKKYQTYRHLDSDSCSGSSLVPRGSPSRS |
| | spacer) | VKLAGNSSLCPVSGWAIYSKDNSVRIGSKGDVFVIR |
| | | EPFISCSPLECRTFFLTQGALLNDKHSNGTIKDRSP |
| | | YRTLMSCPIGEVPSPYNSRFESVAWSASACHDGIN |
| | | WLTIGISGPDSGAVAVLKYNGIITDTIKSWRNNILR |
| | | TQESECACVNGSCFTIMTDGPSDGQASYKIFRIEKG |
| | | KIIKSVEMKAPNYHYEECSCYPDSSEITCVCRDNW |
| | | HGSNRPWVSFNQNLEYQMGYICSGVFGDNPRPND |
| | | KTGSCGPVSSNGANGVKGFSFKYGNGVWIGRTKSI |
| | | SSRKGFEMIWDPNGWTGTDNKFSIKQDIVGINEWS |
| | | GYSGSFVQHPELTGLDCIRPCFWVELIRGRPEENTI |
| | | WTSGSSISFCGVNSDTVGWSWPDGAELPFTIDK |
| | | (signal peptide and his tag are italicized; ACE |
| | | tetramerization domain is underlined; thrombin cleavage site |
| | | plus flanking residues are double underlined; NA globular |
| | | head domain of influenza virus A/Michigan/45/2015 is bold) |

In specific embodiments, a recombinant neuraminidase described herein comprises a Measles virus phosphoprotein tetramerization domain. The Measles virus phosphoprotein tetramerization domain may comprise an amino acid sequence known in the art. The Measles virus phosphoprotein tetramerization domain may comprise SEQ ID NO:8, or an amino acid sequence that is at least 80%, at least 85%, at least 90%, at least 95%, at least 98%, or at least 99% identical to SEQ ID NO:8. In one embodiment, a recombinant neuraminidase described herein comprises an amino acid sequence having at least 80% identity to a Measles virus phosphoprotein tetramerization domain (MPP) described herein (e.g., SEQ ID NO:8) or known in the art. In a specific embodiment, a recombinant neuraminidase described herein comprises an amino acid sequence having at least 85%, at least 90%, at least 95%, or at least 98% identity to a measles virus phosphoprotein tetramerization domain (MPP) described herein (e.g., SEQ ID NO:8) or known in the art. In some embodiments, a recombinant neuraminidase described herein comprises an amino acid sequence having at least 80% identity to SEQ ID NO:8. In a specific embodiment, a recombinant neuraminidase described herein comprises an amino acid sequence having at least 85%, at least 90%, at least 95%, or at least 98% identity to SEQ ID NO:8. In another specific embodiment, a recombinant neuraminidase described herein comprises an amino acid sequence having at least 95%, at least 98%, or at least 99% identity to SEQ ID NO:8. In certain embodiments, a recombinant neuraminidase described herein comprises the amino acid sequence of SEQ ID NO:8.

In some embodiments, a recombinant neuraminidase described herein comprises a tetramerization domain comprising an amino acid sequence having at least 80% identity to a Measles virus phosphoprotein tetramerization domain (MPP) described herein (e.g., SEQ ID NO:8) or known in the art. In some embodiments, a recombinant neuraminidase described herein comprises a tetramerization domain comprising an amino acid sequence having at least 85%, at least 90%, at least 95%, at least 98%, at least 99% identity to a Measles virus phosphoprotein tetramerization domain (MPP) described herein (e.g., SEQ ID NO:8) or known in the art. In some embodiments, a recombinant neuraminidase described herein comprises a tetramerization domain comprising an amino acid sequence having at least 80% identity to SEQ ID NO:8. In some embodiments, a recombinant neuraminidase described herein comprises a tetramerization domain comprising an amino acid sequence having at least 85%, at least 90%, at least 95%, at least 98%, or at least 99% identity to SEQ ID NO:8. In another specific embodiment, a recombinant neuraminidase described herein comprises a tetramerization domain comprising an amino acid sequence having at least 95%, at least 98%, or at least 99% identity to SEQ ID NO:8. In a specific embodiment, a recombinant neuraminidase described herein comprises a tetramerization domain comprising the amino acid sequence of SEQ ID NO:8.

In specific embodiments, a recombinant neuraminidase described herein comprises a Sendai virus phosphoprotein tetramerization domain. The Sendai virus phosphoprotein tetramerization domain may comprise an amino acid sequence known in the art. The Sendai virus phosphoprotein tetramerization domain may comprise SEQ ID NO:6, or an amino acid sequence that is at least 80%, at least 85%, at least 90%, at least 95%, at least 98%, at least 99% identical to SEQ ID NO:6.

In some embodiments, a recombinant neuraminidase described herein comprises a an amino acid sequence having at least 80% identity to a Sendai virus phosphoprotein tetramerization domain (SPP) described herein (e.g., SEQ ID NO:6) or known in the art. In some embodiments, a recombinant neuraminidase described herein comprises an amino acid sequence having at least 80% identity to a Sendai virus phosphoprotein tetramerization domain (SPP) described herein (e.g., SEQ ID NO:6) or known in the art. In some embodiments, a recombinant neuraminidase described herein comprises an amino acid sequence having at least 85%, at least 90%, at least 95%, at least 98%, or at least 99% identity to a Sendai virus phosphoprotein tetramerization domain (SPP) described herein (e.g., SEQ ID NO:6) or known in the art. In some embodiments, a recombinant neuraminidase described herein comprises an amino acid sequence having at least 80% identity to SEQ ID NO:6. In some embodiments, a recombinant neuraminidase described herein comprises an amino acid sequence having at least 85%, at least 90%, at least 95%, at least 98%, or at least 99% identity to SEQ ID NO:6. In some embodiments, a recombinant neuraminidase described herein comprises an amino acid sequence having at least 95%, at least 98%, or at least 99% identity to SEQ ID NO:6. In a specific embodiment, a recombinant neuraminidase described herein comprises the amino acid sequence of SEQ ID NO:6.

In another specific embodiment, a recombinant neuraminidase described herein comprises a tetramerization domain comprising an amino acid sequence having at least 80% identity to a Sendai virus phosphoprotein tetramerization domain (SPP) described herein (e.g., SEQ ID NO:6) or known in the art. In some embodiments, a recombinant neuraminidase described herein comprises a tetramerization domain comprising an amino acid sequence having at least 85%, at least 90%, at least 95%, or at least 98% identity to a Sendai virus phosphoprotein tetramerization domain (SPP) described herein (e.g., SEQ ID NO:6) or known in the art. In another specific embodiment, a recombinant neuraminidase described herein comprises a tetramerization domain comprising an amino acid sequence having at least 80% identity to SEQ ID NO:6. In some embodiments, a recombinant neuraminidase described herein comprises a tetramerization domain comprising an amino acid sequence having at least 85%, at least 90%, at least 95%, or at least 98% identity to SEQ ID NO:6. In some embodiments, a recombinant neuraminidase described herein comprises a tetramerization domain comprising an amino acid sequence having at least 95%, at least 98%, or at least 99% identity to SEQ ID NO:6. In certain embodiments, a recombinant neuraminidase described herein comprises a tetramerization domain comprising the amino acid sequence of SEQ ID NO:6.

In specific embodiments, a recombinant neuraminidase described herein comprises a tetramerization domain from SEPPALLATA-like MADS domain transcription factor from Arabidopsis thaliana (SMDTF). The tetramerization domain from SEPPALLATA-like MADS domain transcription factor from Arabidopsis thaliana (SMDTF) may comprise an amino acid sequence known in the art. The tetramerization domain from SEPPALLATA-like MADS domain transcription factor from Arabidopsis thaliana (SMDTF) may comprise SEQ ID NO:5, or an amino acid sequence that is at least 80%, at least 85%, at least 90%, at least 95%, at least 98%, or a least 99% identical to SEQ ID NO:5.

In some embodiments, a recombinant neuraminidase described herein comprises an amino acid sequence having at least 80% identity to a tetramerization domain from SEPPALLATA-like MADS domain transcription factor from Arabidopsis thaliana (SMDTF) described herein (e.g., SEQ ID NO:5) or known in the art. In some embodiments, a recombinant neuraminidase described herein comprises an amino acid sequence having at least 85%, at least 90%, at least 95%, or at least 98% identity to a tetramerization domain from SEPPALLATA-like MADS domain transcription factor from Arabidopsis thaliana (SMDTF) described herein (e.g., SEQ ID NO:5) or known in the art. In some embodiments, a recombinant neuraminidase described herein comprises an amino acid sequence having at least 80% identity to SEQ ID NO:5. In some embodiments, a recombinant neuraminidase described herein comprises an amino acid sequence having at least 85%, at least 90%, at least 95%, at least 98%, or at least 99% identity to SEQ ID NO:5. In some embodiments, a recombinant neuraminidase described herein comprises an amino acid sequence having at least 95%, at least 98%, or at least 99% identity to SEQ ID NO:5. In some embodiments, a recombinant neuraminidase described herein comprises a tetramerization domain comprising the amino acid sequence of SEQ ID NO:5.

In some embodiments, a recombinant neuraminidase described herein comprises a tetramerization domain from SEPPALLATA-like MADS domain transcription factor from Arabidopsis thaliana (SMDTF). In some embodiments, a recombinant neuraminidase described herein comprises tetramerization domain comprising an amino acid sequence having at least 80% identity to a SEPPALLATA-like MADS domain transcription factor from Arabidopsis thaliana (SMDTF) described herein (e.g., SEQ ID NO:5) or known in the art. In some embodiments, a recombinant neuraminidase described herein comprises tetramerization domain comprising an amino acid sequence having at least 85%, at least 90%, at least 95%, or at least 98% identity to a SEPPALLATA-like MADS domain transcription factor from Arabidopsis thaliana (SMDTF) described herein (e.g., SEQ ID NO:5) or known in the art. In some embodiments, a recombinant neuraminidase described herein comprises tetramerization domain comprising an amino acid sequence having at least 95%, at least 98%, or at least 99% identity to a SEPPALLATA-like MADS domain transcription factor from Arabidopsis thaliana (SMDTF) described herein (e.g., SEQ ID NO:5) or known in the art. In some embodiments, a recombinant neuraminidase described herein comprises tetramerization domain comprising an amino acid sequence having at least 80% identity SEQ ID NO:5. In some embodiments, a recombinant neuraminidase described herein comprises tetramerization domain comprising an amino acid sequence having at least 85%, at least 90%, at least 95%, at least 98%, or at least 99% identity to SEQ ID NO:5. In some embodiments, a recombinant neuraminidase described herein comprises tetramerization domain comprising an amino acid sequence having at least 95%, at least 98%, or at least 99% identity to SEQ ID NO:5. In certain embodiments, a recombinant neuraminidase described herein comprises tetramerization domain comprising the amino acid sequence of SEQ ID NO:5.

In specific embodiments, recombinant neuraminidase described herein comprises a tetramerization domain of a PiLZ structure from Xanthomonas campestris. The tetramerization domain of a PILZ structure from Xanthomonas campestris may comprise an amino acid sequence known in the art. The tetramerization domain of a PILZ structure from Xanthomonas campestris may comprise SEQ ID NO:7, or an amino acid sequence that is at least 80% identical (e.g., at least 85%, at least 90%, at least 95%, at least 98%, or at least 99% identical to SEQ ID NO:7.

In some embodiments, a recombinant neuraminidase described herein comprises an amino acid sequence having at least 80% identity to a tetramerization domain of a PILZ structure from Xanthomonas campestris described herein (e.g., SEQ ID NO:7) or known in the art. In a specific embodiment, a recombinant neuraminidase described herein comprises an amino acid sequence having at least 85%, at least 90%, at least 95%, or at least 98% identity to a tetramerization domain of a PILZ structure from Xanthomonas campestris described herein (e.g., SEQ ID NO:7) or known in the art. In some embodiments, a recombinant neuraminidase described herein comprises an amino acid sequence having at least 80% identity to SEQ ID NO:7. In a specific embodiment, a recombinant neuraminidase described herein comprises an amino acid sequence having at least 85%, at least 90%, at least 95%, at least 98%, or at least 99% identity to SEQ ID NO:7. In certain embodiments, a recombinant neuraminidase described herein comprises the amino acid sequence of SEQ ID NO:7.

In some embodiments, a recombinant neuraminidase described herein comprises a tetramerization domain comprising an amino acid sequence having at least 80% identity to a tetramerization domain of a PiLZ structure from Xanthomonas campestris described herein (e.g., SEQ ID NO:7) or known in the art. In a specific embodiment, a recombinant neuraminidase described herein comprises a tetramerization domain comprising an amino acid sequence having at least 85%, at least 90%, at least 95%, or at least 98% identity to a tetramerization domain of a PiLZ structure from Xanthomonas campestris described herein (e.g., SEQ ID NO:7) or known in the art. In some embodiments, a recombinant neuraminidase described herein comprises a tetramerization domain comprising an amino acid sequence having at least 80% identity to SEQ ID NO:7. In a specific embodiment, a recombinant neuraminidase described herein comprises a tetramerization domain comprising an amino acid sequence having at least 85%, at least 90%, at least 95%, at least 98%, or at least 99% identity to SEQ ID NO:7. In certain embodiments, a recombinant neuraminidase described herein comprises a tetramerization domain comprising the amino acid sequence of SEQ ID NO:7.

In some embodiments, a recombinant neuraminidase described herein comprises a Dictyocaulus viviparus ACE tetramerization domain. The Dictyocaulus viviparus ACE tetramerization domain may comprise an amino acid sequence known in the art. The Dictyocaulus viviparus ACE tetramerization domain may comprise SEQ ID NO:9, or an amino acid sequence that is at least 80% identical (e.g., at least 85%, at least 90%, at least 95%, or at least 98% identical) to SEQ ID NO:9.

In some embodiments, a recombinant neuraminidase described herein comprises an amino acid sequence having at least 80% identity to a Dictyocaulus viviparus ACE tetramerization domain described herein (e.g., SEQ ID NO:9) or known in the art. In some embodiments, a recombinant neuraminidase described herein comprises an amino acid sequence having at least 85%, at least 90%, at least 95%, at least 98%, or at least 99% identity to a Dictyocaulus viviparus ACE tetramerization domain described herein (e.g., SEQ ID NO:9) or known in the art. In some embodiments, a recombinant neuraminidase described herein comprises an amino acid sequence having at least 95%, at least 98%, or at least 99% identity to a Dictyocaulus viviparus ACE tetramerization domain described herein (e.g., SEQ ID NO:9) or known in the art. In some embodiments, a recombinant neuraminidase described herein comprises an amino acid sequence having at least 80% identity to SEQ ID NO:9. In a specific embodiment, a recombinant neuraminidase described herein comprises an amino acid sequence having at least 85%, at least 90%, at least 95%, at least 98%, or at least 99% identity to SEQ ID NO:9. In a specific embodiment, a recombinant neuraminidase described herein comprises an amino acid sequence having at least 95%, at least 98%, or at least 99% identity to SEQ ID NO:9. In certain embodiments, a recombinant neuraminidase described herein comprises the amino acid sequence of SEQ ID NO:9.

In some embodiments, a recombinant neuraminidase described herein comprises a tetramerization domain comprising an amino acid sequence having at least 80% identity to a Dictyocaulus viviparus ACE tetramerization domain described herein (e.g., SEQ ID NO:9) or known in the art. In a specific embodiment, a recombinant neuraminidase described herein comprises a tetramerization domain comprising an amino acid sequence having at least 85%, at least 90%, at least 95%, or at least 98% identity to a Dictyocaulus viviparus ACE tetramerization domain described herein (e.g., SEQ ID NO:9) or known in the art. In some embodiments, a recombinant neuraminidase described herein comprises a tetramerization domain comprising an amino acid sequence having at least 80% identity to SEQ ID NO:9. In some embodiments, a recombinant neuraminidase described herein comprises a tetramerization domain comprising an amino acid sequence having at least 85%, at least 90%, at least 95%, at least 98%, or at least 99% identity to SEQ ID NO:9. In some embodiments, a recombinant neuraminidase described herein comprises a tetramerization domain comprising an amino acid sequence having at least 95%, at least 98%, or at least 99% identity to SEQ ID NO:9. In certain embodiments, a recombinant neuraminidase described herein comprises a tetramerization domain comprising the amino acid sequence of SEQ ID NO:9.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions ×100%). In one embodiment, the two sequences are the same length. In a certain embodiment, the percent identity is determined over the entire length of an amino acid sequence or nucleotide sequence. The determination of percent identity between two sequences (e.g., amino acid sequences or nucleic acid sequences) can also be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). In another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In a specific embodiment, provided herein is a recombinant neuraminidase comprising an influenza virus neuraminidase globular head domain and a paramyxovirus phosphoprotein tetramerization domain, wherein the recombinant neuraminidase lacks influenza virus neuraminidase stalk, transmembrane and cytoplasmic domains, and wherein the tetramerization domain comprises a measles virus phosphoprotein tetramerization domain. In a specific embodiment, a recombinant neuraminidase comprises an influenza virus neuraminidase globular head domain and a paramyxovirus phosphoprotein tetramerization domain, wherein the recombinant neuraminidase lacks influenza virus neuraminidase stalk, transmembrane and cytoplasmic domains, and wherein the tetramerization domain comprises an amino acid sequence at least 80%, at least 85%, at least 90%, at least 95%, at least 98%, or at least 99% identical to SEQ ID NO:8. In a specific embodiment, a recombinant neuraminidase comprises an influenza virus neuraminidase globular head domain and a paramyxovirus phosphoprotein tetramerization domain, wherein the recombinant neuraminidase lacks influenza virus neuraminidase stalk, transmembrane and cytoplasmic domains, and wherein the tetramerization domain comprises an amino acid sequence at least 95%, at least 98%, or at least 99% identical to SEQ ID NO:8. In a specific embodiment, a recombinant neuraminidase comprises an influenza virus neuraminidase globular head domain and a paramyxovirus phosphoprotein tetramerization domain, wherein the recombinant neuraminidase lacks influenza virus neuraminidase stalk, transmembrane and cytoplasmic domains, and wherein the tetramerization domain the amino acid sequence of SEQ ID NO:8.

In some embodiments, recombinant neuraminidase comprising the globular head domain of an influenza virus neuraminidase known to one of skill in the art. In some embodiments, the influenza virus neuraminidase is a human influenza virus neuraminidase. Human influenza virus neuraminidases are known in the art. See below for examples of human influenza virus neuraminidases. In some embodiments, the influenza virus neuraminidase is a swine influenza virus neuraminidase. Swine influenza virus neuraminidases are known in the art. See below for examples of swine influenza virus neuraminidases. In some embodiments, the influenza virus neuraminidase is an equine influenza virus neuraminidase. Equine influenza virus neuraminidases are known in the art. In some embodiments, the influenza virus neuraminidase is an avian influenza virus neuraminidase. Avian influenza virus neuraminidases are known in the art. See below for examples of avian influenza virus neuraminidases. In some embodiments, an influenza virus neuraminidase is a seal influenza virus neuraminidase. Seal influenza virus neuraminidases are known in the art.

In some embodiments, the influenza virus neuraminidase is an influenza A virus neuraminidase. In some embodiments, the influenza virus neuraminidase is from an influenza A virus as described herein (e.g., in Section 5.3, or Example 1). In specific embodiments, the influenza A virus neuraminidase is an N1, N2, N3, N4, N5, N6, N7, N8 or N9 subtype. In certain embodiments, the influenza A virus neuraminidase is subtype N1. In certain embodiments, the influenza A virus neuraminidase is subtype N2. Examples of specific influenza A virus neuraminidases include the neuraminidase of an influenza A virus strains described herein. In a specific embodiment, the influenza A virus neuraminidase is of influenza virus A/Michigan/45/2015 (H3N2) or A/Michigan/45/2015-like. In a specific embodiment, the influenza A virus neuraminidase is influenza virus A/Kansas/14/2017 (H3N2) or A/Kansas/14/2017 (H3N2)-like virus neuraminidase.

In some embodiments, the influenza virus neuraminidase is an influenza B virus neuraminidase. In certain embodiments, the influenza B virus neuraminidase is a human influenza B virus neuraminidase. Human influenza B virus neuraminidases are known in the art. In certain embodiments, the influenza B virus neuraminidase is a seal influenza B virus neuraminidase. Seal influenza B virus neuraminidases are known in the art. Examples of specific influenza B virus neuraminidases include the neuraminidase of an influenza virus B/Victoria/2/87-lineage virus. In a specific embodiment, the influenza B virus neuraminidase is influenza virus B/Colorado/06/2017 or B/Colorado/06/2017-like virus (B/Victoria/2/87 lineage) neuraminidase.

GenBank™ Accession No. AAA43397.1 provides an exemplary amino acid sequence for a human influenza virus neuraminidase. GenBank™ Accession No. ABG23658.1 (GI: 108946273), GenBank™ Accession No. NP_040981.1 (GI: 8486128), GenBank™ Accession No. AAA43412.1 (GI: 324508), GenBank™ Accession No. ABE97720.1 (GI: 93008579), GenBank™ Accession No. ABE97719.1 (GI: 93008577), and GenBank™ Accession No. ABE97718.1 (GI: 93008575) provide exemplary amino acid sequences for human influenza virus neuraminidases. GenBank™ Accession No. CRI06477.1 provides an exemplary amino acid sequence for a swine influenza virus neuraminidase. GenBank™ Accession No. AAQ90293.1 provides an exemplary amino acid sequence for an equine influenza virus neuraminidase. GenBank™ Accession No. AEX30531.1 (GI: 371449652), GenBank™ Accession No. AEX30532.1 (GI: 371449654), GenBank™ Accession No. AIA62041.1 (GI: 641454926), GenBank™ Accession No. AII30325.1 (GI: 670605039), GenBank™ Accession No. AGO18161.1 (GI: 513130855), and GenBank™ Accession No. AAS89005.1 (GI: 46360357) provide exemplary amino acid sequences for avian influenza virus neuraminidases. Sequences of influenza virus genes may also be found in the Influenza Research Database. For example, influenza virus neuraminidase sequences may be found in the Influenza Research Database under Accession No. FJ66084 and Accession No. KF90392.

In some embodiments, the influenza virus neuraminidase globular head domain comprises an amino acid sequence having at least 80% identity to the globular head domain of an influenza A virus neuraminidase described herein. In some embodiments, the influenza virus neuraminidase globular head domain comprises an amino acid sequence having at least 85%, at least 90%, at least 95%, at least 98%, or at least 99% identity to the globular head domain of an influenza A virus neuraminidase described herein. In some embodiments, the influenza virus neuraminidase globular head domain comprises an amino acid sequence having at least 95%, at least 98%, or at least 99% identity to the globular head domain of an influenza A virus neuraminidase described herein. In some embodiment, the influenza virus neuraminidase globular head domain comprises the amino acid sequence of an influenza A virus neuraminidase described herein.

In some embodiments, the influenza virus neuraminidase globular head domain comprises an amino acid sequence having at least 80% identity to the globular head domain of one of the influenza virus neuraminidases in FIG. 1. In some embodiments, the influenza virus neuraminidase globular head domain comprises an amino acid sequence having at least 85%, at least 90%, at least 95%, at least 98%, or at least 99% identity to the globular head domain of one of the influenza virus neuraminidases in FIG. 1. In some embodiments, the influenza virus neuraminidase globular head domain comprises an amino acid sequence having at least 95%, at least 98%, or at least 99% identity to the globular head domain of one of the influenza virus neuraminidases in FIG. 1. In some embodiments, the influenza virus neuraminidase globular head domain comprises the amino acid sequence of the globular head domain of one of the influenza virus neuraminidases in FIG. 1.

In some embodiments, the influenza virus neuraminidase globular head domain comprises an amino acid sequence having at least 80% identity to SEQ ID NO:31. In some embodiments, the influenza virus neuraminidase globular head domain comprises an amino acid sequence having at least 85%, at least 90%, at least 95%, at least 98%, or at least 99% identity to SEQ ID NO:31. In some embodiments, the influenza virus neuraminidase globular head domain comprises an amino acid sequence having at least 95%, at least 98%, or at least 99% identity to SEQ ID NO:31. In some embodiments, the influenza virus neuraminidase globular head domain comprises the amino acid sequence of SEQ ID NO:31. In specific embodiments, an influenza virus neuraminidase globular head domain comprises or consists of the amino acid sequence of

(SEQ ID NO: 31)
SVKLAGNSSLCPVSGWAIYSKDNSVRIGSKGDVFVIREPFISCSPLECR
TFFLTQGALLNDKHSNGTIKDRSPYRTLMSCPIGEVPSPYNSRFESVAW
SASACHDGINWLTIGISGPDSGAVAVLKYNGIITDTIKSWRNNILRTQE
SECACVNGSCFTIMTDGPSDGQASYKIFRIEKGKIIKSVEMKAPNYHYE
ECSCYPDSSEITCVCRDNWHGSNRPWVSFNQNLEYQMGYICSGVFGDNP
RPNDKTGSCGPVSSNGANGVKGFSFKYGNGVWIGRTKSISSRKGFEMIW
DPNGWTGTDNKFSIKQDIVGINEWSGYSGSFVQHPELTGLDCIRPCFWV
ELIRGRPEENTIWTSGSSISFCGVNSDTVGWSWPDGAELPFTIDK.

In some embodiments, the influenza virus neuraminidase globular head domain comprises an amino acid sequence having at least 80% identity to SEQ ID NO:32. In some embodiments, the influenza virus neuraminidase globular head domain comprises an amino acid sequence having at least 85%, at least 90%, at least 95%, at least 98%, or at least 99% identity to SEQ ID NO:32. In some embodiments, the influenza virus neuraminidase globular head domain comprises an amino acid sequence having at least 95%, at least 98%, or at least 99% identity to SEQ ID NO:32. In some embodiments, the influenza virus neuraminidase globular head domain comprises the amino acid sequence of SEQ ID NO:32. In specific embodiments, an influenza virus neuraminidase globular head domain comprises or consists of the amino acid sequence of

(SEQ ID NO: 32)
ICPKPAEYRNWSKPQCGITGFAPFSKDNSIRLSAGGDIWVTREPYVSCD
PDKCYQFALGQGTTINNVHSNNTARDRTPHRTLLMNELGVPFHLGTKQV
CIAWSSSSCHDGKAWLHVCITGDDKNATASFIYNGRLVDSVVSWSKDIL
RTQESECVCINGTCTVVMTDGNATGKADTKILFIEEGKIVHTSKLSGSA
QHVEECSCYPRYPGVRCVCRDNWKGSNRPIVDINIKDHSIVSSYVCSGL
VGDTPRKTDSSSSSHCLNPNNEKGGHGVKGWAFDDGNDVWMGRTINETS
RLGYETFKVVEGWSNPKSKLQINRQVIVDRGDRSGYSGIFSVEGKSCIN
RCFYVELIRGRKEETEVLWTSNSIVVFCGTSGTYGTGSWPDGADLNLMH
I.

In some embodiments, the influenza virus neuraminidase globular head domain comprises an amino acid sequence having at least 80% identity to SEQ ID NO:33. In some embodiments, the influenza virus neuraminidase globular head domain comprises an amino acid sequence having at least 85%, at least 90%, at least 95%, at least 98%, or at least 99% identity to SEQ ID NO:33. In some embodiments, the influenza virus neuraminidase globular head domain comprises an amino acid sequence having at least 95%, at least 98%, or at least 99% identity to SEQ ID NO:33. In some embodiments, the influenza virus neuraminidase globular head domain comprises the amino acid sequence of SEQ ID NO:33. In specific embodiments, an influenza virus neuraminidase globular head domain comprises or consists of the amino acid sequence of

(SEQ ID NO: 33)
LLLPEPEWTYPRLSCPGSTFQKALLISPHRFGETKGNSAPLIIREPFVA
CGPNECKHFALTHYAAQPGGYYNGTRGDRNKLRHLISVKLGKIPTVENS
IFHMAAWSGSACHDGKEWTYIGVDGPDNNALLKVKYGEAYTDTYHSYAN
NILRTQESACNCIGGNCYLMITDGSASGVSECRFLKIREGRIIKEIFPT
GRVKHTEECTCGFASNKTIECACRDNRYTAKRPFVKLNVETDTAEIRLM
CTDTYLDTPRPNDGSITGPCESDGDKGSGGIKGGFVHQRMKSKIGRWYS
RTMSQTERMGMGLYVKYGGDPWADSDALAFSGVMVSMKEPGWYSFGFEI
KDKKCDVPCIGIEMVHDGGKETWHSAATAIYCLMGSGQLLWDTVTGVDM
AL.

In certain embodiments, a recombinant neuraminidase provided herein further comprises one or more polypeptide domains. Useful polypeptide domains include domains that facilitate secretion, purification, folding and cleavage of portions of a polypeptide. Signal polypeptides may be used to direct recombinant protein towards a secretory pathway during expression. In some embodiments, a recombinant neuraminidase provided herein further comprises a signal peptide. In specific embodiments, the signal peptide comprises the amino acid sequence of MLLVNQSHQGFNKEHTSKMVSAIVLYVLLAAAAHSAFAADP (SEQ ID NO:34). In some embodiments, a recombinant neuraminidase provided herein further comprises a purification tag. In specific embodiments, a purification tag comprises a histidine (His) tag, a FLAG tag, or other purification tag that can facilitate purification of an neuraminidase polypeptide provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In specific embodiments, the His tag consists of the amino acid sequence HHHHHH (SEQ ID NO:35). Cleavage sites can be used to facilitate cleavage of a portion of a polypeptide; for example cleavage of a purification tag, signal peptide, tetramerization domain, globular head domain, or any combination thereof from a recombinant neuraminidase provided herein. In some embodiments, a recombinant neuraminidase provided herein further comprises a cleavage site. In specific embodiments, a cleavage site comprises a thrombin cleavage site. In certain embodiments, the cleavage site comprises the amino acid sequence of LVPRGSP (SEQ ID NO:36). In a specific embodiment, the cleavage site comprises the amino acid sequence SLVPRGSPSR (SEQ ID NO:37). In certain embodiments, the cleavage site is a Tobacco Etch Virus (TEV) protease cleavage site. In certain embodiments, the cleavage site comprises the e.g. amino acid sequence of Glu-Asn-Leu-Tyr-Phe-Gln-(Gly/Ser) (SEQ ID NO:38). In some embodiments, a recombinant neuraminidase provided herein does not include a cleavage site. In some embodiments, a recombinant neuraminidase provided herein comprises a signal peptide and a purification tag (e.g., a histidine tag). In some embodiments, a recombinant neuraminidase provided herein comprises a signal peptide, purification tag (e.g., a His tag), a tetramerization domain (e.g., paramyxovirus phosphoprotein, such as, e.g., Measles virus phosphoprotein tetramerization domain), a cleavage site (e.g., a thrombin cleavage site), and the globular head domain of an influenza virus NA.

In a specific embodiment, a recombinant neuraminidase provided herein includes in order a signal peptide, purification tag (e.g., a His tag), a tetramerization domain (e.g., paramyxovirus phosphoprotein, such as, e.g., Measles virus phosphoprotein tetramerization domain), a cleavage site (e.g., a thrombin cleavage site), and the globular head domain of an influenza virus NA. In a specific embodiment, a recombinant neuraminidase provided herein includes in order a purification tag (e.g., a His tag), a tetramerization domain (e.g., paramyxovirus phosphoprotein, such as, e.g., Measles virus phosphoprotein tetramerization domain), a cleavage site (e.g., a thrombin cleavage site), and the globular head domain of an influenza virus NA.

In a specific embodiment, a recombinant neuraminidase provided herein includes in order a signal peptide, a purification tag (e.g., a His tag), a tetramerization domain (e.g., paramyxovirus phosphoprotein, such as, e.g., Measles virus phosphoprotein tetramerization domain), and the globular head domain of an influenza virus NA. In a specific embodiment, a recombinant neuraminidase provided herein includes in order a purification tag (e.g., a His tag), a tetramerization domain (e.g., paramyxovirus phosphoprotein, such as, e.g., Measles virus phosphoprotein tetramerization domain), and the globular head domain of an influenza virus NA.

In a specific embodiment, a recombinant neuraminidase provided herein includes in order a signal peptide, a tetramerization domain (e.g., paramyxovirus phosphoprotein, such as, e.g., Measles virus phosphoprotein tetramerization domain), and the globular head domain of an influenza virus NA. In a specific embodiment, a recombinant neuraminidase provided herein includes in order a tetramerization domain (e.g., paramyxovirus phosphoprotein, such as, e.g., Measles virus phosphoprotein tetramerization domain), and the globular head domain of an influenza virus NA.

In another specific embodiment, a recombinant neuraminidase described herein comprises the components of a signal peptide, a purification tag, a paramyxovirus phosphoprotein tetramerization domain, a cleavage site, and a globular head domain of an influenza virus neuraminidase in the order described in the Example 1 of Section 6, infra.

In some embodiments, provided herein is a recombinant neuraminidase comprising an amino acid sequence having at least 80% identity to SEQ ID NO:40. In some sequence having at least 85%, at least 90%, at least 95%, at least 98%, or at least 99% identity to SEQ ID NO:40. In some embodiments, provided herein is a recombinant neuraminidase comprising an amino acid sequence having at least 95%, at least 98%, or at least 99% identity to SEQ ID NO:40. In some embodiments, provided herein is a recombinant neuraminidase comprising an amino acid sequence having at least 85%, at least 90%, at least 95%, at least 98%, or at least 99% identity to SEQ ID NO:40 without the signal sequence. In some embodiments, provided herein is a recombinant neuraminidase comprising an amino acid sequence having at least 95%, at least 98%, or at least 99% identity to SEQ ID NO:40 without the signal sequence. In some embodiments, provided herein is a recombinant neuraminidase comprising the amino acid sequence of SEQ ID NO:40. In some embodiments, provided herein is a recombinant neuraminidase comprising the amino acid sequence of SEQ ID NO:40 without the signal sequence.

In some embodiments, provided herein is a recombinant neuraminidase comprising an amino acid sequence having at least 80% identity to SEQ ID NO:42. In some embodiments, provided herein is a recombinant neuraminidase comprising an amino acid sequence having at least 85%, at least 90%, at least 95%, at least 98%, or at least 99% identity to SEQ ID NO:42. In some embodiments, provided herein is a recombinant neuraminidase comprising an amino acid sequence having at least 95%, at least 98%, or at least 99% identity to SEQ ID NO:42. In some embodiments, provided herein is a recombinant neuraminidase comprising an amino acid sequence having at least 85%, at least 90%, at least 95%, at least 98%, or at least 99% identity to SEQ ID NO:42 without the signal sequence. In some embodiments, provided herein is a recombinant neuraminidase comprising an amino acid sequence having at least 95%, at least 98%, or at least 99% identity to SEQ ID NO:42 without the signal sequence. In some embodiments, provided herein is a recombinant neuraminidase comprising the amino acid sequence of SEQ ID NO:42. In some embodiments, provided herein is a recombinant neuraminidase comprising the amino acid sequence of SEQ ID NO:42 without the signal sequence.

In some embodiments, provided herein is a recombinant neuraminidase comprising an amino acid sequence having at least 80% identity to SEQ ID NO:44. In some embodiments, provided herein is a recombinant neuraminidase comprising an amino acid sequence having at least 85%, at least 90%, at least 95%, at least 98%, or at least 99% identity to SEQ ID NO:44. In some embodiments, provided herein is a recombinant neuraminidase comprising an amino acid sequence having at least 95%, at least 98%, or at least 99% identity to SEQ ID NO:44. In some embodiments, provided herein is a recombinant neuraminidase comprising an amino acid sequence having at least 85%, at least 90%, at least 95%, at least 98%, or at least 99% identity to SEQ ID NO:44 without the signal sequence. In some embodiments, provided herein is a recombinant neuraminidase comprising an amino acid sequence having at least 95%, at least 98%, or at least 99% identity to SEQ ID NO:44 without the signal sequence. In some embodiments, provided herein is a recombinant neuraminidase comprising the amino acid sequence of SEQ ID NO:44.

In some embodiments, provided herein is a recombinant neuraminidase comprising an amino acid sequence having at least 80% identity to SEQ ID NO:46. In some embodiments, provided herein is a recombinant neuraminidase comprising an amino acid sequence having at least 85%, at least 90%, at least 95%, at least 98%, or at least 99% identity to SEQ ID NO:46. In some embodiments, provided herein is a recombinant neuraminidase comprising an amino acid sequence having at least 95%, at least 98%, or at least 99% identity to SEQ ID NO:46. In some embodiments, provided herein is a recombinant neuraminidase comprising an amino acid sequence having at least 85%, at least 90%, at least 95%, at least 98%, or at least 99% identity to SEQ ID NO:46 without the signal sequence. In some embodiments, provided herein is a recombinant neuraminidase comprising an amino acid sequence having at least 95%, at least 98%, or at least 99% identity to SEQ ID NO:46 without the signal sequence. In some embodiments, provided herein is a recombinant neuraminidase comprising the amino acid sequence of SEQ ID NO:46. In some embodiments, provided herein is a recombinant neuraminidase comprising the amino acid sequence of SEQ ID NO:46 without the signal sequence.

In some embodiments, provided herein is a recombinant neuraminidase comprising an amino acid sequence having at least 80% identity to SEQ ID NO:48. In some embodiments, provided herein is a recombinant neuraminidase comprising an amino acid sequence having at least 85%, at least 90%, at least 95%, at least 98%, or at least 99% identity to SEQ ID NO:48. In some embodiments, provided herein is a recombinant neuraminidase comprising an amino acid sequence having at least 95%, at least 98%, or at least 99% identity to SEQ ID NO:48. In some embodiments, provided herein is a recombinant neuraminidase comprising an amino acid sequence having at least 85%, at least 90%, at least 95%, at least 98%, or at least 99% identity to SEQ ID NO:48 without the signal sequence. In some embodiments, provided herein is a recombinant neuraminidase comprising an amino acid sequence having at least 95%, at least 98%, or at least 99% identity to SEQ ID NO:48 without the signal sequence. In some embodiments, provided herein is a recombinant neuraminidase comprising the amino acid sequence of SEQ ID NO:48.

In some embodiments, provided herein is a recombinant neuraminidase comprising an amino acid sequence having at least 80% identity to SEQ ID NO:50. In some embodiments, provided herein is a recombinant neuraminidase comprising an amino acid sequence having at least 85%, at least 90%, at least 95%, at least 98%, or at least 99% identity to SEQ ID NO:50. In some embodiments, provided herein is a recombinant neuraminidase comprising an amino acid sequence having at least 95%, at least 98%, or at least 99% identity to SEQ ID NO:50. In some embodiments, provided herein is a recombinant neuraminidase comprising an amino acid sequence having at least 85%, at least 90%, at least 95%, at least 98%, or at least 99% identity to SEQ ID NO:50 without the signal sequence. In some embodiments, provided herein is a recombinant neuraminidase comprising an amino acid sequence having at least 95%, at least 98%, or at least 99% identity to SEQ ID NO:50 without the signal sequence. In some embodiments, provided herein is a recombinant neuraminidase comprising the amino acid sequence of SEQ ID NO:50. In some embodiments, provided herein is a recombinant neuraminidase comprising the amino acid sequence of SEQ ID NO:50 without the signal sequence.

In some embodiments, provided herein is a recombinant neuraminidase comprising an amino acid sequence having at least 80% identity to SEQ ID NO:52. In some embodiments, provided herein is a recombinant neuraminidase comprising an amino acid sequence having at least 85%, at least 90%, at least 95%, at least 98%, or at least 99% identity to SEQ ID NO:52. In some embodiments, provided herein is a recombinant neuraminidase comprising an amino acid sequence having at least 95%, at least 98%, or at least 99% identity to SEQ ID NO:52. In some embodiments, provided herein is a recombinant neuraminidase comprising an amino acid sequence having at least 85%, at least 90%, at least 95%, at least 98%, or at least 99% identity to SEQ ID NO:52 without the signal sequence. In some embodiments, provided herein is a recombinant neuraminidase comprising an amino acid sequence having at least 95%, at least 98%, or at least 99% identity to SEQ ID NO:52 without the signal sequence. In some embodiments, provided herein is a recombinant neuraminidase comprising the amino acid sequence of SEQ ID NO:52. In some embodiments, provided herein is a recombinant neuraminidase comprising the amino acid sequence of SEQ ID NO:52 without the signal sequence.

In some embodiments, provided herein is a recombinant neuraminidase comprising an amino acid sequence having at least 80% identity to SEQ ID NO:54. In some sequence having at least 85%, at least 90%, at least 95%, at least 98%, or at least 99% identity to SEQ ID NO:54. In some embodiments, provided herein is a recombinant neuraminidase comprising an amino acid sequence having at least 85%, at least 90%, at least 95%, at least 98%, or at least 99% identity to SEQ ID NO:54. In some embodiments, provided herein is a recombinant neuraminidase comprising an amino acid sequence having at least 85%, at least 90%, at least 95%, at least 98%, or at least 99% identity to SEQ ID NO:54 without the signal sequence. In some embodiments, provided herein is a recombinant neuraminidase comprising an amino acid sequence having at least 95%, at least 98%, or at least 99% identity to SEQ ID NO:54 without the signal sequence. In some embodiments, provided herein is a recombinant neuraminidase comprising the amino acid sequence of SEQ ID NO:54. In some embodiments, provided herein is a recombinant neuraminidase comprising the amino acid sequence of SEQ ID NO:54 without the signal sequence.

In some embodiments, provided herein is a recombinant neuraminidase comprising an amino acid sequence having at least 80% identity to SEQ ID NO:56. In some embodiments, provided herein is a recombinant neuraminidase comprising an amino acid sequence having at least 85%, at least 90%, at least 95%, at least 98%, or at least 99% identity to SEQ ID NO:56. In some embodiments, provided herein is a recombinant neuraminidase comprising an amino acid sequence having at least 95%, at least 98%, or at least 99% identity to SEQ ID NO:56. In some embodiments, provided herein is a recombinant neuraminidase comprising an amino acid sequence having at least 85%, at least 90%, at least 95%, at least 98%, or at least 99% identity to SEQ ID NO:56 without the signal sequence. In some embodiments, provided herein is a recombinant neuraminidase comprising an amino acid sequence having at least 95%, at least 98%, or at least 99% identity to SEQ ID NO:56 without the signal sequence. In some embodiments, provided herein is a recombinant neuraminidase comprising the amino acid sequence of SEQ ID NO:56. In some embodiments, provided herein is a recombinant neuraminidase comprising the amino acid sequence of SEQ ID NO:56 without the signal sequence.

In some embodiments, provided herein is a recombinant neuraminidase comprising an amino acid sequence having at least 80% identity to SEQ ID NO:58. In some embodiments, provided herein is a recombinant neuraminidase comprising an amino acid sequence having at least 85%, at least 90%, at least 95%, at least 98%, or at least 99% identity to SEQ ID NO:58. In some embodiments, provided herein is a recombinant neuraminidase comprising an amino acid sequence having at least 95%, at least 98%, or at least 99% identity to SEQ ID NO:58. In some embodiments, provided herein is a recombinant neuraminidase comprising an amino acid sequence having at least 85%, at least 90%, at least 95%, at least 98%, or at least 99% identity to SEQ ID NO:58 without the signal sequence. In some embodiments, provided herein is a recombinant neuraminidase comprising an amino acid sequence having at least 95%, at least 98%, or at least 99% identity to SEQ ID NO:58 without the signal sequence. In some embodiments, provided herein is a recombinant neuraminidase comprising the amino acid sequence of SEQ ID NO:58. In some embodiments, provided herein is a recombinant neuraminidase comprising the amino acid sequence of SEQ ID NO:58 without the signal sequence.

In some embodiments, provided herein is a recombinant neuraminidase comprising an amino acid sequence having at least 80% identity to SEQ ID NO:60. In some embodiments, provided herein is a recombinant neuraminidase comprising an amino acid sequence having at least 85%, at least 90%, at least 95%, at least 98%, or at least 99% identity to SEQ ID NO:60. In some embodiments, provided herein is a recombinant neuraminidase comprising an amino acid sequence having at least 95%, at least 98%, or at least 99% identity to SEQ ID NO:60. In some embodiments, provided herein is a recombinant neuraminidase comprising an amino acid sequence having at least 85%, at least 90%, at least 95%, at least 98%, or at least 99% identity to SEQ ID NO:60 without the signal sequence. In some embodiments, provided herein is a recombinant neuraminidase comprising an amino acid sequence having at least 95%, at least 98%, or at least 99% identity to SEQ ID NO:60 without the signal sequence. In some embodiments, provided herein is a recombinant neuraminidase comprising the amino acid sequence of SEQ ID NO:60. In some embodiments, provided herein is a recombinant neuraminidase comprising the amino acid sequence of SEQ ID NO:60 without the signal sequence.

In some embodiments, provided herein is a recombinant neuraminidase comprising an amino acid sequence having at least 80% identity to SEQ ID NO:62. In some embodiments, provided herein is a recombinant neuraminidase comprising an amino acid sequence having at least 85%, at least 90%, at least 95%, at least 98%, or at least 99% identity to SEQ ID NO:62. In some embodiments, provided herein is a recombinant neuraminidase comprising an amino acid sequence having at least 95%, at least 98%, or at least 99% identity to SEQ ID NO:62. In some embodiments, provided herein is a recombinant neuraminidase comprising an amino acid sequence having at least 85%, at least 90%, at least 95%, at least 98%, or at least 99% identity to SEQ ID NO:62 without the signal sequence. In some embodiments, provided herein is a recombinant neuraminidase comprising an amino acid sequence having at least 95%, at least 98%, or at least 99% identity to SEQ ID NO:62 without the signal sequence. In some embodiments, provided herein is a recombinant neuraminidase comprising the amino acid sequence of SEQ ID NO:62. In some embodiments, provided herein is a recombinant neuraminidase comprising the amino acid sequence of SEQ ID NO:62 without signal sequence.

In some embodiments, provided herein is a recombinant neuraminidase comprising an amino acid sequence having at least 80% identity to SEQ ID NO:64. In some embodiments, provided herein is a recombinant neuraminidase comprising an amino acid sequence having at least 85%, at least 90%, at least 95%, at least 98%, or at least 99% identity to SEQ ID NO:64. In some embodiments, provided herein is a recombinant neuraminidase comprising an amino acid sequence having at least 95%, at least 98%, or at least 99% identity to SEQ ID NO:64. In some embodiments, provided herein is a recombinant neuraminidase comprising an amino acid sequence having at least 85%, at least 90%, at least 95%, at least 98%, or at least 99% identity to SEQ ID NO:64 without the signal sequence. In some embodiments, provided herein is a recombinant neuraminidase comprising an amino acid sequence having at least 95%, at least 98%, or at least 99% identity to SEQ ID NO:64 without the signal sequence. In some embodiments, provided herein is a recombinant neuraminidase comprising the amino acid sequence of SEQ ID NO:64. In some embodiments, provided herein is a recombinant neuraminidase comprising the amino acid sequence of SEQ ID NO:64 without signal sequence.

In some embodiments, provided herein is a recombinant neuraminidase comprising an amino acid sequence having at least 80% identity to SEQ ID NO:66. In some embodiments, provided herein is a recombinant neuraminidase comprising an amino acid sequence having at least 85%, at least 90%, at least 95%, at least 98%, or at least 99% identity to SEQ ID NO:66. In some embodiments, provided herein is a recombinant neuraminidase comprising an amino acid sequence having at least 95%, at least 98%, or at least 99% identity to SEQ ID NO:66. In some embodiments, provided herein is a recombinant neuraminidase comprising an amino acid sequence having at least 85%, at least 90%, at least 95%, at least 98%, or at least 99% identity to SEQ ID NO:66 without the signal sequence. In some embodiments, provided herein is a recombinant neuraminidase comprising an amino acid sequence having at least 95%, at least 98%, or at least 99% identity to SEQ ID NO:66 without the signal sequence. In some embodiments, provided herein is a recombinant neuraminidase comprising the amino acid sequence of SEQ ID NO:66. In some embodiments, provided herein is a recombinant neuraminidase comprising the amino acid sequence of SEQ ID NO:66 without the signal sequence.

In some embodiments, provided herein is a recombinant neuraminidase comprising an amino acid sequence having at least 80% identity to SEQ ID NO:68. In some embodiments, provided herein is a recombinant neuraminidase comprising an amino acid sequence having at least 85%, at least 90%, at least 95%, at least 98%, or at least 99% identity to SEQ ID NO:68. In some embodiments, provided herein is a recombinant neuraminidase comprising an amino acid sequence having at least 95%, at least 98%, or at least 99% identity to SEQ ID NO:68. In some embodiments, provided herein is a recombinant neuraminidase comprising an amino acid sequence having at least 85%, at least 90%, at least 95%, at least 98%, or at least 99% identity to SEQ ID NO:68 without the signal sequence. In some embodiments, provided herein is a recombinant neuraminidase comprising an amino acid sequence having at least 95%, at least 98%, or at least 99% identity to SEQ ID NO:68 without the signal sequence. In some embodiments, provided herein is a recombinant neuraminidase comprising the amino acid sequence of SEQ ID NO:68. In some embodiments, provided herein is a recombinant neuraminidase comprising the amino acid sequence of SEQ ID NO:68 without the signal sequence.

In some embodiments, provided herein is a recombinant neuraminidase comprising an amino acid sequence having at least 80% identity to SEQ ID NO:70. In some embodiments, provided herein is a recombinant neuraminidase comprising an amino acid sequence having at least 85%, at least 90%, at least 95%, at least 98%, or at least 99% identity to SEQ ID NO:70. In some embodiments, provided herein is a recombinant neuraminidase comprising an amino acid sequence having at least 95%, at least 98%, or at least 99% identity to SEQ ID NO:70. In some embodiments, provided herein is a recombinant neuraminidase comprising an amino acid sequence having at least 85%, at least 90%, at least 95%, at least 98%, or at least 99% identity to SEQ ID NO:70 without the signal sequence. In some embodiments, provided herein is a recombinant neuraminidase comprising an amino acid sequence having at least 95%, at least 98%, or at least 99% identity to SEQ ID NO:70 without the signal sequence. In some embodiments, provided herein is a recombinant neuraminidase comprising the amino acid sequence of SEQ ID NO:70. In some embodiments, provided herein is a recombinant neuraminidase comprising the amino acid sequence of SEQ ID NO:70 without the signal sequence.

In some embodiments, provided herein is a recombinant neuraminidase comprising an amino acid sequence having at least 80% identity to SEQ ID NO:71. In some sequence having at least 85%, at least 90%, at least 95%, at least 98%, or at least 99% identity to SEQ ID NO:71. In some embodiments, provided herein is a recombinant neuraminidase comprising an amino acid sequence having at least 95%, at least 98%, or at least 99% identity to SEQ ID NO:71. In some embodiments, provided herein is a recombinant neuraminidase comprising an amino acid sequence having at least 85%, at least 90%, at least 95%, at least 98%, or at least 99% identity to SEQ ID NO:71 without the signal sequence. In some embodiments, provided herein is a recombinant neuraminidase comprising an amino acid sequence having at least 95%, at least 98%, or at least 99% identity to SEQ ID NO:71 without the signal sequence. In some embodiments, provided herein is a recombinant neuraminidase comprising the amino acid sequence of SEQ ID NO:71. In some embodiments, provided herein is a recombinant neuraminidase comprising the amino acid sequence of SEQ ID NO:71 without the signal sequence.

In some embodiments, provided herein is a recombinant neuraminidase comprising an amino acid sequence having at least 80% identity to SEQ ID NO:72. In some embodiments, provided herein is a recombinant neuraminidase comprising an amino acid sequence having at least 85%, at least 90%, at least 95%, at least 98%, or at least 99% identity to SEQ ID NO:72. In some embodiments, provided herein is a recombinant neuraminidase comprising an amino acid sequence having at least 95%, at least 98%, or at least 99% identity to SEQ ID NO:72. In some embodiments, provided herein is a recombinant neuraminidase comprising an amino acid sequence having at least 85%, at least 90%, at least 95%, at least 98%, or at least 99% identity to SEQ ID NO:72 without the signal sequence. In some embodiments, provided herein is a recombinant neuraminidase comprising an amino acid sequence having at least 95%, at least 98%, or at least 99% identity to SEQ ID NO:72 without the signal sequence. In some embodiments, provided herein is a recombinant neuraminidase comprising the amino acid sequence of SEQ ID NO:72. In some embodiments, provided herein is a recombinant neuraminidase comprising the amino acid sequence of SEQ ID NO:72 without the signal sequence.

In some embodiments, provided herein is a recombinant neuraminidase comprising an amino acid sequence having at least 80% identity to SEQ ID NO:73. In some embodiments, provided herein is a recombinant neuraminidase comprising an amino acid sequence having at least 85%, at least 90%, at least 95%, at least 98%, or at least 99% identity to SEQ ID NO:73. In some embodiments, provided herein is a recombinant neuraminidase comprising an amino acid sequence having at least 95%, at least 98%, or at least 99% identity to SEQ ID NO:73. In some embodiments, provided herein is a recombinant neuraminidase comprising an amino acid sequence having at least 85%, at least 90%, at least 95%, at least 98%, or at least 99% identity to SEQ ID NO:73 without the signal sequence. In some embodiments, provided herein is a recombinant neuraminidase comprising an amino acid sequence having at least 95%, at least 98%, or at least 99% identity to SEQ ID NO:73 without the signal sequence. In some embodiments, provided herein is a recombinant neuraminidase comprising the amino acid sequence of SEQ ID NO:73. In some embodiments, provided herein is a recombinant neuraminidase comprising the amino acid sequence of SEQ ID NO:73 without the signal sequence.

In a specific embodiment, provided herein is a recombinant neuraminidase comprising or consisting of the amino acid sequence of SEQ ID NO:40, 42, 44, 46, or 48. In another specific embodiment, provided herein is a recombinant neuraminidase comprising or consisting of the amino acid sequence of SEQ ID NO:48 without one, two or all of the following: the signal sequence, purification tag, or cleavage site. In certain embodiments, a recombinant neuraminidase provided herein comprises or consists of the amino acid sequence that is encoded by the nucleotide sequence set forth in SEQ ID NO:43 or 47. In another specific embodiment, provided herein is a recombinant neuraminidase comprising or consisting of the amino acid sequence of SEQ ID NO:50, 52, 54, 56, or 58. In another specific embodiment, provided herein is a recombinant neuraminidase comprising or consisting of the amino acid sequence of SEQ ID NO:58 without one, two or all of the following: the signal sequence, purification tag, or cleavage site. In certain embodiments, a recombinant neuraminidase provided herein comprises or consists of the amino acid sequence that is encoded by the nucleotide sequence set forth in SEQ ID NO:59. In another specific embodiment, provided herein is a recombinant neuraminidase comprising or consisting of the amino acid sequence of SEQ ID NO:60, 62, 64, 66, or 68. In another specific embodiment, provided herein is a recombinant neuraminidase comprising or consisting of the amino acid sequence of SEQ ID NO:68 without one, two or all of the following: the signal sequence, purification tag, or cleavage site. In certain embodiments, a recombinant neuraminidase provided herein comprises or consists of the amino acid sequence that is encoded by the nucleotide sequence set forth in SEQ ID NO:69.

In certain embodiments, a recombinant neuraminidase provided herein comprises or consists of the amino acid sequence that is encoded by the nucleotide sequence set forth in SEQ ID NO:43 or 47. In certain embodiments, a recombinant neuraminidase provided herein comprises or consists of the amino acid sequence that is encoded by the nucleotide sequence set forth in SEQ ID NO:59. In certain embodiments, a recombinant neuraminidase provided herein comprises or consists of the amino acid sequence that is encoded by the nucleotide sequence set forth in SEQ ID NO: 69.

In certain embodiments, a recombinant neuraminidase provided herein exists in one, two, three or all of the following forms: monomeric, dimeric, trimeric, or tetrameric. In specific embodiments, a recombinant neuraminidase provided herein is tetrameric as assessed by techniques known in the art or described herein.

In specific embodiments, a recombinant neuraminidase provided herein is capable of forming a three-dimensional structure that is similar to the three-dimensional structure of a native influenza neuraminidase. Structural similarity might be evaluated based on any technique deemed suitable by those of skill in the art. For instance, reaction, e.g. under non-denaturing conditions, of a recombinant neuraminidase with a neutralizing antibody or antiserum that recognizes a native influenza neuraminidase might indicate structural similarity. Useful neutralizing antibodies or antisera are described in, e.g., Shoji et al., Hum. Vaccines, 2011, 7:199-204, Wan et al., J. Virol. 2013, 87:9290-9300, Doyle et al. Antivir. Res. 2013, 100:567-574, Doyle et al., Biochem. Biophys. Res. Commun. 2013, 441:226-229, and Wohlbold et al., 2017, Nat. Microbiol. 2(10): 1415-1424, the contents of which are hereby incorporated by reference in their entireties. In certain embodiments, the antibody or antiserum is an antibody or antiserum that reacts with a non-contiguous epitope (i.e., not contiguous in primary sequence) that is formed by the tertiary or quaternary structure of a neuraminidase.

When designing a recombinant neuraminidase, care should be taken to maintain the stability of the resulting protein. In this regard, it is recommended that cysteine residues capable of forming disulfide bonds be maintained since they contribute to the stability of the neuraminidase protein. See, e.g., Basler et al., 1999, Journal of Virology, 73(10):8095-8103 for non-limiting examples of influenza virus neuraminidase cysteine residues capable of forming disulfide bonds. The stability of a recombinant neuraminidase described herein can be assessed using techniques known in the art, such as sensitivity of the neuraminidase molecules to Ca²⁺, as described in, e.g., Baker and Gandhi, 1976, Archives of Virology, 52:7-18. The stability of a recombinant neuraminidase may be assessed by any method described herein (e.g., in Section 5.7 or Section 6, infra).

In a specific embodiment, a recombinant neuraminidase described herein has neuraminidase enzymatic activity as assessed using an assay known in the art or described herein (e.g., in Section 5.7 or Section 6). In a specific embodiment, a recombinant neuraminidase described herein has enzymatic activity as assessed using an NA-Star Assay.

### 5.4 Nucleic Acids

Provided herein are nucleic acids that encode recombinant neuraminidase described herein (e.g., in Section 5.3 or 6). Due to the degeneracy of the genetic code, any nucleic acid that encodes a recombinant neuraminidase described herein is encompassed herein. In specific embodiments, provided herein is a nucleic acid comprising a nucleotide sequence encoding a recombinant neuraminidase (with or without a signal peptide). In certain embodiment, the nucleotide sequence encoding the recombinant neuraminidase comprises a nucleotide sequence encoding a signal peptide (e.g., a signal peptide/membrane anchor from the NA of the same influenza virus as the influenza virus engineered to express the recombinant neuraminidase polypeptide). In some embodiments, the nucleic acids provided herein are codon optimized.

In certain embodiments, provided herein is a nucleic acid comprising the nucleotide sequence set forth in SEQ ID NO:43 or 47. In another specific embodiment, a nucleic acid comprises a nucleotide sequence having at least 80% identity to the nucleotide sequence of SEQ ID NO:43. In some embodiments, a nucleic acid comprises a nucleotide sequence having at least 85%, at least 90%, at least 95%, at least 98%, or at least 99% identity to the nucleotide sequence of SEQ ID NO:43. In certain embodiments, a nucleic acid comprises the nucleotide sequence of SEQ ID NO:43. In another specific embodiment, a nucleic acid comprises a nucleotide sequence having at least 80% identity to the nucleotide sequence of SEQ ID NO:47. In some embodiments, a nucleic acid comprises a nucleotide sequence having at least 85%, at least 90%, at least 95%, at least 98%, or at least 99% identity to the nucleotide sequence of SEQ ID NO:47. In certain embodiments, a nucleic acid comprises the nucleotide sequence of SEQ ID NO:47.

In certain embodiments, provided herein is a nucleic acid comprising the nucleotide sequence set forth in SEQ ID NO:43 or 47 without the signal sequence. In another specific embodiment, a nucleic acid comprises a nucleotide sequence having at least 80% identity to the nucleotide sequence of SEQ ID NO:43 without the signal sequence. In some embodiments, a nucleic acid comprises a nucleotide sequence having at least 85%, at least 90%, at least 95%, at least 98%, or at least 99% identity to the nucleotide sequence of SEQ ID NO:43 without the signal sequence. In certain embodiments, a nucleic acid comprises the nucleotide sequence of SEQ ID NO:43 without the signal sequence. In another specific embodiment, a nucleic acid comprises a nucleotide sequence having at least 80% identity to the nucleotide sequence of SEQ ID NO:47 without the signal sequence. In some embodiments, a nucleic acid comprises a nucleotide sequence having at least 85%, at least 90%, at least 95%, at least 98%, or at least 99% identity to the nucleotide sequence of SEQ ID NO:47 without the signal sequence. In certain embodiments, a nucleic acid comprises the nucleotide sequence of SEQ ID NO:47 without the signal sequence.

In certain embodiments, provided herein is a nucleic acid comprising the nucleotide sequence set forth in SEQ ID NO:59. In another specific embodiment, a nucleic acid comprises a nucleotide sequence having at least 80% identity to the nucleotide sequence of SEQ ID NO:59. In some embodiments, a nucleic acid comprises a nucleotide sequence having at least 85%, at least 90%, at least 95%, at least 98%, or at least 99% identity to the nucleotide sequence of SEQ ID NO:59. In certain embodiments, a nucleic acid comprises the nucleotide sequence of SEQ ID NO:59.

In certain embodiments, provided herein is a nucleic acid comprising the nucleotide sequence set forth in SEQ ID NO:59 without the signal sequence. In another specific embodiment, a nucleic acid comprises a nucleotide sequence having at least 80% identity to the nucleotide sequence of SEQ ID NO:59 without the signal sequence. In some embodiments, a nucleic acid comprises a nucleotide sequence having at least 85%, at least 90%, at least 95%, at least 98%, or at least 99% identity to the nucleotide sequence of SEQ ID NO:59 without the signal sequence. In certain embodiments, a nucleic acid comprises the nucleotide sequence of SEQ ID NO:59 without the signal sequence.

In certain embodiments, provided herein is a nucleic acid comprising the nucleotide sequence set forth in SEQ ID NO:69. In another specific embodiment, a nucleic acid comprises a nucleotide sequence having at least 80% identity to the nucleotide sequence of SEQ ID NO:69. In some embodiments, a nucleic acid comprises a nucleotide sequence having at least 85%, at least 90%, at least 95%, at least 98%, or at least 99% identity to the nucleotide sequence of SEQ ID NO:69. In certain embodiments, a nucleic acid comprises the nucleotide sequence of SEQ ID NO:69.

In certain embodiments, provided herein is a nucleic acid comprising the nucleotide sequence set forth in SEQ ID NO:69 without the signal sequence. In another specific embodiment, a nucleic acid comprises a nucleotide sequence having at least 80% identity to the nucleotide sequence of SEQ ID NO:69 without the signal sequence. In some embodiments, a nucleic acid comprises a nucleotide sequence having at least 85%, at least 90%, at least 95%, at least 98%, or at least 99% identity to the nucleotide sequence of SEQ ID NO:69 without the signal sequence. In certain embodiments, a nucleic acid comprises the nucleotide sequence of SEQ ID NO:69 without the signal sequence.

Also provided herein are nucleic acids capable of hybridizing to a nucleic acid encoding a recombinant neuraminidase. In some embodiments, provided herein is a nucleic acid capable of hybridizing to the nucleotide sequence set forth in SEQ ID NO:43, 47, 59, or 69. In some embodiments, provided herein is a nucleic acid capable of hybridizing to the nucleotide sequence set forth in SEQ ID NO:43, 47, 59, or 69 without the signal sequence. In certain embodiments, provided herein are nucleic acids capable of hybridizing to a fragment of a nucleic acid encoding a recombinant neuraminidase. In some embodiments, provided herein is a nucleic acid capable of hybridizing to a fragment (e.g., comprising or consisting of 250, 300, 350, 400, 450, 500, 550, 600 or more nucleotides, or between 300 to 600, 400 to 600 or 500 to 700 nucleotides) of SEQ ID NO:43, 47, 59, or 69. In other embodiments, provided herein are nucleic acids capable of hybridizing to the full length of a nucleic acid encoding a recombinant neuraminidase. General parameters for hybridization conditions for nucleic acids are described in Sambrook et al., Molecular Cloning-A Laboratory Manual (2nd Ed.), Vols. 1-3, Cold Spring Harbor Laboratory, Cold Spring Harbor, New York (1989), and in Ausubel et al., Current Protocols in Molecular Biology, vol. 2, Current Protocols Publishing, New York (1994). Hybridization may be performed under high stringency conditions, medium stringency conditions, or low stringency conditions. Those of skill in the art will understand that low, medium, and high stringency conditions are contingent upon multiple factors all of which interact and are also dependent upon the nucleic acids in question. For example, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents.

In some embodiments, a nucleic acid comprising a nucleotide sequence encoding a recombinant neuraminidase is isolated. In certain embodiments, an “isolated” nucleic acid refers to a nucleic acid molecule which is separated from other nucleic acid molecules which are present in the natural source of the nucleic acid. In other words, the isolated nucleic acid can comprise heterologous nucleic acids that are not associated with it in nature. In other embodiments, an “isolated” nucleic acid, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acids in which the nucleic acid sequence is separated from cellular components of the cells from which it is isolated or recombinantly produced. Thus, a nucleic acid molecule that is substantially free of cellular material includes preparations of nucleic acid having less than about 30%, 20%, 10%, or 5% (by dry weight) of other nucleic acids. The term “substantially free of culture medium” includes preparations of nucleic acid in which the culture medium represents less than about 50%, 20%, 10%, or 5% of the volume of the preparation. The term “substantially free of chemical precursors or other chemicals” includes preparations in which the nucleic acid is separated from chemical precursors or other chemicals which are involved in the synthesis of the nucleic acid. In specific embodiments, such preparations of the nucleic acid have less than about 50%, 30%, 20%, 10%, 5%, or 2% (by dry weight) of chemical precursors or compounds other than the nucleic acid of interest.

### 5.5 Expression of Recombinant Neuraminidase

Provided herein are vectors, including expression vectors, containing a nucleic acid comprising a nucleotide sequence encoding a recombinant neuraminidase (NA) described herein. In a specific embodiment, the vector is an expression vector that is capable of directing the expression of a nucleic acid encoding a recombinant neuraminidase. Non-limiting examples of expression vectors include, but are not limited to, plasmids and viral vectors, such as replication defective retroviruses, adenoviruses, vesicular stomatitis virus (VSV), Newcastle disease virus (NDV), vaccinia (e.g., Modified Vaccinia Ankara virus), adeno-associated viruses and baculoviruses. Techniques known to one of skill in the art may be used to engineer such viral vectors to express a recombinant neuraminidase described herein. Expression vectors also may include, without limitation, transgenic animals, and non-mammalian cells/organisms, e.g., non-mammalian cells/organisms that have been engineered to perform mammalian N-linked glycosylation.

In some embodiments, provided herein are expression vectors encoding components of a recombinant neuraminidase (e.g., the globular head domain). Such vectors may be used to express the components in one or more host cells and the components may be isolated and conjugated together with a linker using techniques known to one of skill in the art.

An expression vector comprises a nucleic acid comprising a nucleotide sequence encoding a recombinant neuraminidase described herein and in a form suitable for expression of the nucleic acid in a host cell. In a specific embodiment, an expression vector includes one or more regulatory sequences, selected on the basis of the host cells to be used for expression, which is operably linked to the nucleic acid to be expressed. Within an expression vector, “operably linked” is intended to mean that a nucleic acid of interest is linked to the regulatory sequence(s) in a manner which allows for expression of the nucleic acid (e.g., in an in vitro transcription/translation system or in a host cell when the vector is introduced into the host cell). Regulatory sequences include promoters, enhancers, and other expression control elements (e.g., polyadenylation signals). Regulatory sequences include those which direct constitutive expression of a nucleic acid in many types of host cells, those which direct expression of the nucleic acid only in certain host cells (e.g., tissue-specific regulatory sequences), and those which direct the expression of the nucleic acid upon stimulation with a particular agent (e.g., inducible regulatory sequences). It will be appreciated by those skilled in the art that the design of the expression vector can depend on such factors as the choice of the host cell to be transformed, the level of expression of protein desired, etc. The term “host cell” is intended to include a particular subject cell transformed or transfected with a nucleic acid and the progeny or potential progeny of such a cell. Progeny of such a cell may not be identical to the parent cell transformed or transfected with the nucleic acid due to mutations or environmental influences that may occur in succeeding generations or integration of the nucleic acid into the host cell genome. In specific embodiments, the host cell is a cell line. Examples of host cells (e.g., yeast, avian, insect, plant and/or mammalian cells) that may be used to express a nucleic acid are provided herein.

Expression vectors can be designed for expression of a recombinant neuraminidase described herein using prokaryotic (e.g., E. coli) or eukaryotic cells (e.g., insect cells (using baculovirus expression vectors, see, e.g., Treanor et al., 2007, JAMA, 297(14):1577-1582 incorporated by reference herein in its entirety), yeast cells, plant cells, algae, avian, or mammalian cells). Examples of yeast host cells include, but are not limited to S. pombe and S. cerevisiae and examples, infra. An example of avian cells includes, but is not limited to EB66 cells. Examples of mammalian host cells include, but are not limited to, A549 cells, Crucell Per.C6 cells, Vero cells, CHO cells, VERO cells, BHK cells, HeLa cells, COS cells, MDCK cells, 293 cells, 3T3 cells or WI38 cells. In certain embodiments, the hosts cells are myeloma cells, e.g., NS0 cells, 45.6 TG1.7 cells, AF-2 clone 9B5 cells, AF-2 clone 9B5 cells, J558L cells, MOPC 315 cells, MPC-11 cells, NCI-H929 cells, NP cells, NS0/1 cells, P3 NS1 Ag4 cells, P3/NS1/1-Ag4-1 cells, P3U1 cells, P3X63Ag8 cells, P3X63Ag8.653 cells, P3X63Ag8U.1 cells, RPMI 8226 cells, Sp20-Ag14 cells, U266B1 cells, X63AG8.653 cells, Y3.Ag.1.2.3 cells, and YO cells. Non-limiting examples of insect cells include Sf9, S/21, Trichoplusia ni, Spodoptera frugiperda and Bombyx mori. In a particular embodiment, a mammalian cell culture system (e.g. Chinese hamster ovary or baby hamster kidney cells) is used for expression of a recombinant neuraminidase. In another embodiment, a plant cell culture system is used for expression of a recombinant neuraminidase. See, e.g., U.S. Pat. Nos. 7,504,560; 6,770,799; 6,551,820; 6,136,320; 6,034,298; 5,914,935; 5,612,487; and 5,484,719, and U.S. patent application publication Nos. 2009/0208477, 2009/0082548, 2009/0053762, 2008/0038232, 2007/0275014 and 2006/0204487 for plant cells and methods for the production of proteins utilizing plant cell culture systems. In specific embodiments, plant cell culture systems are not used for expression of a recombinant neuraminidase. The host cells comprising a nucleic acid that encodes a recombinant neuraminidase described herein can be isolated, i.e., the cells are outside of the body of a subject. In certain embodiments, the cells are engineered to express a nucleic acid that encodes a recombinant neuraminidase described herein. In particular embodiments, the cells are engineered to express a recombinant neuraminidase described herein. In specific embodiments, the host cells are cells from a cell line.

In certain embodiments, provided herein is host cell(s) comprising a nucleic acid that comprises a nucleotide sequence encoding a recombinant neuraminidase described herein. In some embodiments, provided herein is a host cell(s) engineered to express a express a recombinant neuraminidase described herein. Host cells include those cells, including cell lines, described herein.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning-A Laboratory Manual, 2nd Edition, Cold Spring Harbor Press, New York, and other laboratory manuals. In certain embodiments, a host cell is transiently transfected with an expression vector containing a nucleic acid encoding a recombinant neuraminidase. In other embodiments, a host cell is stably transfected with an expression vector containing a nucleic acid encoding a recombinant neuraminidase.

For stable transfection of mammalian cells, it is known that, depending upon the expression vector and transfection technique used, only a small fraction of cells may integrate the foreign DNA into their genome. In order to identify and select these integrants, a nucleic acid that encodes a selectable marker (e.g., for resistance to antibiotics) is generally introduced into the host cells along with the nucleic acid of interest. Examples of selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

As an alternative to recombinant expression of a recombinant neuraminidase using a host cell, an expression vector containing a nucleic acid encoding a recombinant neuraminidase can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce a recombinant neuraminidase.

Once a recombinant neuraminidase has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins.

Accordingly, provided herein are methods for producing a recombinant neuraminidase. In one embodiment, the method comprises culturing a host cell containing a nucleic acid comprising a nucleotide sequence encoding a recombinant neuraminidase in a suitable medium such that the recombinant neuraminidase is produced. In some embodiments, the method further comprises isolating the recombinant neuraminidase from the medium or the host cell.

Also provided herein are methods for producing a virus (e.g., a baculovirus) comprising a recombinant neuraminidase described herein, comprising propagating the virus in any substrate that allows the virus to grow to titers that permit their use in accordance with the methods described herein. In one embodiment, the substrate allows the viruses to grow to titers comparable to those determined for the corresponding wild-type viruses. In a specific embodiment, the virus is propagated in embryonated eggs (e.g., chicken eggs).

### 5.6 Prophylactic and Therapeutic Uses

In one aspect, provided herein are methods for inducing an immune response in a subject (e.g., a human subject) utilizing a recombinant neuraminidase described herein, or an immunogenic composition thereof. In a specific embodiment, a method for inducing an immune response to an influenza virus in a subject comprises administering to the subject an effective amount of an immunogenic composition described herein.

In another aspect, provided herein are methods for immunizing a subject (e.g., a human subject) against influenza virus in a subject utilizing a recombinant neuraminidase described herein, or an immunogenic composition thereof. In a specific embodiment, a method for immunizing a subject (e.g., a human subject) against influenza virus comprises administering to the subject an effective amount of an immunogenic composition described herein. In a specific embodiment, provided herein is a method of immunization of a subject (e.g., a human subject) for the prevention of influenza disease comprising administering an immunogenic composition described herein. In a specific embodiment, provided herein is a method of immunization of a subject (e.g., a human subject) for the prevention of influenza disease comprising administering an effective amount of an immunogenic composition described herein.

In another aspect, provided herein are methods for preventing influenza virus disease in a subject (e.g., a human subject) utilizing a recombinant neuraminidase described herein, or an immunogenic composition thereof. In a specific embodiment, a method for preventing influenza virus disease in a subject (e.g., a human subject) comprises administering to the subject an effective amount of an immunogenic composition described herein.

In another aspect, provided herein is a method for immunizing a subject (e.g., a human subject) against influenza virus, comprising administering to the subject a recombinant neuraminidase described herein, or an immunogenic composition comprising a recombinant neuraminidase described herein (see Section 5.1), and administering to the subject a CpG oligonucleotide adjuvant described herein (e.g., in Section 5.2 or Example 1). In one embodiment, provided herein is a method for immunizing a subject (e.g., a human subject) against influenza virus, comprising administering to the subject an effective amount of a recombinant neuraminidase described herein, or an immunogenic composition comprising a recombinant neuraminidase described herein (see Section 5.3) in combination with CpG oligonucleotide adjuvant described herein (e.g., in Section 5.2 or Example 1). The recombinant neuraminidase or the immunogenic composition may be administered to the subject concurrently with, prior to (e.g., less than 5 minutes, less than 10 minutes, less than 15 minutes, less than 30 minutes, less than 45 minutes, less than 60 minutes, less than 1.5 hours, or less than 2 hours prior to), or subsequent to (e.g., less than 5 minutes, less than 10 minutes, less than 15 minutes, less than 30 minutes, less than 45 minutes, less than 60 minutes, less than 1.5 hours, or less than 2 hours after) the administration of a CpG oligonucleotide adjuvant described herein (e.g., in Section 5.2 or Example 1). In a specific embodiment, the recombinant neuraminidase or the immunogenic composition is administered to the subject concurrently with the administration of a CpG oligonucleotide adjuvant described herein (e.g., in Section 5.2 or Example 1). In another specific embodiment, the recombinant neuraminidase or the immunogenic composition is administered to the subject less than 5 minutes prior to the administration of a CpG oligonucleotide adjuvant described herein (e.g., in Section 5.2 or Example 1). In another specific embodiment, the recombinant neuraminidase or the immunogenic composition is administered to the subject less than 10 minutes prior to the administration of a CpG oligonucleotide adjuvant described herein (e.g., in Section 5.2 or Example 1). In another specific embodiment, the recombinant neuraminidase or the immunogenic composition is administered to the subject less than 15 minutes prior to the administration of a CpG oligonucleotide adjuvant described herein (e.g., in Section 5.2 or Example 1). In another specific embodiment, the recombinant neuraminidase or the immunogenic composition is administered to the subject less than 30 minutes prior to the administration of a CpG oligonucleotide adjuvant described herein (e.g., in Section 5.2 or Example 1). In another specific embodiment, the recombinant neuraminidase or the immunogenic composition is administered to the subject less than 45 minutes prior to the administration of a CpG oligonucleotide adjuvant described herein (e.g., in Section 5.2 or Example 1). In another specific embodiment, the recombinant neuraminidase or the immunogenic composition is administered to the subject less than 60 minutes prior to the administration of a CpG oligonucleotide adjuvant described herein (e.g., in Section 5.2 or Example 1). In another specific embodiment, the recombinant neuraminidase or the immunogenic composition is administered to the subject less than 1.5 hours minutes prior to the administration of a CpG oligonucleotide adjuvant described herein (e.g., in Section 5.2 or Example 1). In another specific embodiment, the recombinant neuraminidase or the immunogenic composition is administered to the subject less than 2 hours prior to the administration of a CpG oligonucleotide adjuvant described herein (e.g., in Section 5.2 or Example 1). In another specific embodiment, the recombinant neuraminidase or the immunogenic composition is administered to the subject less than 5 minutes after the administration of a CpG oligonucleotide adjuvant described herein (e.g., in Section 5.2 or Example 1). In another specific embodiment, the recombinant neuraminidase or the immunogenic composition is administered to the subject less than 10 minutes after the administration of a CpG oligonucleotide adjuvant described herein (e.g., in Section 5.2 or Example 1). In another specific embodiment, the recombinant neuraminidase or the immunogenic composition is administered to the subject less than 15 minutes after the administration of a CpG oligonucleotide adjuvant described herein (e.g., in Section 5.2 or Example 1). In another specific embodiment, the recombinant neuraminidase or the immunogenic composition is administered to the subject less than 30 minutes after the administration of a CpG oligonucleotide adjuvant (e.g., as described herein Section 5.2 or Example 1). In another specific embodiment, the immunogenic composition or the recombinant neuraminidase or is administered to the subject less than 45 minutes after the administration of a CpG oligonucleotide adjuvant described herein (e.g., in Section 5.2 or Example 1). In another specific embodiment, the recombinant neuraminidase or the immunogenic composition is administered to the subject less than 60 minutes after the administration of a CpG oligonucleotide adjuvant described herein (e.g., in Section 5.2 or Example 1). In another specific embodiment, the recombinant neuraminidase or the immunogenic composition is administered to the subject less than 1.5 hours minutes after the administration of a CpG oligonucleotide adjuvant described herein (e.g., in Section 5.2 or Example 1). In another specific embodiment, the recombinant neuraminidase or the immunogenic composition is administered to the subject less than 2 hours after the administration of a CpG oligonucleotide adjuvant described herein (e.g., in Section 5.2 or Example 1). In another specific embodiment, the recombinant neuraminidase or the immunogenic composition is administered to the subject 2 hours or more after the administration of a CpG oligonucleotide adjuvant described herein (e.g., in Section 5.2 or Example 1). In a specific embodiment, the recombinant neuraminidase or the immunogenic composition and the CpG oligonucleotide adjuvant described herein are administered via the same route of administration. In other embodiments, the recombinant neuraminidase or the immunogenic composition and the CpG oligonucleotide adjuvant described herein (e.g., in Section 5.2 or Example 1) are administered via different routes of administration. In some embodiments, the immunogenic composition does not comprise a CpG oligonucleotide adjuvant described herein (e.g., in Section 5.2 or Example 1). In some embodiments, the immunogenic composition comprises an aluminum salt (e.g., amorphous aluminum hydroxyphosphate sulfate, aluminum hydroxide, aluminum phosphate, or potassium aluminum sulfate, or a combination thereof). In other embodiments, the immunogenic composition does not comprise an aluminum salt. In some embodiments, the immunogenic composition does not comprise an adjuvant.

In a specific embodiment, provided herein is a method of immunization of a subject (e.g., a human subject) for the prevention of influenza disease comprising administering to the subject a recombinant neuraminidase described herein, or an immunogenic composition comprising a recombinant neuraminidase described herein (see Section 5.3), and administering to the subject a CpG oligonucleotide adjuvant described herein (e.g., in Section 5.2 or Example 1). In one embodiment, provided herein is a method for immunization of a subject (e.g., a human subject) for the prevention of influenza disease comprising administering to the subject an effective amount of a recombinant neuraminidase described herein, or an immunogenic composition comprising a recombinant neuraminidase described herein (see Section 5.1) in combination with CpG oligonucleotide adjuvant described herein (e.g., in Section 5.2 or Example 1). The recombinant neuraminidase or the immunogenic composition may be administered to the subject concurrently with, prior to (e.g., less than 5 minutes, less than 10 minutes, less than 15 minutes, less than 30 minutes, less than 45 minutes, less than 60 minutes, less than 1.5 hours, or less than 2 hours prior to), or subsequent to (e.g., less than 5 minutes, less than 10 minutes, less than 15 minutes, less than 30 minutes, less than 45 minutes, less than 60 minutes, less than 1.5 hours, or less than 2 hours after) the administration of a CpG oligonucleotide adjuvant described herein (e.g., in Section 5.2 or Example 1). In a specific embodiment, the recombinant neuraminidase or the immunogenic composition is administered to the subject concurrently with the administration of a CpG oligonucleotide adjuvant described herein (e.g., in Section 5.2 or Example 1). In another specific embodiment, the recombinant neuraminidase or the immunogenic composition is administered to the subject less than 5 minutes prior to the administration of a CpG oligonucleotide adjuvant described herein (e.g., in Section 5.2 or Example 1). In another specific embodiment, the recombinant neuraminidase or the immunogenic composition is administered to the subject less than 10 minutes prior to the administration of a CpG oligonucleotide adjuvant described herein (e.g., in Section 5.2 or Example 1). In another specific embodiment, the recombinant neuraminidase or the immunogenic composition is administered to the subject less than 15 minutes prior to the administration of a CpG oligonucleotide adjuvant described herein (e.g., in Section 5.2 or Example 1). In another specific embodiment, the recombinant neuraminidase or the immunogenic composition is administered to the subject less than 30 minutes prior to the administration of a CpG oligonucleotide adjuvant described herein (e.g., in Section 5.2 or Example 1). In another specific embodiment, the recombinant neuraminidase or the immunogenic composition is administered to the subject less than 45 minutes prior to the administration of a CpG oligonucleotide adjuvant described herein (e.g., in Section 5.2 or Example 1). In another specific embodiment, the recombinant neuraminidase or the immunogenic composition is administered to the subject less than 60 minutes prior to the administration of a CpG oligonucleotide adjuvant described herein (e.g., in Section 5.2 or Example 1). In another specific embodiment, the recombinant neuraminidase or the immunogenic composition is administered to the subject less than 1.5 hours minutes prior to the administration of a CpG oligonucleotide adjuvant described herein (e.g., in Section 5.2 or Example 1). In another specific embodiment, the recombinant neuraminidase or the immunogenic composition is administered to the subject less than 2 hours prior to the administration of a CpG oligonucleotide adjuvant described herein (e.g., in Section 5.2 or Example 1). In another specific embodiment, the recombinant neuraminidase or the immunogenic composition is administered to the subject 2 hours or more prior to the administration of a CpG oligonucleotide adjuvant described herein (e.g., in Section 5.2 or Example 1). In another specific embodiment, the recombinant neuraminidase or the immunogenic composition is administered to the subject less than 5 minutes after the administration of a CpG oligonucleotide adjuvant described herein (e.g., in Section 5.2 or Example 1). In another specific embodiment, the recombinant neuraminidase or the immunogenic composition is administered to the subject less than 10 minutes after the administration of a CpG oligonucleotide adjuvant described herein (e.g., in Section 5.2 or Example 1). In another specific embodiment, the recombinant neuraminidase or the immunogenic composition is administered to the subject less than 15 minutes after the administration of a CpG oligonucleotide adjuvant described herein (e.g., in Section 5.2 or Example 1). In another specific embodiment, the recombinant neuraminidase or the immunogenic composition is administered to the subject less than 30 minutes after the administration of a CpG oligonucleotide adjuvant (e.g., as described herein Section 5.2 or Example 1). In another specific embodiment, the immunogenic composition or the recombinant neuraminidase is administered to the subject less than 45 minutes after the administration of a CpG oligonucleotide adjuvant described herein (e.g., in Section 5.2 or Example 1). In another specific embodiment, the recombinant neuraminidase or the immunogenic composition is administered to the subject less than 60 minutes after the administration of a CpG oligonucleotide adjuvant described herein (e.g., in Section 5.2 or Example 1). In another specific embodiment, the recombinant neuraminidase or the immunogenic composition is administered to the subject less than 1.5 hours minutes after the administration of a CpG oligonucleotide adjuvant described herein (e.g., in Section 5.2 or Example 1). In another specific embodiment, the recombinant neuraminidase or the immunogenic composition is administered to the subject less than 2 hours after the administration of a CpG oligonucleotide adjuvant described herein (e.g., in Section 5.2 or Example 1). In another specific embodiment, the recombinant neuraminidase or the immunogenic composition is administered to the subject 2 hours or more after the administration of a CpG oligonucleotide adjuvant described herein (e.g., in Section 5.2 or Example 1). In a specific embodiment, the recombinant neuraminidase or the immunogenic composition and the CpG oligonucleotide adjuvant described herein are administered via the same route of administration. In other embodiments, the recombinant neuraminidase or the immunogenic composition and the CpG oligonucleotide adjuvant described herein (e.g., in Section 5.2 or Example 1) are administered via different routes of administration. In some embodiments, the immunogenic composition does not comprise a CpG oligonucleotide adjuvant described herein (e.g., in Section 5.2 or Example 1). In some embodiments, the immunogenic composition comprises an aluminum salt (e.g., amorphous aluminum hydroxyphosphate sulfate, aluminum hydroxide, aluminum phosphate, or potassium aluminum sulfate, or a combination thereof). In other embodiments, the immunogenic composition does not comprise an aluminum salt. In some embodiments, the immunogenic composition does not comprise an adjuvant.

In another aspect, provided herein is a method for preventing influenza virus in a subject, comprising administering to the subject a recombinant neuraminidase described herein, or immunogenic composition comprising a recombinant neuraminidase described herein (see Section 5.1), and administering to the subject a CpG oligonucleotide adjuvant described herein (e.g., in Section 5.2 or Example 1). In one embodiment, provided herein is a method for preventing influenza virus in a subject, comprising administering to the subject an effective amount of a recombinant neuraminidase described herein, or immunogenic composition comprising a recombinant neuraminidase described herein (see, e.g., Section 5.3 or Section 6) in combination with CpG oligonucleotide adjuvant described herein (e.g., in Section 5.2 or Example 1). The recombinant neuraminidase or the immunogenic composition may be administered to the subject concurrently with, prior to (e.g., less than 5 minutes, less than 10 minutes, less than 15 minutes, less than 30 minutes, less than 45 minutes, less than 60 minutes, less than 1.5 hours, or less than 2 hours prior to), or subsequent to (e.g., less than 5 minutes, less than 10 minutes, less than 15 minutes, less than 30 minutes, less than 45 minutes, less than 60 minutes, less than 1.5 hours, or less than 2 hours after) the administration of a CpG oligonucleotide adjuvant described herein (e.g., in Section 5.2 or Example 1). In a specific embodiment, the recombinant neuraminidase or the immunogenic composition is administered to the subject concurrently with the administration of a CpG oligonucleotide adjuvant described herein (e.g., in Section 5.2 or Example 1). In another specific embodiment, the recombinant neuraminidase or the immunogenic composition is administered to the subject less than 5 minutes prior to the administration of a CpG oligonucleotide adjuvant described herein (e.g., in Section 5.2 or Example 1). In another specific embodiment, the recombinant neuraminidase or the immunogenic composition is administered to the subject less than 10 minutes prior to the administration of a CpG oligonucleotide adjuvant described herein (e.g., in Section 5.2 or Example 1). In another specific embodiment, the recombinant neuraminidase or the immunogenic composition is administered to the subject less than 15 minutes prior to the administration of a CpG oligonucleotide adjuvant described herein (e.g., in Section 5.2 or Example 1). In another specific embodiment, the recombinant neuraminidase or the immunogenic composition is administered to the subject less than 30 minutes prior to the administration of a CpG oligonucleotide adjuvant described herein (e.g., in Section 5.2 or Example 1). In another specific embodiment, the recombinant neuraminidase or the immunogenic composition is administered to the subject less than 45 minutes prior to the administration of a CpG oligonucleotide adjuvant described herein (e.g., in Section 5.2 or Example 1). In another specific embodiment, the recombinant neuraminidase or the immunogenic composition is administered to the subject less than 60 minutes prior to the administration of a CpG oligonucleotide adjuvant described herein (e.g., in Section 5.2 or Example 1). In another specific embodiment, the recombinant neuraminidase or the immunogenic composition is administered to the subject less than 1.5 hours minutes prior to the administration of a CpG oligonucleotide adjuvant described herein (e.g., in Section 5.2 or Example 1). In another specific embodiment, the recombinant neuraminidase or the immunogenic composition is administered to the subject less than 2 hours prior to the administration of a CpG oligonucleotide adjuvant described herein (e.g., in Section 5.2 or Example 1). In another specific embodiment, the recombinant neuraminidase or the immunogenic composition is administered to the subject 2 hours or more prior to the administration of a CpG oligonucleotide adjuvant described herein (e.g., in Section 5.2 or Example 1). In another specific embodiment, the immunogenic composition is administered to the subject less than 5 minutes after the administration of a CpG oligonucleotide adjuvant described herein (e.g., in Section 5.2 or Example 1). In another specific embodiment, the recombinant neuraminidase or the immunogenic composition is administered to the subject less than 10 minutes after the administration of a CpG oligonucleotide adjuvant described herein (e.g., in Section 5.2 or Example 1). In another specific embodiment, the recombinant neuraminidase or the immunogenic composition is administered to the subject less than 15 minutes after the administration of a CpG oligonucleotide adjuvant described herein (e.g., in Section 5.2 or Example 1). In another specific embodiment, the recombinant neuraminidase or the immunogenic composition is administered to the subject less than 30 minutes after the administration of a CpG oligonucleotide adjuvant (e.g., as described herein Section 5.2 or Example 1). In another specific embodiment, the immunogenic composition is or the recombinant neuraminidase is administered to the subject less than 45 minutes after the administration of a CpG oligonucleotide adjuvant described herein (e.g., in Section 5.2 or Example 1). In another specific embodiment, the recombinant neuraminidase or the immunogenic composition is administered to the subject less than 60 minutes after the administration of a CpG oligonucleotide adjuvant described herein (e.g., in Section 5.2 or Example 1). In another specific embodiment, the recombinant neuraminidase or the immunogenic composition is administered to the subject less than 1.5 hours minutes after the administration of a CpG oligonucleotide adjuvant described herein (e.g., in Section 5.2 or Example 1). In another specific embodiment, the recombinant neuraminidase or the immunogenic composition is administered to the subject less than 2 hours after the administration of a CpG oligonucleotide adjuvant described herein (e.g., in Section 5.2 or Example 1). In another specific embodiment, the recombinant neuraminidase or the immunogenic composition is administered to the subject 2 hours or more after the administration of a CpG oligonucleotide adjuvant described herein (e.g., in Section 5.2 or Example 1). In a specific embodiment, the recombinant neuraminidase or the immunogenic composition and the CpG oligonucleotide adjuvant described herein are administered via the same route of administration. In other embodiments, the recombinant neuraminidase or the immunogenic composition and the CpG oligonucleotide adjuvant described herein (e.g., in Section 5.2 or Example 1) are administered via different routes of administration. In some embodiments, the immunogenic composition does not comprise a CpG oligonucleotide adjuvant described herein (e.g., in Section 5.2 or Example 1). In some embodiments, the immunogenic composition comprises an aluminum salt (e.g., amorphous aluminum hydroxyphosphate sulfate, aluminum hydroxide, aluminum phosphate, or potassium aluminum sulfate, or a combination thereof). In other embodiments, the immunogenic composition does not comprise an aluminum salt. In some embodiments, the immunogenic composition does not comprise an adjuvant.

In another embodiment, provided herein are immunization regimens involving a first immunization (e.g., priming) with an immunogenic composition (e.g., a vaccine) described herein followed by one, two, or more additional immunization (e.g., boostings) with an immunogenic composition (e.g., a vaccine) described herein. In one embodiment, an immunogenic regimen involves a first immunization (e.g., priming) with an immunogenic composition (e.g., a vaccine) described herein followed by one additional immunizations (e.g., boost) with an immunogenic composition (e.g., a vaccine) described herein. In a specific embodiment, the immunogenic composition (e.g., a vaccine) used in the first immunization is the same immunogenic composition (e.g., a vaccine) used in one, two or more additional immunizations. In other specific embodiments, the immunogenic composition (e.g., vaccine) used in the first immunization is different from the immunogenic composition (e.g., vaccine) used in one, two or more additional immunizations. In a specific embodiment, an immunization regimen is analogous to the regimen described in Example 1, infra.

In a specific embodiment, in accordance with the methods described herein, a subject may be administered one, two or more doses of an immunogenic composition described herein. In some embodiments of the methods described herein, a subject is administered a prime dose and a boost dose of a monovalent immunogenic composition described herein. In some embodiments of the methods described herein, a subject is administered a prime dose and a boost dose of a bivalent immunogenic composition described herein. In some embodiments, the bivalent immunogenic composition comprises an N1 recombinant neuraminidase (e.g., N1-MPP) and an N2 recombinant neuraminidase (e.g., N2-MPP). In some embodiments, the bivalent immunogenic composition comprises an N1 recombinant neuraminidase (e.g., N1-MPP) and an B recombinant neuraminidase (e.g., B-NA-MPP). In some embodiments, the bivalent immunogenic composition comprises an N2 recombinant neuraminidase (e.g., N2-MPP) and an B recombinant neuraminidase (e.g., B-NA-MPP). In some embodiments, the bivalent immunogenic composition comprises an N1 recombinant neuraminidase (e.g., N1-MPP), an N2 recombinant neuraminidase (e.g., N2-MPP), and a CpG oligonucleotide adjuvant described herein (e.g., in Section 5.2 or Example 1). In some embodiments, the bivalent immunogenic composition comprises an N1 recombinant neuraminidase (e.g., N1-MPP), an B recombinant neuraminidase (e.g., B-NA-MPP), and a CpG oligonucleotide adjuvant described herein (e.g., in Section 5.2 or Example 1). In some embodiments, the bivalent immunogenic composition comprises an N2 recombinant neuraminidase (e.g., N2-MPP), an B recombinant neuraminidase (e.g., B-NA-MPP), and a CpG oligonucleotide adjuvant described herein (e.g., in Section 5.2 or Example 1). In some embodiments of the methods described herein, a subject is administered a prime dose and a boost dose of a trivalent immunogenic composition described herein. In some embodiments, the trivalent immunogenic composition comprises an N1 recombinant neuraminidase (e.g., N1-MPP), an N2 recombinant neuraminidase (e.g., N2-MPP), and a B recombinant neuraminidase (e.g., B-NA-MPP). In some embodiments, the trivalent immunogenic composition comprises an N1 recombinant neuraminidase (e.g., N1-MPP), an N2 recombinant neuraminidase (e.g., N2-MPP), a B recombinant neuraminidase (e.g., B-NA-MPP), and a CpG oligonucleotide adjuvant described herein (e.g., in Section 5.2 or Example 1).

In a specific embodiment, a subject is immunized in accordance with a method described herein prior to or during flu season. In some embodiments, a subject is immunized in accordance with a method described herein prior to and during flu season. In a specific embodiment, flu season in the U.S. may be from September or October of one year through March or April of the next year.

In some embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one, two, or more subtypes of influenza A virus. In some embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one, two, three or more strains of influenza A virus. In certain embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by a subtype of influenza virus that belongs to one NA group and not another NA group. In some embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one or more strains within the same subtype of influenza A virus. In certain embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one, two, three or more strains within the same subtype of influenza A virus.

In some embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one, two, or more lineages of influenza B virus. In some embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one, two, three or more strains of influenza B virus. In some embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one or more strains within the same lineage of influenza B virus. In certain embodiments, the immune response induced by an immunogenic composition described herein, or the administration of a recombinant neuraminidase or immunogenic composition thereof in combination with a CpG oligonucleotide adjuvant described herein is effective to prevent an influenza virus disease caused by one, two, three or more strains within the same lineage of influenza B virus.

In some embodiments, the immune response induced by an immunogenic composition described herein, or the administration of a recombinant neuraminidase or immunogenic composition thereof in combination with a CpG oligonucleotide adjuvant described herein is effective to reduce the number of symptoms resulting from an influenza virus disease/infection. In certain embodiments, the immune response induced by an immunogenic composition described herein, or the administration of a recombinant neuraminidase or immunogenic composition thereof in combination with a CpG oligonucleotide adjuvant described herein is effective to reduce the duration of one or more symptoms resulting from an influenza virus disease/infection. Symptoms of influenza virus disease/infection include, but are not limited to, body aches (especially joints and throat), fever, nausea, headaches, irritated eyes, fatigue, sore throat, reddened eyes or skin, and abdominal pain.

In some embodiments, the immune response induced by an immunogenic composition described herein, or the administration of a recombinant neuraminidase or immunogenic composition thereof in combination with a CpG oligonucleotide adjuvant described herein is effective to reduce the hospitalization of a subject suffering from an influenza virus disease/infection. In some embodiments, the immune response induced by an immunogenic composition described herein, or the administration of a recombinant neuraminidase or immunogenic composition thereof in combination with a CpG oligonucleotide adjuvant described herein is effective to reduce the duration of hospitalization of a subject suffering from an influenza virus disease/infection.

In a specific embodiment, the immune response induced by an immunogenic composition described herein, or the administration of a recombinant neuraminidase or immunogenic composition thereof in combination with a CpG oligonucleotide adjuvant described herein induces NA-specific antibodies (e.g., IgG). In another specific embodiment, the immune response induced by an immunogenic composition described herein, or the administration of a recombinant neuraminidase or immunogenic composition thereof in combination with a CpG oligonucleotide adjuvant described herein induces antibodies with ADCC activity as assessed by a technique known to one of skill in the art or described herein. In another specific embodiment, the immune response induced by an immunogenic composition described herein, or the administration of a recombinant neuraminidase or immunogenic composition thereof in combination with a CpG oligonucleotide adjuvant described herein induces antibodies with neuraminidase inhibition activity as assessed by a technique known to one of skill in the art or described herein. In another specific embodiment, the immune response induced by an immunogenic composition described herein, or the administration of a recombinant neuraminidase or immunogenic composition thereof in combination with a CpG oligonucleotide adjuvant described herein induces antibodies with (1) ADCC activity as assessed by a technique known to one of skill in the art; and (2) neuraminidase inhibition activity as assessed by a technique known to one of skill in the art or described herein.

In certain embodiments, the methods for preventing an influenza virus disease in a subject (e.g., a human or non-human animal) provided herein result in a reduction in the replication of the influenza virus in the subject as measured by in vivo and in vitro assays known to those of skill in the art and described herein. In some embodiments, the replication of the influenza virus is reduced by approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. In specific embodiments, the methods for preventing an influenza virus disease in a subject (e.g., a human or non-human animal) provided herein result in a reduction of the titer of an influenza virus detected in the subject. In specific embodiments, the methods for preventing an influenza virus disease in a subject results in one, two, or more of the following: (1) reduces the number of symptoms of the infection/disease, (2) reduces the severity of the symptoms of the infection/disease, (3) reduces the length of the infection/disease, (4) reduces hospitalization or complications resulting from the infection/disease, (5) reduces the length of hospitalization of the subject, (6) reduces organ failure associated with the influenza virus infection/disease, and (7) increases survival of the subject. In a specific embodiment, the methods for preventing an influenza virus disease in a subject inhibits the development or onset of an influenza virus disease or one or more symptoms thereof.

In another aspect, provided herein are methods for preventing an influenza virus disease, or treating an influenza virus infection or an influenza virus disease in a subject comprising administering to the subject an anti-influenza virus NA antibody(ies), wherein the anti-influenza virus NA antibody(ies) was generated utilizing an immunogenic composition described herein, or a recombinant neuraminidase or immunogenic composition thereof in combination with a CpG oligonucleotide adjuvant described herein. For example, an immunogenic composition described herein may be administered to a non-human subject (e.g., a non-human subject that expresses or is capable of expression human antibody) to generate anti-influenza virus NA antibody(ies). In a specific embodiment, provided herein is a method for preventing an influenza virus disease in a human subject comprising administering the subject a human or humanized anti-influenza virus NA antibody(ies), wherein the anti-influenza virus NA antibody(ies) was generated utilizing an immunogenic composition described herein or a recombinant neuraminidase or immunogenic composition thereof in combination with a CpG oligonucleotide adjuvant described herein.

In certain embodiments, provided herein are methods for generating antibodies comprising administering an immunogenic composition described herein, or a recombinant neuraminidase or immunogenic composition thereof in combination with a CpG oligonucleotide adjuvant described herein to a subject (e.g., a non-human subject). In some embodiments, an immunogenic composition described herein, or a recombinant neuraminidase or immunogenic composition thereof in combination with a CpG oligonucleotide adjuvant described herein may be administered to a subject (e.g., a non-human subject) and the antibodies may be isolated. The isolated antibodies may be cloned. The antibodies may be humanized and/or optimized. In some embodiments, hybridomas are produced which produce a particular antibody of interest. In certain embodiments, the non-human subject administered a recombinant neuraminidase described herein or a composition described herein is capable of producing human antibodies. Techniques for isolating, cloning, humanizing, optimizing and for generating hybridomas are known to one of skill in the art. In a specific embodiment, antibodies generated by a method described herein may be utilized in assays (e.g., assays described herein) as well as in passive immunization of a subject (e.g., a human subject). Thus, provided herein, in certain embodiments, are methods for treating influenza virus infection or preventing influenza virus disease, comprising administering antibodies generated by a method described herein.

### 5.6.1 Combination Therapies

In various embodiments, a recombinant neuraminidase described herein or an immunogenic composition described herein may be administered to a subject in combination with one or more other therapies (e.g., an antiviral, antibacterial, or immunomodulatory therapies). In some embodiments, a recombinant neuraminidase described herein or an immunogenic composition described herein may be administered to a subject in combination with one or more therapies (e.g., an antiviral, antibacterial, or immunomodulatory therapies). The one or more other therapies may be beneficial in the prevention of an influenza virus disease or may ameliorate a symptom or condition associated with an influenza virus disease. In some embodiments, the one or more other therapies are pain relievers, anti-fever medications, or therapies that alleviate or assist with breathing. In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patient visit.

### 5.6.2 Patient Populations

In certain embodiments, a recombinant neuraminidase described herein, an immunogenic composition described herein, or a recombinant neuraminidase or immunogenic composition thereof in combination with a CpG oligonucleotide adjuvant described herein may be administered to a naïve subject, i.e., a subject that does not have a disease caused by influenza virus infection or has not been and is not currently infected with an influenza virus infection. In one embodiment, a recombinant neuraminidase described herein, an immunogenic composition described herein, or a recombinant neuraminidase or immunogenic composition thereof in combination with a CpG oligonucleotide adjuvant described herein is administered to a naïve subject that is at risk of acquiring an influenza virus infection.

In another embodiment, a recombinant neuraminidase described herein, an immunogenic composition described herein, or a recombinant neuraminidase or immunogenic composition thereof in combination with a CpG oligonucleotide adjuvant described herein is administered to a subject that does not have a disease caused by the specific influenza virus, or has not been and is not infected with the specific influenza virus to which the recombinant neuraminidase induces an immune response.

In another embodiment, a recombinant neuraminidase described herein, an immunogenic composition described herein, or a recombinant neuraminidase or immunogenic composition thereof in combination with a CpG oligonucleotide adjuvant described herein may also be administered to a subject that is, has been, or is and has been infected with the influenza virus or another type, subtype/lineage, or strain of the influenza virus to which the recombinant neuraminidase induces an immune response.

In certain embodiments, recombinant neuraminidase described herein, an immunogenic composition described herein, or a recombinant neuraminidase or immunogenic composition thereof in combination with a CpG oligonucleotide adjuvant described herein is administered to a patient who has been diagnosed with an influenza virus infection. In some embodiments, a recombinant neuraminidase described herein, an immunogenic composition described herein, or a recombinant neuraminidase or immunogenic composition thereof in combination with a CpG oligonucleotide adjuvant described herein is administered to a patient infected with an influenza virus before symptoms manifest or symptoms become severe (e.g., before the patient requires hospitalization).

In some embodiments, a subject administered a recombinant neuraminidase described herein, an immunogenic composition described herein, or a recombinant neuraminidase or immunogenic composition thereof in combination with a CpG oligonucleotide adjuvant described herein is an animal. In certain embodiments, the animal is a bird. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human.

In specific embodiments, a subject administered a recombinant neuraminidase described herein, an immunogenic composition described herein, or a recombinant neuraminidase or immunogenic composition thereof in combination with a CpG oligonucleotide adjuvant described herein is a human infant. As used herein, the term “human infant” refers to a newborn to 1 year old human.

In specific embodiments, a subject administered a recombinant neuraminidase described herein, an immunogenic composition described herein, or a recombinant neuraminidase or immunogenic composition thereof in combination with a CpG oligonucleotide adjuvant described herein is a human child. As used herein, the term “human child” refers to a human that is 1 year to 18 years old.

In specific embodiments, a subject administered a recombinant neuraminidase described herein, an immunogenic composition described herein, or a recombinant neuraminidase or immunogenic composition thereof in combination with a CpG oligonucleotide adjuvant described herein is a human adult. As used herein, the term “human adult” refers to a human that is 18 years or older.

In specific embodiments, a subject administered a recombinant neuraminidase described herein, an immunogenic composition described herein, or a recombinant neuraminidase or immunogenic composition thereof in combination with a CpG oligonucleotide adjuvant described herein is an elderly human. As used herein, the term “elderly human” refers to a human 65 years or older.

In some embodiments, the human subject to be administered a recombinant neuraminidase described herein, an immunogenic composition described herein, or a recombinant neuraminidase or immunogenic composition thereof in combination with a CpG oligonucleotide adjuvant described herein is any individual at increased risk of influenza virus infection or disease resulting from influenza virus infection (e.g., an immunocompromised or immunodeficient individual).

In some embodiments, the human subject to be administered a recombinant neuraminidase described herein, an immunogenic composition described herein, or a recombinant neuraminidase or immunogenic composition thereof in combination with a CpG oligonucleotide adjuvant described herein is any individual in close contact with an individual with increased risk of influenza virus infection or disease resulting from influenza virus infection (e.g., immunocompromised or immunosuppressed individuals).

In some embodiments, the human subject to be administered a recombinant neuraminidase described herein, an immunogenic composition described herein, or a recombinant neuraminidase or immunogenic composition thereof in combination with a CpG oligonucleotide adjuvant described herein is an individual affected by a condition that increases susceptibility to influenza virus infection or complications or disease resulting from influenza virus infection. In other embodiments, a recombinant neuraminidase described herein, an immunogenic composition described herein, or a recombinant neuraminidase or immunogenic composition thereof in combination with a CpG oligonucleotide adjuvant described herein is administered to a subject in whom an influenza virus infection has the potential to increase complications of another condition that the individual is affected by, or for which they are at risk.

### 5.6.3 Routes of Delivery

A recombinant neuraminidase described herein, an immunogenic composition described herein, or a recombinant neuraminidase or immunogenic composition thereof in combination with a CpG oligonucleotide adjuvant described herein may be delivered to a subject by a variety of routes. These include, but are not limited to, intranasal, pulmonary, intratracheal, oral, intradermal, intramuscular, intraperitoneal, transdermal, intravenous, conjunctival, and subcutaneous routes as well as other routes described herein. In some embodiments, recombinant neuraminidase described herein, an immunogenic composition described herein, or a recombinant neuraminidase or immunogenic composition thereof in combination with a CpG oligonucleotide adjuvant described herein is delivered to a subject intranasally. In some embodiments, a recombinant neuraminidase described herein, an immunogenic composition described herein, or a recombinant neuraminidase or immunogenic composition thereof in combination with a CpG oligonucleotide adjuvant described herein is delivered to a subject intramuscularly. In some embodiments, a recombinant neuraminidase described herein, an immunogenic composition described herein, or a recombinant neuraminidase or immunogenic composition thereof in combination with a CpG oligonucleotide adjuvant described herein is delivered to a subject subcutaneously.

In some embodiments, an immunogenic composition described herein is formulated for topical administration, for example, for application to the skin. In specific embodiments, the route of administration is nasal, e.g., as part of a nasal spray. In certain embodiments, an immunogenic composition described herein is formulated for intramuscular administration. In some embodiments, an immunogenic composition described herein is formulated for subcutaneous administration. In some embodiments, an immunogenic composition described herein is formulated for intranasal administration.

### 5.6.4 Dosage

The amount of a recombinant neuraminidase described herein, an immunogenic composition described herein, or a recombinant neuraminidase described herein or immunogenic composition thereof in combination with a CpG oligonucleotide adjuvant described herein, which will be effective in the prevention of an influenza virus disease will depend on the nature of the disease

As used herein, the term “effective amount” in the context of administering a therapy to a subject refers to the amount of a therapy which may have a prophylactic effect(s), therapeutic effect(s), or both a prophylactic and therapeutic effect(s). In certain embodiments, an “effective amount” in the context of administration of a therapy to a subject refers to the amount of a therapy which is sufficient to achieve one, two, three, four, or more of the following effects: (i) reduce or ameliorate the severity of an influenza virus infection, influenza virus disease, or symptom associated therewith; (ii) reduce the duration of an influenza virus infection, influenza virus disease, or symptom associated therewith; (iii) prevent the progression of an influenza virus infection, influenza virus disease, or symptom associated therewith; (iv) cause regression of an influenza virus infection, influenza virus disease, or symptom associated therewith; (v) prevent the development or onset of an influenza virus infection, influenza virus disease, or symptom associated therewith; (vi) prevent the recurrence of an influenza virus infection, influenza virus disease or symptom associated therewith; (vii) reduce or prevent the spread of an influenza virus from one cell to another cell, one tissue to another tissue, or one organ to another organ; (viii) prevent or reduce the spread of an influenza virus from one subject to another subject; (ix) reduce organ failure associated with an influenza virus infection; (x) reduce hospitalization of a subject; (xi) reduce hospitalization length; (xii) increase the survival of a subject with an influenza virus infection or disease associated therewith; (xiii) eliminate an influenza virus infection or disease associated therewith; (xiv) inhibit or reduce influenza virus replication; (xv) inhibit or reduce the entry of an influenza virus into a host cell(s); (xvi) inhibit or reduce replication of the influenza virus genome; (xvii) inhibit or reduce synthesis of influenza virus proteins; (xviii) inhibit or reduce assembly of influenza virus particles; (xix) inhibit or reduce release of influenza virus particles from a host cell(s); (xx) reduce influenza virus titer; and/or (xxi) enhance or improve the prophylactic or therapeutic effect(s) of another therapy.

In certain embodiments, the effective amount does not result in complete protection from an influenza virus disease, but results in a lower titer or reduced number of influenza viruses compared to an untreated subject with an influenza virus infection. In certain embodiments, the effective amount results in a 0.5 fold, 1 fold, 1.5 fold, 2 fold, 3 fold, 4 fold, 5 fold, 6 fold, 7 fold, 8 fold, 9 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of influenza virus relative to an untreated subject with an influenza virus infection. In some embodiments, the effective amount results in a reduction in titer of influenza virus relative to an untreated subject with an influenza virus infection of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titer, number or total burden of influenza virus include, but are not limited to, less severe symptoms of the infection, fewer symptoms of the infection and a reduction in the length of the disease associated with the infection.

In certain embodiments, an effective amount of a therapy (e.g., a composition thereof) results in an anti-influenza virus NA titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus NA titer in a blood sample from the subject prior to immunization. In certain embodiments, an effective amount of a therapy (e.g., a composition thereof) results in an anti-influenza virus NA stalk titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-influenza virus NA stalk titer in a blood sample from the subject prior to immunization.

In some embodiments, an effective amount of a recombinant neuraminidase described herein (e.g., in Section 5.3 or Section 6) for administration to a subject (e.g., a human subject) is 5 to 30 μg of the recombinant neuraminidase described herein. In some embodiments, an effective amount of an immunogenic composition for administration to a subject (e.g., a human subject) contains 5 to 30 μg of a recombinant neuraminidase described herein (e.g., in Section 5.3 or Section 6).

In some embodiments, a dose of a recombinant neuraminidase described herein (e.g., in Section 5.3 or Section 6) for administration to a subject (e.g., a human subject) may contain 5 to 30 μg of the recombinant neuraminidase described herein. In some embodiments, a dose of an immunogenic composition for administration to a subject (e.g., a human subject) may contain 5 to 30 μg of a recombinant neuraminidase described herein (e.g., in Section 5.3 or Section 6). In some embodiments, a dose of an immunogenic composition for administration to a subject (e.g., a human subject) may contain up to 45 μg of a recombinant neuraminidase described herein (e.g., in Section 5.3 or Section 6). In some embodiments, a dose of an immunogenic composition for administration to a subject (e.g., a human subject) may contain 5 μg to 45 μg of a recombinant neuraminidase described herein (e.g., in Section 5.3 or Section 6).

A unit dose of the immunogenic composition for administration to a human subject, which is typically a 0.5 mL or a 1.0 mL dose, may comprises from about 375 μg to about 6000 μg of a CpG oligonucleotide of a CpG oligonucleotide adjuvant described herein (e.g., in Section 5.2 or Example 1), preferably from about 500 μg to about 5000 μg of the CpG oligonucleotide, preferably from about 750 μg to about 3000 μg of the CpG oligonucleotide. In some embodiments, a unit dose of the immunogenic composition comprises greater than about 250, 500, 750, 1000, or 1250 μg of the CpG oligonucleotide of a CpG oligonucleotide adjuvant described herein (e.g., in Section 5.2 or Example 1), and less than about 6000, 5000, 4000, 3000, or 2000 μg of the CpG oligonucleotide. In some embodiments, a unit dose of the immunogenic composition comprises about 375, 750, 1500, 3000 or 6000 μg of the CpG oligonucleotide of a CpG oligonucleotide adjuvant. In some embodiments, a unit dose of the immunogenic composition comprises about 750 μg of the CpG oligonucleotide adjuvant described herein (e.g., in Section 5.2 or Example 1). In some embodiments, a unit dose of the immunogenic composition comprises about 1500 μg of the CpG oligonucleotide of a CpG oligonucleotide adjuvant described herein (e.g., in Section 5.2 or Example 1). In some embodiments, a unit dose of the immunogenic composition comprises about 3000 μg of the CpG oligonucleotide of a CpG oligonucleotide adjuvant described herein (e.g., in Section 5.2 or Example 1). In some embodiments, a unit dose of the immunogenic composition comprises about 6000 μg of the CpG oligonucleotide of a CpG oligonucleotide adjuvant described herein (e.g., in Section 5.2 or Example 1).

In some embodiments, the amount of CpG oligonucleotide administered to a subject when administered in combination with a recombinant neuraminidase described herein is from about 375 μg to about 6000 μg of a CpG oligonucleotide described herein (e.g., in Section 5.2 or Example 1), preferably from about 500 μg to about 5000 μg of the CpG oligonucleotide, preferably from about 750 μg to about 3000 μg of the CpG oligonucleotide. In some embodiments, the amount of CpG oligonucleotide administered to a subject when administered in combination with a recombinant neuraminidase described herein is greater than about 250, 500, 750, 1000, or 1250 μg of a CpG oligonucleotide described herein (e.g., in Section 5.2 or Example 1), and less than about 6000, 5000, 4000, 3000, or 2000 μg of the CpG oligonucleotide. In some embodiments, the amount of CpG oligonucleotide administered to a subject when administered in combination with a recombinant neuraminidase described herein is about 375, 750, 1500, 3000 or 6000 μg of a CpG oligonucleotide described herein (e.g., in Section 5.2 or Example 1). In some embodiments, the amount of CpG oligonucleotides administered to a subject when administered in combination with a recombinant neuraminidase described herein is about 750 μg of a CpG oligonucleotide described herein (e.g., in Section 5.2 or Example 1). In some embodiments, the amount of CpG oligonucleotides administered to a subject when administered in combination with a recombinant neuraminidase described herein is about 1500 μg of a CpG oligonucleotide described herein (e.g., in Section 5.2 or Example 1). In some embodiments, the amount of CpG oligonucleotides administered to a subject when administered in combination with a recombinant neuraminidase described herein is about 3000 μg of a CpG oligonucleotide described herein (e.g., in Section 5.2 or Example 1). In some embodiments, the amount of CpG oligonucleotides administered to a subject when administered in combination with a recombinant neuraminidase described herein is about 6000 μg of a CpG oligonucleotide described herein (e.g., in Section 5.2 or Example 1). In some embodiments, the CpG oligonucleotide and the recombinant neuraminidase administered to a subject are not in the same composition.

### 5.7 Biological Assays

Also provided herein are biological assays that may be used to characterize a recombinant NA. See, also, Section 6 for particular assays. In a specific embodiment, an assay described in Section 5.7.1 or Section 6 is used to characterize a recombinant NA. In another specific embodiment, an assay described in Section 6 is used to characterize the neuraminidase inhibition activity of antibodies induced by an immunogenic composition described herein. In another specific embodiment, the immunogenicity or effectiveness of an immunogenic composition described herein is assessed using one, two, or more assays described in Section 5.8.1 or Example 1. In another specific embodiment, the immunogenicity or effectiveness of a recombinant neuraminidase described herein, an immunogenic composition described herein, or a recombinant neuraminidase described herein or immunogenic composition thereof in combination with a CpG oligonucleotide adjuvant described herein, is assessed using one, two, or more assays described in Section 5.8.1 or Example 1.

In some embodiments, the protection against influenza virus disease following administration of a recombinant neuraminidase described herein, an immunogenic composition described herein, or a recombinant neuraminidase or immunogenic composition thereof in combination with a CpG oligonucleotide adjuvant described herein to a subject may be assessed in an assay described herein (e.g., in Example 1) or known to one of skill in the art.

In some embodiments, the specificity of antibodies produced following administration of a recombinant neuraminidase described herein, an immunogenic composition described herein, or a recombinant neuraminidase or immunogenic composition thereof in combination with a CpG oligonucleotide adjuvant described herein to a subject may be assessed in an assay described herein (e.g., in Example 1) or known to one of skill in the art. In some embodiments, the cross-reactivity of antibodies produced following administration of a recombinant neuraminidase described herein, an immunogenic composition described herein, or a recombinant neuraminidase or immunogenic composition thereof in combination with a CpG oligonucleotide adjuvant described herein to a subject may be assessed in an assay described herein (e.g., in Example 1) or known to one of skill in the art. For example, an ELISA assay may be conducted.

In some embodiments, the ability of antibodies produced following administration of a recombinant neuraminidase described herein, an immunogenic composition described herein, or a recombinant neuraminidase or immunogenic composition thereof in combination with a CpG oligonucleotide adjuvant described herein to protect against influenza virus disease may be assessed in an assay described herein (e.g., in Example 1) or known to one of skill in the art.

### 5.7.1 Assays for Testing Activity of Recombinant Neuraminidase

Assays for testing the expression of a recombinant neuraminidase disclosed herein may be conducted using any assay known in the art. For example, an immunoassay, such as a Western blot, may be used to assess the expression of a recombinant neuraminidases.

In another embodiment, a recombinant neuraminidase disclosed herein is assayed for proper folding by determination of the structure or conformation of the recombinant neuraminidase using any method known in the art such as, e.g., NMR, X-ray crystallographic methods, or secondary structure prediction methods, e.g., circular dichroism or in-silico prediction.

In another embodiment, a recombinant neuraminidase disclosed herein is assessed for the ability to form tetramers using a technique known in the art or described herein (e.g., in Example 1 of Section 6). In specific embodiments, the ability to form tetramers is assessed using polyacrylamide gel electrophoresis (PAGE). In some embodiments, PAGE is denaturing PAGE (i.e., denaturing conditions). In other embodiments, PAGE is native PAGE (i.e., non-denaturing conditions). In specific embodiments, the ability to form tetramers is assessed using a recombinant neuraminidase which is cross-linked. In certain embodiments, the recombinant neuraminidase is cross-linked using bis-sulfosuccinimidyl suberate (BS3). In another specific embodiment, the ability to form tetramers is assessed using a recombinant neuraminidase which is not cross-linked. In another embodiment, a recombinant neuraminidase disclosed herein or a virus containing or expressing a recombinant neuraminidase disclosed herein is assessed for influenza virus neuraminidase activity using a technique known to one of skill in the art or described herein (e.g., in Example 1 of Section 6, infra).

In specific embodiments, neuraminidase activity is assessed using a cleavage assay (i.e., cleavage of neuraminidase-specific substrate). In some embodiments, the cleavage assay comprises a sialic acid cleavage assay. In some embodiments, the cleavage assay comprises detection of cleavage or a lack thereof. In specific embodiments, detection of cleavage comprises chemiluminescent detection. In certain embodiments, neuraminidase activity is assessed using a NA-star assay (e.g., in Example 1 of Section 6, infra).

### 5.8 Kits

In one aspect, provided herein is a pharmaceutical pack or kit for use in accordance with the methods described herein (e.g., the immunization of a subject against influenza virus, or the prevention of influenza virus disease), the pack or kit comprising one or more containers filled with one or more of the ingredients of an immunogenic compositions described herein, such as an a recombinant neuraminidase. In a specific embodiment, provided herein is a pharmaceutical pack or kit for use in accordance with the methods described herein (e.g., the immunization of a subject against influenza virus, or the prevention of influenza virus disease), the pack or kit comprising a container containing an immunogenic composition comprising a recombinant neuraminidase described herein, and a CpG oligonucleotide adjuvant described herein (e.g., as described herein Section 5.2 or Example 1). In a specific embodiment, provided herein is a pharmaceutical pack or kit for use in accordance with the methods described herein (e.g., the immunization of a subject against influenza virus, or the prevention of influenza virus disease), the pack or kit comprising a container containing an immunogenic composition comprising a recombinant neuraminidase described herein, a CpG oligonucleotide adjuvant described herein (e.g., as described herein Section 5.2 or Example 1), and an influenza vaccine (e.g., a seasonal influenza vaccine). In some embodiments, the immunogenic composition further comprises an aluminum salt (e.g., aluminum hydroxide). In a specific embodiment, provided herein is a pharmaceutical pack or kit for use in accordance with the methods described herein (e.g., the immunization of a subject against influenza virus, or the prevention of influenza virus disease), the pack or kit comprising a first container containing a CpG oligonucleotide adjuvant described herein (e.g., as described herein Section 5.2 or Example 1) and a second container containing a recombinant neuraminidase described herein, or an immunogenic composition described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods described herein. In one embodiment, a kit comprises a recombinant neuraminidase described herein (such as described in Section 5.3 above or Example 1 of Section 6), in one or more containers, and a CpG oligonucleotide adjuvant described herein (e.g., in Section 5.2 or Example 1), in one or more containers. In another embodiment, a kit comprises one or more immunogenic compositions described herein in one or more containers.

### 6. EXAMPLE 1: A CPG 1018® ADJUVANT ADJUVANTED RECOMBINANT NEURAMINIDASE VACCINE PROVIDES ROBUST PROTECTION FROM INFLUENZA VIRUS CHALLENGE IN THE MOUSE MODEL

This example provides data demonstrating that a recombinant influenza virus N1 neuraminidase (NA) adjuvanted with CpG 1018®, a TLR 9 agonist, induces a strong and robust immune response against NA. This combination of recombinant NA adjuvanted with CpG 1018® adjuvant induced robust anti-NA immunity and protection against morbidity and mortality after high lethal challenge in vivo.

### 6.1.1 Introduction

Influenza virus infections pose a significant threat to global health by causing severe respiratory infections. Vaccination is the main countermeasure against influenza virus spread, however the effectiveness of these vaccines is variable. Current seasonal influenza virus vaccines mostly rely on the immunodominant hemagglutinin (HA) glycoprotein on the viral surface, which usually leads to a narrow and strain specific immune response. Since the HA undergoes constant antigenic drift, it can lead to a dramatic loss in vaccine effectiveness, requiring annual administration of influenza virus vaccines.

NA as emerged as an attractive target for vaccine development since it is less prone to undergo antigenic drift and therefore is antigenically more stable. The obstacles of using NA as a vaccine antigen include its lack of standardization in seasonal vaccine preparations as well as its fragile stability. Indeed, the amount of NA in seasonal vaccines usually varies in quality and quantity [14] and it is likely that the structural integrity in current vaccine formulations is suboptimal. Additionally, it has been hypothesized that antigenic competition occurs between HA and NA in vaccine formulations, making the NA immunosubdominant [15]. The anti-NA immunity acquired after vaccination with live attenuated or inactivated vaccines is mediocre at best [16]. Stable, recombinant NA protein has been shown to be immunogenic and protective in animal models [13, 14, 24]. However, recombinant protein vaccines usually tend to induce a lower immune response compared to whole virus vaccines due to the lack of molecules that engage innate immune receptors.

This example describes the results from tests using a recombinant influenza virus N1 neuraminidase vaccine candidate, named N1-MPP, adjuvanted with CpG 1018®, a TLR9 agonist. The N1-MPP protein utilizes the phosphoprotein of measles virus to multimerize and stabilize the NA protein. The tetramerization of the protein is a crucial factor in generating a NA based vaccine, since it has been previously shown that only multimeric but not monomeric protein confers protection in vivo [25, 26]. N1-MPP can form fully enzymatically active NA tetramers which are highly protective in vivo in a mouse model and induce high titers of neuraminidase inhibiting (NI) antibodies after vaccination. This example also describes N2-MPP and B-NA-MPP vaccine constructs, which were generated to cover the range of influenza viruses that are seasonally circulating in humans. These constructs have been characterized in vitro and in vivo regarding their functionality and protective potential. Furthermore, a trivalent NA-MPP mix was tested. Interestingly, no antigenic competition between the individual NA constructs was detected. By adjuvating the recombinant protein constructs with CpG 1018® adjuvant it was possible to induce a strong and robust immune response against the NA, which provided full protection against morbidity and mortality after high lethal challenge in vivo. Data acquired in this study provides an important insight regarding a broadly protective NA based influenza virus vaccine candidate.

### 6.1.2 Materials and Methods

### Cells and Viruses

Madin-Darby canine kidney (MDCK) cells (ATCC #CCL-34) were maintained in Dulbecco's Modified Eagle's medium (DMEM; Gibco) containing 10% fetal bovine serum (FBS; Gibco), 1% penicillin/streptomycin antibiotics mix (100 U/mL penicillin, 100 μg/mL streptomycin; Gibco) and 1% hydroxyethylpiperazine ethane sulfonic acid (HEPES; Gibco). BTN-TN-5B1-4 (Trichoplusia ni, High Five) cells were maintained in Express Five media (Gibco) containing 1% L-glutamine (Gibco) and 1% penicillin/streptomycin antibiotics mix. Sf9 (Spodoptera frugiperda) cells were maintained in Trichoplusia ni medium—Fred Hink (TNM-FH; Gemini Bioproducts) containing 10% FBS, 1% penicillin/streptomycin antibiotics mix and 1% Pluronic F-68 (Sigma Aldrich). For passaging baculovirus stocks in Sf9 cells, the medium was switched to TNM-FH containing 3% FBS, 1% Pluronic F-68 and 1% penicillin/streptomycin antibiotics mix. The reassortant viruses used in this study were grown in 10 day old embryonated chicken eggs (Charles River Laboratories). The H7N1 viruses used in neuraminidase inhibition assay contain the internal genes of A/Puerto Rico/8/1934 H1N1, an exotic H7 hemagglutinin of A/mallard/Alberta/24/01 H7N3, and either the N1 of A/Michigan/45/2015 H1N1 (H7N1Mich15) or A/California/04/09 H1N1 (H7N1Cal09). The challenge virus A/Singapore/GP1908/2015 (H1N1, IVR-180 strain) possesses the internal proteins of A/Texas/1/77 (H3N2) and the surface glycoproteins of A/Singapore/GP1908/15 (pH1N1). A/Switzerland/9715293/2013 (H3N2) and B/New York/PV01181/2018 are based on wild type backbones but are mouse-adapted. A/Vietnam/1203/04 (H5N1) is a reassortant virus with the internal genes of A/Puerto Rico/8/1934 H1N1 and has a deleted polybasic cleavage site.

### Recombinant Proteins

Constructs for producing recombinant neuraminidase fusion proteins were generated by cloning an influenza virus globular head domain and codon optimized tetramerization domain into a pFastBacDual expression vector, which contains a signal peptide sequence, purification tag, and cleavage site. The recombinant N1-MPP, N2-MPP and B-NA-MPP neuraminidases used for animal vaccination were structured, in order: an N-terminal signal peptide, a hexahistidine purification tag, a measles virus phosphoprotein tetramerization domain, a thrombin cleavage site, and either an influenza A neuraminidase subtype N1 globular head domain (A/Michigan/45/2015) or subtype N2 globular head domain (A/Kansas/14/2017 (H3N2)), or an influenza B neuraminidase globular head domain (B/Colorado/6/2017)). Specific nucleotide and amino acid sequences of exemplary recombinant N1-MPP, N2-MPP and B-MPP (sometimes B-MPP is referred to herein as B-NA-MPP) neuraminidases tested are shown below in TABLE 2.

TABLE 2
Exemplary Recombinant Neuraminidase Constructs

| SEQ | | |
|---|---|---|
| ID | | |
| NO: | Construct | Sequence |
| 48 | N1-MPP | MLLVNQSHQGFNKEHTSKMVSAIVLYVLLAAAAHSAFAADPHHHHHHG |
| | | DHYDDELFSDVQDIKTALAKIHEDNQKIISKLESLLLLKGEVESIKKQ |
| | | INRQNISISTLEGHLSSIMIAIPGLSLVPRGSPSRSVKLAGNSSLCPVSG |
| | | WAIYSKDNSVRIGSKGDVFVIREPFISCSPLECRTFFLTQGALLND |
| | | KHSNGTIKDRSPYRTLMSCPIGEVPSPYNSRFESVAWSASACHDG |
| | | INWLTIGISGPDSGAVAVLKYNGIITDTIKSWRNNILRTQESECAC |
| | | VNGSCFTIMTDGPSDGQASYKIFRIEKGKIIKSVEMKAPNYHYEE |
| | | CSCYPDSSEITCVCRDNWHGSNRPWVSFNQNLEYQMGYICSGVF |
| | | GDNPRPNDKTGSCGPVSSNGANGVKGFSFKYGNGVWIGRTKSIS |
| | | SRKGFEMIWDPNGWTGTDNKFSIKQDIVGINEWSGYSGSFVQHP |
| | | ELTGLDCIRPCFWVELIRGRPEENTIWTSGSSISFCGVNSDTVGW |
| | | SWPDGAELPFTIDK* |
| | | (signal peptide and his tag are italicized; MPP tetramerization domain is |
| | | underlined; thrombin cleavage site plus flanking amino acid residues is |
| | | double underline; NA globular head domain of influenza virus |
| | | A/Michigan/45/2015 is bold) |
| 59 | N2-MPP | atgctgctcgtcaaccaatcccaccagggcttcaacaaggaacacacttctaagatggtctccgctatcgtgc |
| | | tctacgtgctgctcgctgccgctgcccactcagctttcgctgccgacccacaccaccaccaccaccacggcg |
| | | atcactacgacgacgaactgttctccgacgtgcaggacatcaagaccgctctggctaagatccacgaggac |
| | | aaccagaagatcatctccaagctggaatccctgctgctgctgaagggcgaagtcgagtccatcaagaagca |
| | | gatcaaccgccagaacatctccatctccaccttggagggtcacctgtcctccatcatgatcgctatccctggcc |
| | | tgtctctcgtgcccaggggatcaccttctagaatatgccccaaaccagcagaatacagaaattggtcaaaacc |
| | | gcaatgtggcattacaggatttgcacctttctctaaggacaattcgattaggctttccgctggtggggacatctg |
| | | ggtgacaagagaaccttatgtgtcatgcgatcctgacaagtgttatcaatttgcccttggacagggaacaaca |
| | | ataaacaacgtgcattcaaataacacagcacgtgataggacccctcatcggactctattgatgaatgagttgg |
| | | gtgttcctttccatctggggaccaagcaagtgtgcatagcatggtccagctcaagttgtcacgatggaaaagc |
| | | atggctgcatgtttgtataacgggggatgataaaaatgcaactgctagtttcatttacaatgggaggcttgtaga |
| | | tagtgttgtttcatggtccaaagatattctcaggacccaggagtcagaatgcgtttgtatcaatggaacttgtaca |
| | | gtagtaatgactgatggaaatgctacaggaaaagctgatactaaaatattattcattgaggaggggaaaatcgt |
| | | tcatactagcaaattgtcaggaagtgctcagcatgtcgaagagtgctcttgctatcctcgataccctggtgtca |
| | | gatgtgtctgcagagacaactggaaaggatccaaccggcccatcgtagatataaacataaaggatcatagca |
| | | ttgtttccagttatgtgtgttcaggacttgttggagacacacccagaaaaaccgacagctccagcagcagcca |
| | | ttgcttgaatcctaacaatgaaaaaggtggtcatggagtgaaaggctgggcctttgatgatggaaatgacgtg |
| | | tggatggggagaacaatcaacgagacgtcacgcttagggtatgaaaccttcaaagtcgttgaaggctggtcc |
| | | aaccctaagtccaaattgcagataaataggcaagtcatagttgacagaggtgataggtccggttattctggtat |
| | | tttctctgttgaaggcaaaagctgcatcaatcggtgcttttatgtggagttgattaggggaagaaaagaggaaa |
| | | ctgaagtcttgtggacctcaaacagtattgttgtgttttgtggcacctcaggtacatatggaacaggctcatggc |
| | | ctgatggggcggacctcaatctcatgcatatataa |
| 58 | N2-MPP | MLLVNQSHQGFNKEHTSKMVSAIVLYVLLAAAAHSAFAADPHHHHHHG |
| | | DHYDDELFSDVQDIKTALAKIHEDNQKIISKLESLLLLKGEVESIKKQ |
| | | INRQNISISTLEGHLSSIMIAIPGLSLVPRGSPSRICPKPAEYRNWSKP |
| | | QCGITGFAPFSKDNSIRLSAGGDIWVTREPYVSCDPDKCYQFALG |
| | | QGTTINNVHSNNTARDRTPHRTLLMNELGVPFHLGTKQVCIAW |
| | | SSSSCHDGKAWLHVCITGDDKNATASFIYNGRLVDSVVSWSKDI |
| | | LRTQESECVCINGTCTVVMTDGNATGKADTKILFIEEGKIVHTS |
| | | KLSGSAQHVEECSCYPRYPGVRCVCRDNWKGSNRPIVDINIKDH |
| | | SIVSSYVCSGLVGDTPRKTDSSSSSHCLNPNNEKGGHGVKGWAF |
| | | DDGNDVWMGRTINETSRLGYETFKVVEGWSNPKSKLQINRQVI |
| | | VDRGDRSGYSGIFSVEGKSCINRCFYVELIRGRKEETEVLWTSNS |
| | | IVVFCGTSGTYGTGSWPDGADLNLMHI* |
| | | (signal peptide and his tag are italicized; MPP tetramerization domain is |
| | | underlined; thrombin cleavage site plus flanking amino acid residues is |
| | | double underline; NA globular domain of influenza virus A/Kansas/14/2017 |
| | | is bold) |
| 69 | B-NA- | atgctgctcgtcaaccaatcccaccagggcttcaacaaggaacacacttctaagatggtctccgctatcgtgc |
| | MPP cr B- | tctacgtgctgctcgctgccgctgcccactcagctttcgctgccgacccacaccaccaccaccaccacggcg |
| | MPP | atcactacgacgacgaactgttctccgacgtgcaggacatcaagaccgctctggctaagatccacgaggac |
| | | aaccagaagatcatctccaagctggaatccctgctgctgctgaagggcgaagtcgagtccatcaagaagca |
| | | gatcaaccgccagaacatctccatctccaccttggagggtcacctgtcctccatcatgatcgctatccctggcc |
| | | tgtctctcgtgcccaggggatcaccttctagacttcttctcccagaaccggagtggacatacccgcgtttatctt |
| | | gcccgggctcaacctttcagaaagcactcctaattagccctcatagattcggagaaaccaaaggaaactcag |
| | | ctcccttgataataagggaaccttttgttgcttgtggaccaaatgaatgcaaacactttgctttaacccattatgca |
| | | gcccaaccagggggatactacaatggaacaagaggagacagaaacaagctgaggcatctaatttcagtca |
| | | aattgggcaaaatcccaacagtagagaactccattttccacatggcagcatggagcgggtccgcgtgccatg |
| | | atggtaaggaatggacatatatcggagttgatggccctgacaataatgcattgctcaaagtaaaatatggaga |
| | | agcatatactgacacataccattcctatgcaaacaacatcctaagaacacaagaaagtgcctgcaattgcatc |
| | | gggggaaattgttatctaatgataactgatggctcagcttcaggtgttagtgaatgcagatttcttaagattcgag |
| | | agggccgaataataaaagaaatatttccaacaggaagagtaaaacacactgaggaatgcacatgcggatttg |
| | | ccagcaataaaaccatagaatgtgcctgtagagacaacaggtacacagcaaaaagaccttttgtcaaattaaa |
| | | cgtggagactgatacagcagaaataaggttgatgtgcacagatacctatttggacacccccagaccaaatga |
| | | tggaagcataacaggcccttgtgaatctgatggggacaaagggagtggaggcatcaagggaggatttgttc |
| | | atcaaagaatgaaatccaagattggaaggtggtactctcgaacgatgtctcaaactgaaaggatggggatgg |
| | | gactgtatgtcaagtatggtggagacccatgggctgacagtgatgccctagcttttagtggagtaatggtttca |
| | | atgaaagaacctggttggtattcctttggcttcgaaataaaagataagaaatgcgatgtcccctgtattgggata |
| | | gagatggtacatgatggtggaaaagagacttggcactcagcagcaacagccatttactgtttaatgggctcag |
| | | gacagctgctgtgggacactgtcacaggtgttgacatggctctgtaa |
| 68 | B-NA- | MLLVNQSHQGFNKEHTSKMVSAIVLYVLLAAAAHSAFAADPHHHHHHG |
| | MPP or B- | DHYDDELFSDVQDIKTALAKIHEDNQKIISKLESLLLLKGEVESIKKQ |
| | MPP | INRQNISISTLEGHLSSIMIAIPGLSLVPRGSPSRLLLPEPEWTYPRLS |
| | | CPGSTFQKALLISPHRFGETKGNSAPLIIREPFVACGPNECKHFA |
| | | LTHYAAQPGGYYNGTRGDRNKLRHLISVKLGKIPTVENSIFHMA |
| | | AWSGSACHDGKEWTYIGVDGPDNNALLKVKYGEAYTDTYHSY |
| | | ANNILRTQESACNCIGGNCYLMITDGSASGVSECRFLKIREGRIIK |
| | | EIFPTGRVKHTEECTCGFASNKTIECACRDNRYTAKRPFVKLNV |
| | | ETDTAEIRLMCTDTYLDTPRPNDGSITGPCESDGDKGSGGIKGGF |
| | | VHQRMKSKIGRWYSRTMSQTERMGMGLYVKYGGDPWADSDA |
| | | LAFSGVMVSMKEPGWYSFGFEIKDKKCDVPCIGIEMVHDGGKE |
| | | TWHSAATAIYCLMGSGQLLWDTVTGVDMAL* |
| | | (signal peptide and his tag are italicized; MPP tetramerization domain is |
| | | underlined; thrombin cleavage site plus flanking amino acid residues is |
| | | double underline; NA globular domain of influenza virus |
| | | B/Colorado/06/2017 is bold) |

Recombinant neuraminidases from A/New Caledonia/20/99 H1N1 (NC99), A/Puerto Rico/8/1934 H1N1 (PR8), A/Brisbane/02/2018 H1N1 (Bris18), A/California/04/09 (Cal09) H1N1, A/Vietnam/1203/04 H5N1 (Vn04), A/Kansas/14/2017 H3N2 (Kansas17), B/Colorado/6/2017 (Colorado17) and A/Michigan/45/2015 H1N1 were designed in the same way as the N1-MPP, N2-MPP, and B-NA-MPP constructs, except that they contained a vasodilator stimulated phosphoprotein (Mich15-VASP) tetramerization domain instead of an MPP domain. Influenza B virus HA of B/Malaysia/2506/24 (B-Mal-HA) served as a negative control, which was designed as described previously [27].

After cloning, recombinant neuraminidase constructs were transformed into competent E. coli DH10 bacs and the isolated DNA was used to transfect Sf9 insect cells following established protocol [27]. The obtained baculoviruses, were then passaged in Sf9 cells to reach higher titers using 3% TNM-FH media. After passaging the virus for three times, the resulting stock solutions were used to infect High Five cells to express the recombinant neuraminidases. High Five cells were infected for 3 days at 27° C. without CO₂ using ExpressFive media (Gibco), supplemented with 10% L-glutamine (Gibco) and 1% penicillin/streptomycin (Gibco). Afterwards, the cell supernatant containing the respective soluble recombinant neuraminidase was separated from the cells by centrifugation and the recombinant neuraminidase protein was purified using an established protocol [27, 28]. Purified protein concentrations were measured using Quick Start™ Bradford 1× Dye Reagent (BioRad). The proteins were stored at −80° C. until usage.

### Sodium Dodecyl Sulfate Polyacrylamide Gel Electrophoresis (SDS-PAGE)

To confirm integrity of the recombinant neuraminidases, SDS-PAGE was performed under reducing conditions using a bis-sulfosuccinimidyl suberate (BS3; ThermoFisher) crosslinker. For the BS3 crosslinker SDS-PAGE, the proteins were treated with the crosslinker according to the manufacturer's instruction. For the SDS-PAGE 1.5 μg of the respective NA protein was mixed 1:1 with 2× Laemmli loading buffer (Bio-Rad) supplemented with 5% beta-mercaptoethanol. The samples were then heated for 10 minutes at 95 C and then loaded onto a polyacrylamide gradient gel (4-20% Mini-PROTEAN® TGX™ Precast Protein Gels, BioRad). After running in SDS buffer, the gels were stained with Coomassie blue (ThermoFisher) for 1 hour and destained with distilled water to visualize the proteins. Bovine serum albumin (BSA) was used as a monomeric control.

### Animal Work

All animal experiments were performed under protocols approved by the Icahn School of Medicine at Mount Sinai Institutional Animal Care and Use Committee. For all animal experiments conducted, female 6-8 week old BALB/c mice (Jackson laboratories, n=5 per group) were used, unless otherwise mentioned. The adjuvant, CpG 1018® adjuvant (TLR-9 agonist), was provided by Dynavax Technologies and administered at a dosage of 3 μg per mouse. Seasonal quadrivalent influenza vaccine (QIV) used in these experiments was Flucelvax (LOT 252380, season 2017/18). To observe if two vaccinations are required to induce a robust protective immune response, mice were intramuscularly (IM) primed or prime-boosted (after a 3-week interval) with 3 μg N1-MPP, 3 μg N1-MPP+3 μg CpG 1018®, or 3 μg of an irrelevant protein (B-Mal-HA). Six weeks after the prime, mice were intranasally challenged with 10× the mouse median lethal dose 50% (mLD50) of A/Singapore/GP1908/2015 H1N1 (IVR-180). Weight loss and survival were monitored over a period of 14 days. Mice were euthanized if they lost more than 25% of their initial body weight. Blood was taken from each mouse retroorbitally on day 21 and day 42 post prime.

In addition, a passive serum transfer was performed using serum from mice previously vaccinated with N1-MPP, N1-MPP+CpG 1018® adjuvant and B-Mal-HA (donor serum). Naïve mice received 200 μl of donor serum intraperitoneally. After 2 hours, the mice were intranasally challenged with 5×mLD₅₀ of A/Singapore/GP1908/2015 H1N1 (IVR-180) and weight loss and survival were monitored over 14 days.

To assess the effect of CpG 1018® adjuvant in more detail, mice were vaccinated IM using a prime/boost regimen according to the following groups: (1) 3 μg N1-MPP; (2) 3 μg N1-MPP+3 μg CpG 1018® adjuvant; (3) QIV (1 μg of each HA); (4) QIV+3 μg CpG 1018® adjuvant; (5) 3 μg N1-MPP admixed with QIV; (6) 3 μg N1-MPP admixed with QIV and 3 μg CpG 1018® adjuvant; (7) 3 μg N1-MPP given in the right leg and QIV given in the left leg at the same time (N1-MPP (r)/QIV (1)); (8) N1-MPP+3 μg CpG 1018® adjuvant (r)/QIV+3 μg CpG 1018® adjuvant (1); and (9) 3 μg B-Mal-HA. The vaccination scheme is shown in FIG. 4A and described in its legend. Mice were then challenged intranasally with 25×mLD₅₀ of A/Singapore/GP1908/2015 H1N1 (IVR-180). Weight loss and survival were monitored over 14 days. Blood was obtained on day 21 and day 42 after prime. Mice were euthanized if they lost 25% or more of their initial body weight.

To determine viral load in murine lung tissues, mice were vaccinated using the same regimen as described above and in the legend for FIG. 4A but with the difference that they were challenged with a reduced challenge dose of 1×mLD₅₀ of A/Singapore/GP1908/2015 H1N1 (IVR-180) in order to gain better resolution. Lungs were taken on day 3 and day 6 post challenge, homogenized and the viral titer determined via standard plaque assay [13].

To assess if the amount of N1-MPP protein could be reduced while still inducing a protective immune response, mice were vaccinated IM in a prime-boost regimen with different doses of N1-MPP. Mice received either 3 μg, 1 μg, 0.3 μg or 0.1 μg of N1-MPP, N1-MPP+the matching amount of CpG 1018® adjuvant or irrelevant B-Mal-HA protein. Mice were then challenged with 25×mLD50 of A/Singapore/GP1908/2015 H1N1 (IVR-180) and weight loss and survival were monitored over 14 days. Mice were euthanized if they lost more than 25% of their initial body weight.

To observe the protective potential of N2-MPP and B-NA-MPP, mice were vaccinated IM in a prime-boost regimen with 3 μg N2/B-NA-MPP, 3 μg N2/B-NA-MPP+CpG 1018® adjuvant or 3 μg B-Mal-HA. Vaccination with N1-MPP in the same set up was included as a positive control. Mice were then either challenged with 25×mLD₅₀ of A/Singapore/GP1908/2015 H1N1 (IVR-180) or 25×mLD₅₀ of B/New York/PV01181/2018. For the challenge with 25×mLD50 of A/Switzerland/9715293/2013 (H3N2) female DBA/2J mice were used. Weight loss and survival were monitored over 14 days. Mice were euthanized if they lost more than 25% of their initial body weight. Blood was obtained on day 21 and day 42 after prime.

To determine if there would be antigenic competition between the individual NA-MPP proteins when combined in one vaccination, mice were vaccinated IM in a prime-boost regimen either with 3 μg N1-MPP, 3 μg N1-MPP+CpG 1018® adjuvant, 3 μg of each N1, N2 and B-NA-MPP, 3 μg of each N1, N2 and B-NA-MPP+CpG 1018® adjuvant or 3 μg of B-Mal-HA. Mice were then challenged either with 25×mLD50 of A/Singapore/GP1908/2015 H1N1 (IVR-180) or 5×mLD50 of A/Vietnam/1203/04 (H5N1). Weight loss and survival were monitored over 14 days. Mice were euthanized if they lost more than 25% of their initial body weight. Blood was obtained on day 21 and day 42 after prime.

### NA-Star Assay

Enzymatic activity of the recombinant neuraminidases was determined using the NA-Star™ Influenza Neuraminidase Inhibitor Resistance Detection Kit (ThermoFisher) following the manufacturer's instructions. Briefly, a starting concentration of 10 μg/mL of recombinant neuraminidase was serially diluted 1:3 across the plate. After incubating the plates for 15 minutes at room temperature, the provided substrate was added and the plates placed in the dark for 30 minutes at 37 C. Afterwards, the provided enhancer solution was added to the wells and the plate immediately read based on luminescence signal using a Synergy H1 hybrid multimode microplate reader (BioTek). The data was analyzed using GraphPad Prism 8.

### Enzyme-Linked Immunosorbent Assay (ELISA)

ELISAs were conducted as described previously [26]. Briefly, flat-bottom 96-well plates (Immulon 4 HBX plates, ThermoFisher) were coated overnight at 4° C. with 50 μL/well of the respective recombinant protein at a concentration of 2 μg/mL diluted in phosphate buffered saline (PBS, pH-7.4; Gibco). On the next day, the coating solution was removed and the plates blocked for 1 hr at room temperature (RT) with 100 μL/well of 3% milk mixed with 0.1% Tween 20 (PBST). The blocking solution was discarded and the serum samples diluted to a starting concentration of 1:50 in 1% milk/PBST followed by a 1:3 dilution across the plate. The samples were then incubated on the plate for 2 hr at RT. For ELISAs which were performed using mAbs, primary antibodies were diluted to a start concentration of 30 μg/mL and then serially diluted 1:3 across the plate, and incubated for 1 hr at RT. For the N2-MPP epitope testing a broad panel of human derived N2-specific mAbs was used including 229-1B05, 229-1F06, 229-2E02, 235-1E06, 228-2D04, 229-1G03, 220-1D05, 229-2C06 [29]. In case of the B-MPP epitope testing the human B-NA specific antibodies 3C01, 2H09, 2E01, 2D10, 1G05, 1D05 and 1A03 [30] were used. Afterwards, plates were washed three times with PBST and incubated with the secondary antibody anti-mouse IgG H&L peroxidase-conjugated (Rockland) or anti-human IgG Fab-specific horseradish-peroxidase (HRP) (Sigma Aldrich). The secondary antibody was diluted 1:3000 in 1% milk/PBST and 100 μL/well was added to the plate and incubated for 1 hr at RT. The plates were washed again three times with PBST and then developed by adding 100 μL/well of SigmaFast o-phenylenediamine dihydrochloride (OPD) solution (Sigma Aldrich) and incubating for 10 minutes. The reaction was stopped by adding 50 μL/well of 3M hydrochloric acid (HCl). The signal was read using a Synergy H1 hybrid multimode microplate reader (BioTek) at an optical density of 490 nm. The data was analyzed using GraphPad Prism 8 software and values were expressed as the area under the curve (AUC). The cutoff value was defined as the average of all blank wells plus three times the standard deviation of the blank wells.

### Neuraminidase Inhibition (NI) Assay

The NI assay was conducted as described previously [26]. Briefly, flat-bottom 96 well plates (Immulon 4 HBX plates, ThermoFisher) were coated overnight at 4 C with 150 μL/well of fetuin (50 μg/mL; Sigma Aldrich). The next day, serum samples were heat inactivated for 1 hr at 56 C and then diluted to a starting concentration of 1:100 in PBS. The samples were diluted 1:2 across a fresh 96-well plate. The reassortant viruses used in this assay, H7N1_{Cal09} and H7N1_{Mich15}, were diluted in PBS and then added to the serum dilution at 2× the 50% effective concentration (EC₅₀) for 1 hr 45 min shaking at RT. In the meantime, the fetuin coated plates were washed three times with PBST and blocked with 200 μL/well of 5% BSA/PBS for 1 hr at RT. The plates were washed three times with PBST and 100 μL of the serum-virus mixture was transferred and incubated for 2 hr at 37 C. The plates were washed three times with PBST and 100 μL/well of peanut agglutinin conjugated to horseradish peroxidase (HRP) (PNA 5 μg/mL; Sigma Aldrich) were added for 1 hr 45 at RT. The plates were washed three times with PBST and developed by adding 100 μL/well of SigmaFast OPD developing solution. The reaction was stopped after 7 minutes by adding 50 μL/well of 3M HCl. The signal was measured by using a Synergy 4 plate reader at a wavelength of 490 nm and the individual inhibitory concertation 50 (IC₅₀) was calculated using GraphPad Prism 8.

### Lung Titers

Plaque assays for virus titration have been conducted as described previously [13]. Briefly, confluent MDCK monolayers were infected with different sample dilutions (1:10-1:1.000.000) of homogenized lung tissues diluted in 1× minimum essential medium (MEM) ((1% penicillin/streptomycin antibiotics mix, 1% HEPES, 1% L-glutamine and 1% sodium-bicarbonate (Gibco)) for 1 hr at 37 C. Afterwards, the virus dilution was removed and an overlay consisting out of 2% Oxoid agar (ThermoFisher), H₂O, 2×MEM, diethylaminoethyl (0.1% (wt/vol) DEAE) and N-p-Tosyl-L-phenylalanine chloromethyl ketone (TPCK)-treated trypsin (1 μg/mL) was added to the cells. Plates were incubated for 2 days at 37 C and then fixed using 10% paraformaldehyde overnight at 4 C. Afterwards, the agar overlay was carefully removed, and the plaques visualized by immunostaining. Plates were blocked for 1 hr at RT with 3% milk/PBS. The blocking solution was discarded, and the plates incubated with primary antibody (anti-N1 mAb 4A5¹⁴) diluted 1:3000 in 1% milk/PBS for 1 hr at RT. The plates were washed three times with PBS and the secondary antibody (anti-mouse IgG H&L peroxidase-conjugated (Rockland)) was added for 1 hr at RT. The plates were washed three times with PBS and then developed by adding KPL TrueBlue Peroxidase Substrate (SeraCare). The number of plaques was counted (n=3 per group) and the virus titers presented as the log₁₀ PFU/mL. The limit of detection for the assay was at 125 PFU/mL. The graphs were generated by using GraphPad Prism 8.

### Statistical Analysis

Titers were compared using a one-way ANOVA corrected for multiple comparisons. Survival was compared using a Mantel-Cox log rank test. All statistical analysis was performed in GraphPad Prism 9.0.1.

### 6.1.3 Results

A Prime-Boost Regimen with N1-MPP is Required to Achieve Full Protection in a Naive Mouse Model

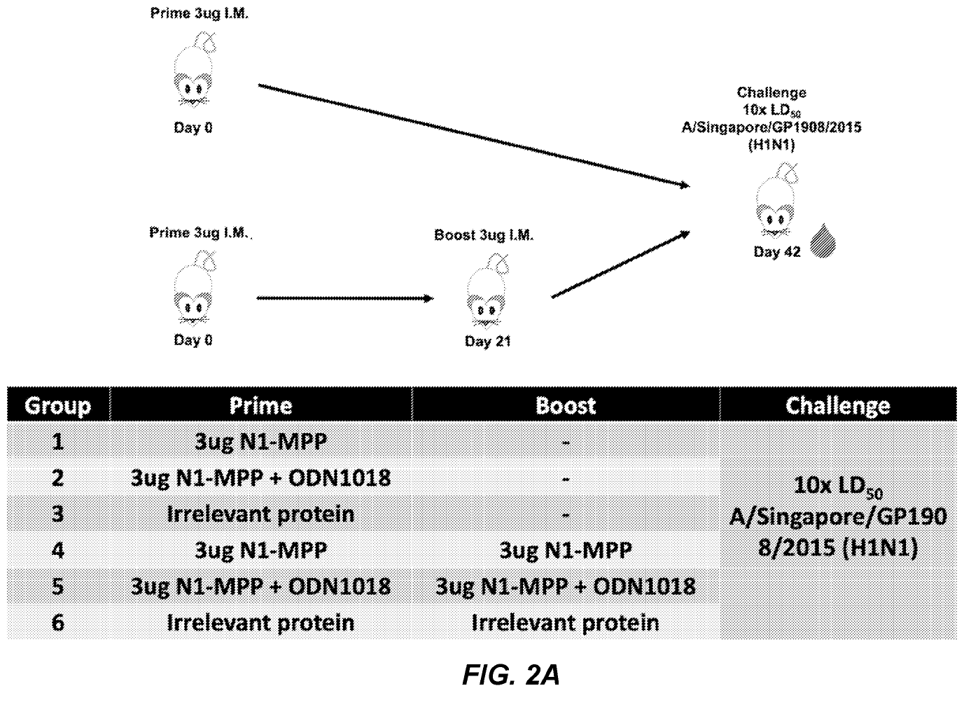

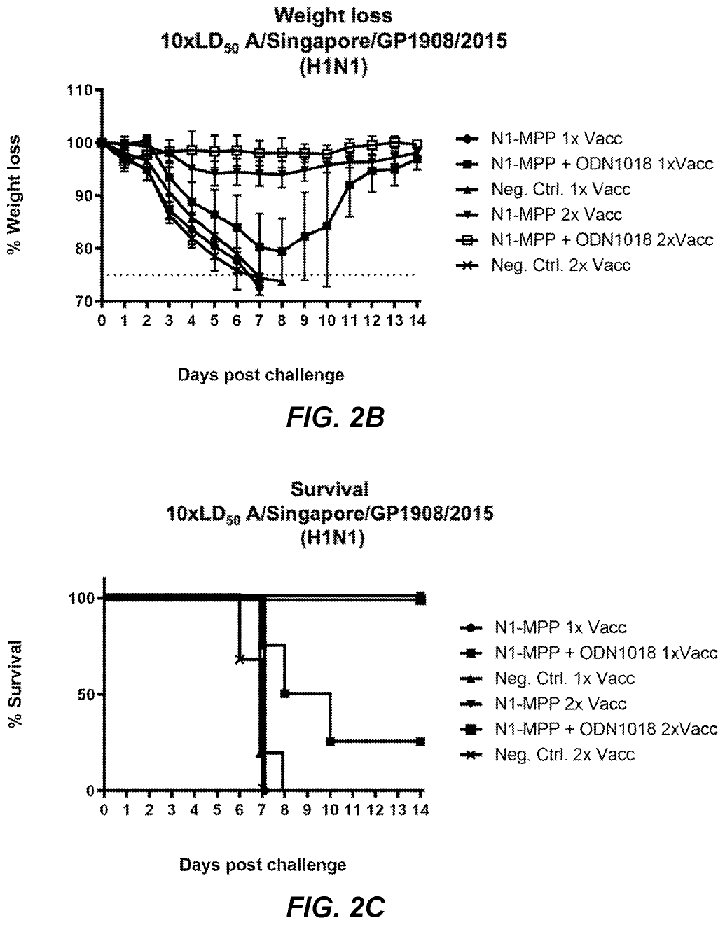

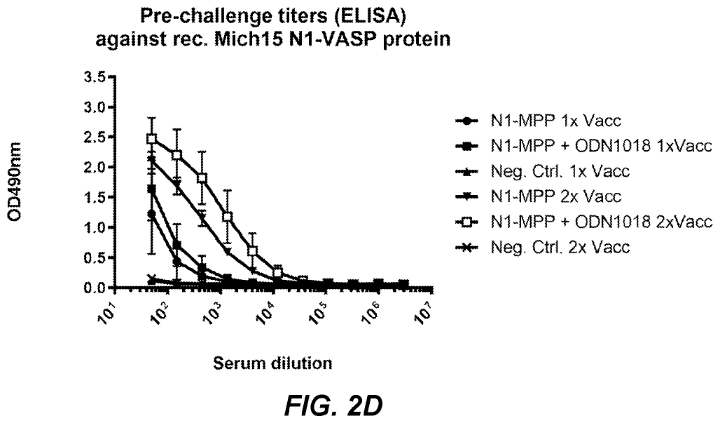

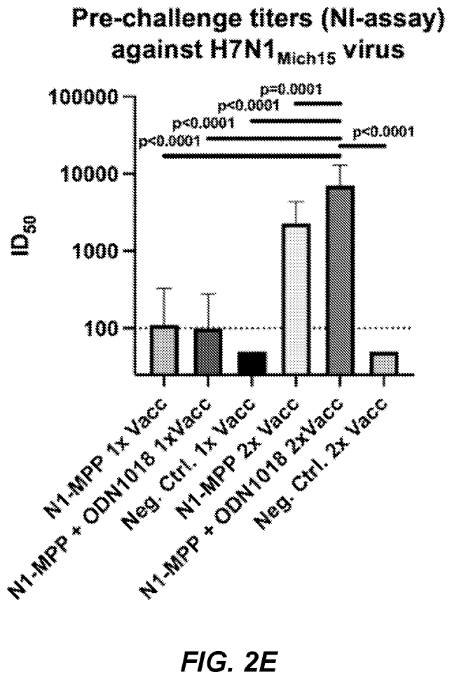

To assess the adjuvant effect of CpG 1018® adjuvant on the N1-MPP antigen in a prime-only or prime-boost vaccination regimen, naive female 6-8 week old BALB/c mice (n=5 per group) were either vaccinated once with 3 μg N1-MPP, 3 μg N1-MPP+3 μg CpG 1018® adjuvant, or 3 μg of an irrelevant protein, or twice with the same formulations after a three week interval (FIG. 2A). Three weeks post boost, mice were then challenged with 10×mLD₅₀ of A/Singapore/GP1908/2015 H1N1 (IVR-180, this virus is antigenically equivalent to A/Michigan/45/15) virus and weight loss and survival was monitored over a 14-day period. As shown in FIG. 2B, mice which only received one vaccination with non-adjuvanted N1-MPP or irrelevant protein all succumbed to infection around day 8 post challenge. Mice vaccinated once with N1-MPP+CpG 1018® adjuvant experienced high weight loss (approximately 20%) and 4 out of 5 mice succumbed to infection around day 10 post challenge. Nevertheless, mice vaccinated with the adjuvanted formulation survived significantly longer (p=0.0143). However, groups of mice which received two vaccinations of N1-MPP or N1-MPP+CpG 1018® adjuvant did not experience any weight loss and all animal in these groups survived the challenge. All mice in the negative control group succumbed to infection around day 8 (FIGS. 2B, 2C). To assess serological characteristics of the serum antibodies induced after vaccination, the serum was tested via ELISA against recombinant Mich15 N1-VASP protein. This protein containing a different tetramerization domain was used to avoid detection of antibodies induced against the MPP tetramerization domain. After the prime, only a low antibody response against the NA was detectable, with slightly higher levels in the N1-MPP+CpG 1018® adjuvant group (FIG. 2D). Serum from mice vaccinated with the prime-boost regimen showed a strong increase in N1 specific antibodies, with N1-MPP+CpG 1018® adjuvant performing the best (FIG. 2D). The same trend was observed in a NI assay using an H7N1_{Mich15} virus, which contains an irrelevant HA and the N1 of A/Michigan/45/2015. Mice vaccinated with the prime-boost regimen showed high levels of NI active antibodies in their serum with the N1-MPP+CpG 1018® adjuvant group performing best (geometric mean 50% inhibitory dilution (ID₅₀)=7046) indicating that the CpG 1018® adjuvant adjuvant leads to a more robust immune response to the antigen (FIG. 2E). The NI results also indicated a statistically significant difference between adjuvanted and non-adjuvanted groups in the prime-boost regimen.

Passive Serum Transfer from Vaccinated to Naïve Mice Protects from Lethal Challenge

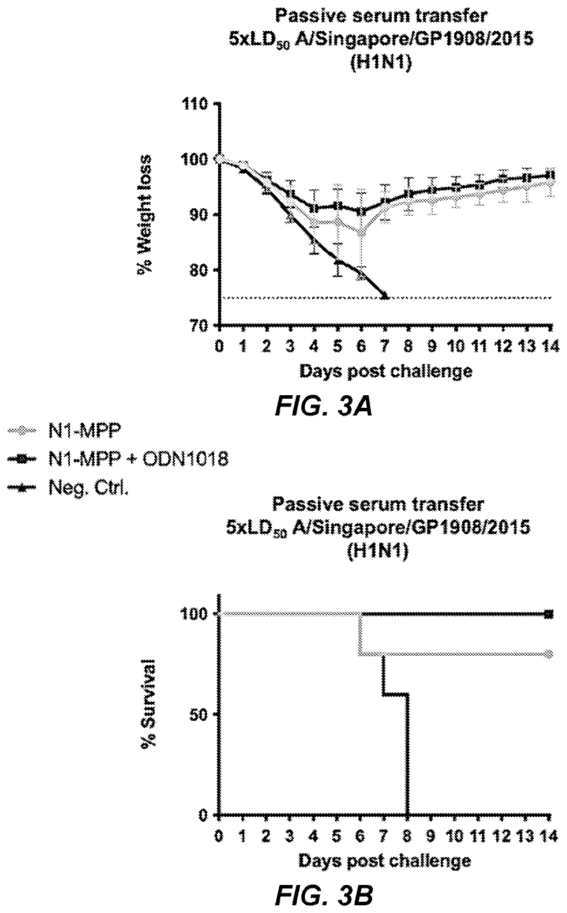

Serum obtained from mice vaccinated with the prime-boost regimen with N1-MPP, N1-MPP+3 μg CpG 1018® adjuvant and an irrelevant protein was used to perform a passive serum transfer into 6-8 week old naïve BALB/c mice (n=5 per group) followed by a challenge with 5×mLD₅₀ of A/Singapore/GP1908/2015 H1N1 (IVR-180). Weight loss and survival was monitored over a 14-day period. Mice which received N1-MPP serum showed approximately 10% weight loss, with one mouse succumbing to infection on day 6 post challenge. Mice which received N1-MPP+CpG 1018® adjuvant experienced slightly less weight loss than the N1-MPP group and experienced no mortality (FIGS. 3A, 3B). The negative control group succumbed to infection by day 8 post challenge.

### CpG 1018® Adjuvant Adjuvantation Partially Breaks the Immunodominance of HA Over NA in a QIV+rNA Formulation in Terms of Anti-NA Antibody Titers

It has been previously shown, that—while recombinant NA on its own is immunogenic—admixture with QIV leads to reduced immunogenicity, likely due to the immunodominance of HA over NA [31]. To test if CpG 1018® adjuvant would improve NA immunogenicity, even when admixed with QIV, naïve female 6-8 week old BALB/c mice (n=5 per group) were vaccinated IM in a prime-boost regimen. Mice were divided into the following nine groups for vaccination with the indicated prime-boost regimen: (1) 3 μg of N1-MPP; (2) 3 μg N1-MPP+3 μg CpG 1018® adjuvant; (3) QIV (matched with the challenge virus); (4) QIV+3 μg CpG 1018® adjuvant; (5) 3 μg N1-MPP admixed with QIV; (6) 3 μg N1-MPP admixed with QIV and 3 μg CpG 1018® adjuvant; (7) 3 μg of N1-MPP given in the right leg and QIV at the same time in the left leg (N1-MPP (r)+QIV (1)); (8) 3 μg of N1-MPP+3 μg CpG 1018® adjuvant given in the right leg and QIV+3 μg CpG 1018® adjuvant given in the left leg at the same time (N1-MPP+3 μg CpG 1018® adjuvant (r)+QIV+3 μg CpG 1018® adjuvant (1)); and (9) 3 μg of irrelevant protein. The vaccination scheme is shown in FIG. 4A and described in its legend. Following vaccination, mice were challenged with 25×mLD₅₀ of A/Singapore/GP1908/2015 H1N1 (IVR-180). Weight loss and survival was monitored over a 14-day period. The non-adjuvanted N1-MPP group showed the highest post-challenge weight loss of approximately 10% and one mouse in the group succumbed to infection on day 6 post infection. All mice in the negative control group succumbed to infection on day 6 post infection. The remaining groups did not experience any morbidity or mortality (FIGS. 4B, 4C).

To observe the effect of CpG 1018® adjuvant on the reduction of viral load in the lungs, a subset of mice was infected with a lower challenge dose of 1×mLD50 A/Singapore/GP1908/2015 H1N1 (IVR-180). Lungs were extracted on day 3 (FIG. 4D) and day 6 (FIG. 4E) post infection and the viral titers were determined. On day 3, the negative control group showed the highest viral titer (7.8×10⁵ pfu/mL), followed by the N1-MPP group (5.1×10⁴ pfu/mL), the N1-MPP+CpG 1018® adjuvant (5.0×10³ pfu/mL) and the QIV groups (1.0×10³ pfu/mL). In the remaining groups, no virus was detectable. On day 6, the virus cleared out in the N1-MPP+CpG 1018® adjuvant group as well as in the QIV group. The viral titer remained detectible in the N1-MPP group (8.5×10³ pfu/mL) but was lower than in the negative control group (2.4×10⁵ pfu/mL).

Since it is known that high titers of NI active antibodies correlate with reduction of viral replication as well as a less severe disease outcome, the level of NI active antibodies induced after vaccination with N1-MPP and QIV in combination with CpG 1018® adjuvant was assessed. NI-assays were performed with H7N1_{Mich15} virus, which contains the matching NA component to the vaccine antigen, and with H7N1_{Cal09} virus to observe if cross-reactive NI antibodies were induced in vaccinated mice. Against H7N1_{Mich15}, vaccination with N1-MPP+CpG 1018® adjuvant induced the highest level of NI antibodies (ID₅₀=62209), followed by N1-MPP+CpG 1018® adjuvant (r) and QIV+CpG 1018® adjuvant (1) (ID₅₀=17602), N1-MPP+QIV+CpG 1018® adjuvant admixed (ID₅₀=15954) and unadjuvanted N1-MPP (ID₅₀=15409) (FIG. 5A). Of note, the N1-MPP+CpG 1018® adjuvant, N1-MPP+CpG 1018® adjuvant (r) and QIV+CpG 1018® adjuvant (1) and N1-MPP+QIV+CpG 1018® adjuvant group NI activity were not significantly different suggesting that both administration of HA and NA containing vaccine in different limbs as well as co-administration in the presence of adjuvant may induce similar immune responses while this is not the case when recombinant NA is admixed with QIV without adjuvant. In case of the heterologous H7N1Cal09 virus, N1-MPP+CpG 1018® adjuvant had the highest NI inhibition potential (ID₅₀=4585), followed by the unadjuvanted N1-MPP group (ID₅₀=1479) (FIG. 5B).

To test if antibodies induced through vaccination with N1-MPP would induce N1 subtype cross reactive antibodies, the serum was tested in ELISAs against recombinant Mich15 N1-VASP protein (FIG. 5C) matching the vaccine antigen strain, and against NC99 N1 (pre-pandemic N1, FIG. 5D), PR8 N1 (prototype N1, FIG. 5E), Cal09 N1 (FIG. 5F), Bris18 N1 (pre-pandemic N1, FIG. 5G) and Vn04 N1 (avian N1, FIG. 5H). Overall, the same trend was seen as in the NI-assay, with N1-MPP+CpG 1018® adjuvant performing the best, followed by N1-MPP+ODN1018 (r) and QIV+CpG1018 (l) and N1-MPP+QIV+CpG 1018® adjuvant admixed. This underscores that CpG 1018® adjuvant is beneficial in inducing a strong and robust antibody response, which appears to be cross-reactive within the N1 subgroup. N1-MPP+QIV admixed almost did not induce any N1 specific antibody response as well as no NI active antibodies, underlining the antigenic competition between HA and NA.

### Antigen Dose De-Escalation Shows a Clear Adjuvant Effect of ODN1018

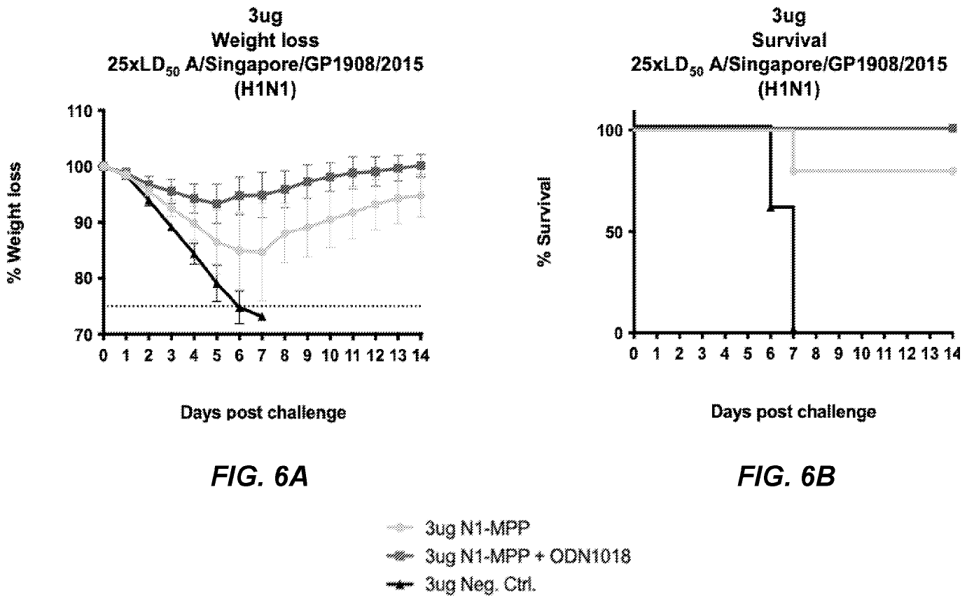

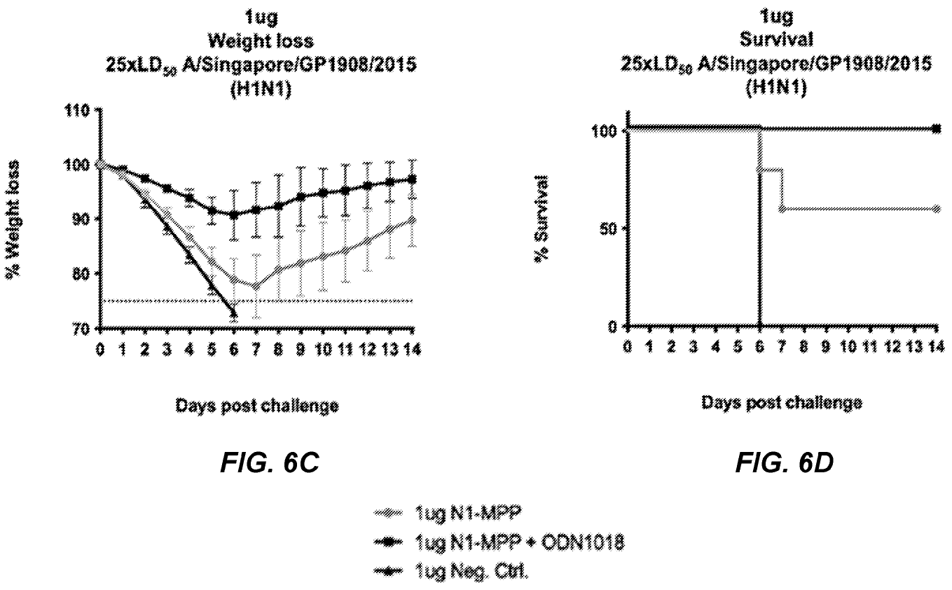

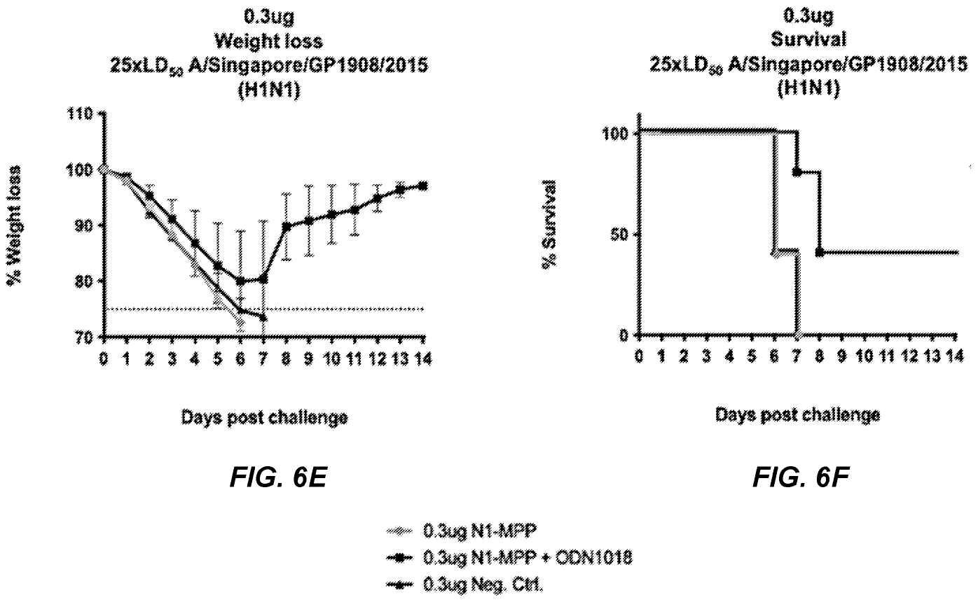

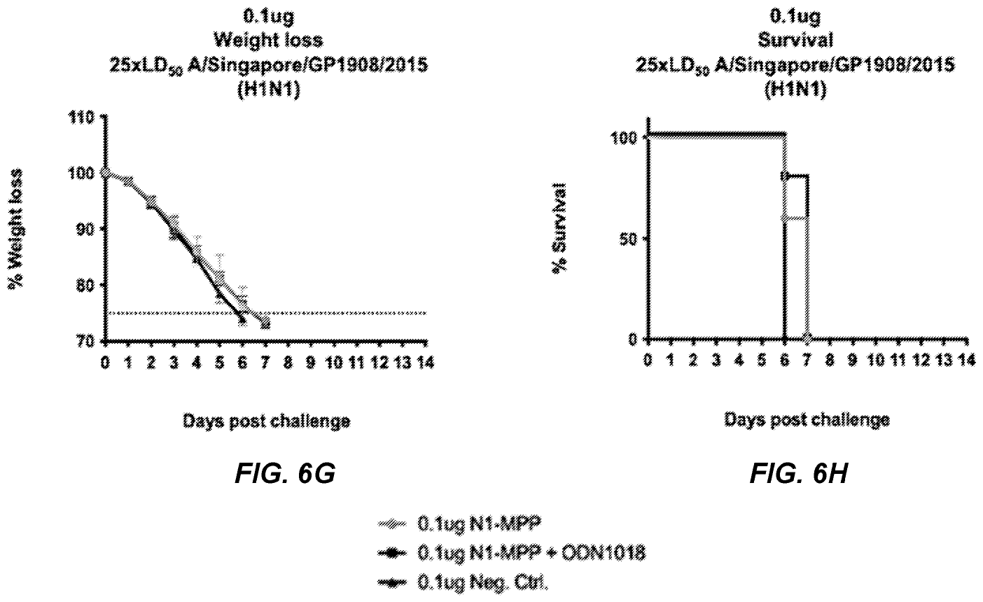

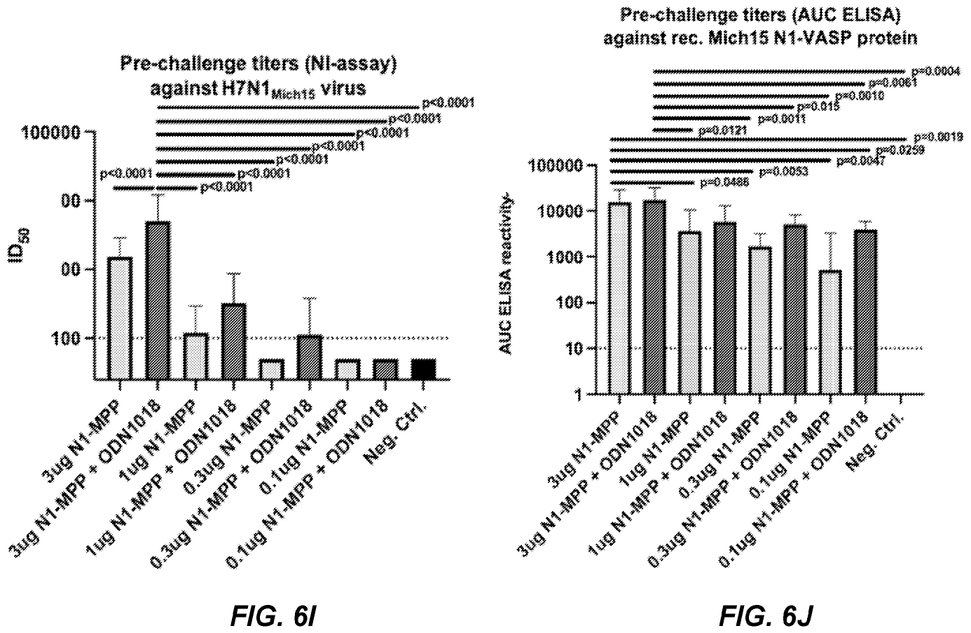

In the previous animal experiments described above, a standard amount of 3 μg N1-MPP protein per vaccination dose was used in a prime-boost regimen. Using this quantity of NA protein, it was observed that mice vaccinated with unadjuvanted N1-MPP, experienced approximately 10% weight loss (FIG. 6A), with one mouse which succumbed to infection on day 7 post challenge (FIG. 6B). However, mice which received N1-MPP+CpG 1018® adjuvant did not experience any significant morbidity or mortality. To assess if this trend could be maintained with a reduced amount of N1-MPP, a dose de-escalation study was performed. Mice (n=4-5 per group) were vaccinated IM in a prime/boost regimen with different amounts of antigen and challenged three weeks after the boost with 25×mLD₅₀ of A/Singapore/GP1908/2015 H1N1 (IVR-180). Mice were vaccinated with 1 μg, 0.3 μg, or 0.1 μg of N1-MPP, N1-MPP+CpG 1018® adjuvant or an irrelevant protein. Mice which received 1 μg of N1-MPP experienced around 20% weight loss (FIG. 6C), with two mice succumbing to infection on day 6 and 7 post challenge (FIG. 6D). However, the 1 μg N1-MPP+CpG 1018® adjuvant experienced only 5% weight loss and showed full protection. When vaccinated with 0.3 μg of non-adjuvanted N1-MPP, all mice succumb to infection by day 7 similar to the negative control group (FIGS. 6E, 6F). The 0.3 μg N1-MPP+CpG 1018® adjuvant group experienced high weight loss of almost 20% and three mice succumbed to infection by day 8. Vaccination with 0.1 μg of antigen, no matter if adjuvanted or non-adjuvanted, failed to protect mice against viral challenge and all mice succumbed to infection by day 7 (FIGS. 6G, 6H). In terms of serological responses, there was a high level of N1 specific antibodies detectable via ELISA (against Mich15 N1-VASP protein) in mice which received 3 μg (FIG. 6C) and 1 μg (FIG. 6G) of antigen. Even though vaccination with 0.3 μg (FIG. 6K) and 0.1 μg (FIG. 6J) failed to induce protection in vivo, serological analysis revealed that there was still an N1 antibody response detectable, albeit only at low levels. In terms of NI activity, there were bigger differences detected between individual dose groups than in ELISA. After vaccination with 3 μg of N1-MPP+CpG 1018® adjuvant, the ID₅₀ was 3767 (FIG. 6I). Once the dose was reduced to 1 μg, the ID₅₀ dropped to 121 for the N1-MPP+CpG 1018® adjuvant group and to 51 for the non-adjuvanted N1-MPP group (FIG. 6I). For mice which received 0.3 μg of antigen, the N1-MPP+CpG 1018® adjuvant group showed an ID₅₀ of approximately 68 (FIG. 6I). In case of the 0.1 μg group, the ID₅₀ level decreased to approximately 11 (FIG. 6I). In general, while binding antibodies were present, these antibodies had little to no NI activity in groups which received low amounts of NA protein.

### N2-MPP and B-NA-MPP Form Stable Tetramers and Exhibit Full Enzymatic Activity

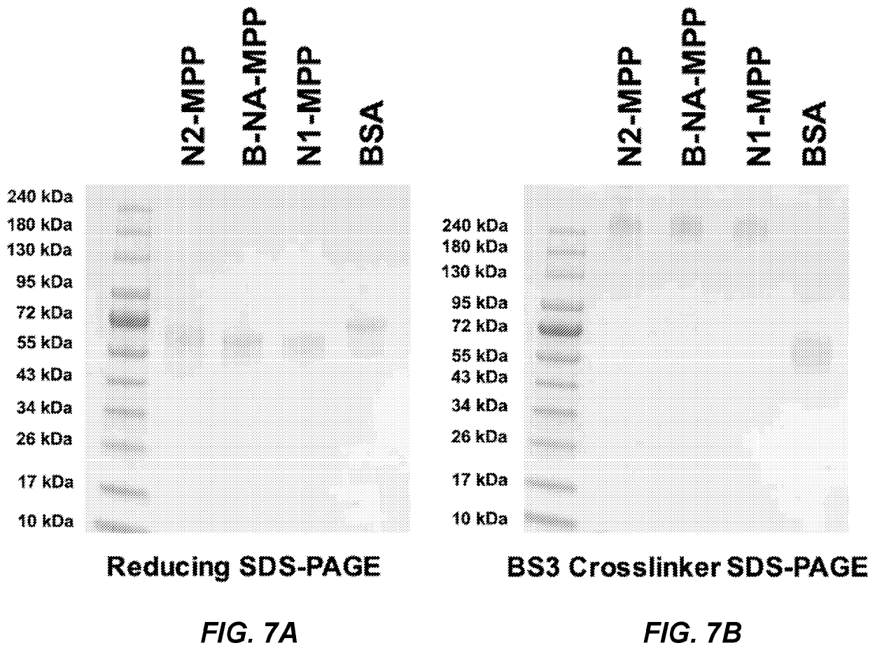

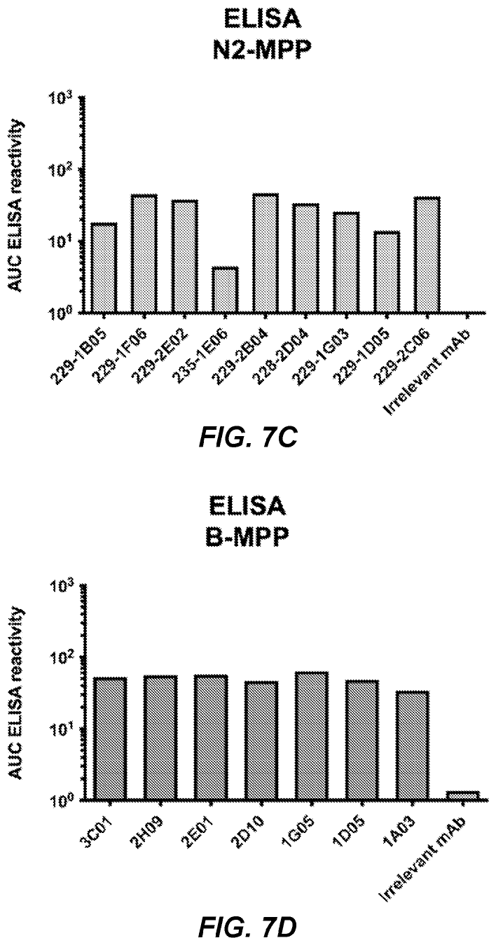

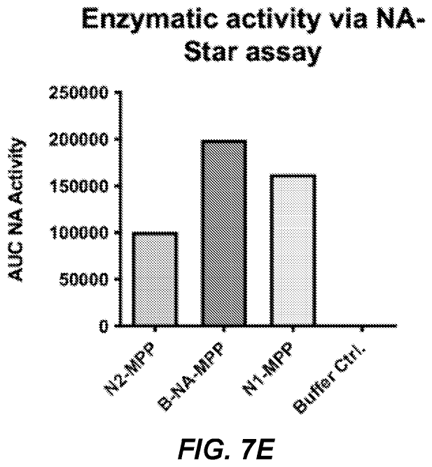

Current circulating influenza viruses in humans include H1N1, H3N2 and influenza B viruses. Therefore, N2-MPP and B-NA-MPP constructs were generated in addition to the N1-MPP construct. Sequences encoding the influenza A-NA subtype N2 of A/Kansas/14/2017 (H3N2) and influenza B-NA of B/Colorado/6/2017 were cloned into a pFastBac Dual vector containing a measles virus phosphoprotein tetramerization domain [31]. The constructs were expressed in insect cells and purified via an N-terminal hexahistidine tag. Structural integrity of the proteins was verified by visualizing them on an SDS-PAGE. Under reducing conditions, recombinant neuraminidases N1-MPP, N2-MPP, and B-NA-MPP were visible as monomers at an expected size of approximately 60 kDa (FIG. 7A). By adding BS3 crosslinker, which cross-links primary amines, it was possible to confirm successful tetramerization of the N2-MPP and the B-NA-MPP protein. The tetramers were detectable at a size of approximately 240 kDa (FIG. 7B), which is comparable to the already established N1-MPP construct which was included as a control. BSA served as monomeric control.

To confirm that the NA-MPP proteins present the correct antigenic epitopes, an ELISA was performed using a broad panel of N2 (FIG. 7C) [29] and B-NA (FIG. 7D) [30] specific human monoclonal antibodies (mAbs). The broad human anti-NA antibody 1G01 was included as a positive control [32] and an irrelevant anti-Lassa antibody KL-AV-1A12 was included as negative control. All mAbs showed strong binding to N2-MPP and B-NA-MPP suggesting that the probed epitopes are presented in a native-like conformation.

To confirm whether the recombinant neuraminidases were enzymatically active, a standard NA-Star assay was performed. N1-MPP was included as a positive control. N2-MPP and B-NA-MPP were observed to have high enzymatic activity, comparable to the already established N1-MPP construct (FIG. 7E). However, the NA activity significantly varied between N1-, N2- and B-NA-MPP which is not surprising since the different NA subtypes are known to have varying enzymatic activity based on the strain of origin [33].

Vaccination with Recombinant N2-MPP and B-NA-MPP Provides Full Protection Against Lethal Influenza Virus Challenge in the Mouse Model

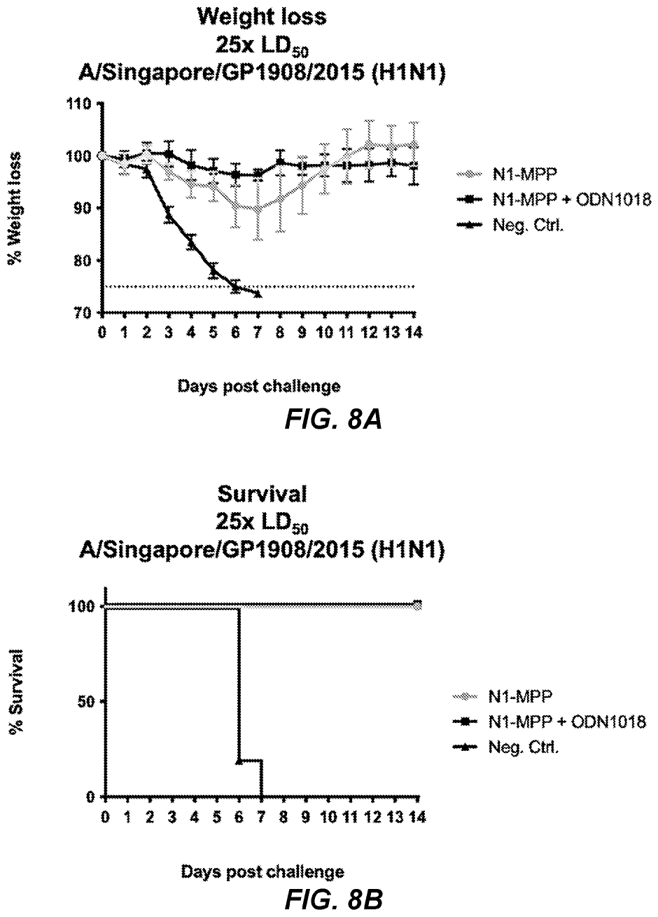

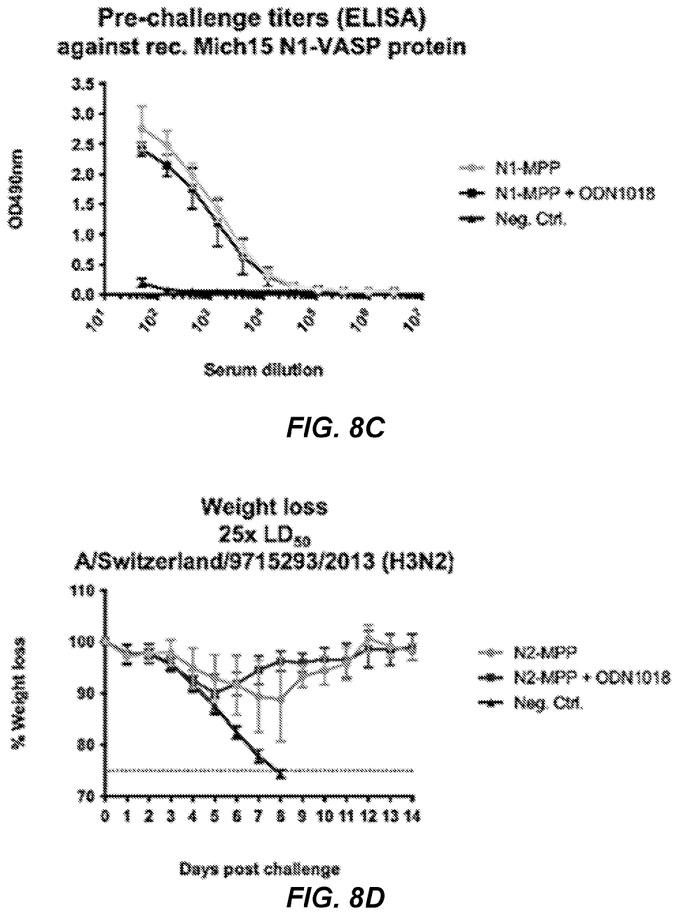

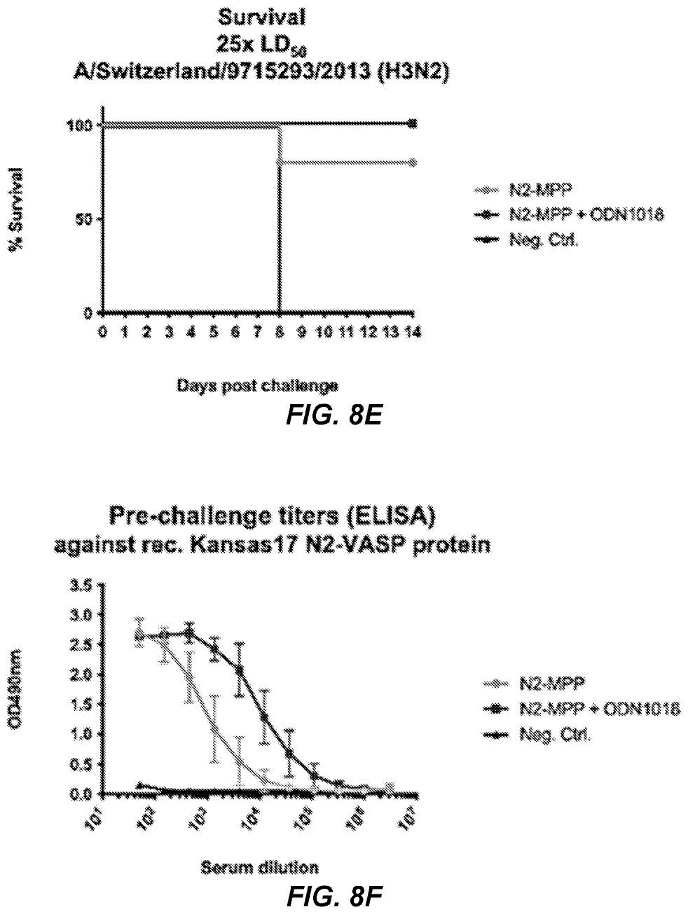

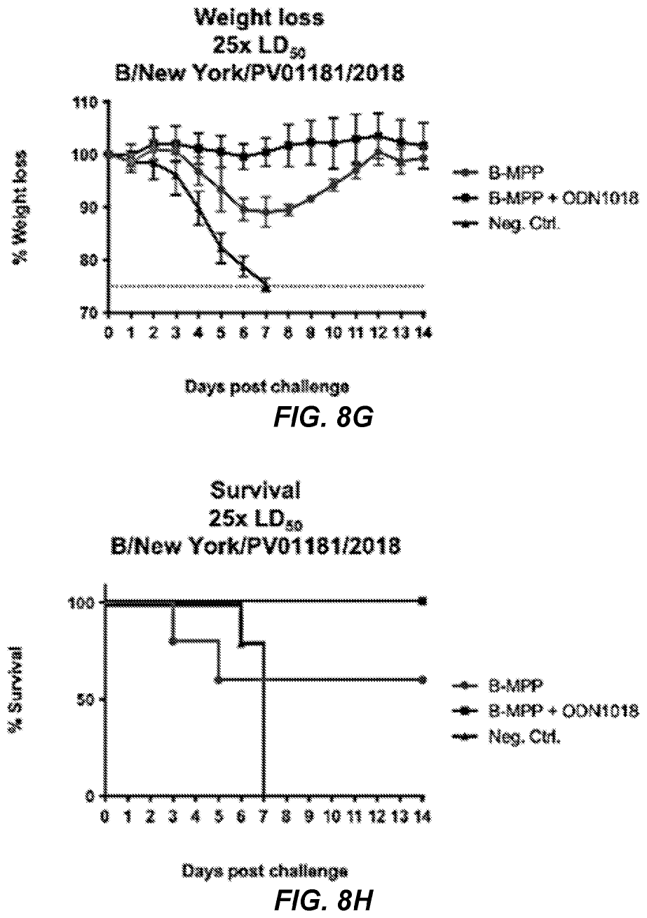

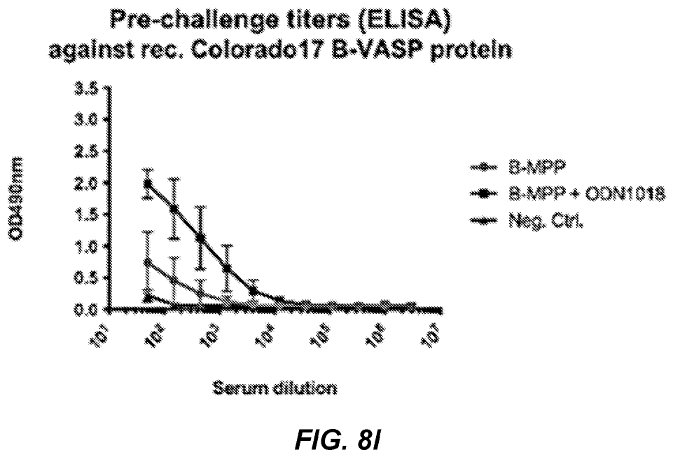

To test if the recombinant N2-MPP and B-NA-MPP could induce a protective immune response, mice were vaccinated IM with 3 μg of the respective antigen (n=5 per group) in a prime-boost regimen. The protein was either given non-adjuvanted or supplemented with CpG 1018® adjuvant. Irrelevant influenza B virus HA protein was administered as negative control. Vaccination with N1-MPP was included as a positive control. Following vaccination, mice were challenged either with 25×mLD₅₀ of A/Singapore/GP1908/2015 (H1N1), A/Switzerland/9715293/2013 (H3N2) or B/New York/PV01181/2018. Mice vaccinated with N1-MPP alone experienced a weight loss of around 10%, whereas the N1-MPP+CpG 1018® adjuvant group did not show any morbidity or mortality (FIGS. 8A, 8B). In case of N2-MPP vaccination, both the non-adjuvanted and adjuvanted group showed approximately 10% weight loss (FIG. 8D). However, the N2-MPP+CpG 1018® adjuvant group did not experience any mortality, whereas in the N2-MPP group one mouse succumb to infection on day 8 post challenge (FIG. 8E). For the B-NA-MPP vaccination, the unadjuvanted group experienced 10% weight loss, with 2 mice succumbing to infection on day 3 and 5 post challenge (FIGS. 8G, 8H). However, the B-NA-MPP+CpG 1018® adjuvant group did not show any morbidity or mortality. The antibody response against the individual NA-MPP antigens was tested via ELISA. In general, non-adjuvanted groups induced a lower immune response compared to the groups receiving NA protein with CpG 1018® adjuvant (FIGS. 8C, 8F, 8I).

A Trivalent NA-MPP Vaccine Formulation does not Induce Antigenic Competition Between the Individual NAs and is Capable of Inducing a Strong Immune Response

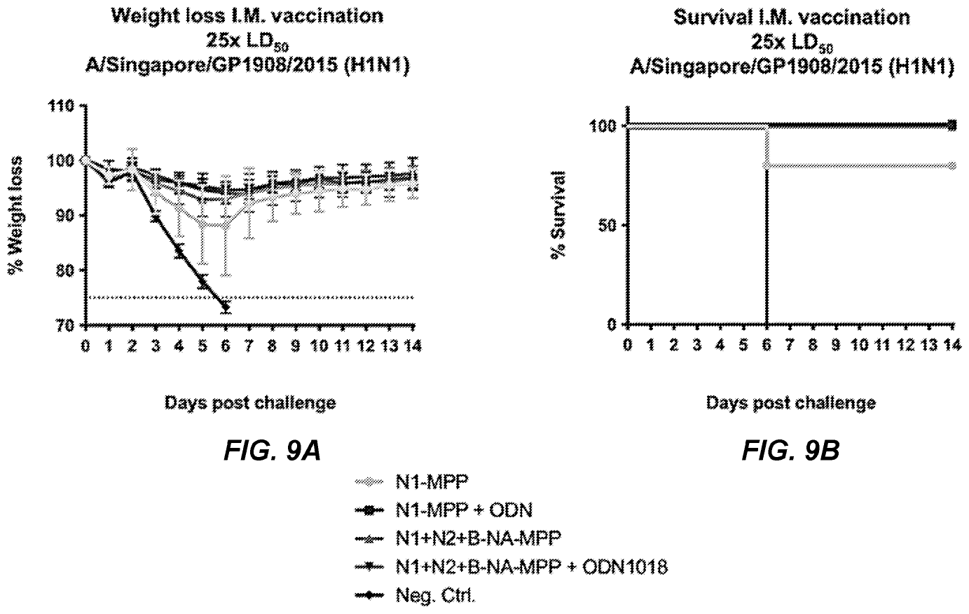

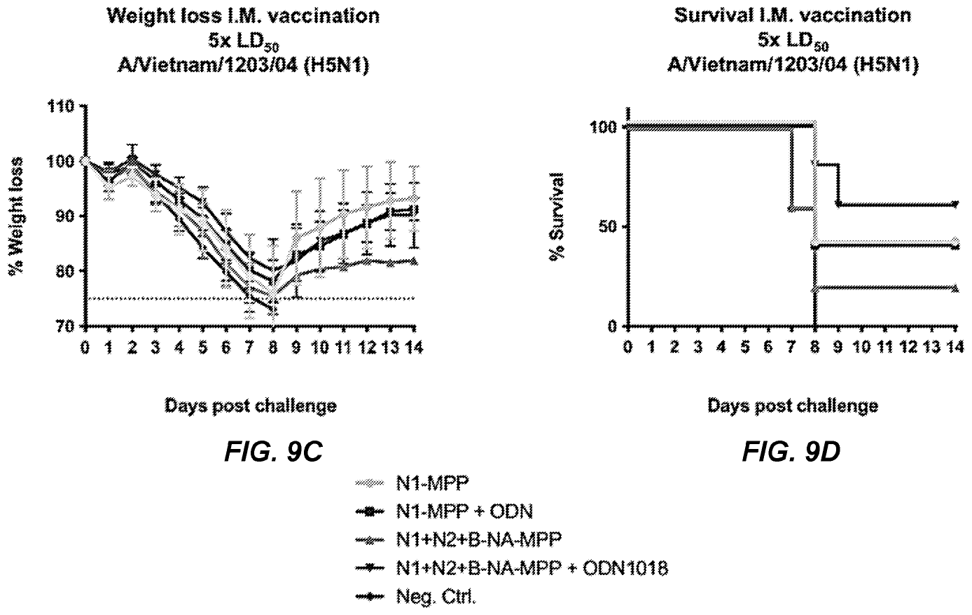

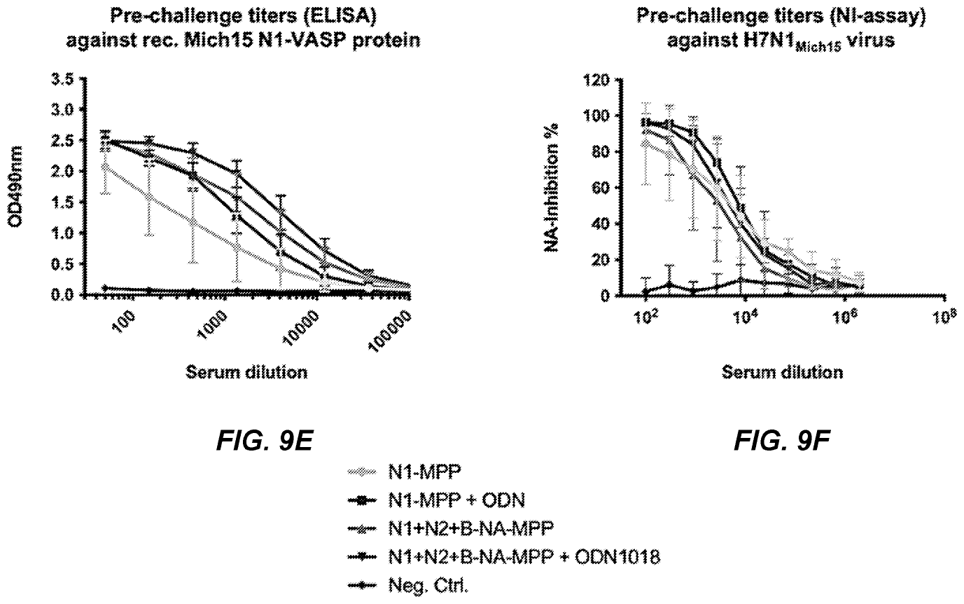

Since H1N1, H3N2 and influenza B viruses are all circulating in humans, a trivalent vaccine formulation containing all three NAs would be necessary for protection against all three types of viruses. For this reason, mice (n=5 per group) were vaccinated either with N1-MPP, N1-MPP+CpG 1018® adjuvant, N1+N2+B-NA-MPP, N1+N2+B-NA-MPP+CpG 1018® adjuvant or influenza B virus HA protein in a prime-boost regimen. After vaccination, mice were challenged with 25×mLD50 of A/Singapore/GP1908/2015 (H1N1). The group which received N1-MPP alone experienced approximately 10% weight loss with one mouse which succumb to infection on day 6 (FIGS. 9A, 9B). The other groups did not show any morbidity or mortality. To test if vaccination would also protect against challenge with a heterologous N1 virus, another subset of mice was challenged with 5×mLD50 of A/Vietnam/1203/04 (H5N1). While all groups experienced weight loss of around 20%, partial protection was observed in all vaccinated groups with the trivalent, adjuvanted formulation which was performing best (FIGS. 9C, 9D). In terms of serology, mice which had been vaccinated with N1+N2+B-NA-MPP+CpG 1018® adjuvant had the strongest antibody response in ELISA against recombinant Mich15-VASP protein (FIG. 9E). In NI-assay using H7N1_{Mich15} the inhibition potential of groups N1-MPP+CpG 1018® adjuvant and N1+N2+B-NA-MPP+CpG 1018® adjuvant was the highest (FIG. 9F).

### 6.1.4 Discussion

Influenza virus vaccines provide significant protection against influenza virus infections but the current vaccines are impacted by antigenic drift [34-38]. In addition, seasonal influenza virus vaccines induce an unbalanced immune response mostly targeting the HA but not the NA of the virus [14, 29]. However, NA undergoes somewhat slower antigenic drift [16] and anti-NA immunity can be highly protective in animal models and humans [9-14,24,25,39-44]. Several factors may influence this lack of immunogenicity of NA in seasonal vaccines, including non-standardized amounts of NA in the vaccine preparations, instability of conformational epitopes on the NA in the formulations, and HA immunodominance over NA. Vaccination with stable, tetrameric recombinant NA could overcome these issues, either when used as a standalone vaccine or when admixed to current seasonal vaccines. Recombinant neuraminidase constructs based on influenza A virus NA subtype N1 fused to a measles virus phosphoprotein tetramerization domain have been reported [24]. Here, recombinant neuraminidases prepared using influenza A virus NA subtype N2 and influenza B virus NA globular head domains are demonstrated to be immunogenic and protective in the mouse model, especially when used with a TLR9 adjuvant, CpG 1018® adjuvant. Importantly, mixing these three recombinant neuraminidases into a trivalent vaccine formulation did not decrease the immune response to the N1 component or protection from challenge with H1N1 or H5N1 viruses. Through passive transfer experiments, it was demonstrated that the induced humoral immune response is sufficient for influenza challenge protection, even though cellular immune responses to NA may contribute to protection as well. For initial experiments, a 3 μg dose of recombinant NA was used for vaccination of mice. This dose was selected because split vaccine containing 1 μg of HA per dose is typically used for mouse models. The human dose is 1 μg, so 1/15th dose of the human dose was used. Recombinant HA-based vaccines for humans contain 45 μg of HA per subtype and since the vaccine tested here is a recombinant protein vaccine, 1/15th of 45 μg for the recombinant NA was used, resulting in a 3 μg dose. As shown by dose de-escalation, the 3 μg dose performed better in vaccination against influenza challenge than lower doses.

A main focus of this study was to explore the combination of an oligonucleotide TLR9 adjuvant with recombinant NA constructs. The TLR9 agonist CpG 1018® adjuvant was selected as the adjuvant since it has an extensive safety record and is currently used in a licensed hepatitis B virus vaccine [17,45]. Here CpG 1018® adjuvant was shown to have a significant adjuvant effect in terms of the induced immune response, protection from challenge, and antigen sparing. In addition, a strong cross-reactivity to heterologous N1 NAs was detected, particularly in the adjuvanted groups. The adjuvanted trivalent NA vaccine showed the highest degree of protection against H5N1 challenge. The adjuvanted recombinant NA vaccine was observed to consistently outperform the non-adjuvanted recombinant NA vaccine. It has been shown that even when recombinant NA is admixed to seasonal vaccine preparations, HA dominates over NA and suppresses a robust anti-NA response [31]. By administering seasonal vaccine into one leg of the mice and recombinant NA into another leg this could be partially circumvented and led to a strong NA response. However, this approach is less practical for a vaccine product. Here it was found that addition of CpG 1018® adjuvant to the seasonal vaccine/recombinant NA admixture at least partially broke the immunodominance of the HA, making it possible to just administer one shot that still resulted in robust anti-NA immunity. The mechanism behind this effect is so far unclear, and without ascribing to any particular theory, could be from the attraction of additional immune cells to the injection side and providing an innate immune trigger that enhances the immune response in general.

In summary, it is shown that the combination of recombinant NA with CpG 1018® adjuvant induces robust anti-NA immunity and protection from influenza challenge in the mouse model. This surprising observation warrants further clinical development of the combination. In particular, this combination may result in a seasonal influenza virus vaccine with more resistance to antigenic drift, which may also partially protect from emerging pandemic influenza virus subtypes.

### 6.1.5 References Cited in the Background, Detailed Description, and Examples

1. Palese and Shaw (2007) Orthomyxoviridae: The Viruses and Their Replication, 5th ed. Fields' Virology, edited by B. N. Fields, D. M. Knipe, and P. M. Howley. Wolters Kluwer Health/Lippincott Williams & Wilkins, Philadelphia, USA, p 1647-1689)
2. Gross P A, Hermogenes A W, Sacks H S, Lau J, Levandowski R A. The efficacy of influenza vaccine in elderly persons. A meta-analysis and review of the literature. Ann Intern Med. 1995 Oct. 1; 123(7):518-27. doi: 10.7326/0003-4819-123-7-199510010-00008. PMID: 7661497.
3 Ogburn T, Espey E L, Contreras V, Arroyo P. Impact of clinic interventions on the rate of influenza vaccination in pregnant women. J Reprod Med. 2007 September; 52(9):753-6. PMID: 17939588.
4. Jefferson T, Rivetti D, Rivetti A, Rudin M, Di Pietrantonj C, Demicheli V. Efficacy and effectiveness of influenza vaccines in elderly people: a systematic review. Lancet. 2005 Oct. 1; 366(9492):1165-74. doi: 10.1016/S0140-6736(05)67339-4. Epub 2005 Sep. 22. Erratum in: Lancet. 2006 Mar. 25; 367(9515):986. PMID: 16198765.
5. Beyer W E, McElhaney J, Smith D J, Monto A S, Nguyen-Van-Tam J S, Osterhaus A D. Cochrane re-arranged: support for policies to vaccinate elderly people against influenza. Vaccine. 2013 Dec. 5; 31(50):6030-3. doi: 10.1016/j.vaccine.2013.09.063. Epub 2013 Oct. 3. PMID: 24095882.
6. Nichol K L, Lind A, Margolis K L, Murdoch M, McFadden R, Hauge M, Magnan S, Drake M. The effectiveness of vaccination against influenza in healthy, working adults. N Engl J Med. 1995 Oct. 5; 333(14):889-93. doi: 10.1056/NEJM199510053331401. PMID: 7666874.
7. Jefferson T, Smith S, Demicheli V, Harnden A, Rivetti A, Di Pietrantonj C. Assessment of the efficacy and effectiveness of influenza vaccines in healthy children: systematic review. Lancet. 2005 February 26-March 4; 365(9461):773-80. doi: 10.1016/S0140-6736(05)17984-7. PMID: 15733718.
8. Flannery B, Chung J R, Belongia E A, McLean H Q, Gaglani M, Murthy K, Zimmerman R K, Nowalk M P, Jackson M L, Jackson L A, Monto A S, Martin E T, Foust A, Sessions W, Berman L, Barnes J R, Spencer S, Fry A M. Interim Estimates of 2017-18 Seasonal Influenza Vaccine Effectiveness-United States, February 2018. MMWR Morb Mortal Wkly Rep. 2018 Feb. 16; 67(6):180-185. doi: 10.15585/mmwr.mm6706a2. PMID: 29447141; PMCID: PMC5815489.
9. Memoli M J, Shaw P A, Han A, Czajkowski L, Reed S, Athota R, Bristol T, Fargis S, Risos K, Powers J H, Davey R T Jr, Taubenberger J K. Evaluation of Antihemagglutinin and Antineuraminidase Antibodies as Correlates of Protection in an Influenza A/H1N1 Virus Healthy Human Challenge Model. mBio. 2016 Apr. 19; 7(2):e00417-16. doi: 10.1128/mBio.00417-16. PMID: 27094330; PMCID: PMC4959521.
10. Couch R B, Atmar R L, Franco L M, Quarles J M, Wells J, Arden N, Niño D, Belmont J W. Antibody correlates and predictors of immunity to naturally occurring influenza in humans and the importance of antibody to the neuraminidase. J Infect Dis. 2013 Mar. 15; 207(6):974-81. doi: 10.1093/infdis/jis935. Epub 2013 Jan. 10. PMID: 23307936; PMCID: PMC3633450.
11. Monto A S, Petrie J G, Cross R T, Johnson E, Liu M, Zhong W, Levine M, Katz J M, Ohmit S E. Antibody to Influenza Virus Neuraminidase: An Independent Correlate of Protection. J Infect Dis. 2015 Oct. 15; 212(8):1191-9. doi: 10.1093/infdis/jiv195. Epub 2015 Apr. 8. PMID: 25858957.
12. Maier H E, Nachbagauer R, Kuan G, Ng S, Lopez R, Sanchez N, Stadlbauer D, Gresh L, Schiller A, Rajabhathor A, Ojeda S, Guglia A F, Amanat F, Balmaseda A, Krammer F, Gordon A. Pre-existing Antineuraminidase Antibodies Are Associated With Shortened Duration of Influenza A (H1N1) pdm Virus Shedding and Illness in Naturally Infected Adults. Clin Infect Dis. 2020 May 23; 70(11):2290-2297. doi: 10.1093/cid/ciz639. PMID: 31300819; PMCID: PMC7245146.
13. McMahon M, Kirkpatrick E, Stadlbauer D, Strohmeier S, Bouvier N M, Krammer F. Mucosal Immunity against Neuraminidase Prevents Influenza B Virus Transmission in Guinea Pigs. mBio. 2019 May 21; 10(3):e00560-19. doi: 10.1128/mBio.00560-19. PMID: 31113896; PMCID: PMC6529633.
14. Wohlbold T J, Nachbagauer R, Xu H, Tan G S, Hirsh A, Brokstad K A, Cox R J, Palese P, Krammer F. Vaccination with adjuvanted recombinant neuraminidase induces broad heterologous, but not heterosubtypic, cross-protection against influenza virus infection in mice. mBio. 2015 Mar. 10; 6(2):e02556. doi: 10.1128/mBio.02556-14. PMID: 25759506; PMCID: PMC4453582.
15. Johansson B E, Moran T M, Kilbourne E D. Antigen-presenting B cells and helper T cells cooperatively mediate intravirionic antigenic competition between influenza A virus surface glycoproteins. Proc Natl Acad Sci USA. 1987 October; 84(19):6869-73. doi: 10.1073/pnas.84.19.6869. PMID: 2958849; PMCID: PMC299186.
16. Krammer F, Fouchier RAM, Eichelberger M C, Webby R J, Shaw-Saliba K, Wan H, Wilson P C, Compans R W, Skountzou I, Monto A S. NAction! How Can Neuraminidase-Based Immunity Contribute to Better Influenza Virus Vaccines? mBio. 2018 Apr. 3; 9(2):e02332-17. doi: 10.1128/mBio.02332-17. PMID: 29615508; PMCID: PMC5885027.
17. Campbell J D. Development of the CpG Adjuvant 1018: A Case Study. Methods Mol Biol. 2017; 1494:15-27. doi: 10.1007/978-1-4939-6445-1_2. PMID: 27718183.
18. Braun R P, Lee J S. Immunogenic duplex nucleic acids are nuclease resistant. J Immunol. 1988 Sep. 15; 141(6):2084-9. PMID: 3171172.
19. Latimer L J, Agazie Y M, Braun R P, Hampel K J, Lee J S. Specificity of monoclonal antibodies produced against phosphorothioate and ribo modified DNAs. Mol Immunol. 1995 October; 32(14-15):1057-64. doi: 10.1016/0161-5890(95)00086-0. PMID: 8544855.
20. Fields S, Winter G, Brownlee G G. Structure of the neuraminidase gene in human influenza virus A/PR/8/34. Nature. 1981 Mar. 19; 290(5803):213-7. doi: 10.1038/290213a0. PMID: 7010182.
21. Ward C W, Elleman T C, Azad A A. Amino acid sequence of the Pronase-released heads of neuraminidase subtype N2 from the Asian strain A/Tokyo/3/67 of influenza virus. Biochem J. 1982 Oct. 1; 207(1):91-5. doi: 10.1042/bj2070091. PMID: 7181865; PMCID: PMC1153827.
22. Palese P, Tobita K, Ueda M, Compans R W. Characterization of temperature sensitive influenza virus mutants defective in neuraminidase. Virology. 1974 October; 61(2):397-410. doi: 10.1016/0042-6822(74)90276-1. PMID: 4472498.
23. McAuley J L, Gilbertson B P, Trifkovic S, Brown L E, McKimm-Breschkin J L. Influenza Virus Neuraminidase Structure and Functions. Front Microbiol. 2019 Jan. 29; 10:39. doi: 10.3389/fmicb.2019.00039. PMID: 30761095; PMCID: PMC6362415.
24. Liu W C, Lin C Y, Tsou Y T, Jan J T, Wu S C. Cross-Reactive Neuraminidase-Inhibiting Antibodies Elicited by Immunization with Recombinant Neuraminidase Proteins of H5N1 and Pandemic H1N1 Influenza A Viruses. J Virol. 2015 July; 89(14):7224-34. doi: 10.1128/JVI.00585-15. Epub 2015 May 6. PMID: 25948745; PMCID: PMC4473581.
25. McMahon M, Strohmeier S, Rajendran M, Capuano C, Ellebedy A H, Wilson P C, Krammer F. Correctly folded—but not necessarily functional—influenza virus neuraminidase is required to induce protective antibody responses in mice. Vaccine. 2020 Oct. 21; 38(45):7129-7137. doi: 10.1016/j.vaccine.2020.08.067. Epub 2020 Sep. 15. PMID: 32943267.
26. Strohmeier S, Carreño J M, Brito R N, Krammer F. Introduction of Cysteines in the Stalk Domain of Recombinant Influenza Virus N1 Neuraminidase Enhances Protein Stability and Immunogenicity in Mice. Vaccines (Basel). 2021 Apr. 19; 9(4):404. doi: 10.3390/vaccines9040404. PMID: 33921722; PMCID: PMC8072926.
27. Krammer F, Margine I, Tan G S, Pica N, Krause J C, Palese P. A carboxy-terminal trimerization domain stabilizes conformational epitopes on the stalk domain of soluble recombinant hemagglutinin substrates. PLOS One. 2012; 7(8):e43603. doi: 10.1371/journal.pone.0043603. Epub 2012 Aug. 23. PMID: 22928001; PMCID: PMC3426533.
28. Margine I, Palese P, Krammer F. Expression of functional recombinant hemagglutinin and neuraminidase proteins from the novel H7N9 influenza virus using the baculovirus expression system. J Vis Exp. 2013 Nov. 6; (81):e51112. doi: 10.3791/51112. PMID: 24300384; PMCID: PMC3970794.
29. Chen Y Q, Wohlbold T J, Zheng N Y, Huang M, Huang Y, Neu K E, Lee J, Wan H, Rojas K T, Kirkpatrick E, Henry C, Palm A E, Stamper C T, Lan L Y, Topham D J, Treanor J, Wrammert J, Ahmed R, Eichelberger M C, Georgiou G, Krammer F, Wilson P C. Influenza Infection in Humans Induces Broadly Cross-Reactive and Protective Neuraminidase-Reactive Antibodies. Cell. 2018 Apr. 5; 173(2):417-429.e10. doi: 10.1016/j.cell.2018.03.030. PMID: 29625056; PMCID: PMC5890936.
30. Madsen A, Dai Y N, McMahon M, Schmitz A J, Turner J S, Tan J, Lei T, Alsoussi W B, Strohmeier S, Amor M, Mohammed B M, Mudd P A, Simon V, Cox R J, Fremont D H, Krammer F, Ellebedy A H. Human Antibodies Targeting Influenza B Virus Neuraminidase Active Site Are Broadly Protective. Immunity. 2020 Oct. 13; 53(4):852-863.e7. doi: 10.1016/j.immuni.2020.08.015. Epub 2020 Sep. 24. PMID: 32976769; PMCID: PMC7572813.
31. Strohmeier S, Amanat F, Zhu X, McMahon M, Deming M E, Pasetti M F, Neuzil K M, Wilson I A, Krammer F. A Novel Recombinant Influenza Virus Neuraminidase Vaccine Candidate Stabilized by a Measles Virus Phosphoprotein Tetramerization Domain Provides Robust Protection from Virus Challenge in the Mouse Model. mBio. 2021 Dec. 21; 12(6):e0224121. doi: 10.1128/mBio.02241-21. Epub 2021 Nov. 23. PMID: 34809451; PMCID: PMC8609353.
32. Stadlbauer D, Zhu X, McMahon M, Turner J S, Wohlbold T J, Schmitz A J, Strohmeier S, Yu W, Nachbagauer R, Mudd P A, Wilson I A, Ellebedy A H, Krammer F. Broadly protective human antibodies that target the active site of influenza virus neuraminidase. Science. 2019 Oct. 25; 366(6464):499-504. doi: 10.1126/science.aay0678. PMID: 31649200; PMCID: PMC7105897.
33. Lai J C C, Karunarathna H M T K, Wong H H, Peiris J S M, Nicholls J M. Neuraminidase activity and specificity of influenza A virus are influenced by haemagglutinin-receptor binding. Emerg Microbes Infect. 2019; 8(1):327-338. doi: 10.1080/22221751.2019.1581034. PMID: 30866786; PMCID: PMC6455212.
34. Chambers B S, Parkhouse K, Ross™, Alby K, Hensley S E. Identification of Hemagglutinin Residues Responsible for H3N2 Antigenic Drift during the 2014-2015 Influenza Season. Cell Rep. 2015 Jul. 7; 12(1):1-6. doi: 10.1016/j.celrep.2015.06.005. Epub 2015 Jun. 25. PMID: 26119736; PMCID: PMC4487778.
35. de Jong J C, Beyer W E, Palache A M, Rimmelzwaan G F, Osterhaus A D. Mismatch between the 1997/1998 influenza vaccine and the major epidemic A (H3N2) virus strain as the cause of an inadequate vaccine-induced antibody response to this strain in the elderly. J Med Virol. 2000 May; 61(1):94-9. PMID: 10745239.
36. Heikkinen T, Ikonen N, Ziegler T. Impact of influenza B lineage-level mismatch between trivalent seasonal influenza vaccines and circulating viruses, 1999-2012. Clin Infect Dis. 2014 Dec. 1; 59(11):1519-24. doi: 10.1093/cid/ciu664. Epub 2014 Aug. 19. PMID: 25139969.
37. Skowronski D M, Sabaiduc S, Leir S, Rose C, Zou M, Murti M, Dickinson J A, Olsha R, Gubbay J B, Croxen M A, Charest H, Bastien N, Li Y, Jassem A, Krajden M, De Serres G. Paradoxical clade- and age-specific vaccine effectiveness during the 2018/19 influenza A (H3N2) epidemic in Canada: potential imprint-regulated effect of vaccine (I-REV). Euro Surveill. 2019 November; 24(46):1900585. doi: 10.2807/1560-7917.ES.2019.24.46.1900585. PMID: 31771709; PMCID: PMC6864978.
38. Xie H, Wan X F, Ye Z, Plant E P, Zhao Y, Xu Y, Li X, Finch C, Zhao N, Kawano T, Zoueva O, Chiang M J, Jing X, Lin Z, Zhang A, Zhu Y. H3N2 Mismatch of 2014-15 Northern Hemisphere Influenza Vaccines and Head-to-head Comparison between Human and Ferret Antisera derived Antigenic Maps. Sci Rep. 2015 Oct. 16; 5:15279. doi: 10.1038/srep15279. PMID: 26472175; PMCID: PMC4607887.
39. Easterbrook J D, Schwartzman L M, Gao J, Kash J C, Morens D M, Couzens L, Wan H, Eichelberger M C, Taubenberger J K. Protection against a lethal H5N1 influenza challenge by intranasal immunization with virus-like particles containing 2009 pandemic H1N1 neuraminidase in mice. Virology. 2012 Oct. 10; 432(1):39-44. doi: 10.1016/j.virol.2012.06.003. Epub 2012 Jun. 22. PMID: 22727831; PMCID: PMC3725556.
40. Deroo T, Jou W M, Fiers W. Recombinant neuraminidase vaccine protects against lethal influenza. Vaccine. 1996 April; 14(6):561-9. doi: 10.1016/0264-410x(95)00157-v. PMID: 8782356.
41. Kilbourne E D, Pokorny B A, Johansson B, Brett I, Milev Y, Matthews J T. Protection of mice with recombinant influenza virus neuraminidase. J Infect Dis. 2004 Feb. 1; 189(3):459-61. doi: 10.1086/381123. Epub 2004 Jan. 13. PMID: 14745703.
42. Marcelin G, DuBois R, Rubrum A, Russell C J, McElhaney J E, Webby R J. A contributing role for anti-neuraminidase antibodies on immunity to pandemic H1N1 2009 influenza A virus. PLOS One. 2011; 6(10):e26335. doi: 10.1371/journal.pone.0026335. Epub 2011 Oct. 24. PMID: 22039464; PMCID: PMC3200314.
43. Sandbulte M R, Jimenez G S, Boon A C, Smith L R, Treanor J J, Webby R J. Cross-reactive neuraminidase antibodies afford partial protection against H5N1 in mice and are present in unexposed humans. PLOS Med. 2007 February; 4(2):e59. doi: 10.1371/journal.pmed.0040059. PMID: 17298168; PMCID: PMC1796909.
44. Sylte M J, Hubby B, Suarez D L. Influenza neuraminidase antibodies provide partial protection for chickens against high pathogenic avian influenza infection. Vaccine. 2007 May 10; 25(19):3763-72. doi: 10.1016/j.vaccine.2007.02.011. Epub 2007 Feb. 23. PMID: 17350145.
45. Jackson S, Lentino J, Kopp J, Murray L, Ellison W, Rhee M, Shockey G, Akella L, Erby K, Heyward W L, Janssen R S; HBV-23 Study Group. Immunogenicity of a two-dose investigational hepatitis B vaccine, HBsAg-1018, using a toll-like receptor 9 agonist adjuvant compared with a licensed hepatitis B vaccine in adults. Vaccine. 2018 Jan. 29; 36(5):668-674. doi: 10.1016/j.vaccine.2017.12.038. Epub 2017 Dec. 27. PMID: 29289383.

### 7. EMBODIMENTS

1. An immunogenic composition, comprising:
   a) a recombinant neuraminidase, wherein the recombinant neuraminidase
      i) comprises an influenza virus neuraminidase globular head domain, and a paramyxovirus phosphoprotein tetramerization domain; and
      ii) lacks each of influenza virus neuraminidase stalk domain, transmembrane domain, and cytoplasmic domain, and
   b) an adjuvant, wherein the adjuvant comprises an oligonucleotide of from 10 to 35 nucleotides in length comprising the nucleotide sequence of

5′-GAACGTTCG-3′,

in an admixture with a pharmaceutically acceptable carrier.
2. The immunogenic composition of embodiment 1, wherein the paramyxovirus phosphoprotein tetramerization domain comprises a measles virus phosphoprotein tetramerization domain.
3. The immunogenic composition of embodiment 2, wherein the measles virus phosphoprotein tetramerization domain comprises the amino acid sequence of

(SEQ ID NO: 8)
GDHYDDELFSDVQDIKTALAKIHEDNQKIISKLESLLLLKGEVESIKKQ
INRQNISISTLEGHLSSIMIAIPGL.

4. The immunogenic composition of embodiment 2, wherein the measles virus phosphoprotein tetramerization domain comprises an amino acid sequence having at least 80% identity to the amino acid sequence of SEQ ID NO:8.
5. The immunogenic composition of any one of embodiments 1 to 4, wherein the influenza virus neuraminidase globular head domain comprises an influenza A virus neuraminidase globular head domain.
6. The immunogenic composition of embodiment 5, wherein the influenza A virus neuraminidase is subtype N1 or subtype N2.
7. The immunogenic composition of any one of embodiments 1 to 4, wherein the influenza virus neuraminidase globular head domain comprises an influenza B virus neuraminidase globular head domain.
8. The immunogenic composition of embodiment 6, wherein
   a) the subtype N1 is influenza virus A/Michigan/45/2015; or
   b) the subtype N2 is influenza virus A/Kansas/14/2017.
9. The immunogenic composition of embodiment 7, wherein the influenza B virus is influenza virus B/Colorado/6/2017.
10. The immunogenic composition of any one of embodiments 1 to 4, wherein the influenza virus neuraminidase globular head domain comprises the amino acid sequence of:

a)
(SEQ ID NO: 31)
SVKLAGNSSLCPVSGWAIYSKDNSVRIGSKGDVFVIREPFISCSPLECR
TFFLTQGALLNDKHSNGTIKDRSPYRTLMSCPIGEVPSPYNSRFESVAW
SASACHDGINWLTIGISGPDSGAVAVLKYNGIITDTIKSWRNNILRTQE
SECACVNGSCFTIMTDGPSDGQASYKIFRIEKGKIIKSVEMKAPNYHYE
ECSCYPDSSEITCVCRDNWHGSNRPWVSFNQNLEYQMGYICSGVFGDNP
RPNDKTGSCGPVSSNGANGVKGFSFKYGNGVWIGRTKSISSRKGFEMIW
DPNGWTGTDNKFSIKQDIVGINEWSGYSGSFVQHPELTGLDCIRPCFWV
ELIRGRPEENTIWTSGSSISFCGVNSDTVGWSWPDGAELPFTIDK;
b)
(SEQ ID NO: 32)
ICPKPAEYRNWSKPQCGITGFAPFSKDNSIRLSAGGDIWVTREPYVSCD
PDKCYQFALGQGTTINNVHSNNTARDRTPHRTLLMNELGVPFHLGTKQV
CIAWSSSSCHDGKAWLHVCITGDDKNATASFIYNGRLVDSVVSWSKDIL
RTQESECVCINGTCTVVMTDGNATGKADTKILFIEEGKIVHTSKLSGSA
QHVEECSCYPRYPGVRCVCRDNWKGSNRPIVDINIKDHSIVSSYVCSGL
VGDTPRKTDSSSSSHCLNPNNEKGGHGVKGWAFDDGNDVWMGRTINETS
RLGYETFKVVEGWSNPKSKLQINRQVIVDRGDRSGYSGIFSVEGKSCIN
RCFYVELIRGRKEETEVLWTSNSIVVFCGTSGTYGTGSWPDGADLNLMH
I;
or
c)
(SEQ ID NO: 33)
LLLPEPEWTYPRLSCPGSTFQKALLISPHRFGETKGNSAPLIIREPFVA
CGPNECKHEALTHYAAQPGGYYNGTRGDRNKLRHLISVKLGKIPTVENS
IFHMAAWSGSACHDGKEWTYIGVDGPDNNALLKVKYGEAYTDTYHSYAN
NILRTQESACNCIGGNCYLMITDGSASGVSECRFLKIREGRIIKEIFPT
GRVKHTEECTCGFASNKTIECACRDNRYTAKRPFVKLNVETDTAEIRLM
CTDTYLDTPRPNDGSITGPCESDGDKGSGGIKGGFVHQRMKSKIGRWYS
RTMSQTERMGMGLYVKYGGDPWADSDALAFSGVMVSMKEPGWYSFGFEI
KDKKCDVPCIGIEMVHDGGKETWHSAATAIYCLMGSGQLLWDTVTGVDM
AL.

11. The immunogenic composition of any one of embodiments 1 to 4, wherein the influenza virus neuraminidase globular head domain comprises an amino acid sequence having at least 80% identity to SEQ ID NO:31, 32, or 33.
12. The immunogenic composition of any one of embodiments 1 to 11, wherein the recombinant neuraminidase further comprises a cleavage site.
13. The immunogenic composition of embodiment 12, wherein the cleavage site in the recombinant neuraminidase is between the tetramerization domain and influenza virus neuraminidase globular head domain.
14. The immunogenic composition of embodiment 12 or 13, wherein the cleavage site comprises a thrombin cleavage site.
15. The immunogenic composition of embodiment 12 or 13, wherein the cleavage site comprises the amino acid sequence of LVPRGSP (SEQ ID NO:36) or SLVPRGSPSR (SEQ ID NO:37).
16. The immunogenic composition of any one of embodiments 1 to 15, wherein the recombinant neuraminidase further comprises a signal peptide.
17. The immunogenic composition of embodiment 16, wherein the signal peptide comprises the amino acid sequence of

(SEQ ID NO: 34)
MLLVNQSHQGFNKEHTSKMVSAIVLYVLLAAAAHSAFAADP.

18. The immunogenic composition of any one of embodiments 1 to 17, wherein the recombinant neuraminidase further comprises a purification tag.
19. The immunogenic composition of embodiment 18, wherein the purification tag is a histidine tag or flag tag.
20. The immunogenic composition of any one of embodiments 1 to 19, wherein the recombinant neuraminidase is enzymatically active.
21. The immunogenic composition of embodiment 20, wherein neuraminidase enzymatic activity is assessed by an NA-Star assay.
22. An immunogenic composition comprising:
   a) a recombinant neuraminidase comprising the amino acid sequence of SEQ ID NO:40, 42, 44, 46 or 48, or SEQ ID NO:40, 42, 44, 46 or 48 without the signal sequence; and
   b) an adjuvant, wherein the adjuvant comprises an oligonucleotide of from 10 to 35 nucleotides in length comprising the nucleotide sequence of 5′-GAACGTTCG-3′,
   in an admixture with a pharmaceutically acceptable carrier.
23. An immunogenic composition comprising:
   a) a recombinant neuraminidase comprising an amino acid sequence having at least 80% identity to SEQ ID NO:50, 52, 54, 56 or 58, or SEQ ID NO:50, 52, 54, 56 or 58 without the signal sequence; and
   b) an adjuvant, wherein the adjuvant comprises an oligonucleotide of from 10 to 35 nucleotides in length comprising the nucleotide sequence of 5′-GAACGTTCG-3′,
   in an admixture with a pharmaceutically acceptable carrier.
24. An immunogenic composition comprising:
   a) a recombinant neuraminidase comprising an amino acid sequence having at least 80% identity to SEQ ID NO:60, 62, 64, 66 or 68, or SEQ ID NO:60, 62, 64, 66 or 68 without the signal sequence; and
   an adjuvant, wherein the adjuvant comprises an oligonucleotide of from 10 to 35 nucleotides in length comprising the nucleotide sequence of 5′-GAACGTTCG-3′, in an admixture with a pharmaceutically acceptable carrier.
25. An immunogenic composition, comprising
   a) two or three recombinant neuraminidases, wherein each recombinant neuraminidase:
      i) comprises an influenza virus neuraminidase globular head domain, and a paramyxovirus phosphoprotein tetramerization domain; and
      ii) lacks each of influenza virus neuraminidase stalk domain, transmembrane domain, and cytoplasmic domain, and
   b) an adjuvant, wherein the adjuvant comprises an oligonucleotide of from 10 to 35 nucleotides in length comprising the nucleotide sequence of

5′-GAACGTTCG-3′,

in an admixture with a pharmaceutically acceptable carrier.
26. The immunogenic composition of embodiment 25, wherein the paramyxovirus phosphoprotein tetramerization domain comprises a measles virus phosphoprotein tetramerization domain.
27. The immunogenic composition of embodiment 26, wherein the measles virus phosphoprotein tetramerization domain comprises the amino acid sequence of

(SEQ ID NO: 8)
GDHYDDELFSDVQDIKTALAKIHEDNQKIISKLESLLLLKGEVESIKKQ
INRQNISISTLEGHLSSIMIAIPGL.

28. The immunogenic composition of embodiment 26, wherein the measles virus phosphoprotein tetramerization domain comprises an amino acid sequence having at least 80% identity to the amino acid sequence of SEQ ID NO:8.
29. The immunogenic composition of any one of embodiments 25 to 28, which comprises two recombinant neuraminidases.
30. The immunogenic composition of any one of embodiments 25 to 28, which comprises three recombinant neuraminidases.
31. The immunogenic composition of embodiment 29, wherein each of the recombinant neuraminidases comprises an influenza A virus neuraminidase globular head domain.
32. The immunogenic composition of embodiment 29, wherein one of the recombinant neuraminidases comprises an influenza A virus neuraminidase globular head domain of subtype N1, and the second recombinant neuraminidase comprises an influenza A virus neuraminidase domain of subtype N2.
33. The immunogenic composition of embodiment 32, wherein:
   a) the subtype N1 is influenza virus A/Michigan/45/2015; and
   b) the subtype N2 is influenza virus A/Kansas/14/2017.
34. The immunogenic composition of embodiment 29, wherein the influenza virus neuraminidase globular head domain of one recombinant neuraminidase comprises the amino acid sequence of SEQ ID NO:31, and the influenza virus neuraminidase globular head domain of the second recombinant neuraminidase comprises the amino acid sequence of SEQ ID NO:32.
35. The immunogenic composition of embodiment 29, wherein the influenza virus neuraminidase globular head domain of one recombinant neuraminidase comprises an amino acid sequence having at least 80% identity to SEQ ID NO:31, and the influenza virus neuraminidase globular head domain of the second recombinant neuraminidase comprises an amino acid sequence having at least 80% identity to SEQ ID NO:32.
36 The immunogenic composition of embodiment 30, wherein one of the recombinant neuraminidases comprises an influenza A virus neuraminidase globular head domain of subtype N1, the second recombinant neuraminidase comprises an influenza A virus neuraminidase domain of subtype N2, and the third recombinant neuraminidase comprises an influenza B virus neuraminidase globular head domain.
37. The immunogenic composition of embodiment 36, wherein:
   a) the subtype N1 is influenza virus A/Michigan/45/2015;
   b) the subtype N2 is influenza virus A/Kansas/14/2017; and
   c) the influenza B virus is influenza virus B/Colorado/6/2017.
38. The immunogenic composition of embodiment 30, wherein the influenza virus neuraminidase globular head domain of one recombinant neuraminidase comprises the amino acid sequence of SEQ ID NO:31, the influenza virus neuraminidase globular head domain of the second recombinant neuraminidase comprises the amino acid sequence of SEQ ID NO:32, and the influenza virus neuraminidase globular head domain of the third recombinant neuraminidase comprises the amino acid sequence of SEQ ID NO:33.
39. The immunogenic composition of embodiment 30, wherein the influenza virus neuraminidase globular head domain of one recombinant neuraminidase comprises an amino acid sequence having at least 80% identity to SEQ ID NO:31, the influenza virus neuraminidase globular head domain of the second recombinant neuraminidase comprises an amino acid sequence having at least 80% identity to SEQ ID NO:32, and the influenza virus neuraminidase globular head domain of the third recombinant neuraminidase comprises an amino acid sequence having at least 80% identity to SEQ ID NO:33.
40. The immunogenic composition of any one of embodiments 25 to 39, wherein each recombinant neuraminidase further comprises a cleavage site.
41. The immunogenic composition of embodiment 40, wherein the cleavage site is between the tetramerization domain and influenza virus neuraminidase globular head domain.
42. The immunogenic composition of embodiment 40 or 41, wherein the cleavage site comprises a thrombin cleavage site.
43. The immunogenic composition of embodiment 40 or 41, wherein the cleavage site comprises the amino acid sequence of LVPRGSP (SEQ ID NO:36) or SLVPRGSPSR (SEQ ID NO:37).
44 The immunogenic composition of any one of embodiments 25 to 43, wherein each recombinant neuraminidase further comprises a signal peptide.
45. The immunogenic composition of embodiment 44, wherein the signal peptide comprises the amino acid sequence of

(SEQ ID NO: 34)
MLLVNQSHQGFNKEHTSKMVSAIVLYVLLAAAAHSAFAADP.

46. The immunogenic composition of any one of embodiments 25 to 45, wherein each recombinant neuraminidase further comprises a purification tag.
47. The immunogenic composition of embodiment 46, wherein the purification tag is a histidine tag or flag tag.
48. The immunogenic composition of any one of embodiments 25 to 47, wherein each recombinant neuraminidase is enzymatically active.
49. The immunogenic composition of embodiment 48, wherein neuraminidase enzymatic activity is assessed by an NA-Star assay.
50. An immunogenic composition, comprising
   a) three recombinant neuraminidases, wherein:
      i) the first recombinant neuraminidase comprises the amino acid sequence of SEQ ID NO:40, 42, 44, 46, or 48, or SEQ ID NO:40, 42, 44, 46, or 48 without the signal sequence;
      ii) the second recombinant neuraminidase comprises the amino acid sequence of SEQ ID NO:50, 52, 54, 56, or 58, or SEQ ID NO:50, 52, 54, 56, or 58 without the signal sequence; and
      iii) the third recombinant neuraminidase comprises the amino acid sequence of SEQ ID NO:60, 62, 64, 66, or 68, or SEQ ID NO:60, 62, 64, 66, or 68 without the signal sequence, and
   b) an adjuvant, wherein the adjuvant comprises an oligonucleotide comprising the nucleotide sequence of 5′-GAACGTTCG-3′,
   in an admixture with a pharmaceutically acceptable carrier.
51. The immunogenic composition of any one of embodiments 1 to 50, wherein the oligonucleotide comprises the nucleotide sequence of 5′-TGACTGTGAACGTTCGAGATGA-3′ (SEQ ID NO:4).
52. The immunogenic composition of embodiment 51, wherein the oligonucleotide is 22, 23, 24, 25, 26, 27, 28, 29, 30, 31, 32, 33, 34, or 35 nucleotides in length.
53. The immunogenic composition of any one of embodiments 1 to 52, wherein the oligonucleotide is a single stranded oligodeoxynucleotide.
54. The immunogenic composition of any one of embodiments 1 to 53, wherein the oligonucleotide comprises only phosphorothioate linkages, or a combination of one or more phosphodiester linkages and one or more phosphorothioate linkages.
55. The immunogenic composition of embodiment 54, wherein the oligonucleotide is fully RNA or is an RNA/DNA chimera.
56. The immunogenic composition of any one of embodiments 1 to 55, wherein the adjuvant further comprises an aluminum salt.
57. The immunogenic composition of embodiment 56, wherein the aluminum salt comprises aluminum hydroxide.
58. The immunogenic composition of any one of embodiments 1 to 57, which further comprises an influenza vaccine.
59. The immunogenic composition of embodiment 58, wherein the influenza vaccine is a seasonal influenza vaccine.
60. A method of immunizing a subject against influenza virus, comprising administering to the subject a dose of the immunogenic composition of any one of embodiments 1 to 59.
61. A method of inducing an immune response in a subject against influenza virus, comprising administering to the subject a dose of the immunogenic composition of any one of embodiments 1 to 59.
62. A method of preventing an influenza virus disease in a subject, comprising administering to the subject a dose of the immunogenic composition of any one of embodiments 1 to 59.
63. The method of any one of embodiments 60 to 62, wherein the subject is administered at least one subsequent dose of the immunogenic composition.
64. The method of any one of embodiments 60 to 62, wherein the subject is administered two subsequent doses of the immunogenic composition.
65. The method of any one of embodiments 60 to 64, wherein the immunogenic composition is administered intramuscularly to the subject.
66. The method of any one of embodiments 60 to 64, wherein the immunogenic composition is administered intranasally to the subject.
67. The method of any one of embodiments 60 to 66, wherein the subject is human.
68. A method of immunizing a subject against influenza virus, comprising administering to the subject an immunogenic composition and an adjuvant, wherein the immunogenic composition comprises a recombinant neuraminidase in an admixture with a pharmaceutically acceptable carrier, wherein the adjuvant comprises an oligonucleotide of from 10 to 35 nucleotides in length comprising the nucleotide sequence of 5′-GAACGTTCG-3′, and wherein recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain.
69. A method of inducing an immune response in a subject against influenza virus, comprising administering to the subject an immunogenic composition and an adjuvant, wherein the immunogenic composition comprises a recombinant neuraminidase in an admixture with a pharmaceutically acceptable carrier, wherein the adjuvant comprises an oligonucleotide of from 10 to 35 nucleotides in length comprising the nucleotide sequence of 5′-GAACGTTCG-3′, and wherein recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain.
70. A method of preventing an influenza virus disease in a subject, comprising administering to the subject an immunogenic composition and an adjuvant, wherein the immunogenic composition comprises a recombinant neuraminidase in an admixture with a pharmaceutically acceptable carrier, wherein the adjuvant comprises an oligonucleotide of from 10 to 35 nucleotides in length comprising the nucleotide sequence of 5′-GAACGTTCG-3′, and wherein recombinant neuraminidase (i) comprises an influenza virus neuraminidase globular head domain, and a paramyxovirus phosphoprotein tetramerization domain; and (ii) lacks each of influenza virus neuraminidase stalk domain, transmembrane domain, and cytoplasmic domain.
71. The method of any one of embodiments 68 to 70, wherein the paramyxovirus phosphoprotein tetramerization domain comprises a measles virus phosphoprotein tetramerization domain.
72. The method of embodiment 71, wherein the measles virus phosphoprotein tetramerization domain comprises the amino acid sequence of

(SEQ ID NO: 8)
GDHYDDELFSDVQDIKTALAKIHEDNQKIISKLESLLLLKGEVESIKKQ
INRQNISISTLEGHLSSIMIAIPGL.

73. The method of embodiment 71, wherein the measles virus phosphoprotein tetramerization domain comprises an amino acid sequence having at least 80% identity to the amino acid sequence of SEQ ID NO:8.
74. The method of any one of embodiments 68 to 73, wherein the influenza virus neuraminidase globular head domain comprises an influenza A virus neuraminidase globular head domain.
75 The method of embodiment 74, wherein the influenza A virus neuraminidase is subtype N1 or subtype N2.
76. The method of any one of embodiments 68 to 73, wherein the influenza virus neuraminidase globular head domain comprises an influenza B virus neuraminidase globular head domain.
77. The method of embodiment 75, wherein
   a) the subtype N1 is influenza virus A/Michigan/45/2015; or
   b) the subtype N2 is influenza virus A/Kansas/14/2017.
78. The method of embodiment 76, wherein the influenza B virus is influenza virus B/Colorado/6/2017.
79. The method of any one of embodiments 68 to 73, wherein the influenza virus neuraminidase globular head domain comprises the amino acid sequence of:

a)
(SEQ ID NO: 31)
SVKLAGNSSLCPVSGWAIYSKDNSVRIGSKGDVFVIREPFISCSPLECR
TFFLTQGALLNDKHSNGTIKDRSPYRTLMSCPIGEVPSPYNSRFESVAW
SASACHDGINWLTIGISGPDSGAVAVLKYNGIITDTIKSWRNNILRTQE
SECACVNGSCFTIMTDGPSDGQASYKIFRIEKGKIIKSVEMKAPNYHYE
ECSCYPDSSEITCVCRDNWHGSNRPWVSFNQNLEYQMGYICSGVFGDNP
RPNDKTGSCGPVSSNGANGVKGFSFKYGNGVWIGRTKSISSRKGFEMIW
DPNGWTGTDNKFSIKQDIVGINEWSGYSGSFVQHPELTGLDCIRPCFWV
ELIRGRPEENTIWTSGSSISFCGVNSDTVGWSWPDGAELPFTIDK;
b)
(SEQ ID NO: 32)
ICPKPAEYRNWSKPQCGITGFAPFSKDNSIRLSAGGDIWVTREPYVSCD
PDKCYQFALGQGTTINNVHSNNTARDRTPHRTLLMNELGVPFHLGTKQV
CIAWSSSSCHDGKAWLHVCITGDDKNATASFIYNGRLVDSVVSWSKDIL
RTQESECVCINGTCTVVMTDGNATGKADTKILFIEEGKIVHTSKLSGSA
QHVEECSCYPRYPGVRCVCRDNWKGSNRPIVDINIKDHSIVSSYVCSGL
VGDTPRKTDSSSSSHCLNPNNEKGGHGVKGWAFDDGNDVWMGRTINETS
RLGYETFKVVEGWSNPKSKLQINRQVIVDRGDRSGYSGIFSVEGKSCIN
RCFYVELIRGRKEETEVLWTSNSIVVFCGTSGTYGTGSWPDGADLNLMH
I;
or
c)
(SEQ ID NO: 33)
LLLPEPEWTYPRLSCPGSTFQKALLISPHRFGETKGNSAPLIIREPFVA
CGPNECKHEALTHYAAQPGGYYNGTRGDRNKLRHLISVKLGKIPTVENS
IFHMAAWSGSACHDGKEWTYIGVDGPDNNALLKVKYGEAYTDTYHSYAN
NILRTQESACNCIGGNCYLMITDGSASGVSECRFLKIREGRIIKEIFPT
GRVKHTEECTCGFASNKTIECACRDNRYTAKRPFVKLNVETDTAEIRLM
CTDTYLDTPRPNDGSITGPCESDGDKGSGGIKGGFVHQRMKSKIGRWYS
RTMSQTERMGMGLYVKYGGDPWADSDALAFSGVMVSMKEPGWYSFGFEI
KDKKCDVPCIGIEMVHDGGKETWHSAATAIYCLMGSGQLLWDTVTGVDM
AL.

80. The method of any one of embodiments 68 to 73, wherein the influenza virus neuraminidase globular head domain comprises an amino acid sequence having at least 80% identity to SEQ ID NO:31, 32, or 33.
81. The method of any one of embodiments 68 to 80, wherein the recombinant neuraminidase further comprises a signal peptide.
82. The method of embodiment 81, wherein the signal peptide comprises the amino acid sequence of MLLVNQSHQGFNKEHTSKMVSAIVLYVLLAAAAHSAFAADP (SEQ ID NO:34).
83. The method of any one of embodiments 68 to 82, wherein the recombinant neuraminidase is enzymatically active.
84. The immunogenic composition of embodiment 83, wherein neuraminidase enzymatic activity is assessed by an NA-Star assay.
85. The method of any one of embodiments 68 to 84, wherein the oligonucleotide comprises the nucleotide sequence of 5′-TGACTGTGAACGTTCGAGATGA-3′ (SEQ ID NO: 4).
86 The method of embodiment 85, wherein the oligonucleotide is 22, 23, 24, 25, 26, 27, 28, 29, 30, 31, 32, 33, 34, or 35 nucleotides in length.
87. The method of any one of embodiments 68 to 86, wherein the oligonucleotide is a single stranded oligodeoxynucleotide.
88. The method of any one of embodiments 68 to 87, wherein the oligonucleotide comprises only phosphorothioate linkages, or a combination of one or more phosphodiester linkages and one or more phosphorothioate linkages.
89. The method of embodiment 88, wherein the oligonucleotide is fully RNA or is an RNA/DNA chimera.
90. The method of any one of embodiments 68 to 89, wherein the adjuvant further comprises an aluminum salt.
91. The method of embodiment 90, wherein the aluminum salt comprises aluminum hydroxide.
92. The method of any one of embodiments 68 to 91, wherein the immunogenic composition, the adjuvant, or both are administered intramuscularly to the subject.
93. The method of any one of embodiments 68 to 91, wherein the immunogenic composition, the adjuvant, or both are administered intranasally to the subject.
94 The method of any one of embodiments 68 to 93, wherein the subject is human.

The foregoing is not to be limited in scope by the specific embodiments described herein. Indeed, various modifications of the antibodies and methods provided herein and their equivalents, in addition to those described herein will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1. An immunogenic composition, comprising:
a) a recombinant neuraminidase, wherein the recombinant neuraminidase
i) comprises an influenza virus neuraminidase globular head domain, and a paramyxovirus phosphoprotein tetramerization domain; and
ii) lacks an influenza virus neuraminidase stalk domain, transmembrane domain, and cytoplasmic domain, and
b) an adjuvant, wherein the adjuvant comprises an oligonucleotide of from 10 to 35 nucleotides in length comprising the nucleotide sequence of 5′-GAACGTTCG-3′,
in an admixture with a pharmaceutically acceptable carrier.

2. The immunogenic composition of claim 1, wherein the paramyxovirus phosphoprotein tetramerization domain comprises a measles virus phosphoprotein tetramerization domain.

3. The immunogenic composition of claim 2, wherein the measles virus phosphoprotein tetramerization domain comprises: (a) the amino acid sequence of GDHYDDELFSDVQDIKTALAKIHEDNQKIISKLESLLLLKGEVESIKKQINRQNISI STLEGHLSSIMIAIPGL (SEQ ID NO:8); or (b) an amino acid sequence having at least 80% identity to the amino acid sequence of SEQ ID NO:8.

4. (canceled)

5. The immunogenic composition of claim 1, wherein the influenza virus neuraminidase globular head domain comprises an influenza A virus neuraminidase globular head domain, or an influenza B virus neuraminidase globular head domain.

6. (canceled)

7. (canceled)

8. The immunogenic composition of claim 5, wherein
a) the influenza A virus-subtype NI is influenza virus A/Michigan/45/2015;
b) the influenza A virus-subtype N2 is influenza virus A/Kansas/14/2017; or
c) the influenza B virus is influenza virus B/Colorado/6/2017.

9. (canceled)

10. The immunogenic composition of claim 1, wherein the influenza virus neuraminidase globular head domain comprises:
a) the amino acid sequence of
(SEQ ID NO: 31)
SVKLAGNSSLCPVSGWAIYSKDNSVRIGSKGDVFVIREPFISCSPLECR
TFFLTQGALLNDKHSNGTIKDRSPYRTLMSCPIGEVPSPYNSRFESVAW
SASACHDGINWLTIGISGPDSGAVAVLKYNGIITDTIKSWRNNILRTQE
SECACVNGSCFTIMTDGPSDGQASYKIFRIEKGKIIKSVEMKAPNYHYE
ECSCYPDSSEITCVCRDNWHGSNRPWVSFNQNLEYQMGYICSGVFGDNP
RPNDKTGSCGPVSSNGANGVKGFSFKYGNGVWIGRTKSISSRKGFEMIW
DPNGWTGTDNKFSIKQDIVGINEWSGYSGSFVQHPELTGLDCIRPCFWV
ELIRGRPEENTIWTSGSSISFCGVNSDTVGWSWPDGAELPFTIDK;
b) the amino acid sequence of
(SEQ ID NO: 32)
ICPKPAEYRNWSKPQCGITGFAPFSKDNSIRLSAGGDIWVTREPYVSCD
PDKCYQFALGQGTTINNVHSNNTARDRTPHRTLLMNELGVPFHLGTKQV
CIAWSSSSCHDGKAWLHVCITGDDKNATASFIYNGRLVDSVVSWSKDIL
RTQESECVCINGTCTVVMTDGNATGKADTKILFIEEGKIVHTSKLSGSA
QHVEECSCYPRYPGVRCVCRDNWKGSNRPIVDINIKDHSIVSSYVCSGL
VGDTPRKTDSSSSSHCLNPNNEKGGHGVKGWAFDDGNDVWMGRTINETS
RLGYETFKVVEGWSNPKSKLQINRQVIVDRGDRSGYSGIFSVEGKSCIN
RCFYVELIRGRKEETEVLWTSNSIVVFCGTSGTYGTGSWPDGADLNLMH
I;
c) the amino acid sequence of
(SEQ ID NO: 33)
LLLPEPEWTYPRLSCPGSTFQKALLISPHRFGETKGNSAPLIIREPFVA
CGPNECKHEALTHYAAQPGGYYNGTRGDRNKLRHLISVKLGKIPTVENS
IFHMAAWSGSACHDGKEWTYIGVDGPDNNALLKVKYGEAYTDTYHSYAN
NILRTQESACNCIGGNCYLMITDGSASGVSECRFLKIREGRIIKEIFPT
GRVKHTEECTCGFASNKTIECACRDNRYTAKRPFVKLNVETDTAEIRLM
CTDTYLDTPRPNDGSITGPCESDGDKGSGGIKGGFVHQRMKSKIGRWYS
RTMSQTERMGMGLYVKYGGDPWADSDALAFSGVMVSMKEPGWYSFGFEI
KDKKCDVPCIGIEMVHDGGKETWHSAATAIYCLMGSGQLLWDTVTGVDM
AL;
or
d) an amino acid sequence having at least 80% identity to SEQ ID NO:31, 32, or 33.

11. (canceled)

12. The immunogenic composition of claim 1, wherein the recombinant neuraminidase further comprises a cleavage site.

13. (canceled)

14. The immunogenic composition of claim 12, wherein the cleavage site comprises: (a) a thrombin cleavage site; or (b) the amino acid sequence of
(SEQ ID NO: 36)
LVPRGSP
or
(SEQ ID NO: 37)
SLVPRGSPSR.

15-19. (canceled)

20. The immunogenic composition of claim 1, wherein the recombinant neuraminidase is enzymatically active.

21. (canceled)

22. An immunogenic composition comprising:
A) a) a recombinant neuraminidase comprising the amino acid sequence of SEQ ID NO:40, 42, 44, 46 or 48, or SEQ ID NO:40, 42, 44, 46 or 48 without the signal sequence; and
b) an adjuvant, wherein the adjuvant comprises an oligonucleotide of from 10 to 35 nucleotides in length comprising the nucleotide sequence of 5′-GAACGTTCG-3′,
in an admixture with a pharmaceutically acceptable carrier; or
B) a) a recombinant neuraminidase comprising an amino acid sequence having at least 80% identity to SEQ ID NO:50, 52, 54, 56 or 58, or SEQ ID NO:50, 52, 54, 56 or 58 without the signal sequence; and
b) an adjuvant, wherein the adjuvant comprises an oligonucleotide of from 10 to 35 nucleotides in length comprising the nucleotide sequence of 5′-GAACGTTCG-3′,
in an admixture with a pharmaceutically acceptable carrier; or
C) a) a recombinant neuraminidase comprising an amino acid sequence having at least 80% identity to SEQ ID NO:60, 62, 64, 66 or 68, or SEQ ID NO:60, 62, 64, 66 or 68 without the signal sequence; and
b) an adjuvant, wherein the adjuvant comprises an oligonucleotide of from 10 to 35 nucleotides in length comprising the nucleotide sequence of 5′-GAACGTTCG-3′,
in an admixture with a pharmaceutically acceptable carrier.

23. (canceled)

24. (canceled)

25. An immunogenic composition, comprising
a) two or three recombinant neuraminidases, wherein each recombinant neuraminidase:
i) comprises an influenza virus neuraminidase globular head domain, and a paramyxovirus phosphoprotein tetramerization domain; and
ii) lacks an influenza virus neuraminidase stalk domain, transmembrane domain, and cytoplasmic domain, and
b) an adjuvant, wherein the adjuvant comprises an oligonucleotide of from 10 to 35 nucleotides in length comprising the nucleotide sequence of 5′-GAACGTTCG-3′, in an admixture with a pharmaceutically acceptable carrier.

26. The immunogenic composition of claim 25, wherein the paramyxovirus phosphoprotein tetramerization domain comprises a measles virus phosphoprotein tetramerization domain.

27. The immunogenic composition of claim 26, wherein the measles virus phosphoprotein tetramerization domain comprises: (a) the amino acid sequence of GDHYDDELFSDVQDIKTALAKIHEDNQKIISKLESLLLLKGEVESIKKQINRQNISI STLEGHLSSIMIAIPGL (SEQ ID NO:8); or (b) an amino acid sequence having at least 80% identity to the amino acid sequence of SEQ ID NO:8.

28-31. (canceled)

32. The immunogenic composition of claim 25, wherein one of the recombinant neuraminidases comprises an influenza A virus neuraminidase globular head domain of subtype N1, and the second recombinant neuraminidase comprises an influenza A virus neuraminidase domain of subtype N2.

33. The immunogenic composition of claim 32, wherein:
a) the subtype N1 is influenza virus A/Michigan/45/2015; and
b) the subtype N2 is influenza virus A/Kansas/14/2017.

34. The immunogenic composition of claim 25, wherein: (a) the influenza virus neuraminidase globular head domain of one recombinant neuraminidase comprises the amino acid sequence of SEQ ID NO:31, and the influenza virus neuraminidase globular head domain of the second recombinant neuraminidase comprises the amino acid sequence of SEQ ID NO:32; or (b) the influenza virus neuraminidase globular head domain of one recombinant neuraminidase comprises an amino acid sequence having at least 80% identity to SEQ ID NO:31, and the influenza virus neuraminidase globular head domain of the second recombinant neuraminidase comprises an amino acid sequence having at least 80% identity to SEQ ID NO:32.

35. (canceled)

36. The immunogenic composition of claim 25, wherein one of the recombinant neuraminidases comprises an influenza A virus neuraminidase globular head domain of subtype N1, the second recombinant neuraminidase comprises an influenza A virus neuraminidase domain of subtype N2, and the third recombinant neuraminidase comprises an influenza B virus neuraminidase globular head domain.

37. The immunogenic composition of claim 36, wherein:
a) the subtype N1 is influenza virus A/Michigan/45/2015;
b) the subtype N2 is influenza virus A/Kansas/14/2017; and
c) the influenza B virus is influenza virus B/Colorado/6/2017.

38. The immunogenic composition of claim 25, wherein: (a) the influenza virus neuraminidase globular head domain of one recombinant neuraminidase comprises: (a) the amino acid sequence of SEQ ID NO:31, the influenza virus neuraminidase globular head domain of the second recombinant neuraminidase comprises the amino acid sequence of SEQ ID NO:32, and the influenza virus neuraminidase globular head domain of the third recombinant neuraminidase comprises the amino acid sequence of SEQ ID NO:33; or (b) the influenza virus neuraminidase globular head domain of one recombinant neuraminidase comprises an amino acid sequence having at least 80% identity to SEQ ID NO:31, the influenza virus neuraminidase globular head domain of the second recombinant neuraminidase comprises an amino acid sequence having at least 80% identity to SEQ ID NO:32, and the influenza virus neuraminidase globular head domain of the third recombinant neuraminidase comprises an amino acid sequence having at least 80% identity to SEQ ID NO:33.

39. (canceled)

40. The immunogenic composition of claim 25, wherein each recombinant neuraminidase further comprises a cleavage site.

41. (canceled)

42. The immunogenic composition of claim 40, wherein the cleavage site comprises: (a) a thrombin cleavage site; or (b) the amino acid sequence of
(SEQ ID NO: 36)
LVPRGSP
or
(SEQ ID NO: 37)
SLVPRGSPSR.

43-47. (canceled)

48. The immunogenic composition of claim 25, wherein each recombinant neuraminidase is enzymatically active.

49. (canceled)

50. An immunogenic composition, comprising
a) three recombinant neuraminidases, wherein:
i) the first recombinant neuraminidase comprises the amino acid sequence of SEQ ID NO:40, 42, 44, 46, or 48, or SEQ ID NO:40, 42, 44, 46, or 48 without the signal sequence;
ii) the second recombinant neuraminidase comprises the amino acid sequence of SEQ ID NO:50, 52, 54, 56, or 58, or SEQ ID NO:50, 52, 54, 56, or 58 without the signal sequence; and
iii) the third recombinant neuraminidase comprises the amino acid sequence of SEQ ID NO:60, 62, 64, 66, or 68, or SEQ ID NO:60, 62, 64, 66, or 68 without the signal sequence, and
b) an adjuvant, wherein the adjuvant comprises an oligonucleotide comprising the nucleotide sequence of 5′-GAACGTTCG-3′,
in an admixture with a pharmaceutically acceptable carrier.

51. The immunogenic composition of claim 1, wherein the oligonucleotide comprises the nucleotide sequence of 5′-TGACTGTGAACGTTCGAGATGA-3′ (SEQ ID NO:4).

52-55. (canceled)

56. The immunogenic composition of claim 1, wherein the adjuvant further comprises an aluminum salt.

57. (canceled)

58. The immunogenic composition of claim 1, which further comprises an influenza vaccine.

59. (canceled)

60. A method of immunizing a subject against influenza virus, inducing an immune response in a subject against influenza virus, or preventing an influenza virus disease in a subject, comprising administering to the subject a dose of the immunogenic composition of claim 1.

61-66. (canceled)

67. The method of claim 60, wherein the subject is human.

68. A method of immunizing a subject against influenza virus, inducing an immune response in a subject against influenza virus, or preventing an influenza virus disease in a subject, comprising administering to the subject an immunogenic composition and an adjuvant, wherein the immunogenic composition comprises a recombinant neuraminidase in an admixture with a pharmaceutically acceptable carrier, wherein the adjuvant comprises an oligonucleotide of from 10 to 35 nucleotides in length comprising the nucleotide sequence of 5′-GAACGTTCG-3′, and wherein recombinant neuraminidase (i) comprises an influenza virus neuraminidase globular head domain, and a paramyxovirus phosphoprotein tetramerization domain; and (ii) lacks an influenza virus neuraminidase stalk domain, transmembrane domain, and cytoplasmic domain.

69. (canceled)

70. (canceled)

71. The method of claim 68, wherein the paramyxovirus phosphoprotein tetramerization domain comprises a measles virus phosphoprotein tetramerization domain.

72. The method of claim 71, wherein the measles virus phosphoprotein tetramerization domain comprises: (a) the amino acid sequence of GDHYDDELFSDVQDIKTALAKIHEDNQKIISKLESLLLLKGEVESIKKQINRQNISI STLEGHLSSIMIAIPGL (SEQ ID NO:8); or (b) an amino acid sequence having at least 80% identity to the amino acid sequence of SEQ ID NO:8.

73. (canceled)

74. The method of claim 68, wherein the influenza virus neuraminidase globular head domain comprises an influenza A virus neuraminidase globular head domain, or an influenza B virus neuraminidase globular head domain.

75. (canceled)

76. (canceled)

77. The method of claim 74, wherein
a) the influenza A virus is influenza virus A/Michigan/45/2015;
b) the influenza A virus is influenza virus A/Kansas/14/2017; or
c) the influenza B virus is influenza virus B/Colorado/6/2017.

78. (canceled)

79. The method of claim 68, wherein the influenza virus neuraminidase globular head domain comprises:
a) the amino acid sequence of
(SEQ ID NO: 31)
SVKLAGNSSLCPVSGWAIYSKDNSVRIGSKGDVFVIREPFISCSPLECR
TFFLTQGALLNDKHSNGTIKDRSPYRTLMSCPIGEVPSPYNSRFESVAW
SASACHDGINWLTIGISGPDSGAVAVLKYNGIITDTIKSWRNNILRTQE
SECACVNGSCFTIMTDGPSDGQASYKIFRIEKGKIIKSVEMKAPNYHYE
ECSCYPDSSEITCVCRDNWHGSNRPWVSFNQNLEYQMGYICSGVFGDNP
RPNDKTGSCGPVSSNGANGVKGFSFKYGNGVWIGRTKSISSRKGFEMIW
DPNGWTGTDNKFSIKQDIVGINEWSGYSGSFVQHPELTGLDCIRPCFWV
ELIRGRPEENTIWTSGSSISFCGVNSDTVGWSWPDGAELPFTIDK;
b) the amino acid sequence of
(SEQ ID NO: 32)
ICPKPAEYRNWSKPQCGITGFAPFSKDNSIRLSAGGDIWVTREPYVSCD
PDKCYQFALGQGTTINNVHSNNTARDRTPHRTLLMNELGVPFHLGTKQV
CIAWSSSSCHDGKAWLHVCITGDDKNATASFIYNGRLVDSVVSWSKDIL
RTQESECVCINGTCTVVMTDGNATGKADTKILFIEEGKIVHTSKLSGSA
QHVEECSCYPRYPGVRCVCRDNWKGSNRPIVDINIKDHSIVSSYVCSGL
VGDTPRKTDSSSSSHCLNPNNEKGGHGVKGWAFDDGNDVWMGRTINETS
RLGYETFKVVEGWSNPKSKLQINRQVIVDRGDRSGYSGIFSVEGKSCIN
RCFYVELIRGRKEETEVLWTSNSIVVFCGTSGTYGTGSWPDGADLNLMH
I;
c) the amino acid sequence of
(SEQ ID NO: 33)
LLLPEPEWTYPRLSCPGSTFQKALLISPHRFGETKGNSAPLIIREPFVA
CGPNECKHEALTHYAAQPGGYYNGTRGDRNKLRHLISVKLGKIPTVENS
IFHMAAWSGSACHDGKEWTYIGVDGPDNNALLKVKYGEAYTDTYHSYAN
NILRTQESACNCIGGNCYLMITDGSASGVSECRFLKIREGRIIKEIFPT
GRVKHTEECTCGFASNKTIECACRDNRYTAKRPFVKLNVETDTAEIRLM
CTDTYLDTPRPNDGSITGPCESDGDKGSGGIKGGFVHQRMKSKIGRWYS
RTMSQTERMGMGLYVKYGGDPWADSDALAFSGVMVSMKEPGWYSFGFEI
KDKKCDVPCIGIEMVHDGGKETWHSAATAIYCLMGSGQLLWDTVTGVDM
AL;
or
d) an amino acid sequence having at least 80% identity to SEQ ID NO:31, 32, or 33.

80-82. (canceled)

83. The method of claim 68, wherein the recombinant neuraminidase is enzymatically active.

84. (canceled)

85. The method of claim 68, wherein the oligonucleotide comprises the nucleotide sequence of 5′-TGACTGTGAACGTTCGAGATGA-3′ (SEQ ID NO:4).

86-89. (canceled)

90. The method of claim 68, wherein the adjuvant further comprises an aluminum salt.

91-93. (canceled)

94. The method of claim 68, wherein the subject is human.
